(19) United States
(12) Patent Application Publication
(10) Pub. No.: US 2025/0017101 A1
(43) Pub. Date: Jan. 9, 2025

(54) ORGANIC COMPOUND AND ORGANIC LIGHT-EMITTING DEVICE COMPRISING SAME

(71) Applicant: P&H TECH Co., Ltd, Yongin-si (KR)

(72) Inventors: Seo-Yong HYUN, Yongin-si (KR); Gwan-hee PARK, Yongin-si (KR)

(73) Assignee: P&H TECH Co., Ltd, Yongin-si S (KR)

(21) Appl. No.: 18/690,071

(22) PCT Filed: Sep. 14, 2022
(86) PCT No.: PCT/KR2022/013677

§ 371 Date: Mar. 7, 2024

(30) Foreign Application Priority Data
Sep. 15, 2021 (KR) 10-2021-0122955

(51) Int. Cl. H10K 85/60; C07C 15/14; C07C 22/08; C07C 25/18; C07C 255/51; C07D 207/267; H10K 50/858

(52) U.S. Cl. CPC H10K 85/60; C07C 15/14; C07C 22/08; C07C 25/18; C07C 255/51; C07D 207/267; C07B 2200/05; C07C 2601/14; H10K 50/858

(57) ABSTRACT

The present invention relates to an organic compound and an organic light-emitting device comprising same. The organic compound is used in a light efficiency improving layer included in an organic light-emitting device to increase the light efficiency of the device so as to be able to implement excellent device characteristics such as low voltage driving, excellent color purity, and improved luminescence efficiency of the device. Same can be industrially and usefully used for various lighting and display devices.

### TECHNICAL FIELD

The present invention relates to an organic compound that is employed as a material for a light efficiency improving layer (capping layer) provided in an organic light-emitting device, and an organic light-emitting device that employs the same, thus achieving greatly improved luminescent properties such as low-voltage driving of the device and excellent luminous efficiency.

### BACKGROUND ART

The organic light-emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and read, and thus has recently become a subject of intense interest as a next-generation display device.

However, in order for such an organic light-emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light-emitting device has not yet been sufficiently made.

Thus, further improvements in terms of efficiency and life characteristics are required for good stability, high efficiency, long lifetime, and large size of organic light-emitting devices. Particularly, there is a strong need to develop materials constituting each organic layer of organic light-emitting devices.

In addition, recently, research aimed at improving the characteristics of organic light-emitting devices by changes in the performance of each organic layer material, as well as a technique for improving the color purity and enhancing the luminous efficiency by optimizing the optical thickness between an anode and a cathode are considered as one of the crucial factors for improving the device performance. As an example of this method, an increase in light efficiency and excellent color purity are achieved by using a capping layer on an electrode.

### DISCLOSURE

### Technical Problem

Thus, the present invention has been made in an effort to provide a novel organic compound which may be employed in a light efficiency improving layer (capping layer) provided in an organic light-emitting device to implement excellent luminescent properties such as low-voltage driving of the device and improved luminous efficiency, and an organic light-emitting device including the same.

### Technical Solution

An aspect of the present invention provides an organic compound represented by Formula I below.

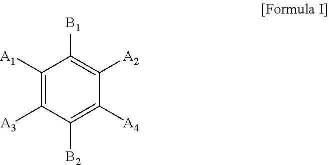

Characteristic structures of Formula I above and specific compounds, A₁ to A₄, and B₁ to B₂ implemented thereby will be described below.

Another aspect of the present invention provides an organic light-emitting device including a first electrode, a second electrode, and one or more organic layers arranged between the first and second electrodes, wherein the organic light-emitting device further includes a light efficiency improving layer (capping layer) formed on at least one side opposite to the organic layer among the upper or lower portions of the first electrode and the second electrode, and the light efficiency improving layer includes an organic compound represented by Formula I above.

### Advantageous Effects

According to the present invention, an organic compound is employed as a material for a light efficiency improving layer provided in an organic light-emitting device to implement low-voltage driving of the organic light-emitting device and improved luminescent properties such as excellent luminous efficiency, color purity, etc., and thus can be effectively used in various display devices.

### BEST MODE FOR CARRYING OUT INVENTION

Hereinafter, the present invention will be described in more detail.

The present invention relates to an organic light emitting compound represented by Formula I below, which is employed as a material for a light efficiency improving layer provided in an organic light-emitting device to achieve low-voltage driving of the device and luminescent properties such as excellent luminous efficiency, color purity, etc.

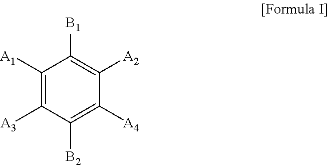

In Formula I above,
(i) A₁ and A₂ are the same or different from each other, and each independently selected from the following Structural Formula 1.

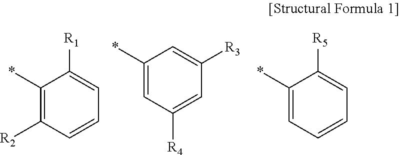

in Structural Formula 1 above,
R₁ to R₅ are the same as or different from each other, and each independently selected from deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted heterocycloalkyl group having 2 to 20 carbon atoms, a substituted or unsubstituted aryl group having 6 to 30 carbon atoms, a substituted or unsubstituted heteroaryl group having 2 to 30 carbon atoms, and Structural Formula 2 below.

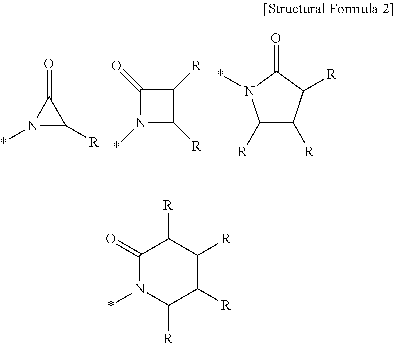

in Structural Formula 2 above,
in each structure, R is each independently selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms, and in each structure of Structural Formula 2, a plurality of R are the same as or different from each other.
(ii) B₁ and B₂ are the same as or different from each other, and each independently selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or an unsubstituted halogenated alkoxy group having 1 to 20 carbon atoms and a substituted or unsubstituted cycloalkyl group having 3 to 20 carbon atoms.
(iii) In the definition of R, R₁ to R₅, B₁ and B₂, the ‘substituted or unsubstituted’ means substitution of R, R₁ to R₅, B₁ and B₂ above with one or at least two substituents selected from the group consisting of deuterium, a halogen group, a cyano group, an alkyl group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylsilyl group and an arylsilyl group, substitution with a substituent to which two or more of the substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. The number of carbon atoms in the alkyl groups is not particularly limited but is preferably from 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. The number of carbon atoms in the alkoxy groups is not particularly limited but is preferably from 1 to 20 as long as steric hindrance is avoided. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In addition, in an embodiment of the present invention, the fluorenyl groups refer to structures in which two cyclic organic compounds are linked through one atom, and examples thereof include

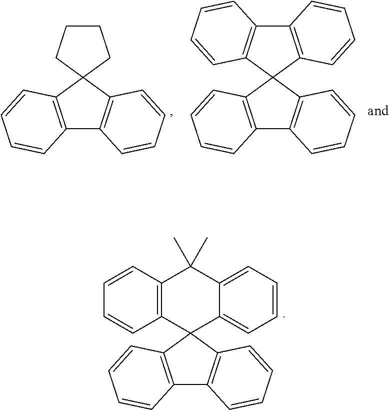

In an embodiment of the present invention, the fluorenyl groups include open structures in which one of the two cyclic organic compounds linked through one atom is cleaved, and examples thereof include

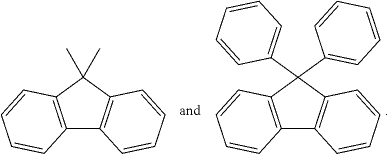

In addition, carbon atoms of the ring may be substituted with any one or more heteroatoms selected from among N, S and O, and examples thereof include

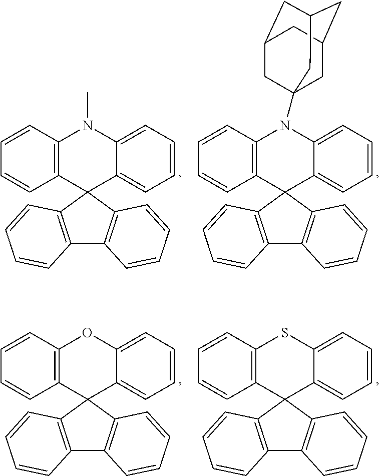

and the like.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the amine group may be —NH₂, an alkylamine group, an arylamine group, a heteroaryl amino group, an arylheteroarylamine group, etc., the aryl (heteroaryl)amine group means an amine substituted with an aryl group and/or heteroaryl group, and the alkylamine group means an amine substituted with an alkyl group. Examples of the aryl (heteroaryl)amine group include a substituted or unsubstituted mono aryl (heteroaryl)amine group, a substituted or unsubstituted diaryl (heteroaryl)amine group, or a substituted or unsubstituted triaryl (heteroaryl)amine group, wherein the aryl group and the heteroaryl group in the aryl (heteroaryl)amine group are the same as the definition of the aryl group and the heteroaryl group, and the alkyl group in the alkylamine group is also the same as the definition of the alkyl group.

For example, the arylamine group includes a phenylamine group, a naphthylamine group, a biphenylamine group, an anthracenylamine group, a 3-methyl-phenylamine group, a 4-methyl-naphthylamine group, and a 2-methyl-biphenyl amine group, a 9-methyl-anthracenylamine group, a diphenyl amine group, a phenylnaphthylamine group, a ditolylamine group, a phenyltolylamine group, a triphenylamine group, etc., but is not limited thereto.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (CI), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

The organic compound according to the present invention represented by Formula I may be used as a material for the light efficiency improving layer (capping layer) provided in the organic light-emitting device due to its structural specificity.

Preferred specific examples of the organic compound represented by Formula I according to the present invention include the following compounds but are not limited thereto.

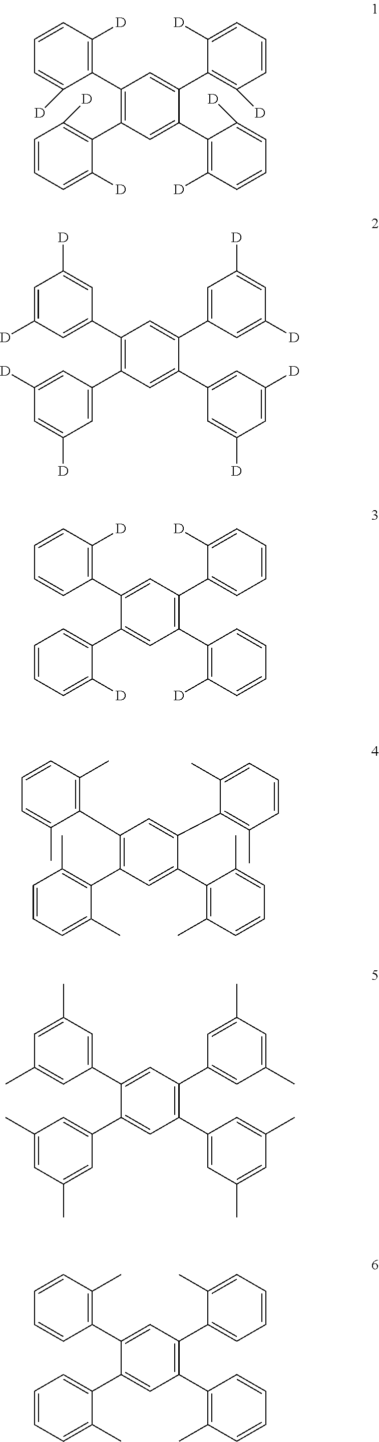

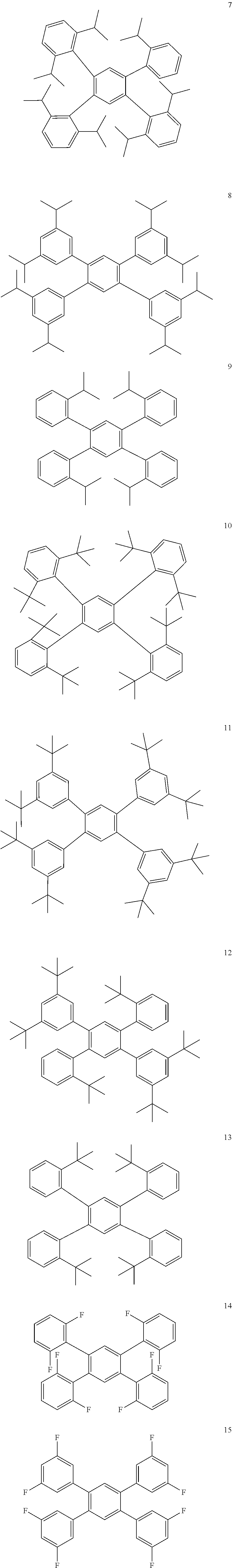

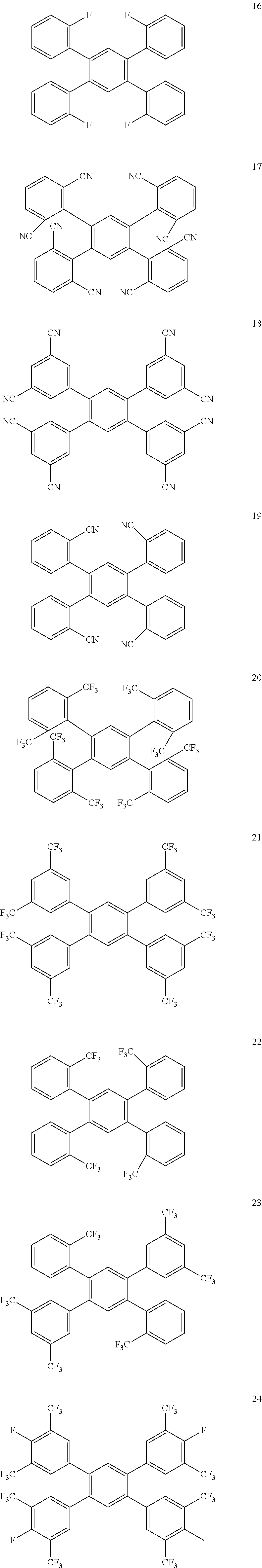

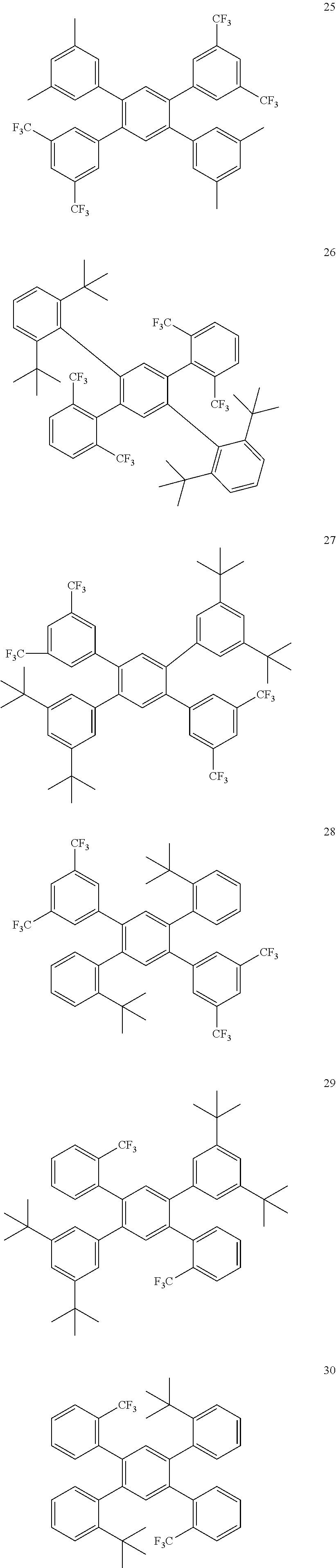

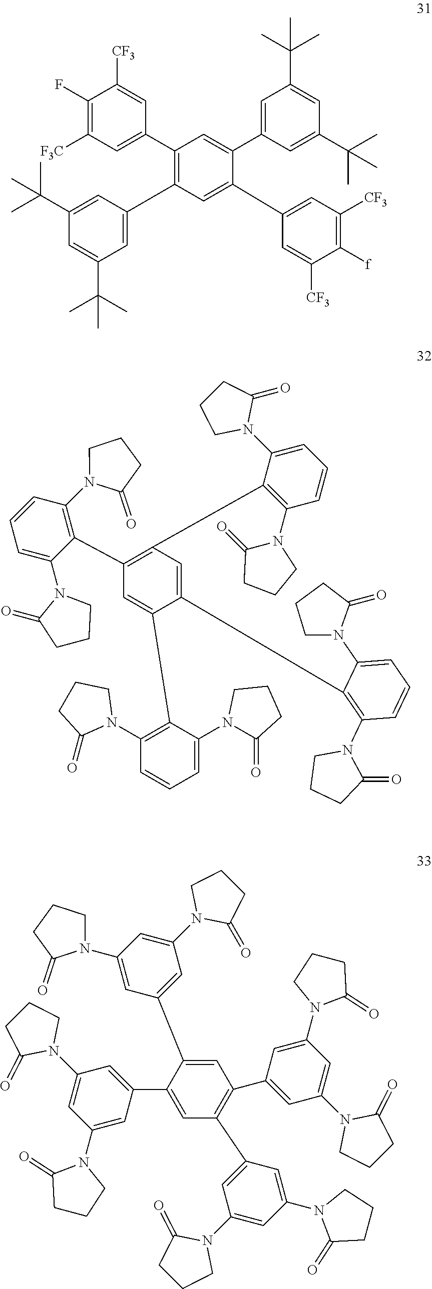

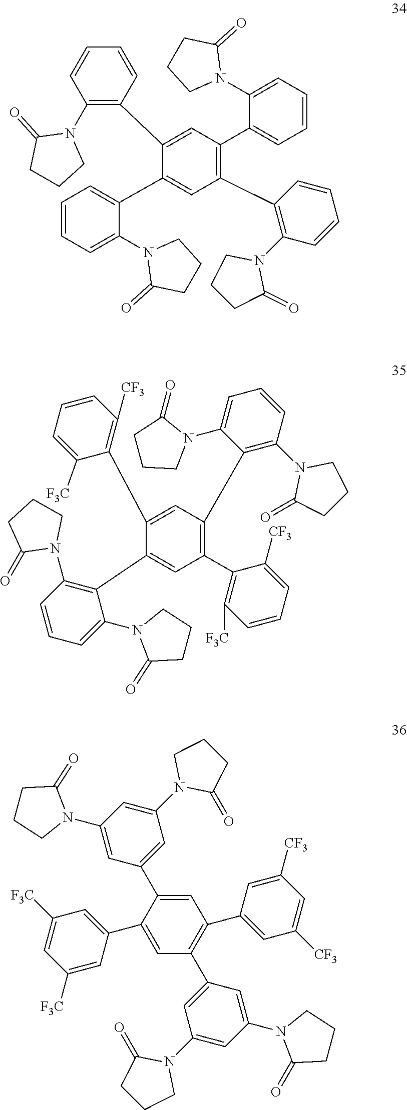 [SMILES: O=C1CCCN1c1ccccc1-c1cc(-c2ccccc2N2CCCC2=O)c(-c2ccccc2N2CCCC2=O)cc1-c1ccccc1N1CCCC1=O ; O=C1CCCN1c1cccc(N2CCCC2=O)c1-c1cc(-c2c(C(F)(F)F)cccc2C(F)(F)F)c(-c2c(N3CCCC3=O)cccc2N2CCCC2=O)cc1-c1c(C(F)(F)F)cccc1C(F)(F)F ; O=C1CCCN1c1cc(-c2cc(-c3cc(C(F)(F)F)cc(C(F)(F)F)c3)c(-c3cc(N4CCCC4=O)cc(N4CCCC4=O)c3)cc2-c2cc(C(F)(F)F)cc(C(F)(F)F)c2)cc(N2CCCC2=O)c1]

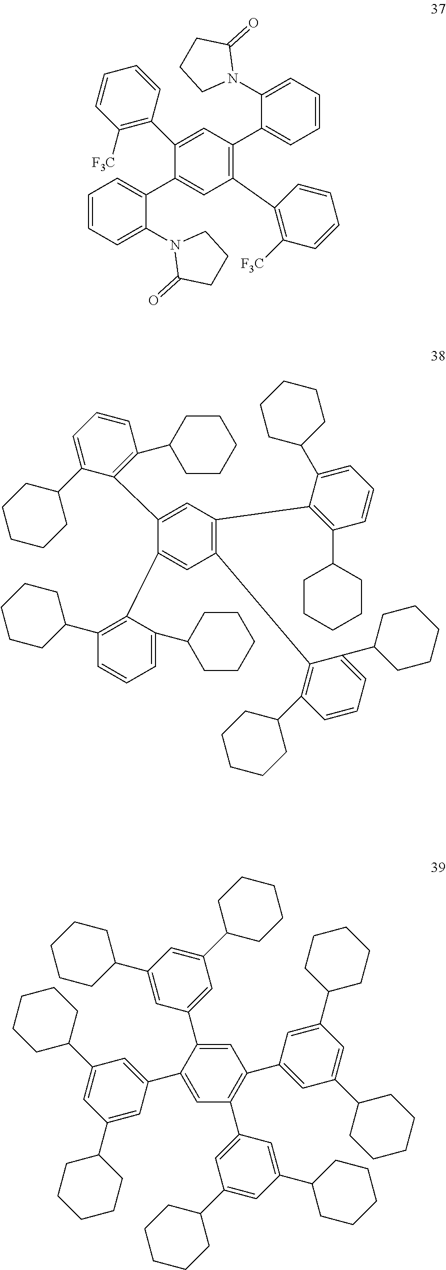

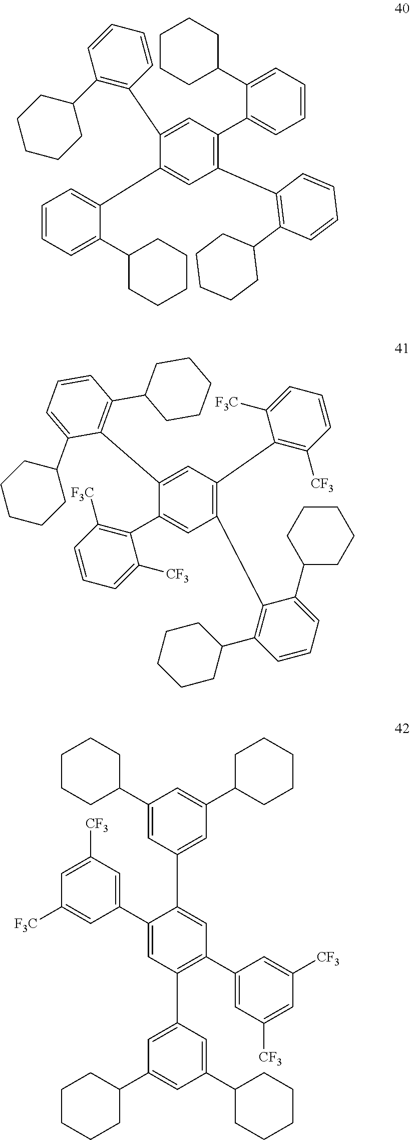

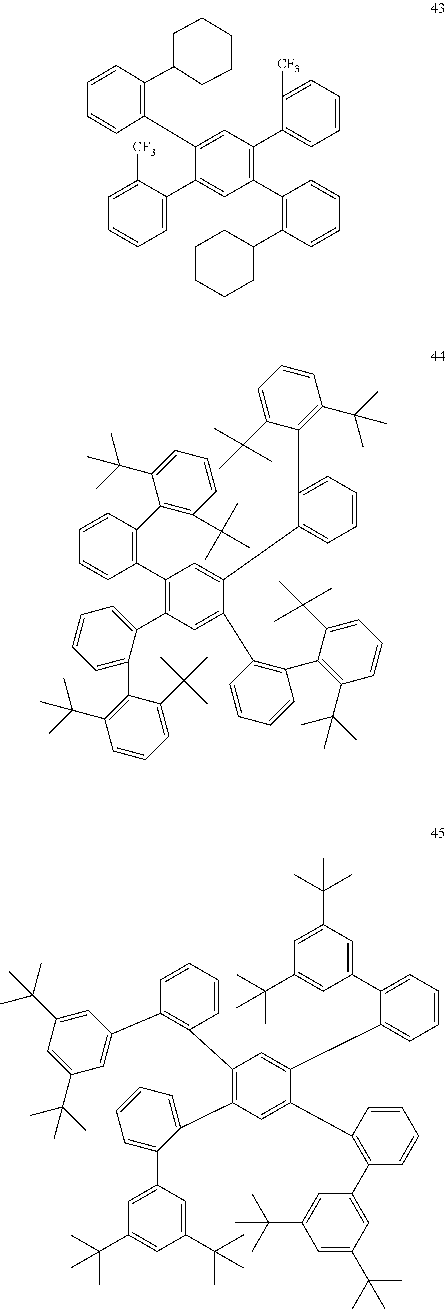

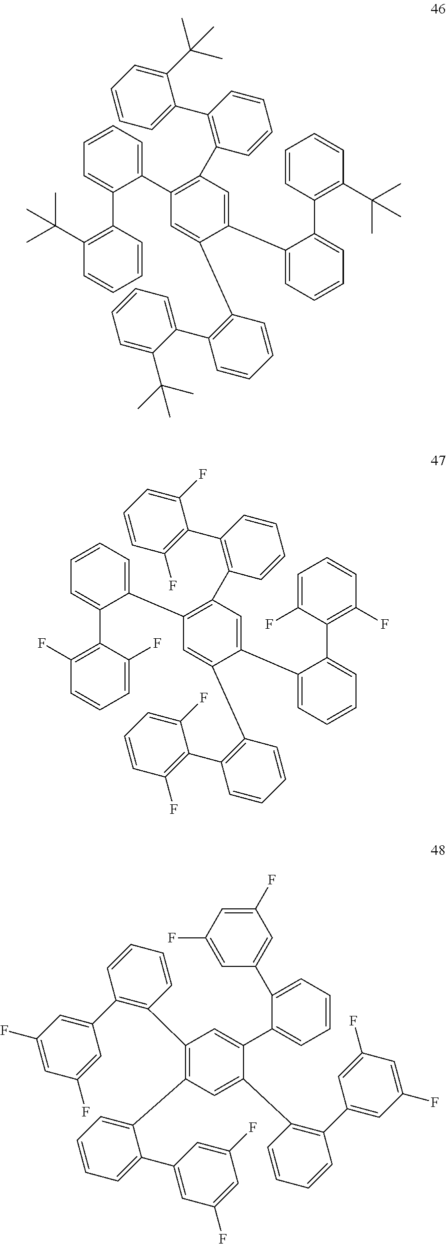

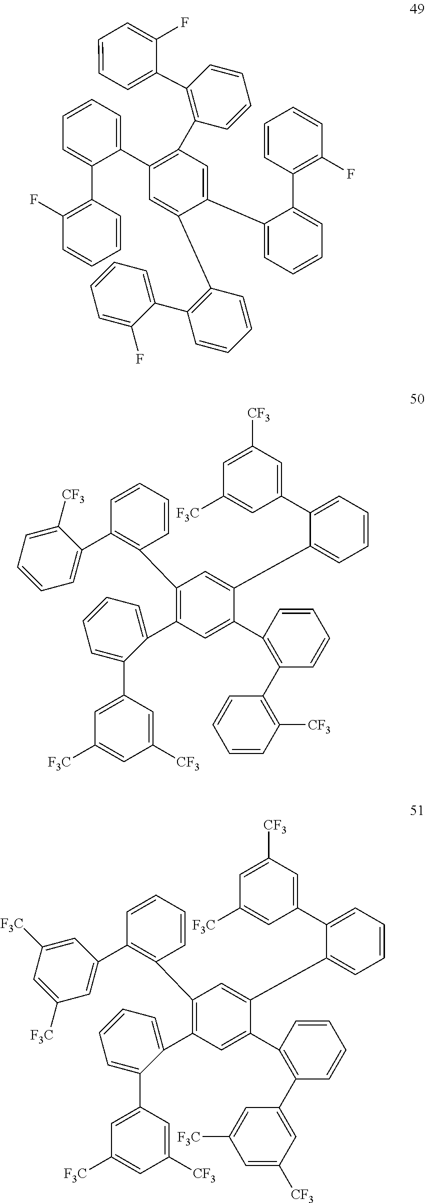 [SMILES: Fc1ccccc1-c1ccccc1-c1cc(-c2ccccc2-c2ccccc2F)c(-c2ccccc2-c2ccccc2F)cc1-c1ccccc1-c1ccccc1F ; FC(F)(F)c1cc(-c2ccccc2-c2cc(-c3ccccc3-c3ccccc3C(F)(F)F)c(-c3ccccc3-c3cc(C(F)(F)F)cc(C(F)(F)F)c3)cc2-c2ccccc2-c2ccccc2C(F)(F)F)cc(C(F)(F)F)c1 ; FC(F)(F)c1cc(-c2ccccc2-c2cc(-c3ccccc3-c3cc(C(F)(F)F)cc(C(F)(F)F)c3)c(-c3ccccc3-c3cc(C(F)(F)F)cc(C(F)(F)F)c3)cc2-c2ccccc2-c2cc(C(F)(F)F)cc(C(F)(F)F)c2)cc(C(F)(F)F)c1]

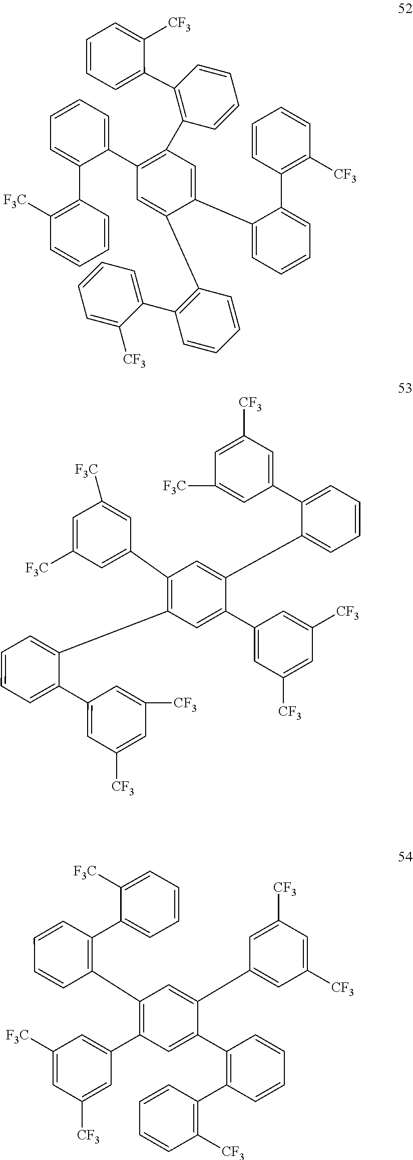 [SMILES: FC(F)(F)c1ccccc1-c1ccccc1-c1cc(-c2ccccc2-c2ccccc2C(F)(F)F)c(-c2ccccc2-c2ccccc2C(F)(F)F)cc1-c1ccccc1-c1ccccc1C(F)(F)F ; FC(F)(F)c1cc(-c2ccccc2-c2cc(-c3cc(C(F)(F)F)cc(C(F)(F)F)c3)c(-c3ccccc3-c3cc(C(F)(F)F)cc(C(F)(F)F)c3)cc2-c2cc(C(F)(F)F)cc(C(F)(F)F)c2)cc(C(F)(F)F)c1 ; FC(F)(F)c1cc(-c2cc(-c3ccccc3-c3ccccc3C(F)(F)F)c(-c3cc(C(F)(F)F)cc(C(F)(F)F)c3)cc2-c2ccccc2-c2ccccc2C(F)(F)F)cc(C(F)(F)F)c1]

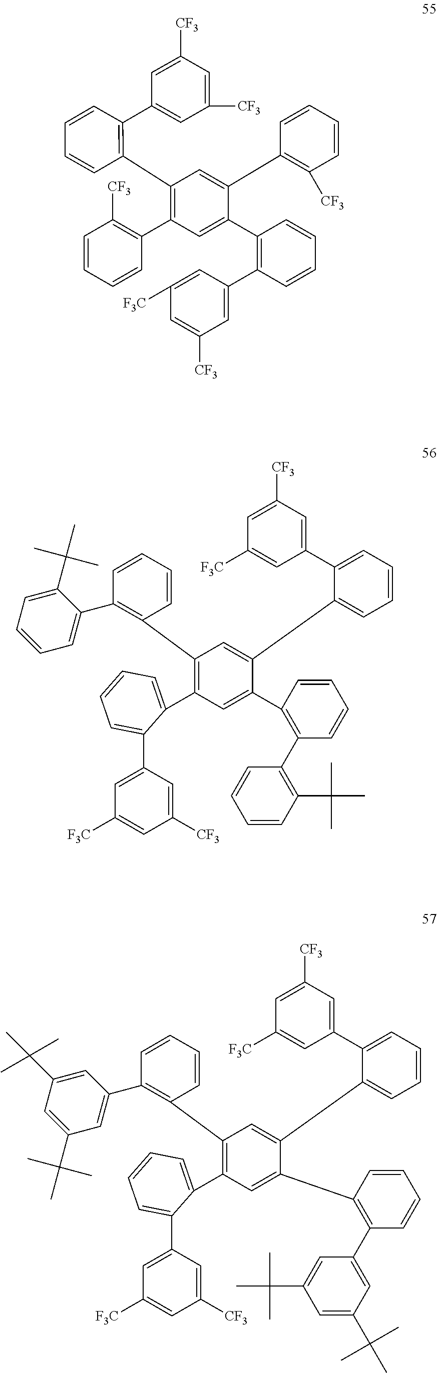 [SMILES: FC(F)(F)c1cc(-c2ccccc2-c2cc(-c3ccccc3C(F)(F)F)c(-c3ccccc3-c3cc(C(F)(F)F)cc(C(F)(F)F)c3)cc2-c2ccccc2C(F)(F)F)cc(C(F)(F)F)c1 ; CC(C)(C)c1ccccc1-c1ccccc1-c1cc(-c2ccccc2-c2cc(C(F)(F)F)cc(C(F)(F)F)c2)c(-c2ccccc2-c2ccccc2C(C)(C)C)cc1-c1ccccc1-c1cc(C(F)(F)F)cc(C(F)(F)F)c1 ; CC(C)(C)c1cc(-c2ccccc2-c2cc(-c3ccccc3-c3cc(C(F)(F)F)cc(C(F)(F)F)c3)c(-c3ccccc3-c3cc(C(C)(C)C)cc(C(C)(C)C)c3)cc2-c2ccccc2-c2cc(C(F)(F)F)cc(C(F)(F)F)c2)cc(C(C)(C)C)c1]

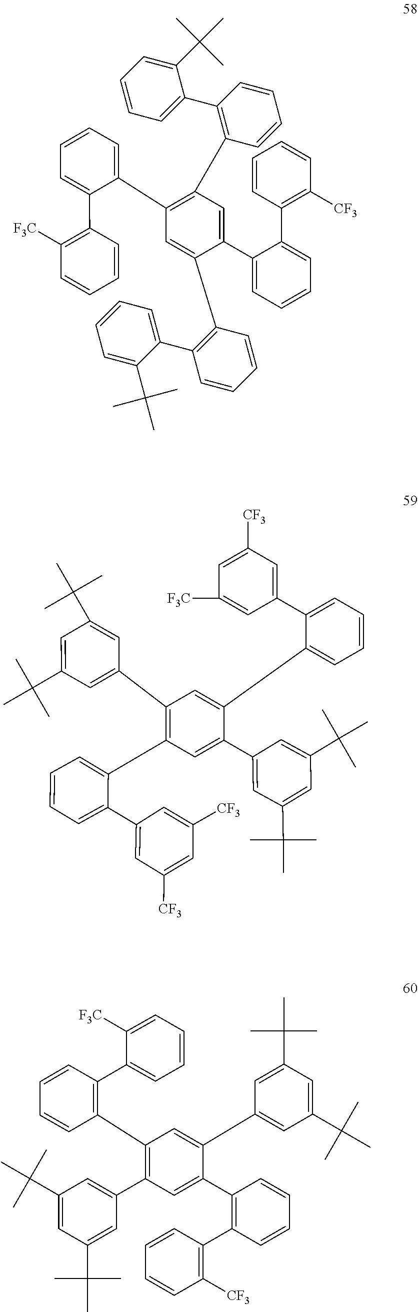

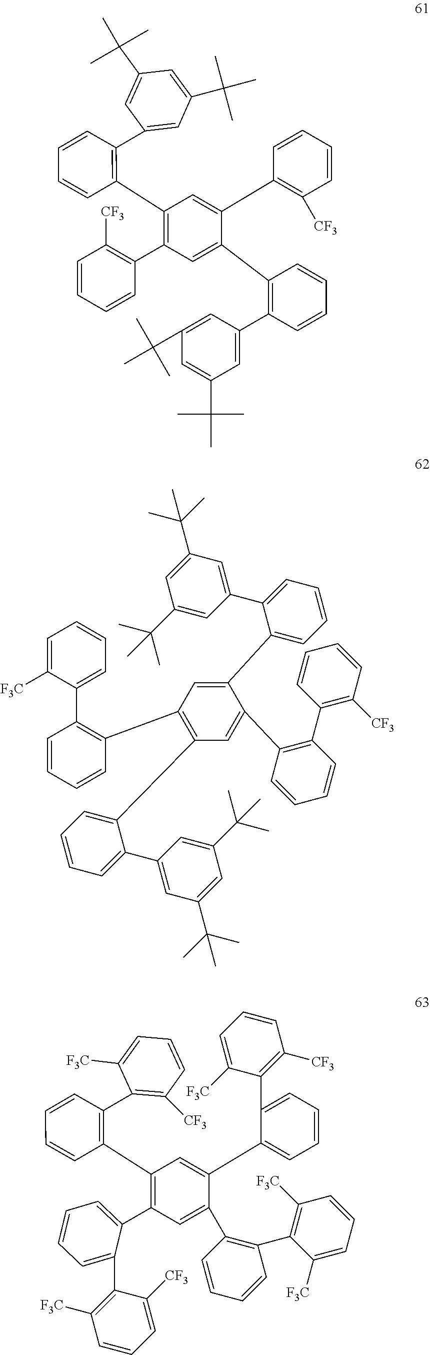 [SMILES: CC(C)(C)c1cc(-c2ccccc2-c2cc(-c3ccccc3C(F)(F)F)c(-c3ccccc3-c3cc(C(C)(C)C)cc(C(C)(C)C)c3)cc2-c2ccccc2C(F)(F)F)cc(C(C)(C)C)c1 ; CC(C)(C)c1cc(-c2ccccc2-c2cc(-c3ccccc3-c3ccccc3C(F)(F)F)c(-c3ccccc3-c3cc(C(C)(C)C)cc(C(C)(C)C)c3)cc2-c2ccccc2-c2ccccc2C(F)(F)F)cc(C(C)(C)C)c1 ; FC(F)(F)c1cccc(C(F)(F)F)c1-c1ccccc1-c1cc(-c2ccccc2-c2c(C(F)(F)F)cccc2C(F)(F)F)c(-c2ccccc2-c2c(C(F)(F)F)cccc2C(F)(F)F)cc1-c1ccccc1-c1c(C(F)(F)F)cccc1C(F)(F)F]

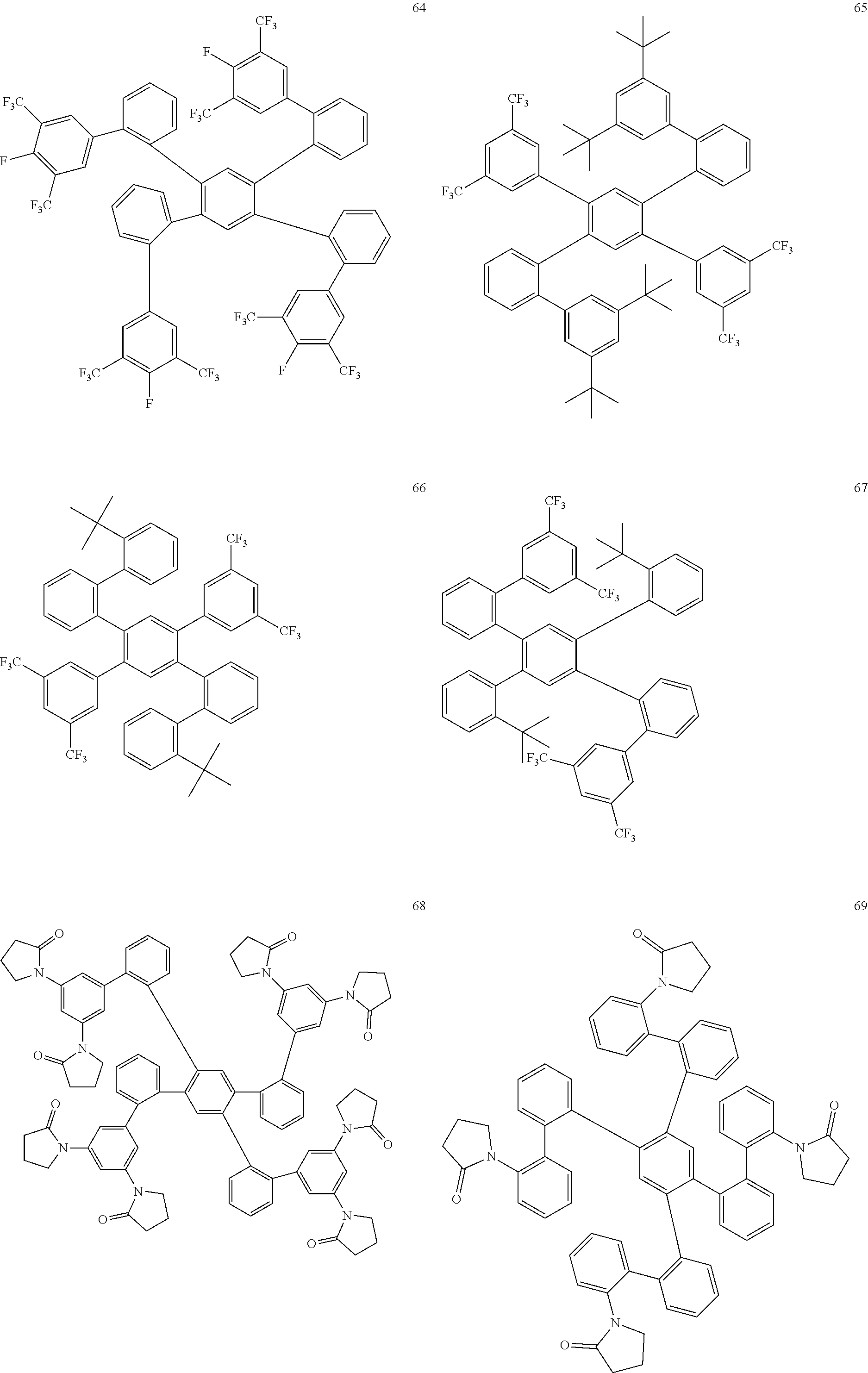

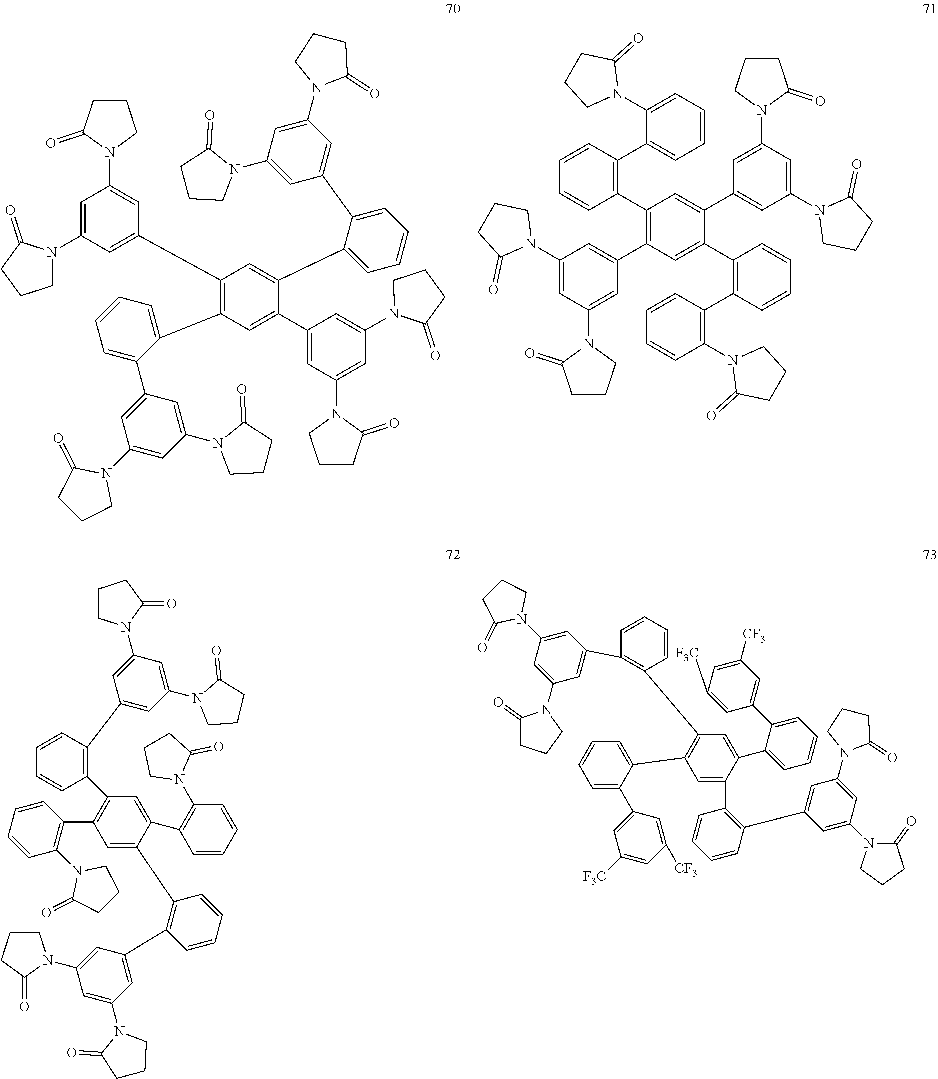

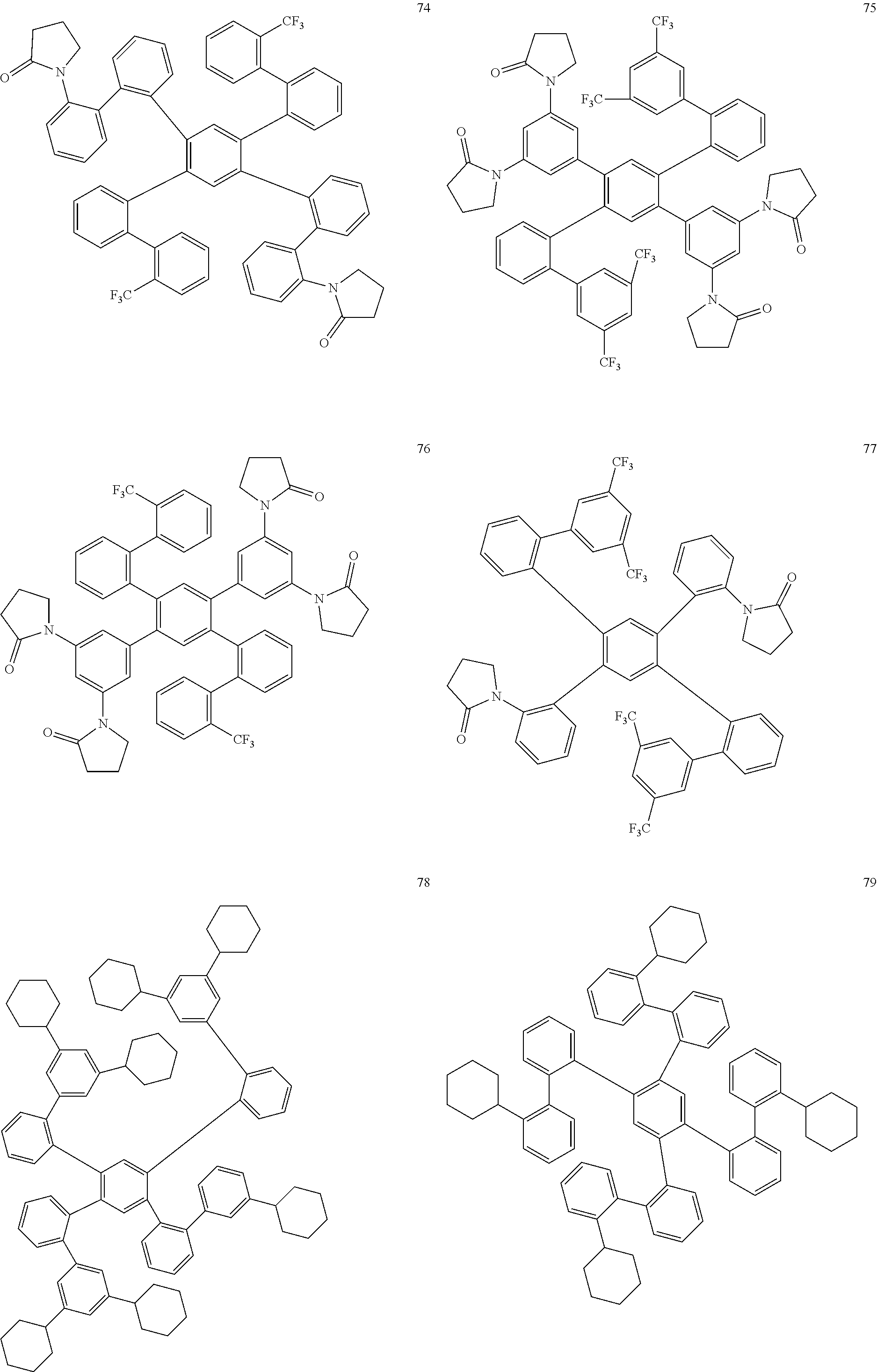

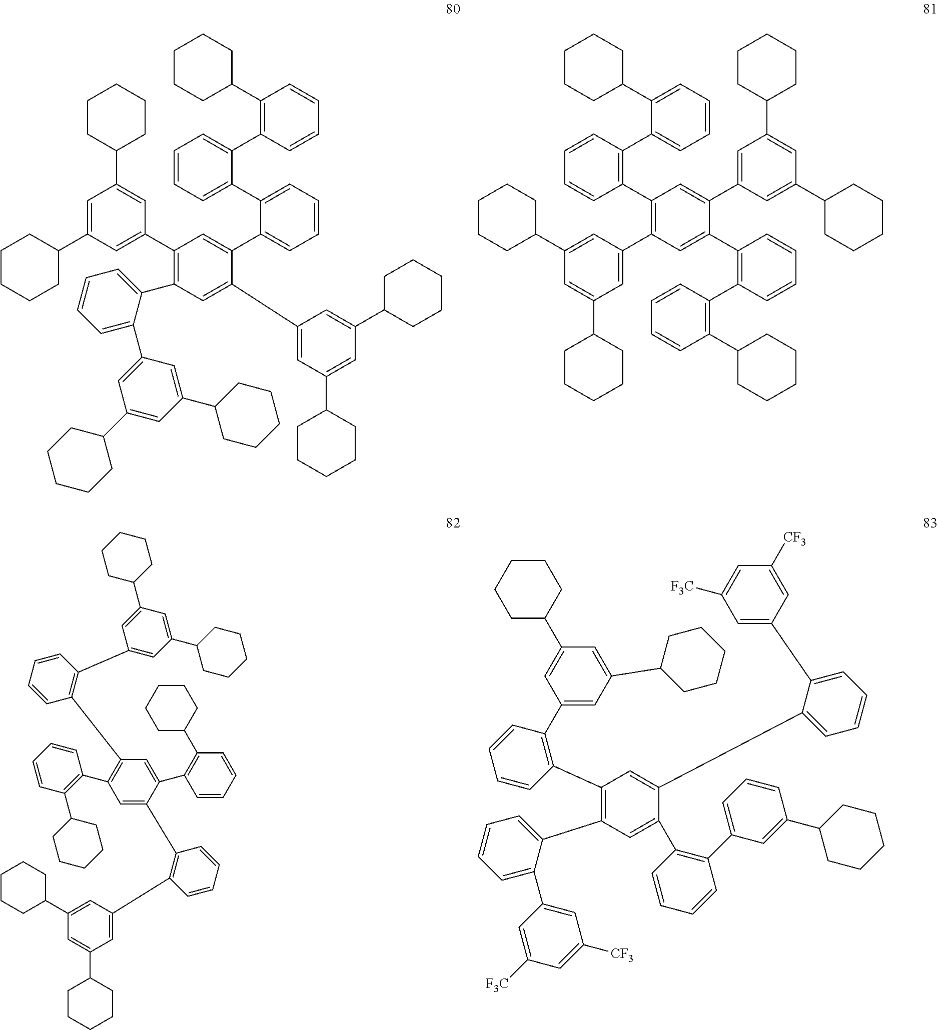

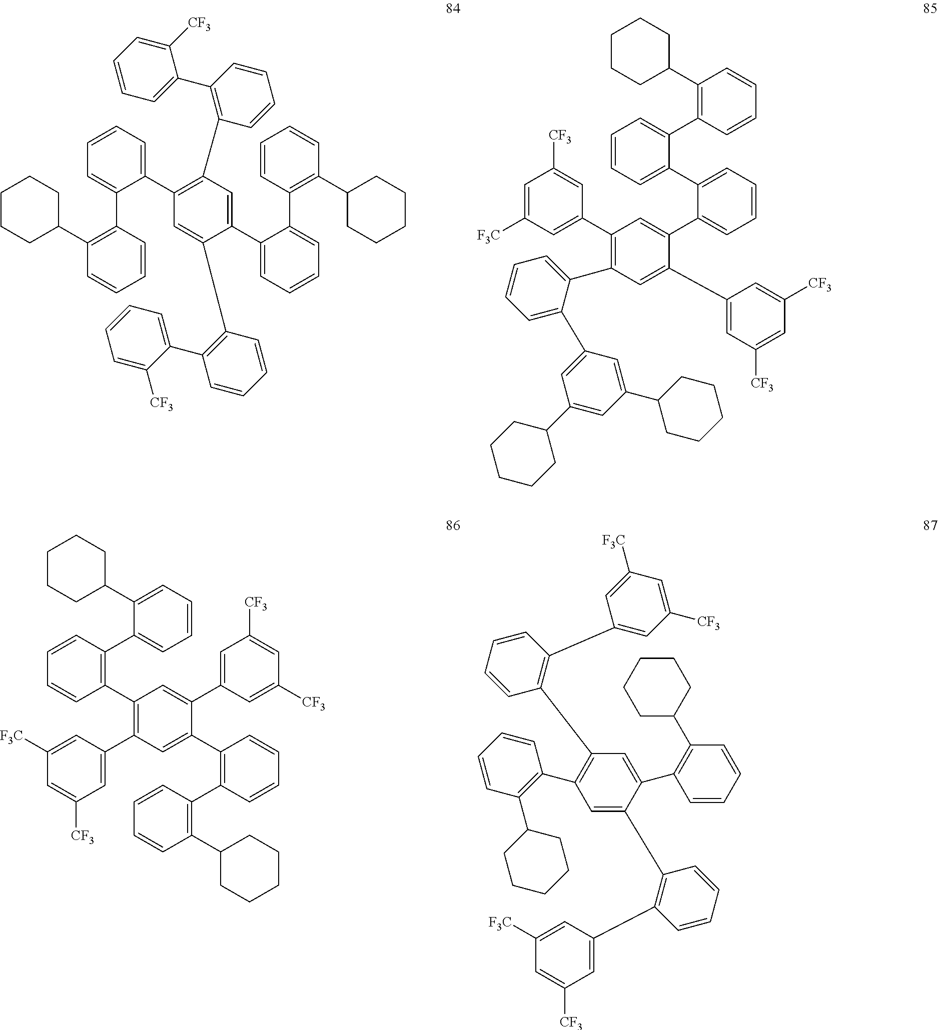

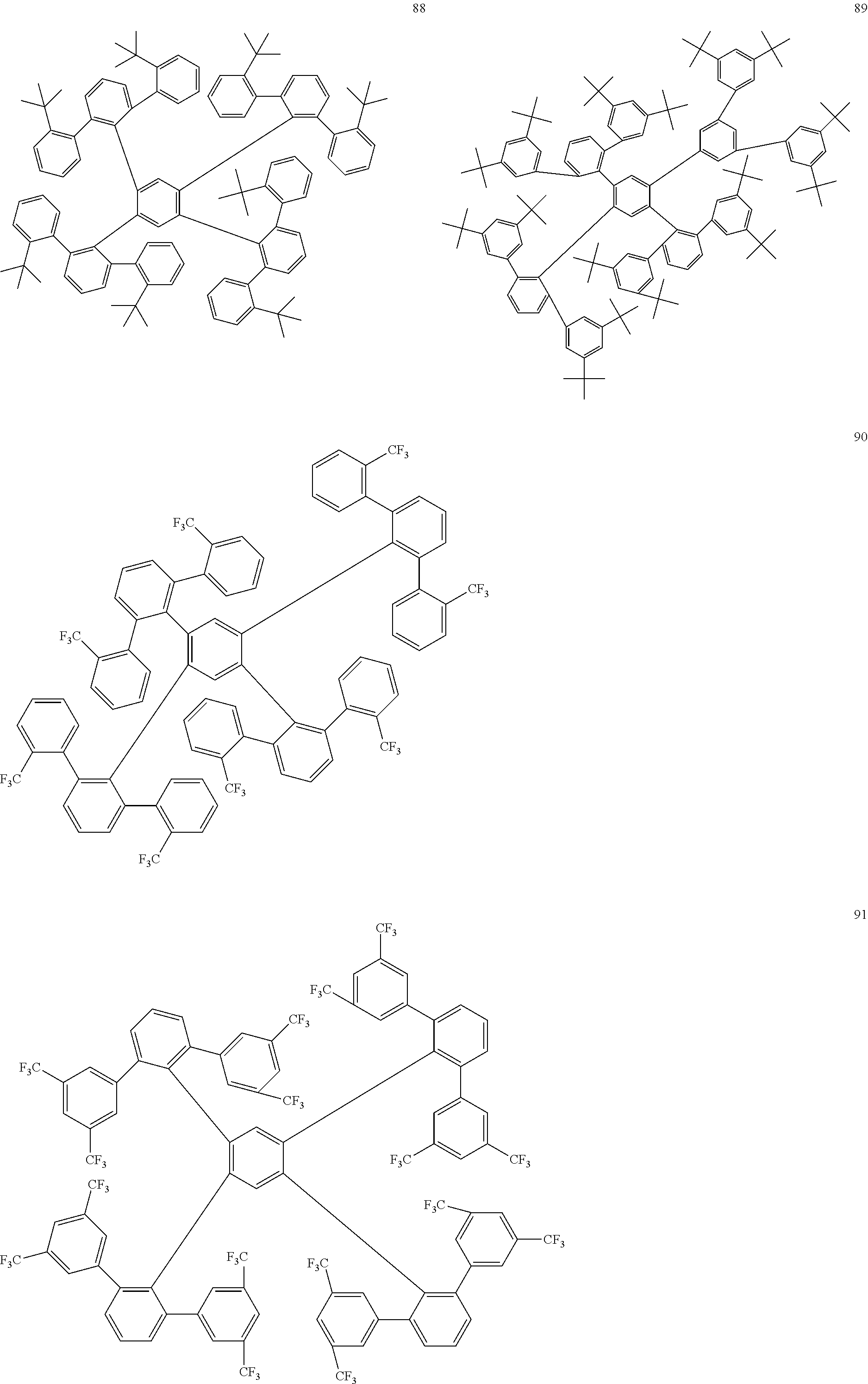

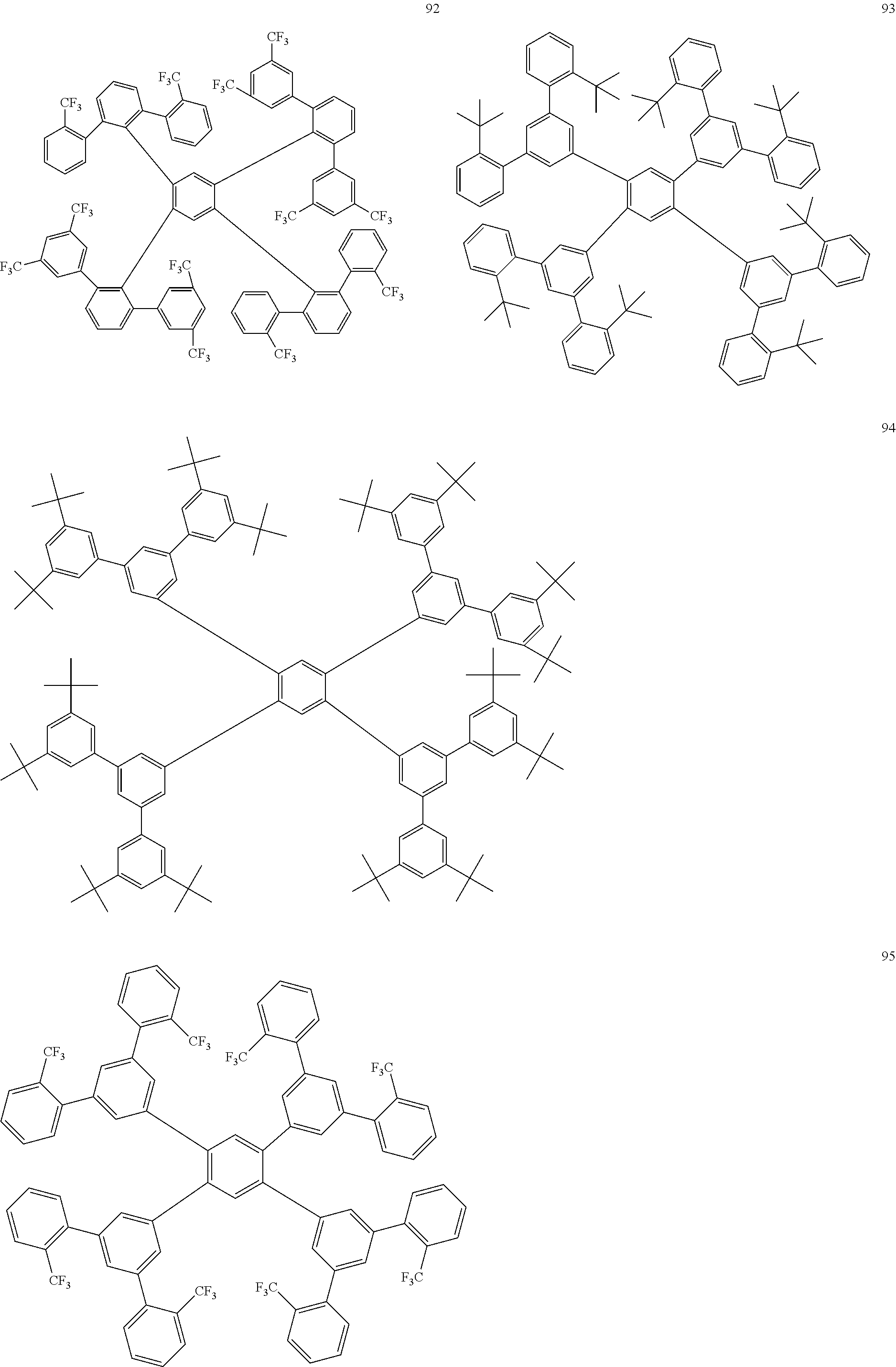

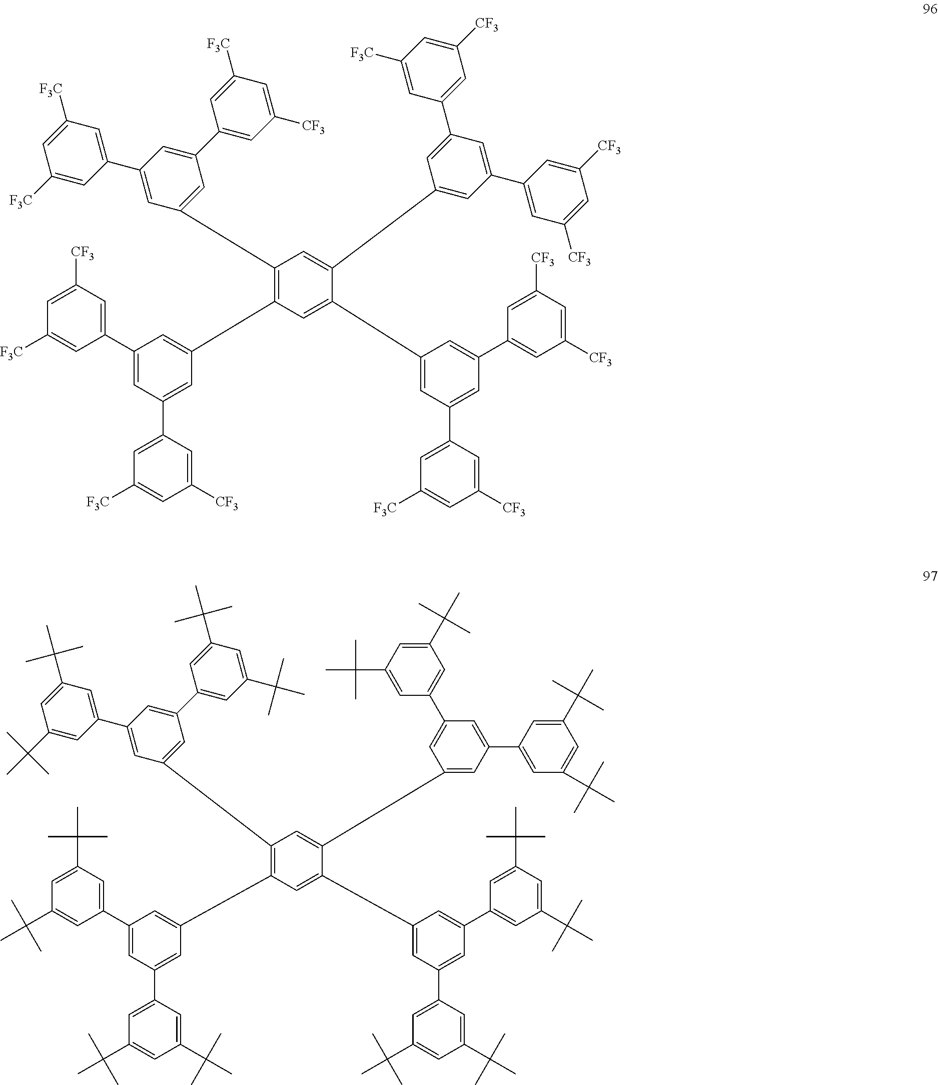

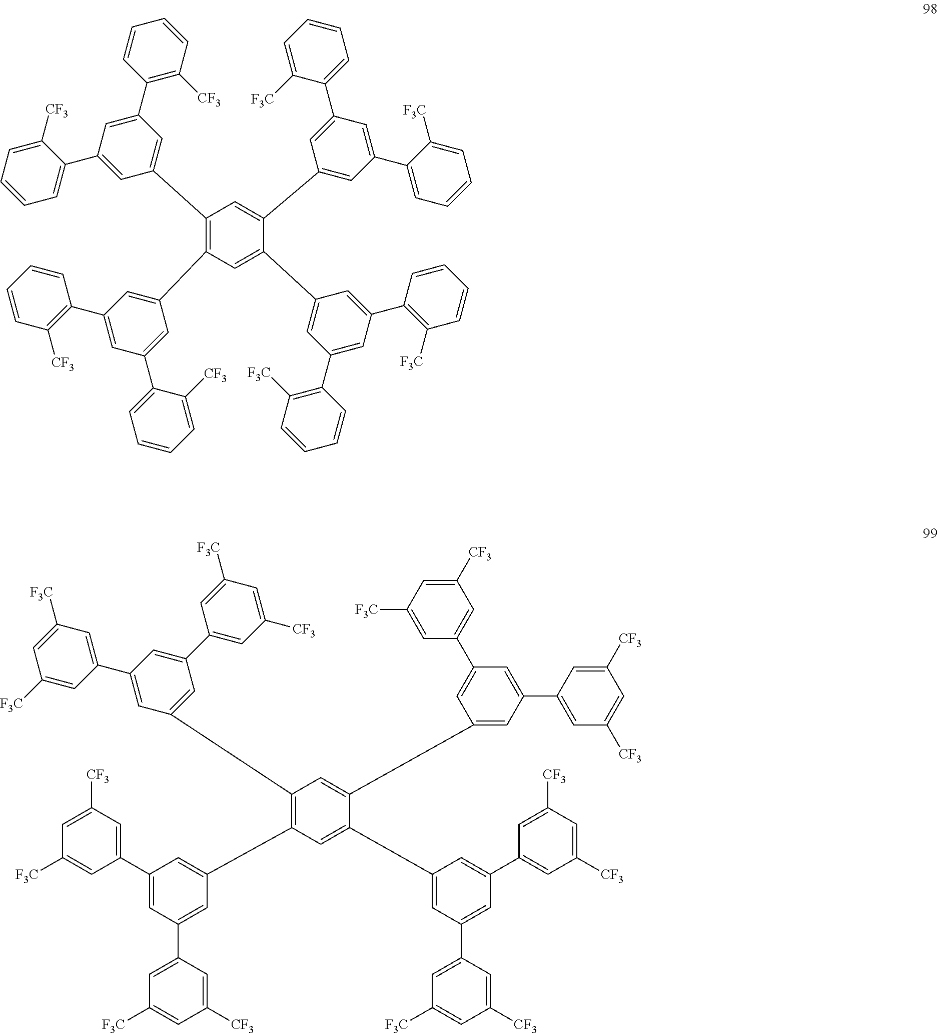 [SMILES: FC(F)(F)c1ccccc1-c1cc(-c2cc(-c3cc(-c4ccccc4C(F)(F)F)cc(-c4ccccc4C(F)(F)F)c3)c(-c3cc(-c4ccccc4C(F)(F)F)cc(-c4ccccc4C(F)(F)F)c3)cc2-c2cc(-c3ccccc3C(F)(F)F)cc(-c3ccccc3C(F)(F)F)c2)cc(-c2ccccc2C(F)(F)F)c1 ; FC(F)(F)c1cc(-c2cc(-c3cc(C(F)(F)F)cc(C(F)(F)F)c3)cc(-c3cc(-c4cc(-c5cc(C(F)(F)F)cc(C(F)(F)F)c5)cc(-c5cc(C(F)(F)F)cc(C(F)(F)F)c5)c4)c(-c4cc(-c5cc(C(F)(F)F)cc(C(F)(F)F)c5)cc(-c5cc(C(F)(F)F)cc(C(F)(F)F)c5)c4)cc3-c3cc(-c4cc(C(F)(F)F)cc(C(F)(F)F)c4)cc(-c4cc(C(F)(F)F)cc(C(F)(F)F)c4)c3)c2)cc(C(F)(F)F)c1]

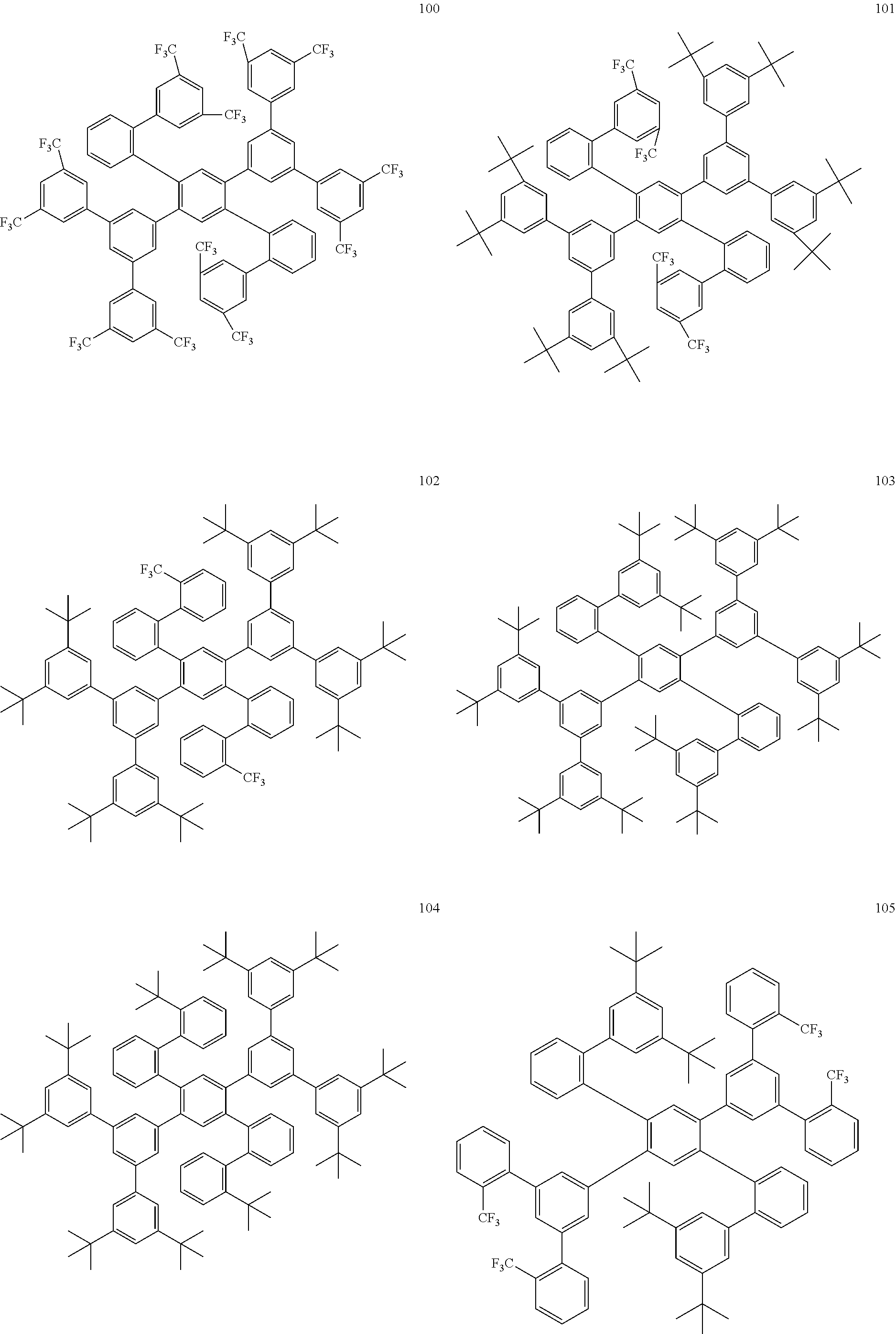

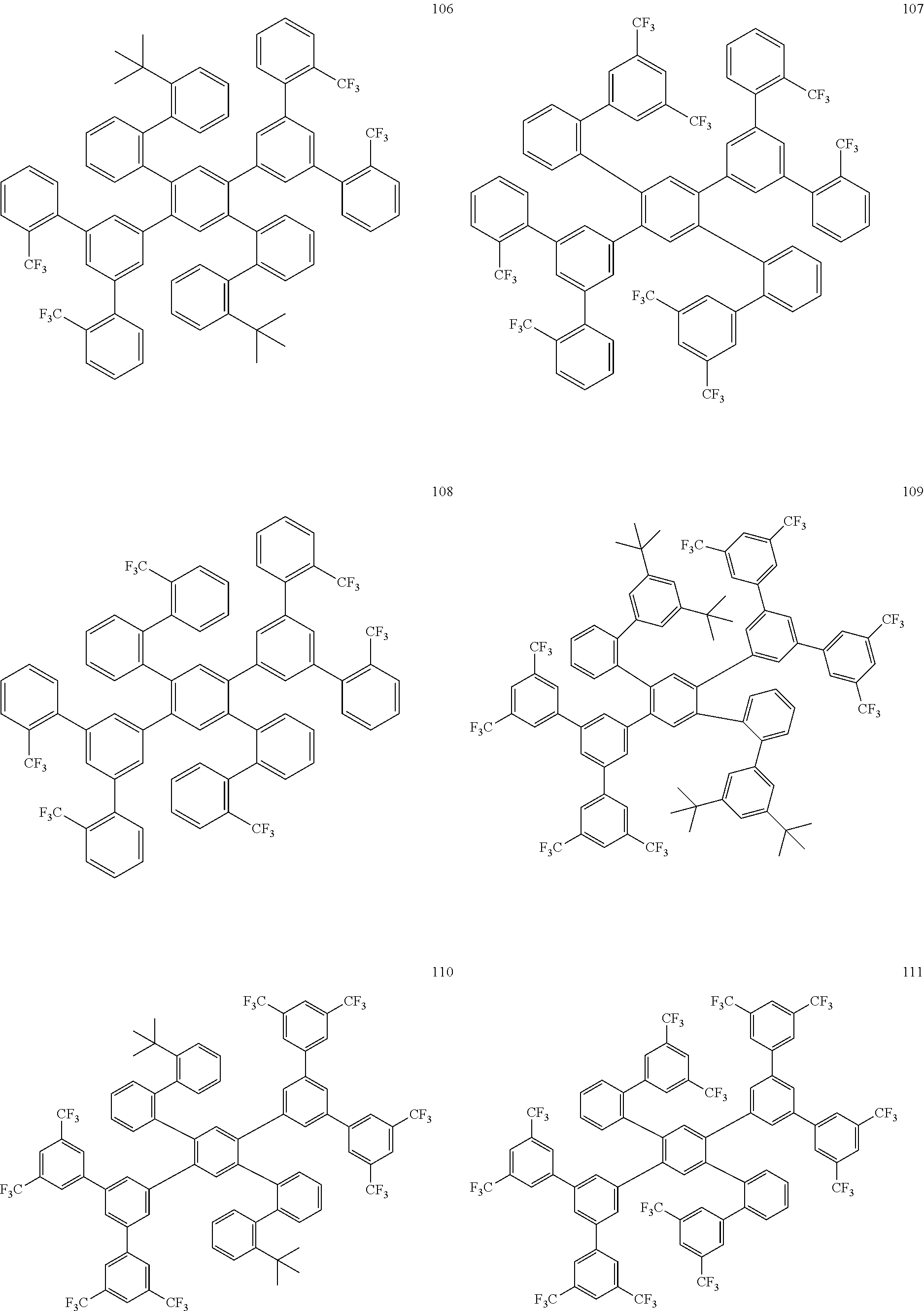

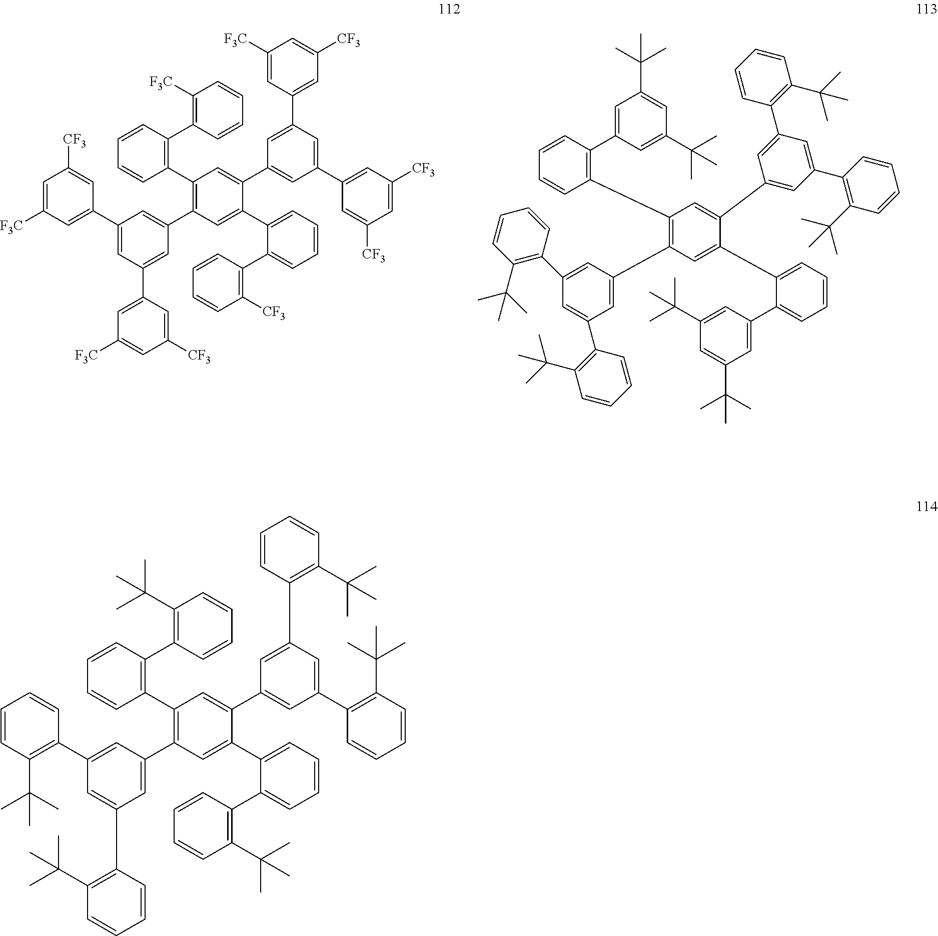

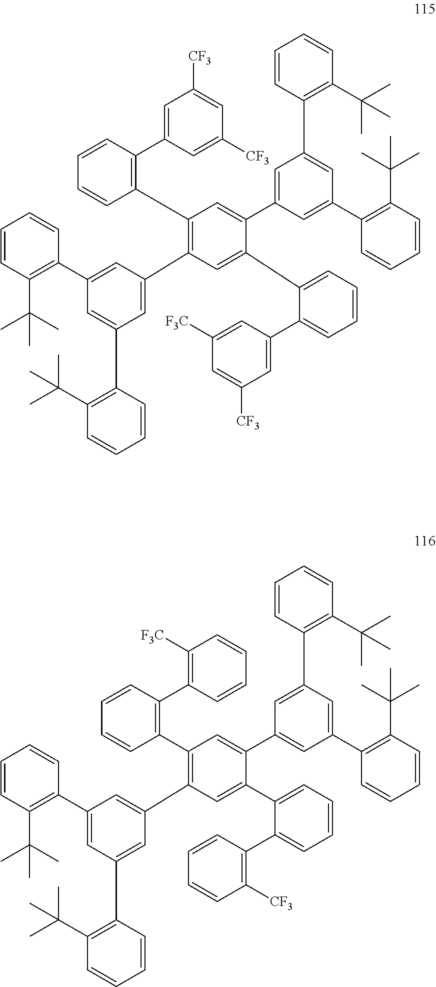 [SMILES: CC(C)(C)c1ccccc1-c1cc(-c2cc(-c3ccccc3-c3cc(C(F)(F)F)cc(C(F)(F)F)c3)c(-c3cc(-c4ccccc4C(C)(C)C)cc(-c4ccccc4C(C)(C)C)c3)cc2-c2ccccc2-c2cc(C(F)(F)F)cc(C(F)(F)F)c2)cc(-c2ccccc2C(C)(C)C)c1 ; CC(C)(C)c1ccccc1-c1cc(-c2cc(-c3ccccc3-c3ccccc3C(F)(F)F)c(-c3cc(-c4ccccc4C(C)(C)C)cc(-c4ccccc4C(C)(C)C)c3)cc2-c2ccccc2-c2ccccc2C(F)(F)F)cc(-c2ccccc2C(C)(C)C)c1]

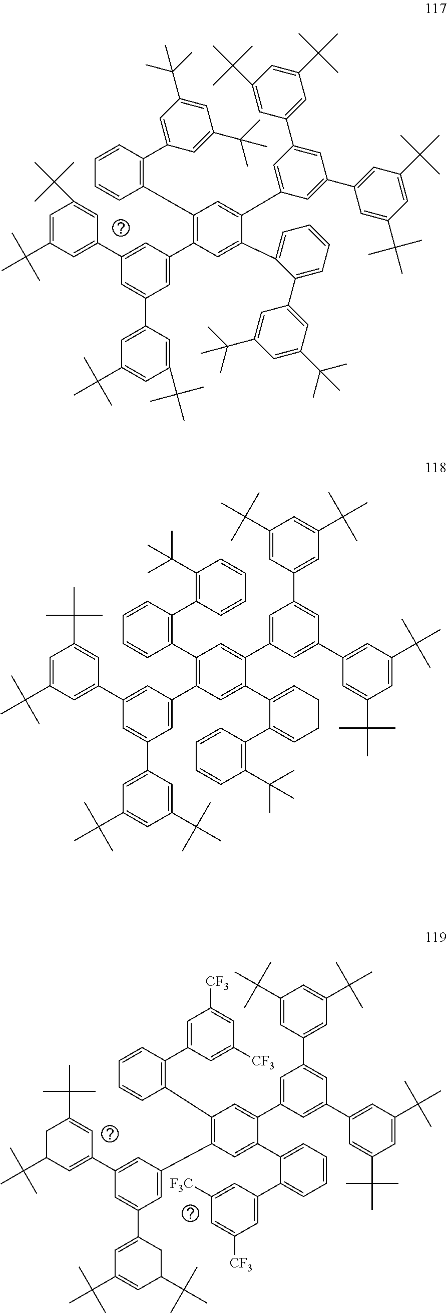

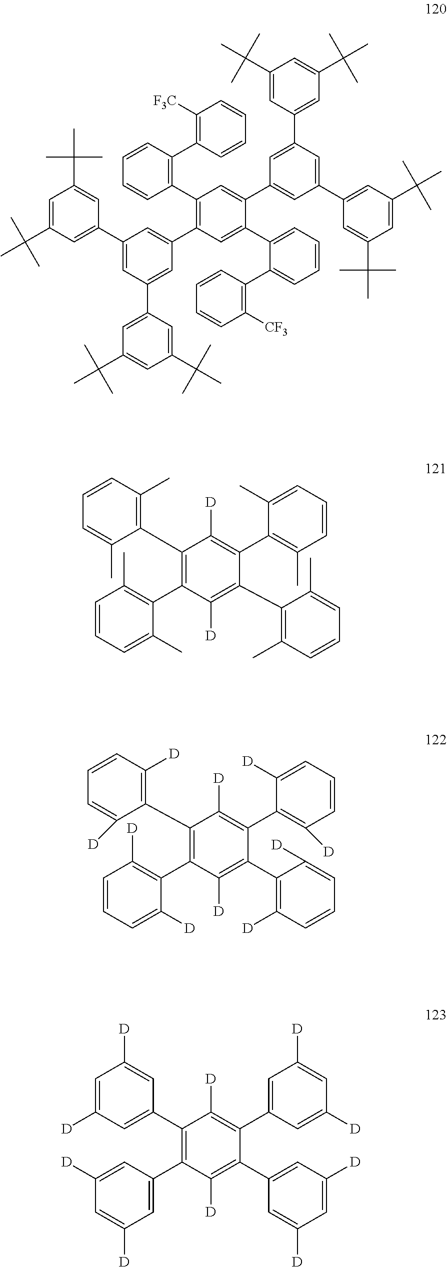 [SMILES: CC(C)(C)c1cc(-c2cc(-c3cc(C(C)(C)C)cc(C(C)(C)C)c3)cc(-c3cc(-c4ccccc4-c4ccccc4C(F)(F)F)c(-c4cc(-c5cc(C(C)(C)C)cc(C(C)(C)C)c5)cc(-c5cc(C(C)(C)C)cc(C(C)(C)C)c5)c4)cc3-c3ccccc3-c3ccccc3C(F)(F)F)c2)cc(C(C)(C)C)c1 ; [2H]c1c(-c2c(C)cccc2C)c(-c2c(C)cccc2C)c([2H])c(-c2c(C)cccc2C)c1-c1c(C)cccc1C ; [2H]c1cccc([2H])c1-c1c([2H])c(-c2c([2H])cccc2[2H])c(-c2c([2H])cccc2[2H])c([2H])c1-c1c([2H])cccc1[2H] ; [2H]c1cc([2H])cc(-c2c([2H])c(-c3cc([2H])cc([2H])c3)c(-c3cc([2H])cc([2H])c3)c([2H])c2-c2cc([2H])cc([2H])c2)c1]

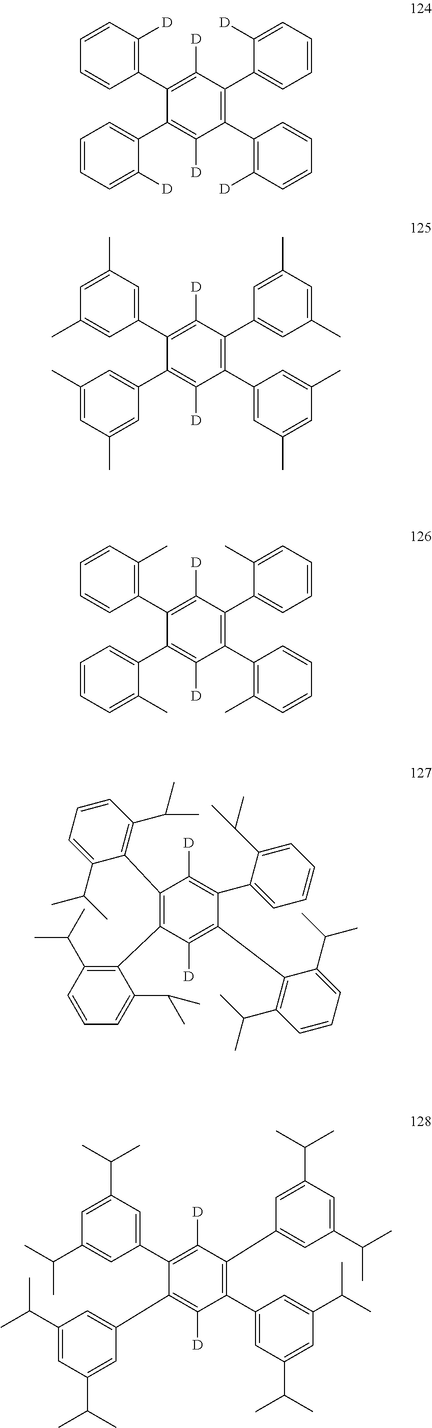

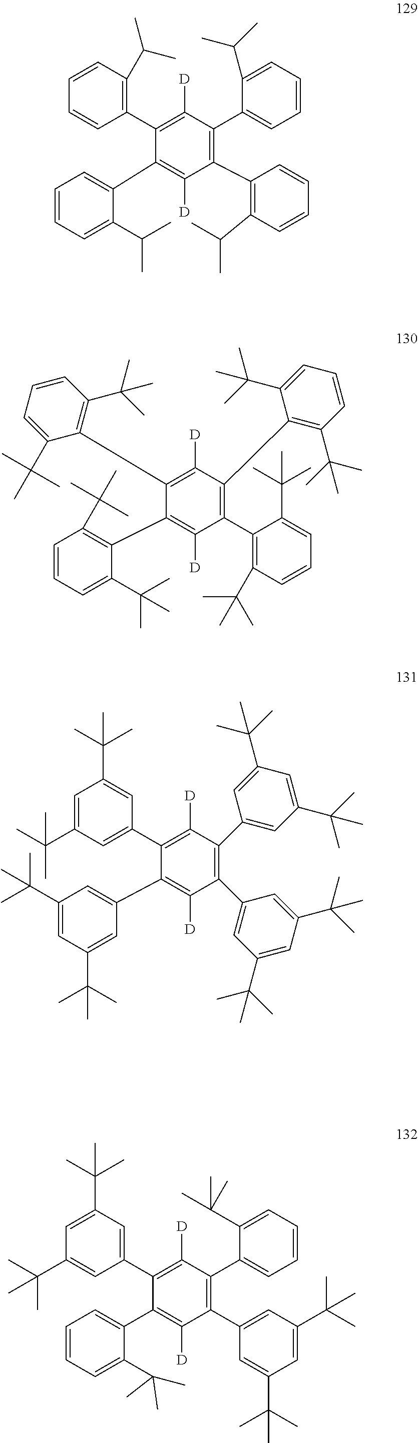

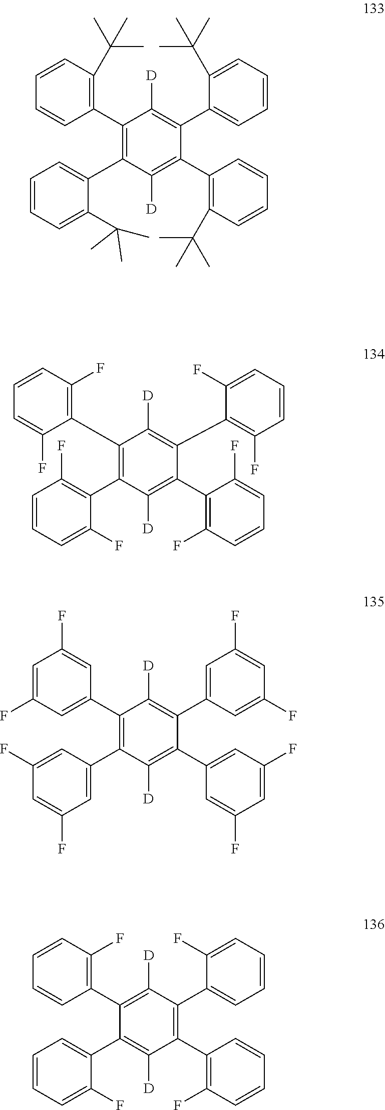 [SMILES: [2H]c1c(-c2ccccc2C(C)(C)C)c(-c2ccccc2C(C)(C)C)c([2H])c(-c2ccccc2C(C)(C)C)c1-c1ccccc1C(C)(C)C ; [2H]c1c(-c2c(F)cccc2F)c(-c2c(F)cccc2F)c([2H])c(-c2c(F)cccc2F)c1-c1c(F)cccc1F ; [2H]c1c(-c2cc(F)cc(F)c2)c(-c2cc(F)cc(F)c2)c([2H])c(-c2cc(F)cc(F)c2)c1-c1cc(F)cc(F)c1 ; [2H]c1c(-c2ccccc2F)c(-c2ccccc2F)c([2H])c(-c2ccccc2F)c1-c1ccccc1F]

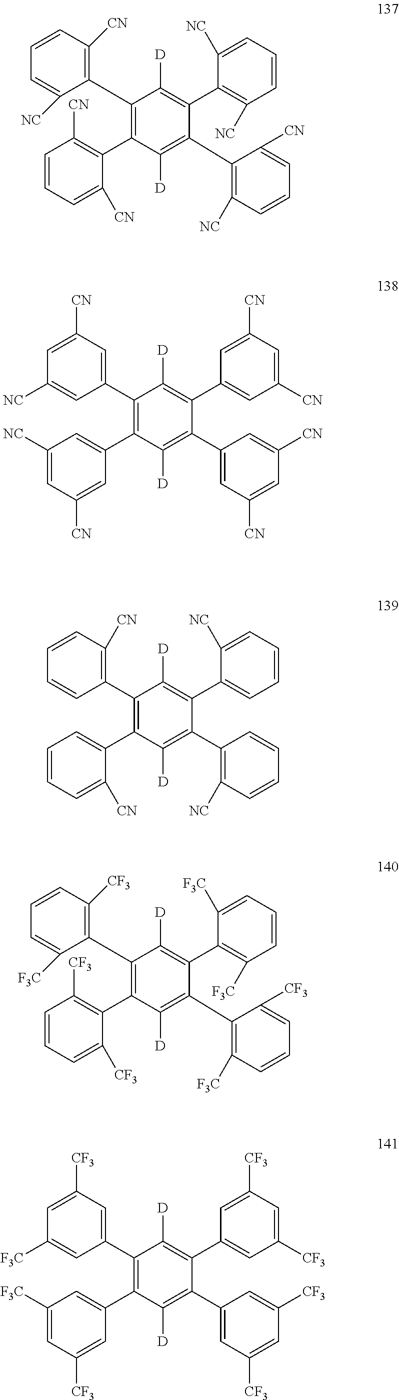

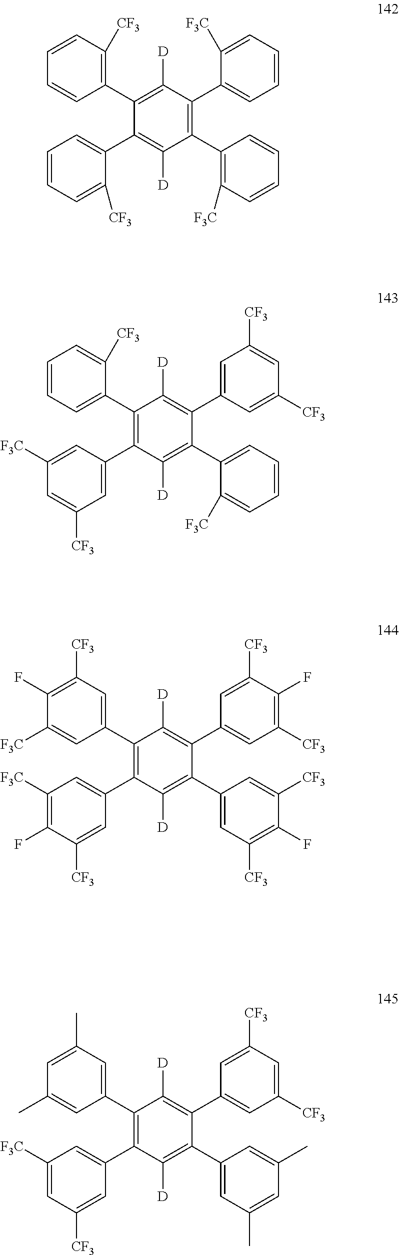 [SMILES: [2H]c1c(-c2ccccc2C(F)(F)F)c(-c2ccccc2C(F)(F)F)c([2H])c(-c2ccccc2C(F)(F)F)c1-c1ccccc1C(F)(F)F ; [2H]c1c(-c2cc(C(F)(F)F)cc(C(F)(F)F)c2)c(-c2ccccc2C(F)(F)F)c([2H])c(-c2cc(C(F)(F)F)cc(C(F)(F)F)c2)c1-c1ccccc1C(F)(F)F ; [2H]c1c(-c2cc(C(F)(F)F)c(F)c(C(F)(F)F)c2)c(-c2cc(C(F)(F)F)c(F)c(C(F)(F)F)c2)c([2H])c(-c2cc(C(F)(F)F)c(F)c(C(F)(F)F)c2)c1-c1cc(C(F)(F)F)c(F)c(C(F)(F)F)c1 ; [2H]c1c(-c2cc(C)cc(C)c2)c(-c2cc(C(F)(F)F)cc(C(F)(F)F)c2)c([2H])c(-c2cc(C)cc(C)c2)c1-c1cc(C(F)(F)F)cc(C(F)(F)F)c1]

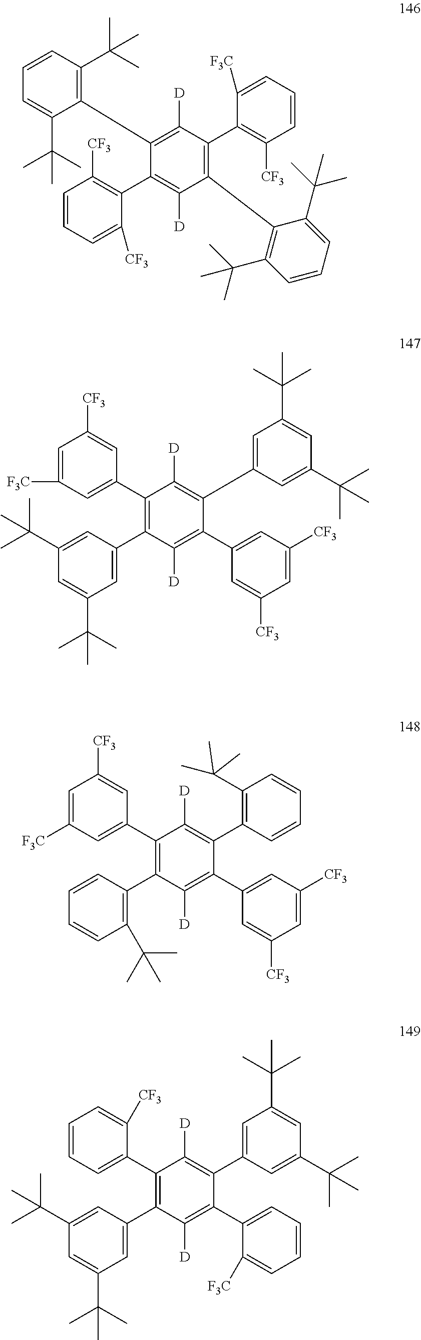

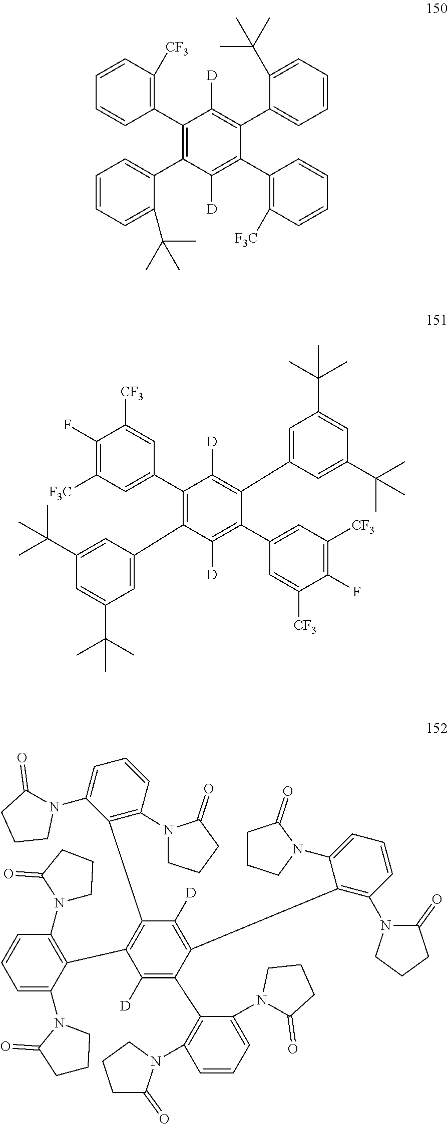 [SMILES: [2H]c1c(-c2ccccc2C(C)(C)C)c(-c2ccccc2C(F)(F)F)c([2H])c(-c2ccccc2C(C)(C)C)c1-c1ccccc1C(F)(F)F ; [2H]c1c(-c2cc(C(C)(C)C)cc(C(C)(C)C)c2)c(-c2cc(C(F)(F)F)c(F)c(C(F)(F)F)c2)c([2H])c(-c2cc(C(C)(C)C)cc(C(C)(C)C)c2)c1-c1cc(C(F)(F)F)c(F)c(C(F)(F)F)c1 ; [2H]c1c(-c2c(N3CCCC3=O)cccc2N2CCCC2=O)c(-c2c(N3CCCC3=O)cccc2N2CCCC2=O)c([2H])c(-c2c(N3CCCC3=O)cccc2N2CCCC2=O)c1-c1c(N2CCCC2=O)cccc1N1CCCC1=O]

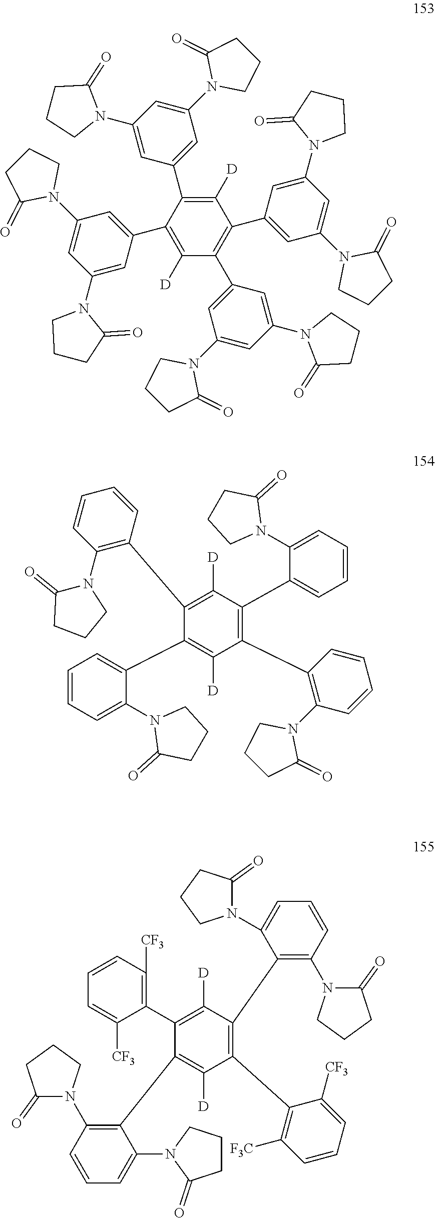 [SMILES: [2H]c1c(-c2cc(N3CCCC3=O)cc(N3CCCC3=O)c2)c(-c2cc(N3CCCC3=O)cc(N3CCCC3=O)c2)c([2H])c(-c2cc(N3CCCC3=O)cc(N3CCCC3=O)c2)c1-c1cc(N2CCCC2=O)cc(N2CCCC2=O)c1 ; [2H]c1c(-c2ccccc2N2CCCC2=O)c(-c2ccccc2N2CCCC2=O)c([2H])c(-c2ccccc2N2CCCC2=O)c1-c1ccccc1N1CCCC1=O ; [2H]c1c(-c2c(N3CCCC3=O)cccc2N2CCCC2=O)c(-c2c(C(F)(F)F)cccc2C(F)(F)F)c([2H])c(-c2c(N3CCCC3=O)cccc2N2CCCC2=O)c1-c1c(C(F)(F)F)cccc1C(F)(F)F]

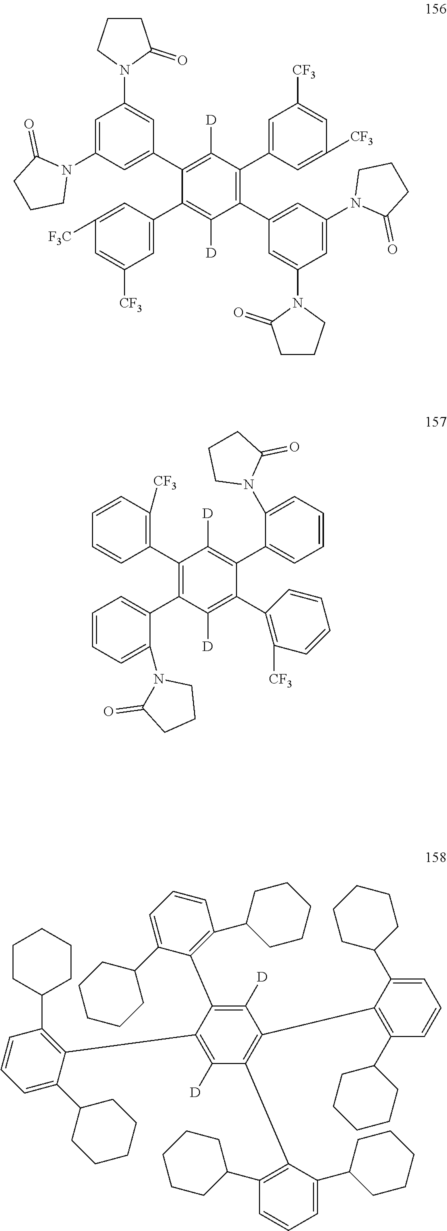 [SMILES: [2H]c1c(-c2cc(N3CCCC3=O)cc(N3CCCC3=O)c2)c(-c2cc(C(F)(F)F)cc(C(F)(F)F)c2)c([2H])c(-c2cc(N3CCCC3=O)cc(N3CCCC3=O)c2)c1-c1cc(C(F)(F)F)cc(C(F)(F)F)c1 ; [2H]c1c(-c2ccccc2N2CCCC2=O)c(-c2ccccc2C(F)(F)F)c([2H])c(-c2ccccc2N2CCCC2=O)c1-c1ccccc1C(F)(F)F ; [2H]c1c(-c2c(C3CCCCC3)cccc2C2CCCCC2)c(-c2c(C3CCCCC3)cccc2C2CCCCC2)c([2H])c(-c2c(C3CCCCC3)cccc2C2CCCCC2)c1-c1c(C2CCCCC2)cccc1C1CCCCC1]

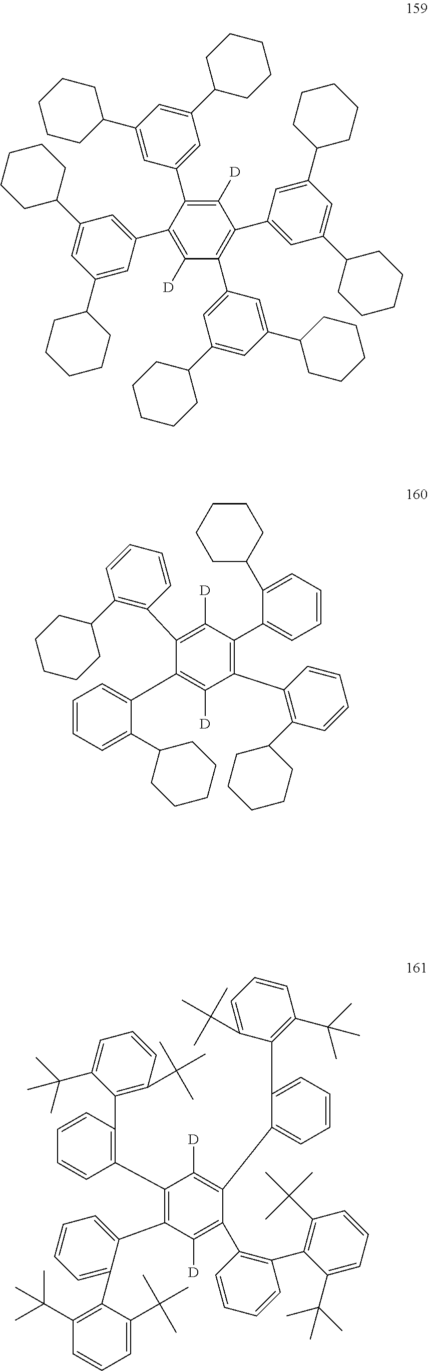 [SMILES: [2H]c1c(-c2cc(C3CCCCC3)cc(C3CCCCC3)c2)c(-c2cc(C3CCCCC3)cc(C3CCCCC3)c2)c([2H])c(-c2cc(C3CCCCC3)cc(C3CCCCC3)c2)c1-c1cc(C2CCCCC2)cc(C2CCCCC2)c1 ; [2H]c1c(-c2ccccc2C2CCCCC2)c(-c2ccccc2C2CCCCC2)c([2H])c(-c2ccccc2C2CCCCC2)c1-c1ccccc1C1CCCCC1 ; [2H]c1c(-c2ccccc2-c2c(C(C)(C)C)cccc2C(C)(C)C)c(-c2ccccc2-c2c(C(C)(C)C)cccc2C(C)(C)C)c([2H])c(-c2ccccc2-c2c(C(C)(C)C)cccc2C(C)(C)C)c1-c1ccccc1-c1c(C(C)(C)C)cccc1C(C)(C)C]

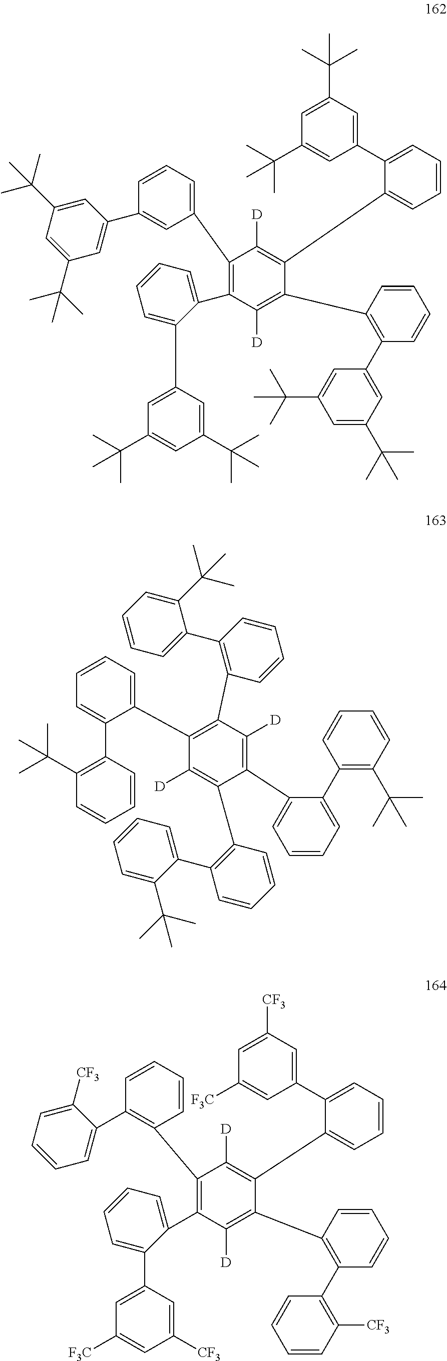 [SMILES: [2H]c1c(-c2cccc(-c3cc(C(C)(C)C)cc(C(C)(C)C)c3)c2)c(-c2ccccc2-c2cc(C(C)(C)C)cc(C(C)(C)C)c2)c([2H])c(-c2ccccc2-c2cc(C(C)(C)C)cc(C(C)(C)C)c2)c1-c1ccccc1-c1cc(C(C)(C)C)cc(C(C)(C)C)c1 ; [2H]c1c(-c2ccccc2-c2ccccc2C(C)(C)C)c(-c2ccccc2-c2ccccc2C(C)(C)C)c([2H])c(-c2ccccc2-c2ccccc2C(C)(C)C)c1-c1ccccc1-c1ccccc1C(C)(C)C ; [2H]c1c(-c2ccccc2-c2cc(C(F)(F)F)cc(C(F)(F)F)c2)c(-c2ccccc2-c2ccccc2C(F)(F)F)c([2H])c(-c2ccccc2-c2cc(C(F)(F)F)cc(C(F)(F)F)c2)c1-c1ccccc1-c1ccccc1C(F)(F)F]

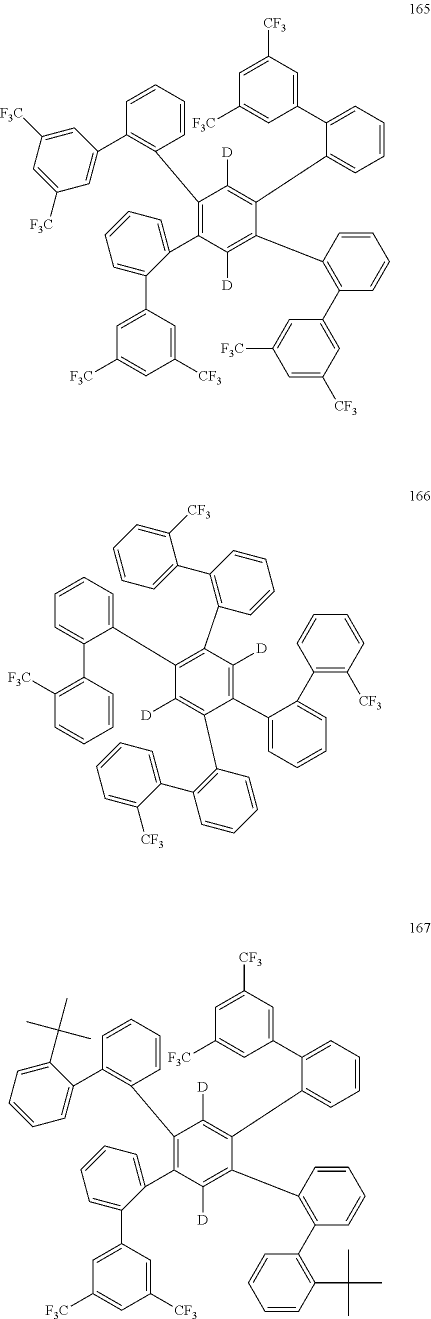 [SMILES: [2H]c1c(-c2ccccc2-c2cc(C(F)(F)F)cc(C(F)(F)F)c2)c(-c2ccccc2-c2cc(C(F)(F)F)cc(C(F)(F)F)c2)c([2H])c(-c2ccccc2-c2cc(C(F)(F)F)cc(C(F)(F)F)c2)c1-c1ccccc1-c1cc(C(F)(F)F)cc(C(F)(F)F)c1 ; [2H]c1c(-c2ccccc2-c2ccccc2C(F)(F)F)c(-c2ccccc2-c2ccccc2C(F)(F)F)c([2H])c(-c2ccccc2-c2ccccc2C(F)(F)F)c1-c1ccccc1-c1ccccc1C(F)(F)F ; [2H]c1c(-c2ccccc2-c2cc(C(F)(F)F)cc(C(F)(F)F)c2)c(-c2ccccc2-c2ccccc2C(C)(C)C)c([2H])c(-c2ccccc2-c2cc(C(F)(F)F)cc(C(F)(F)F)c2)c1-c1ccccc1-c1ccccc1C(C)(C)C]

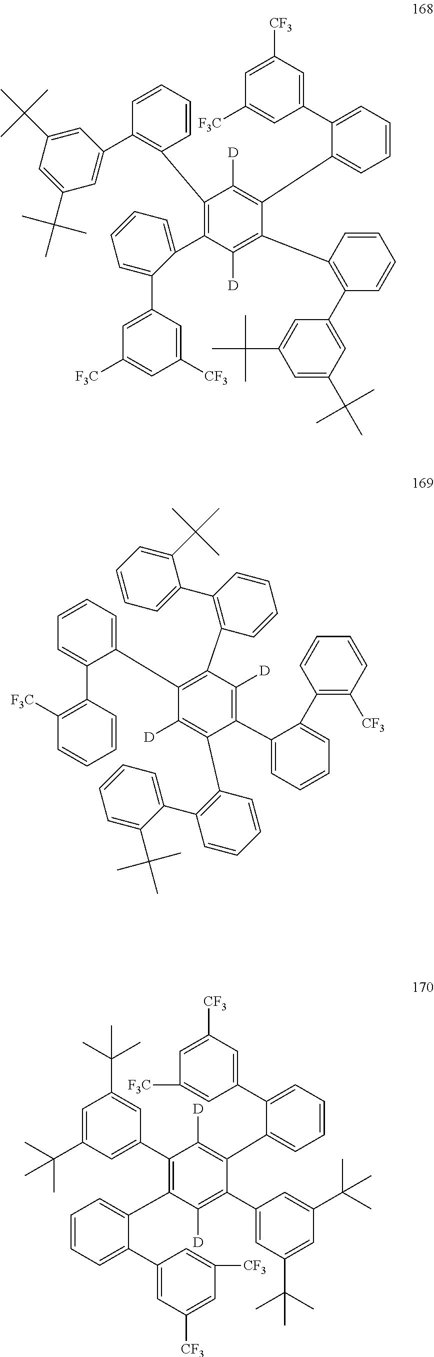 [SMILES: [2H]c1c(-c2ccccc2-c2cc(C(C)(C)C)cc(C(C)(C)C)c2)c(-c2ccccc2-c2cc(C(F)(F)F)cc(C(F)(F)F)c2)c([2H])c(-c2ccccc2-c2cc(C(C)(C)C)cc(C(C)(C)C)c2)c1-c1ccccc1-c1cc(C(F)(F)F)cc(C(F)(F)F)c1 ; [2H]c1c(-c2ccccc2-c2ccccc2C(C)(C)C)c(-c2ccccc2-c2ccccc2C(F)(F)F)c([2H])c(-c2ccccc2-c2ccccc2C(C)(C)C)c1-c1ccccc1-c1ccccc1C(F)(F)F ; [2H]c1c(-c2cc(C(C)(C)C)cc(C(C)(C)C)c2)c(-c2ccccc2-c2cc(C(F)(F)F)cc(C(F)(F)F)c2)c([2H])c(-c2cc(C(C)(C)C)cc(C(C)(C)C)c2)c1-c1ccccc1-c1cc(C(F)(F)F)cc(C(F)(F)F)c1]

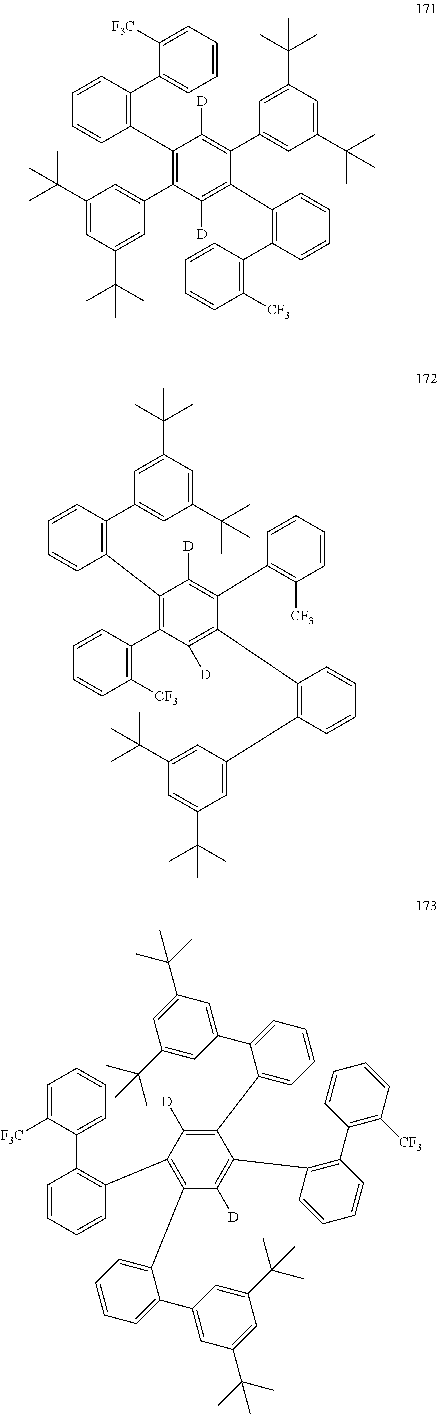 [SMILES: [2H]c1c(-c2cc(C(C)(C)C)cc(C(C)(C)C)c2)c(-c2ccccc2-c2ccccc2C(F)(F)F)c([2H])c(-c2cc(C(C)(C)C)cc(C(C)(C)C)c2)c1-c1ccccc1-c1ccccc1C(F)(F)F ; [2H]c1c(-c2ccccc2-c2cc(C(C)(C)C)cc(C(C)(C)C)c2)c(-c2ccccc2C(F)(F)F)c([2H])c(-c2ccccc2-c2cc(C(C)(C)C)cc(C(C)(C)C)c2)c1-c1ccccc1C(F)(F)F ; [2H]c1c(-c2ccccc2-c2cc(C(C)(C)C)cc(C(C)(C)C)c2)c(-c2ccccc2-c2ccccc2C(F)(F)F)c([2H])c(-c2ccccc2-c2cc(C(C)(C)C)cc(C(C)(C)C)c2)c1-c1ccccc1-c1ccccc1C(F)(F)F]

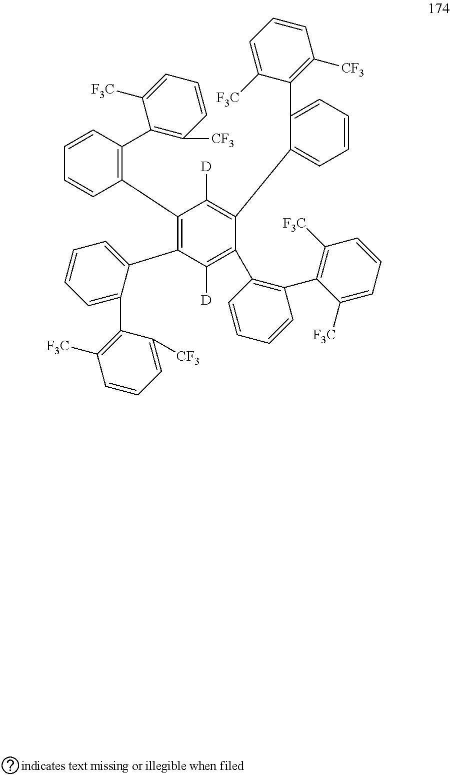 [SMILES: [2H]c1c(-c2ccccc2-c2c(C(F)(F)F)cccc2C(F)(F)F)c(-c2ccccc2-c2c(C(F)(F)F)cccc2C(F)(F)F)c([2H])c(-c2ccccc2-c2c(C(F)(F)F)cccc2C(F)(F)F)c1-c1ccccc1-c1c(C(F)(F)F)cccc1C(F)(F)F]

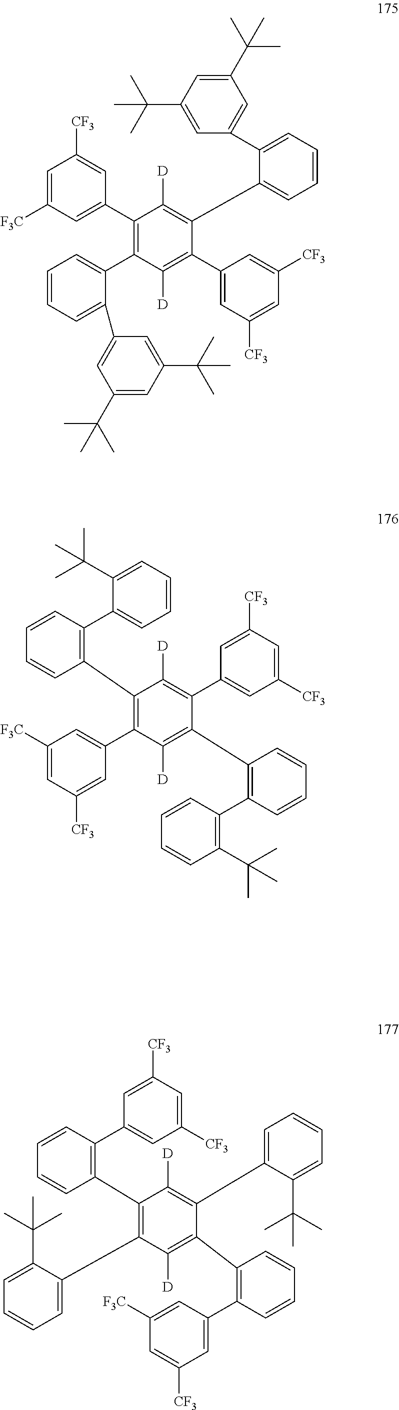 [SMILES: [2H]c1c(-c2cc(C(F)(F)F)cc(C(F)(F)F)c2)c(-c2ccccc2-c2cc(C(C)(C)C)cc(C(C)(C)C)c2)c([2H])c(-c2cc(C(F)(F)F)cc(C(F)(F)F)c2)c1-c1ccccc1-c1cc(C(C)(C)C)cc(C(C)(C)C)c1 ; [2H]c1c(-c2cc(C(F)(F)F)cc(C(F)(F)F)c2)c(-c2ccccc2-c2ccccc2C(C)(C)C)c([2H])c(-c2cc(C(F)(F)F)cc(C(F)(F)F)c2)c1-c1ccccc1-c1ccccc1C(C)(C)C ; [2H]c1c(-c2ccccc2-c2cc(C(F)(F)F)cc(C(F)(F)F)c2)c(-c2ccccc2C(C)(C)C)c([2H])c(-c2ccccc2-c2cc(C(F)(F)F)cc(C(F)(F)F)c2)c1-c1ccccc1C(C)(C)C]

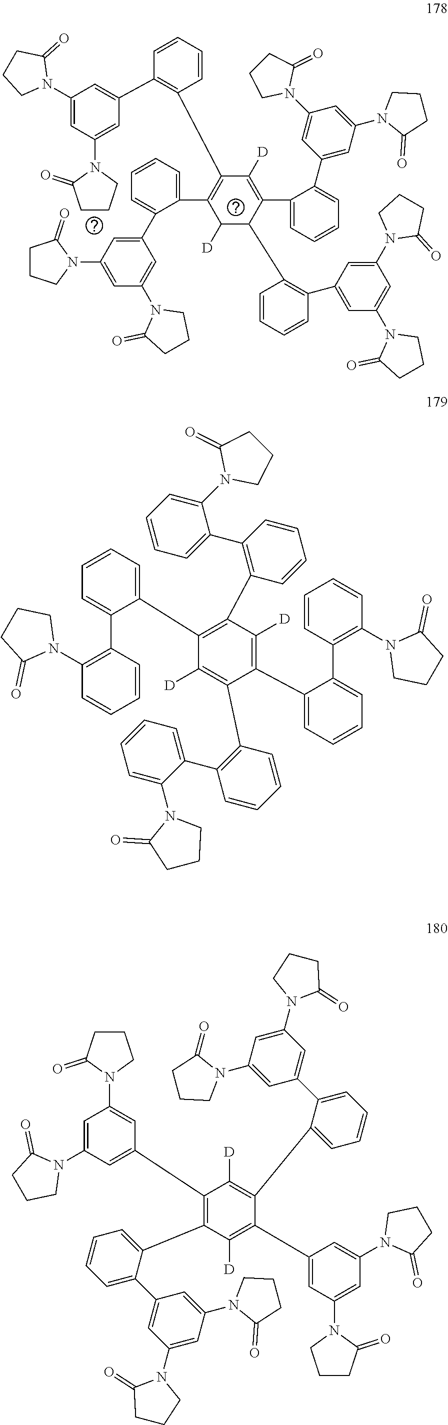 [SMILES: [2H]C1=C(c2ccccc2-c2cc(N3CCCC3=O)cc(N3CCCC3=O)c2)C(c2ccccc2-c2cc(N3CCCC3=O)cc(N3CCCC3=O)c2)C([2H])C(c2ccccc2-c2cc(N3CCCC3=O)cc(N3CCCC3=O)c2)=C1c1ccccc1-c1cc(N2CCCC2=O)cc(N2CCCC2=O)c1 ; [2H]c1c(-c2ccccc2-c2ccccc2N2CCCC2=O)c(-c2ccccc2-c2ccccc2N2CCCC2=O)c([2H])c(-c2ccccc2-c2ccccc2N2CCCC2=O)c1-c1ccccc1-c1ccccc1N1CCCC1=O ; [2H]c1c(-c2cc(N3CCCC3=O)cc(N3CCCC3=O)c2)c(-c2ccccc2-c2cc(N3CCCC3=O)cc(N3CCCC3=O)c2)c([2H])c(-c2cc(N3CCCC3=O)cc(N3CCCC3=O)c2)c1-c1ccccc1-c1cc(N2CCCC2=O)cc(N2CCCC2=O)c1]

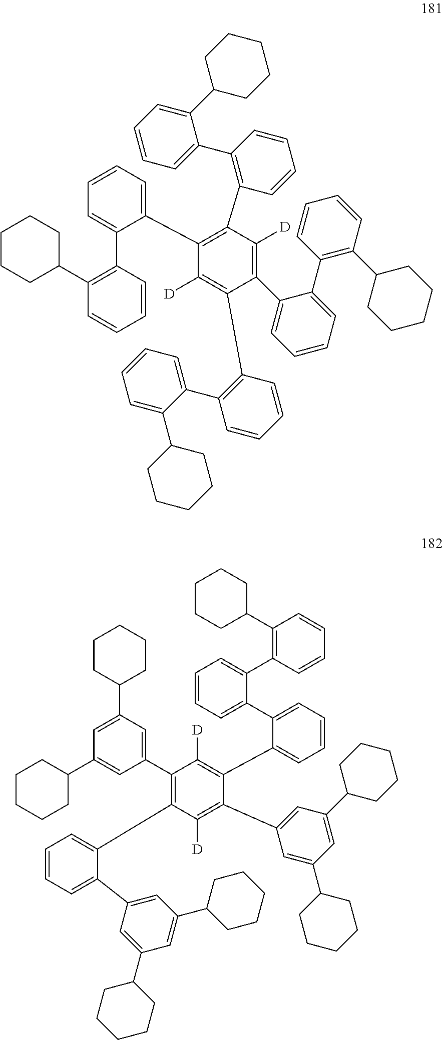 [SMILES: [2H]c1c(-c2ccccc2-c2ccccc2C2CCCCC2)c(-c2ccccc2-c2ccccc2C2CCCCC2)c([2H])c(-c2ccccc2-c2ccccc2C2CCCCC2)c1-c1ccccc1-c1ccccc1C1CCCCC1 ; [2H]c1c(-c2cc(C3CCCCC3)cc(C3CCCCC3)c2)c(-c2ccccc2-c2ccccc2-c2ccccc2C2CCCCC2)c([2H])c(-c2cc(C3CCCCC3)cc(C3CCCCC3)c2)c1-c1ccccc1-c1cc(C2CCCCC2)cc(C2CCCCC2)c1]

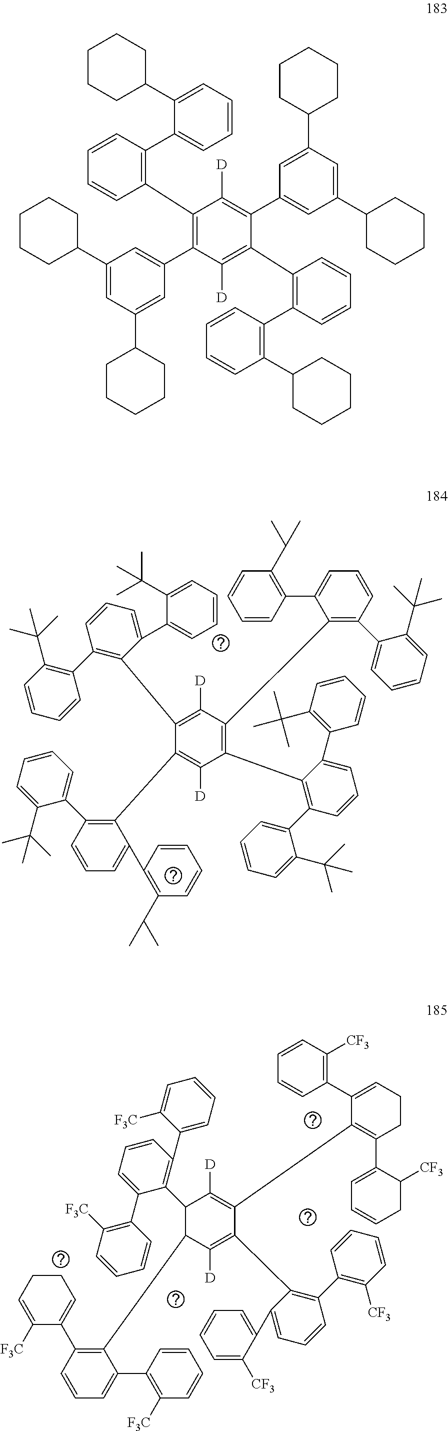

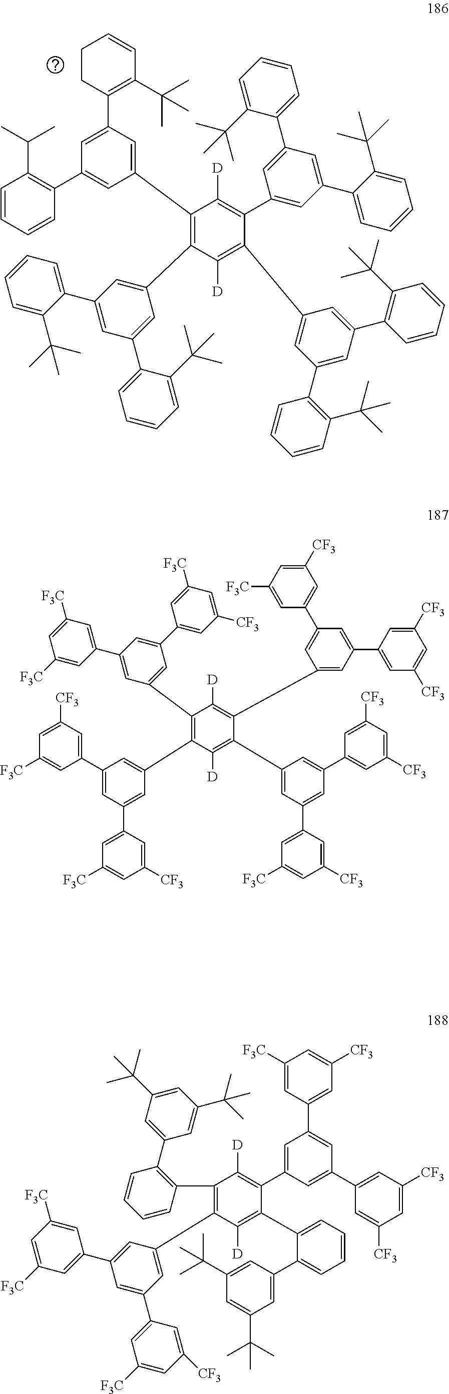

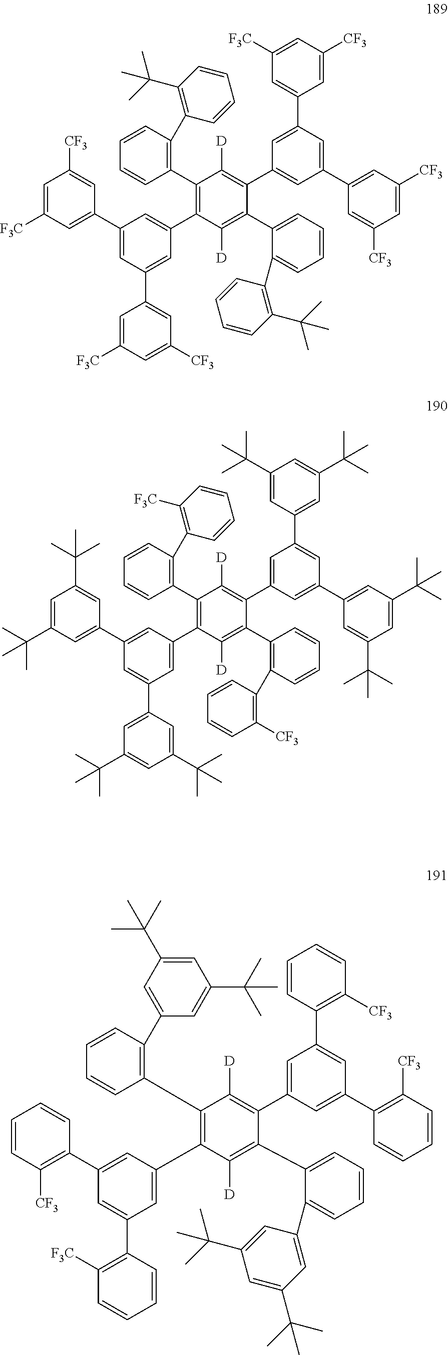

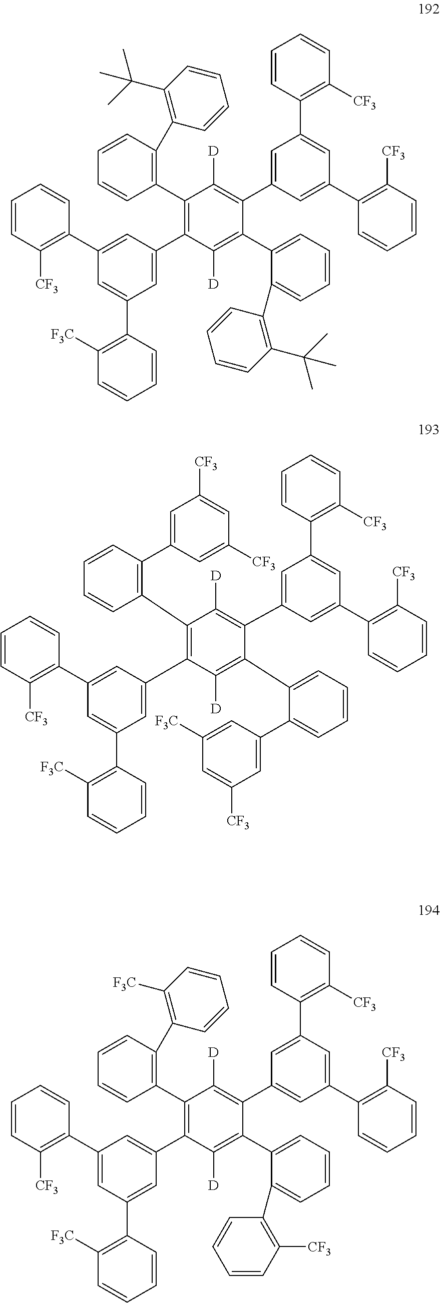

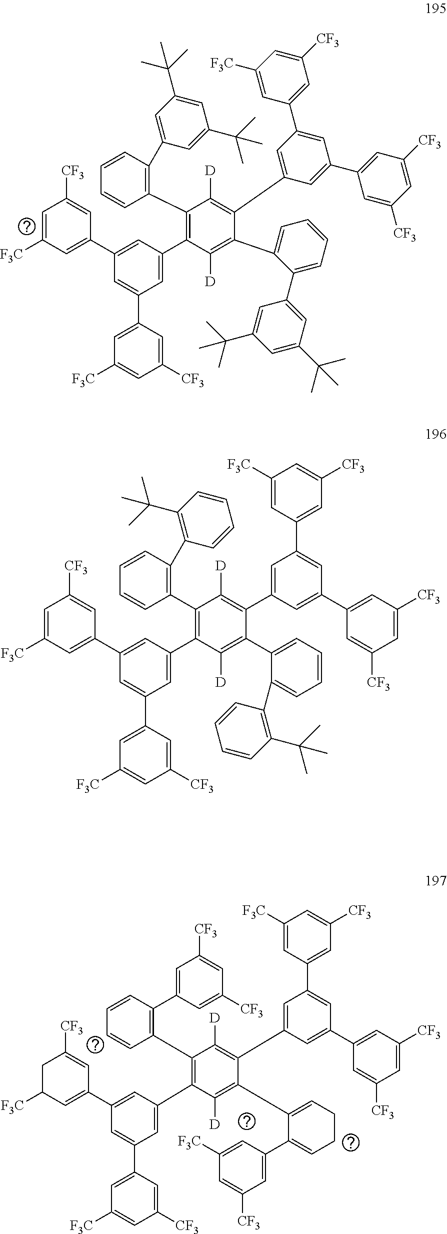

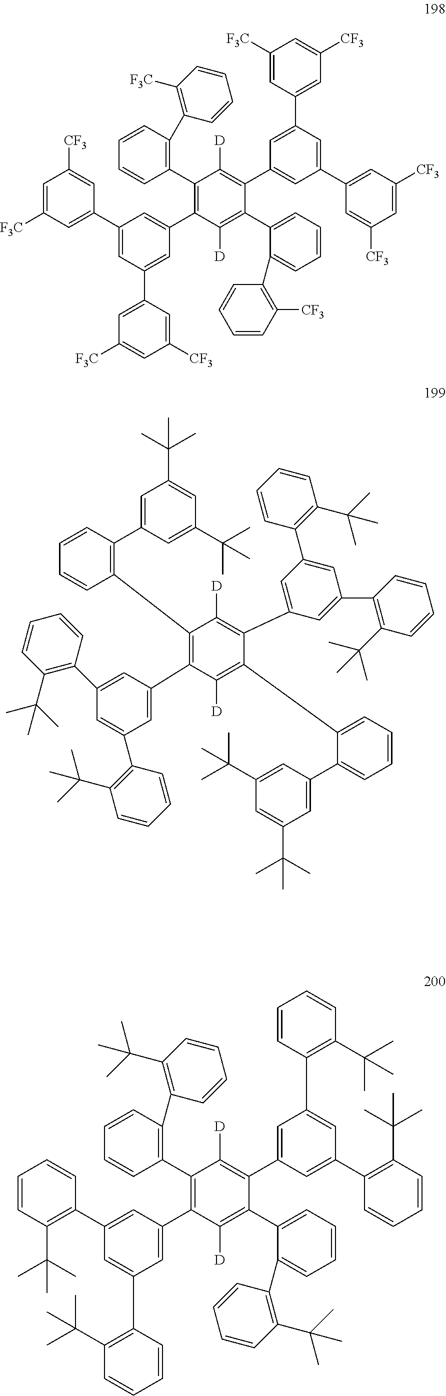

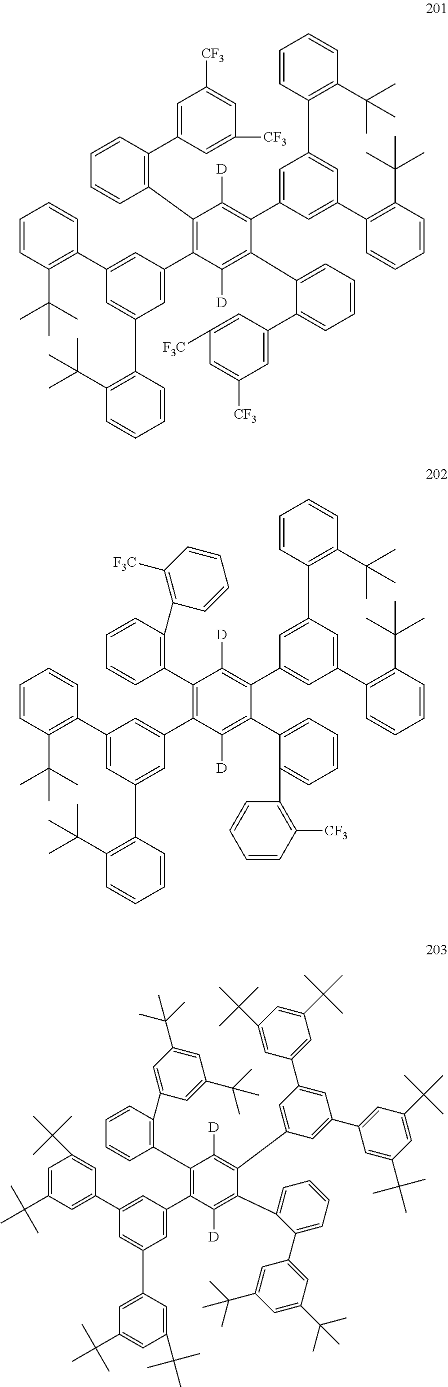

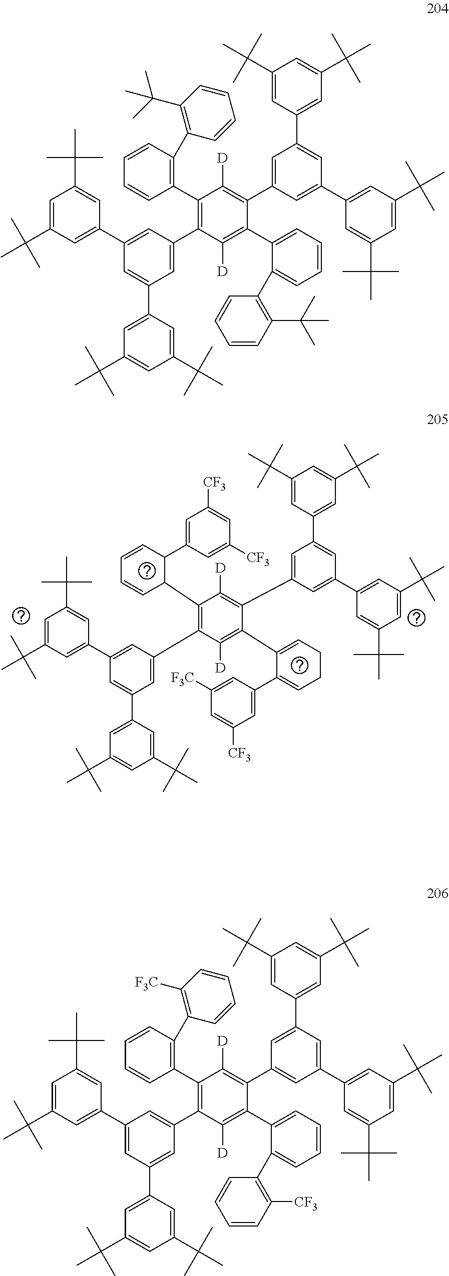

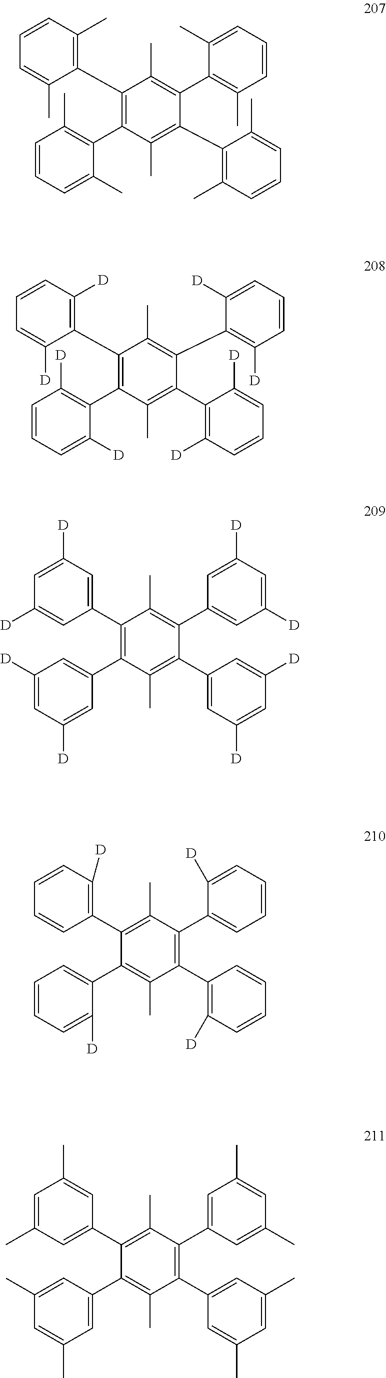

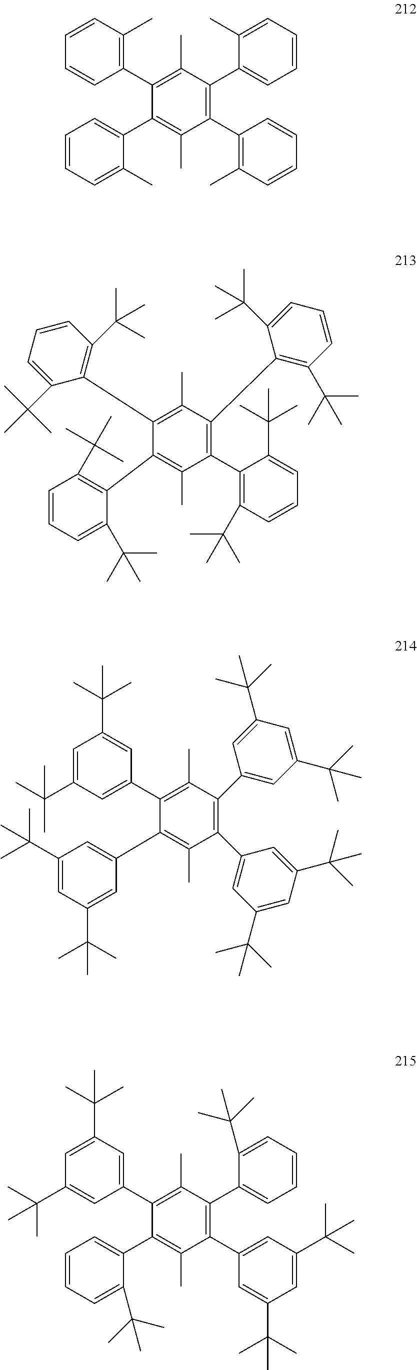

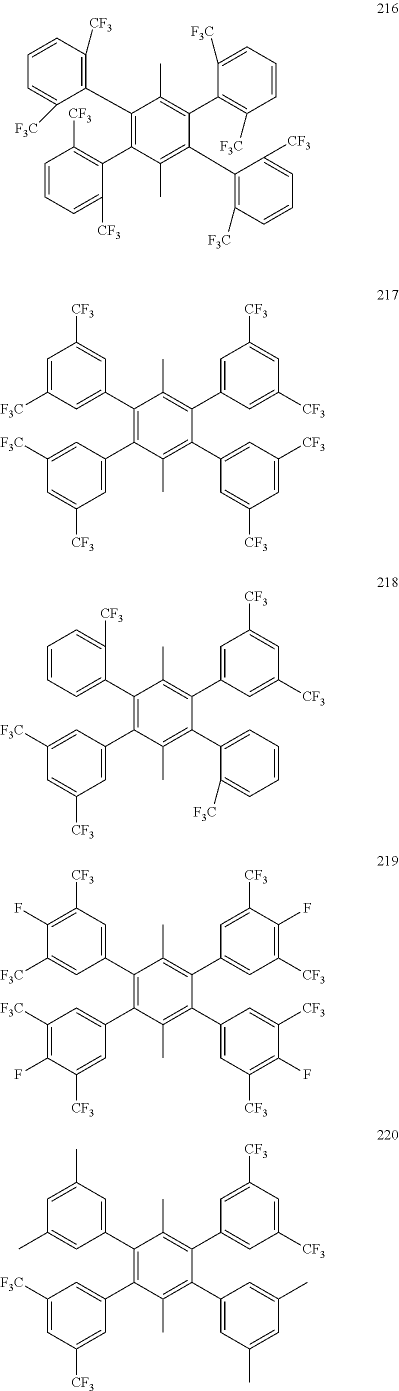

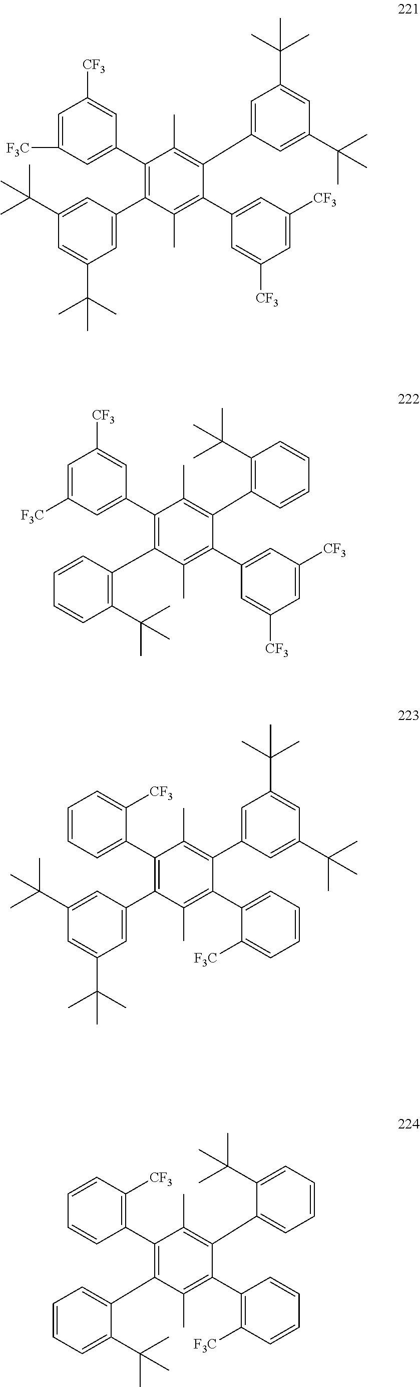

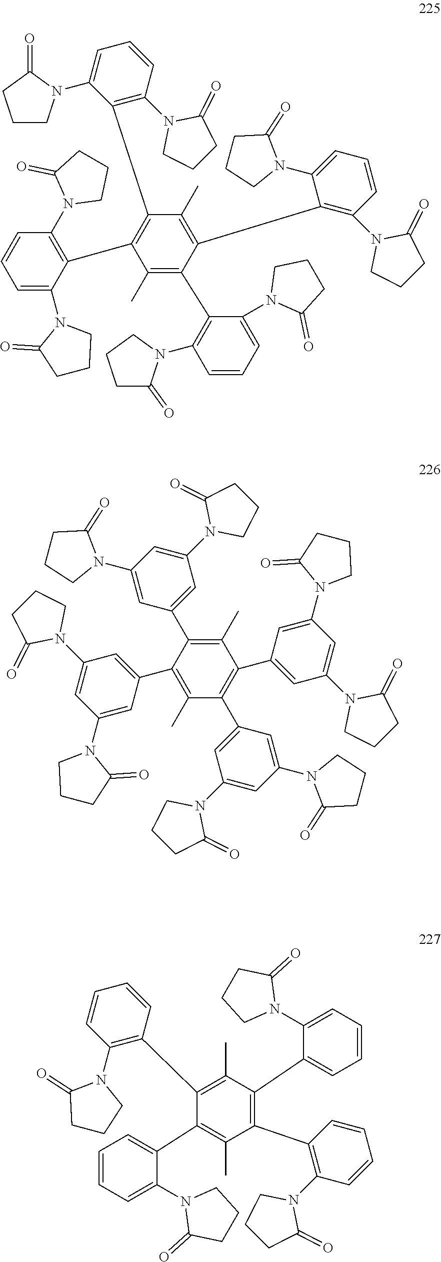 [SMILES: Cc1c(-c2c(N3CCCC3=O)cccc2N2CCCC2=O)c(-c2c(N3CCCC3=O)cccc2N2CCCC2=O)c(C)c(-c2c(N3CCCC3=O)cccc2N2CCCC2=O)c1-c1c(N2CCCC2=O)cccc1N1CCCC1=O ; Cc1c(-c2cc(N3CCCC3=O)cc(N3CCCC3=O)c2)c(-c2cc(N3CCCC3=O)cc(N3CCCC3=O)c2)c(C)c(-c2cc(N3CCCC3=O)cc(N3CCCC3=O)c2)c1-c1cc(N2CCCC2=O)cc(N2CCCC2=O)c1 ; Cc1c(-c2ccccc2N2CCCC2=O)c(-c2ccccc2N2CCCC2=O)c(C)c(-c2ccccc2N2CCCC2=O)c1-c1ccccc1N1CCCC1=O]

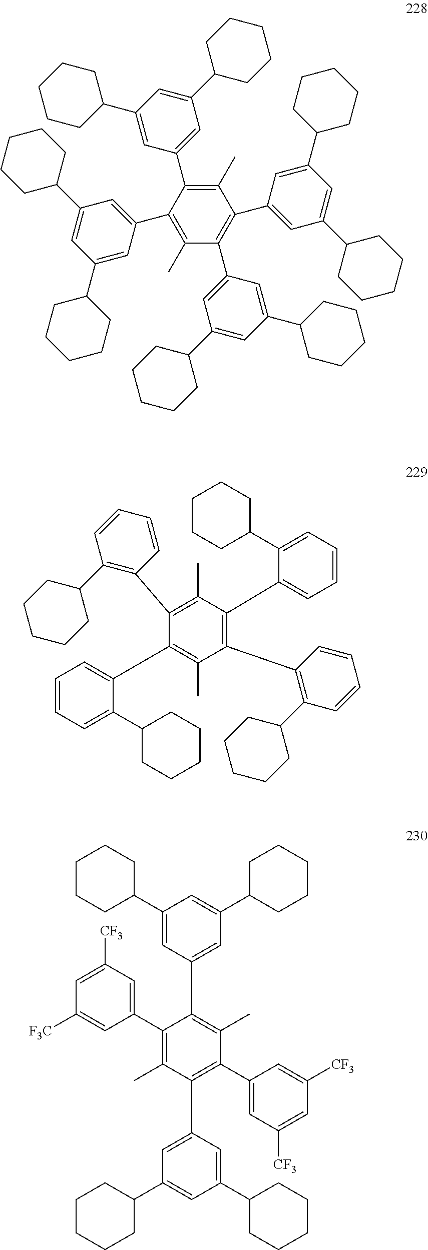

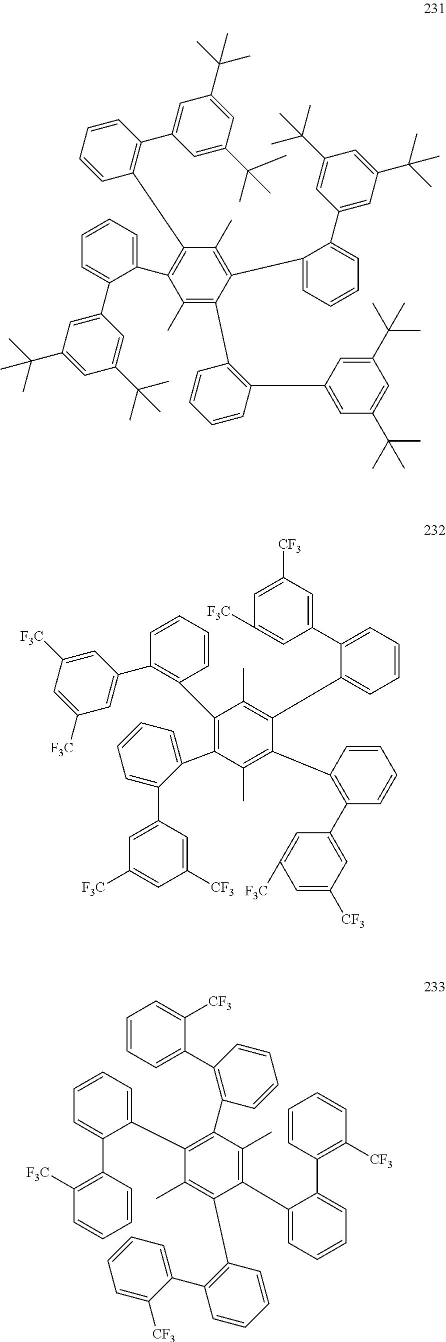

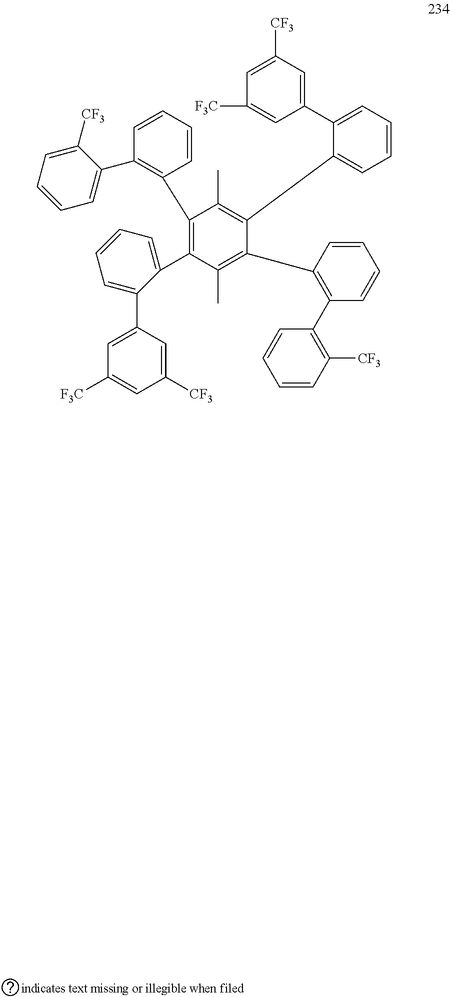 [SMILES: Cc1c(-c2ccccc2-c2cc(C(F)(F)F)cc(C(F)(F)F)c2)c(-c2ccccc2-c2ccccc2C(F)(F)F)c(C)c(-c2ccccc2-c2cc(C(F)(F)F)cc(C(F)(F)F)c2)c1-c1ccccc1-c1ccccc1C(F)(F)F]

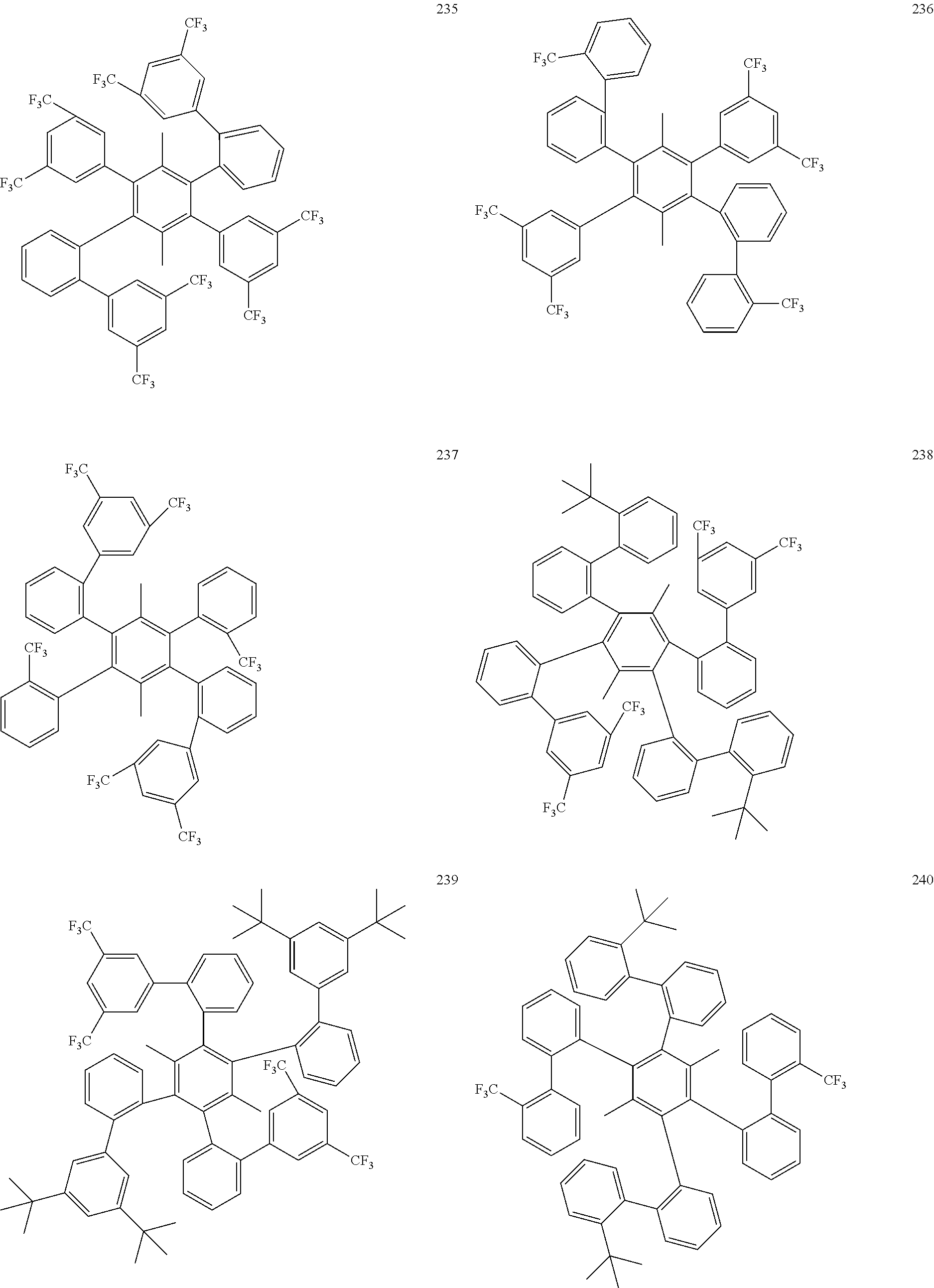

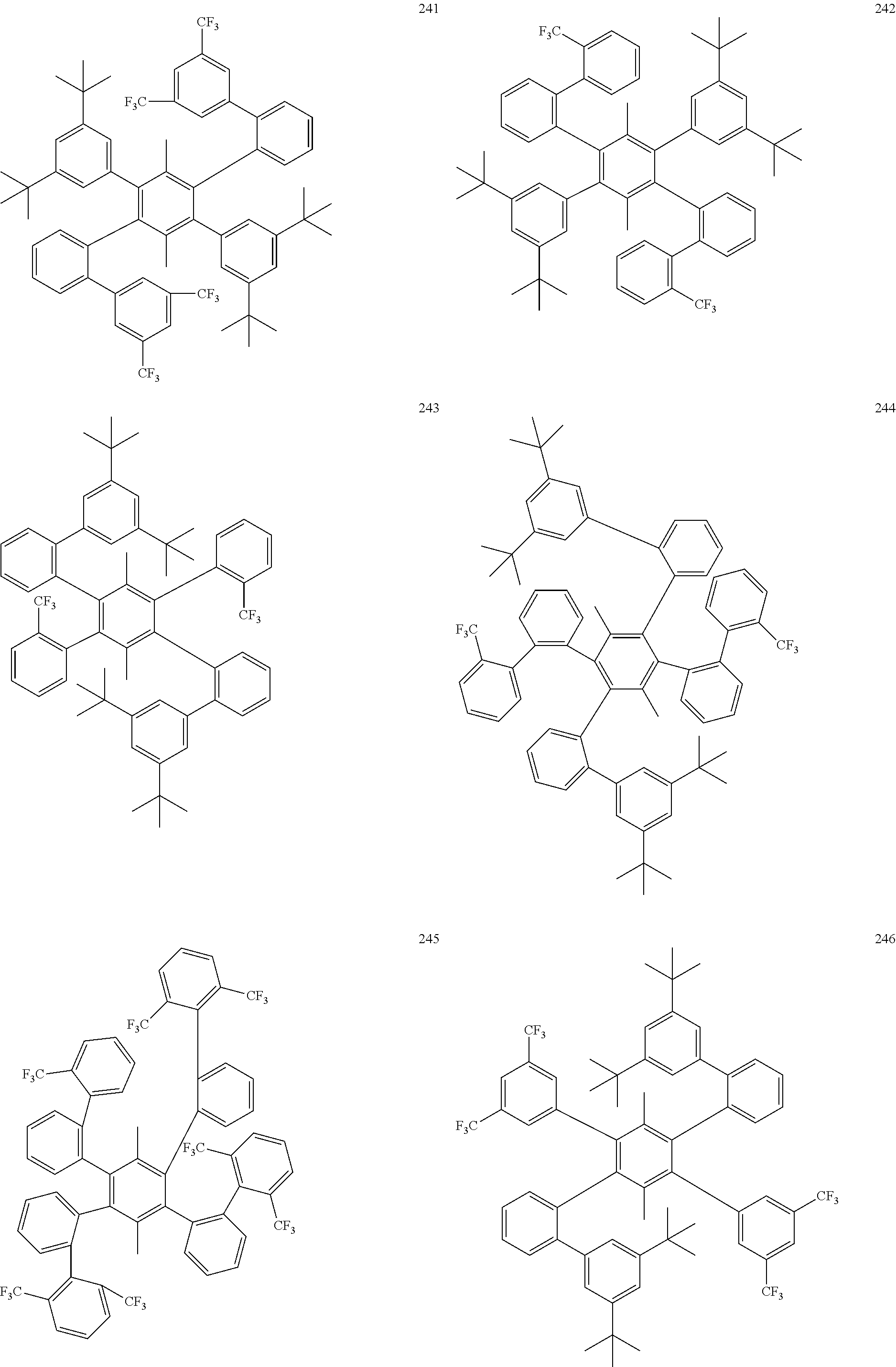

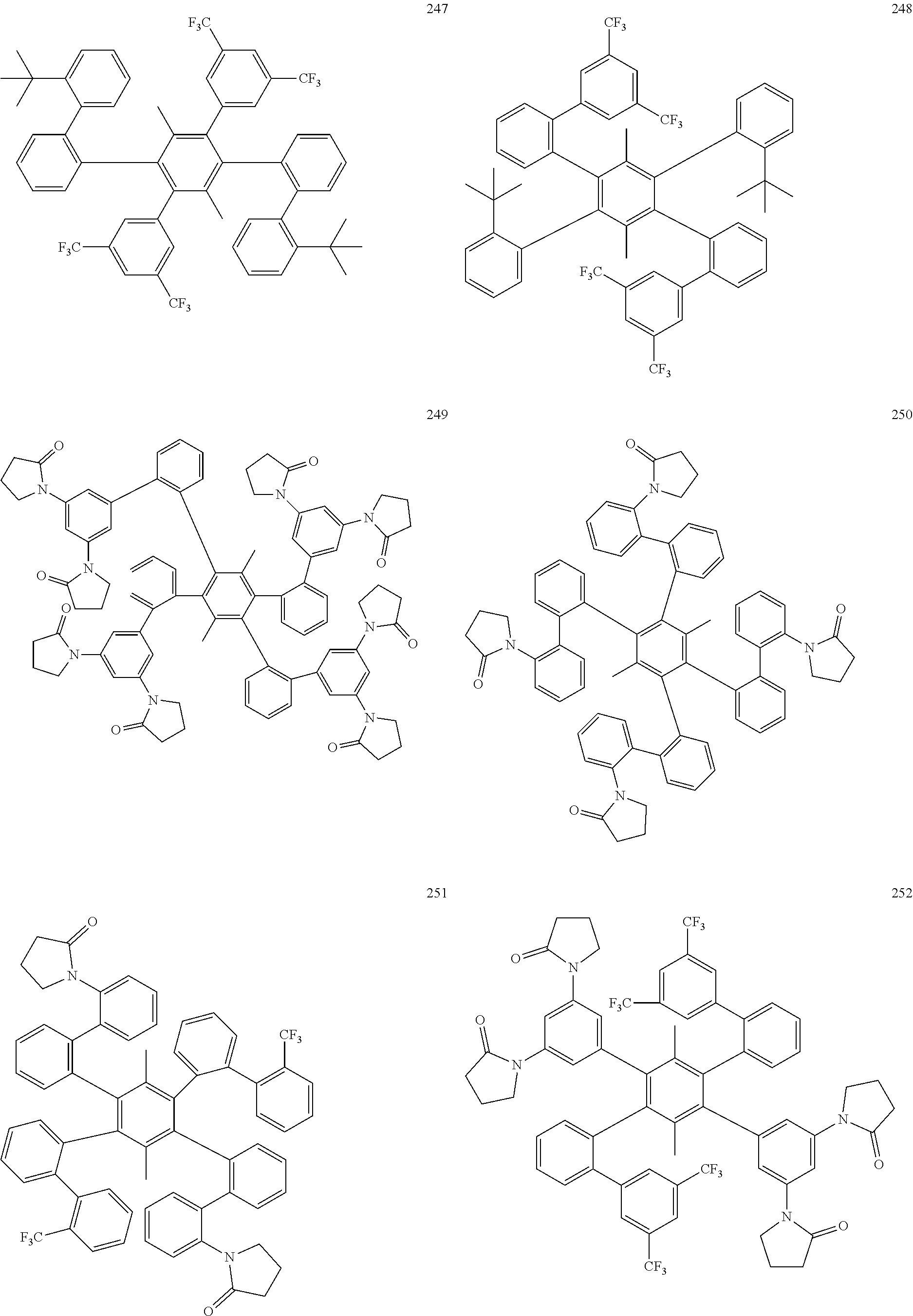

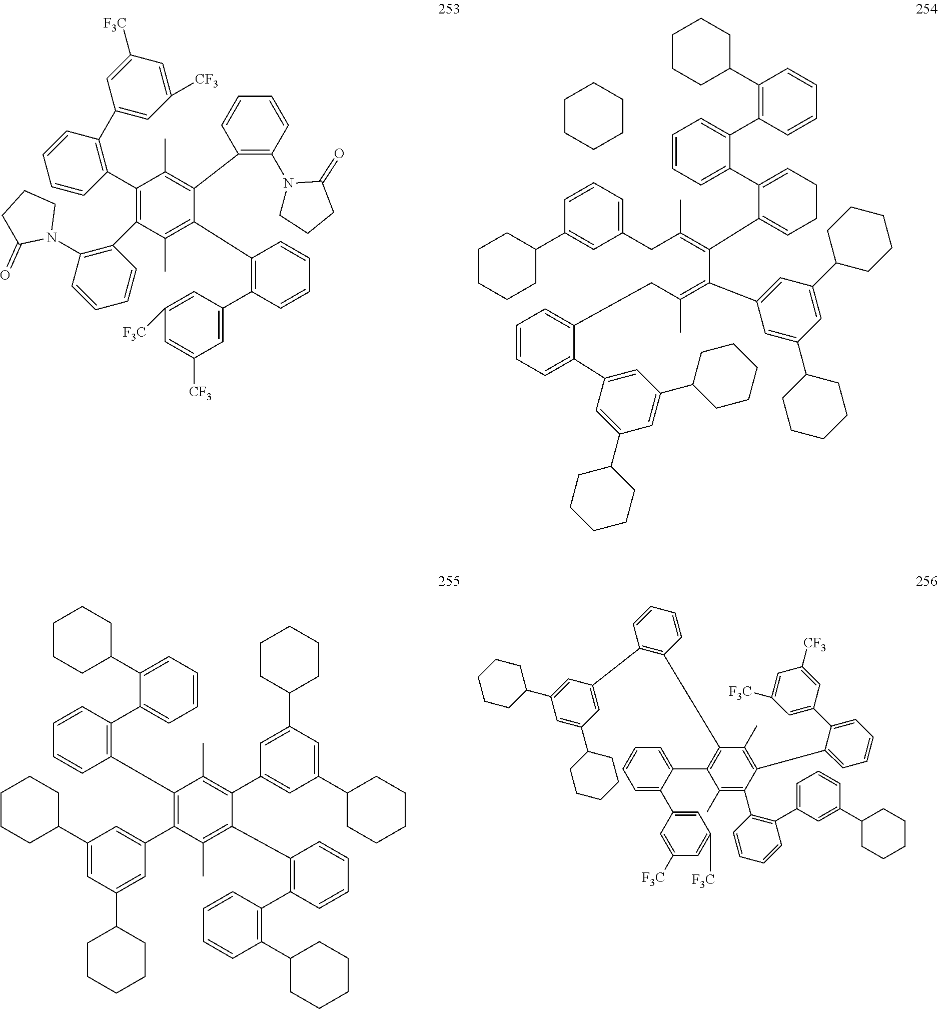

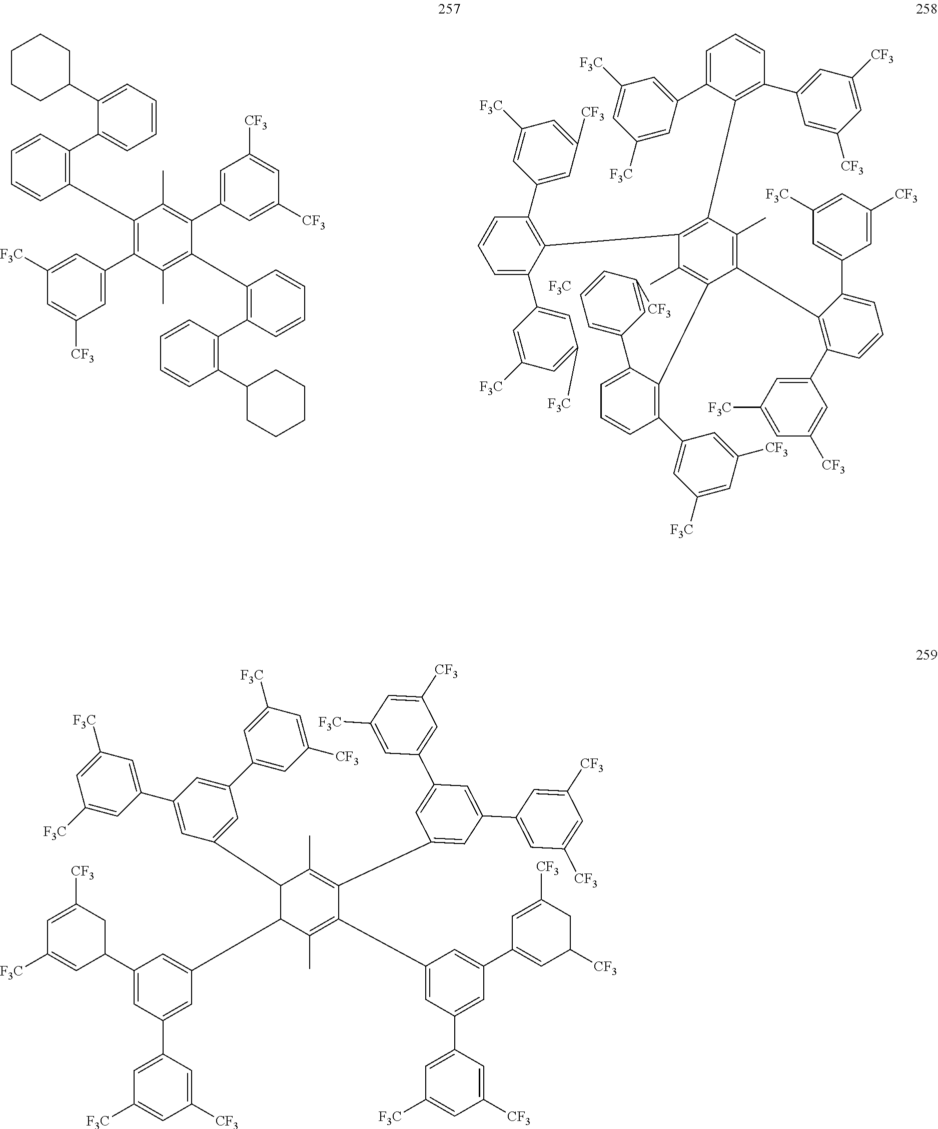

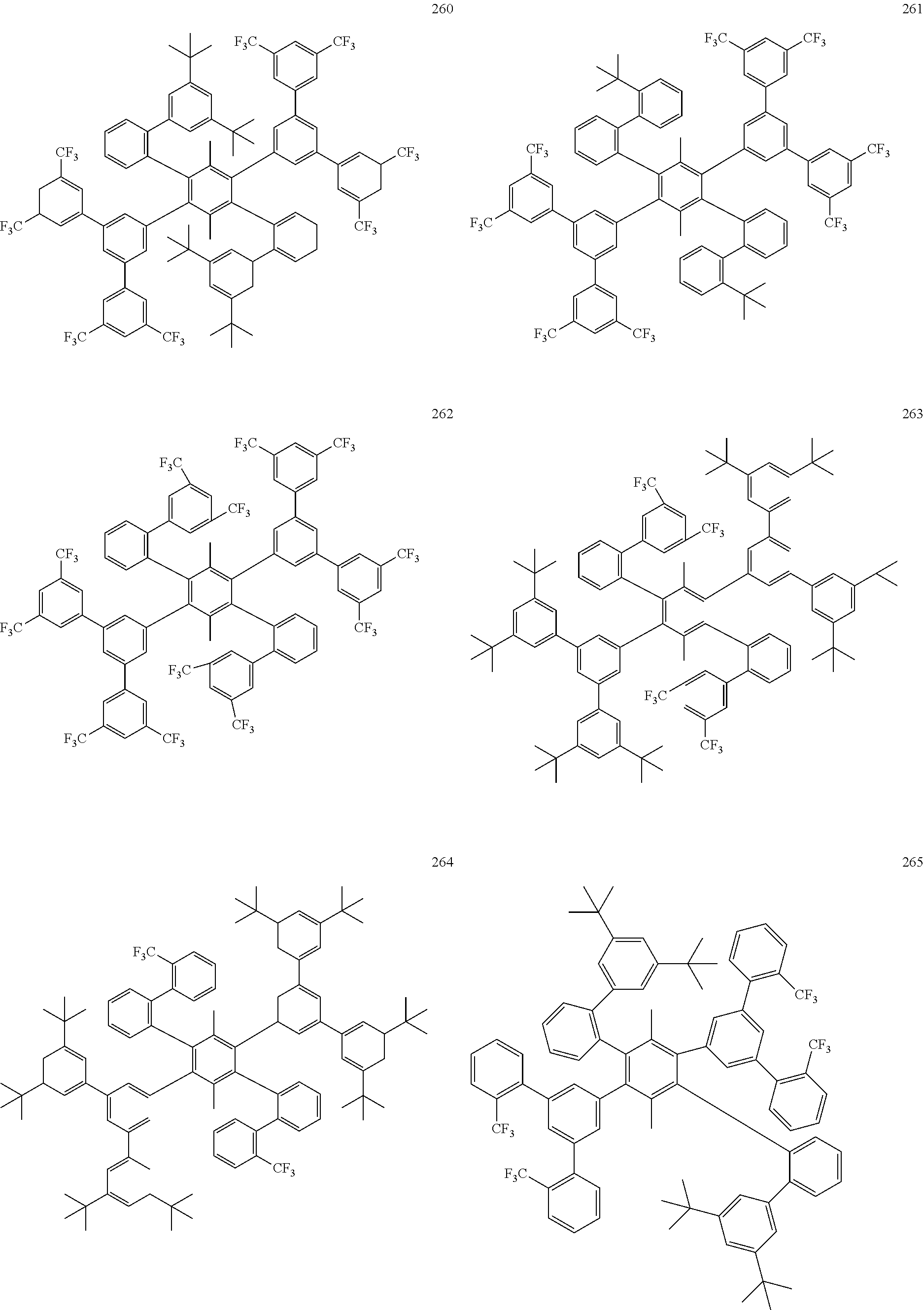

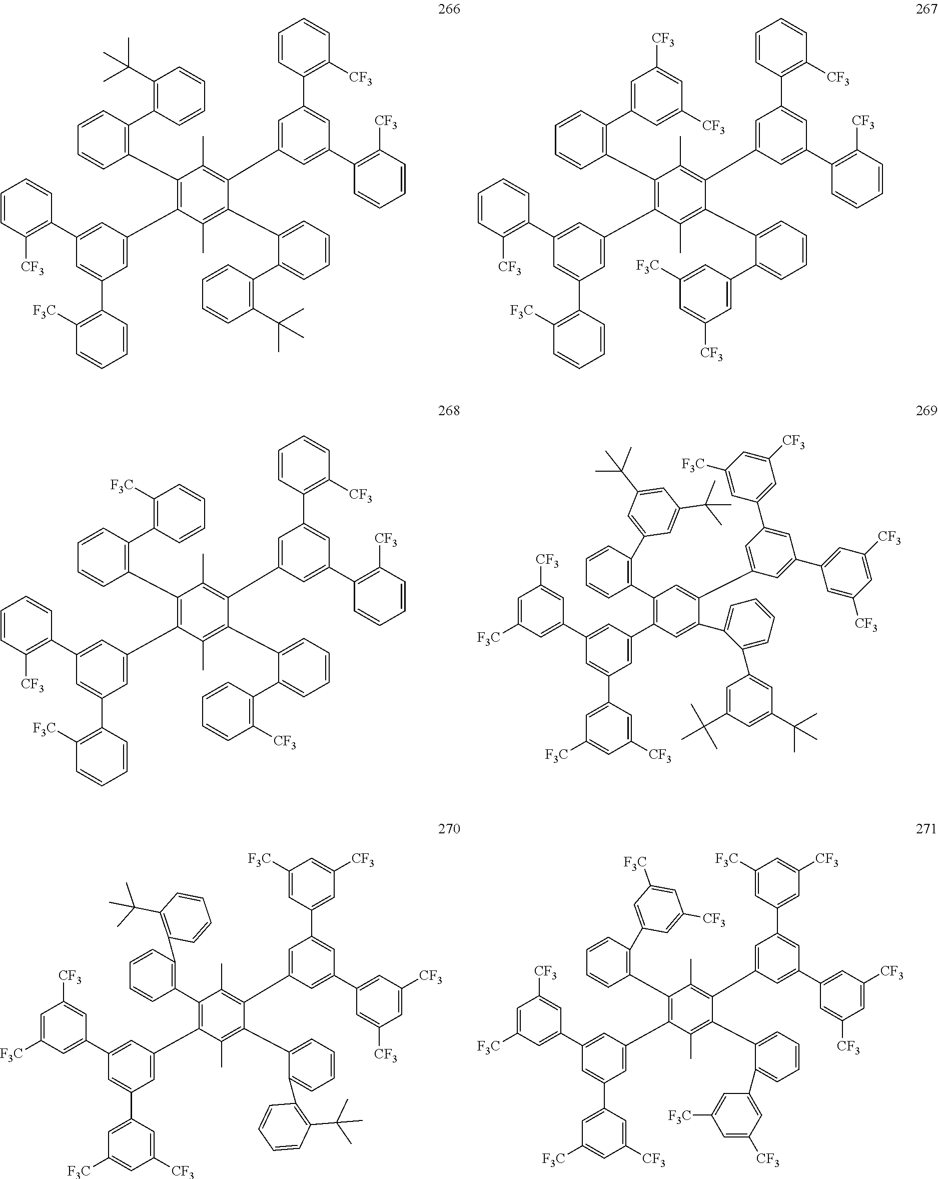

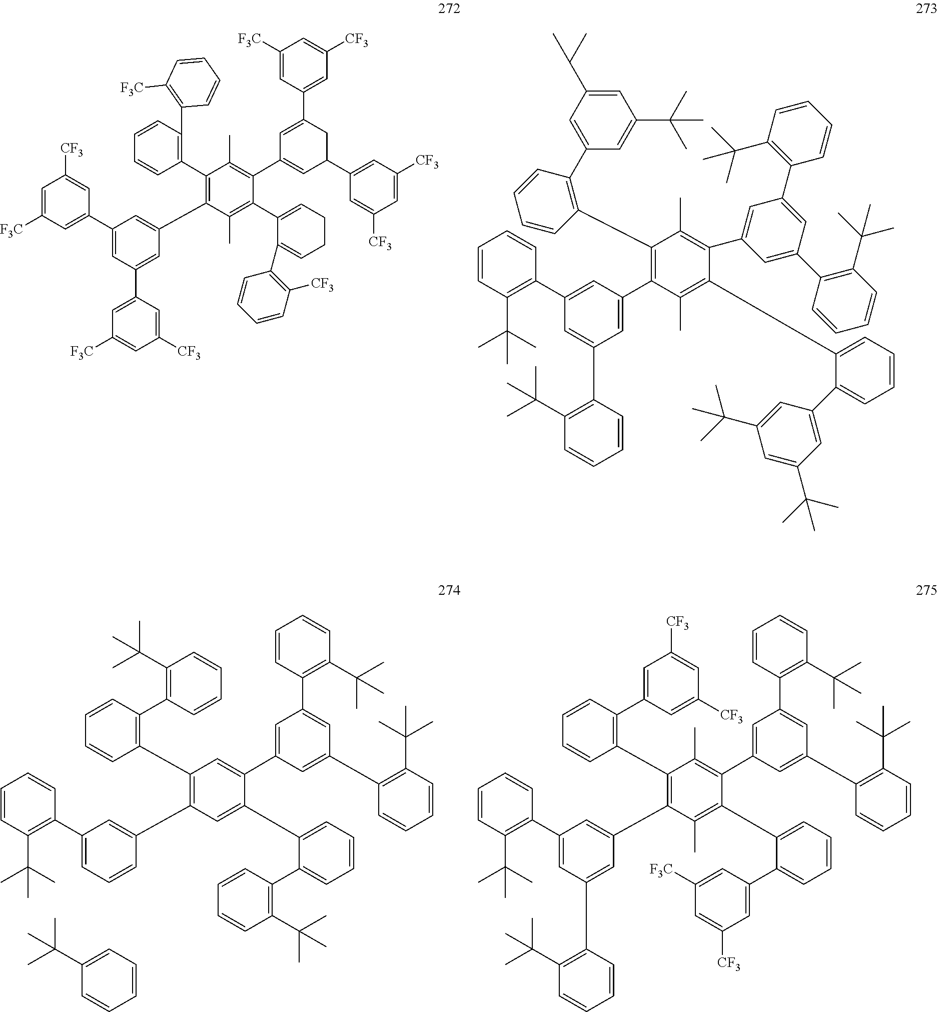

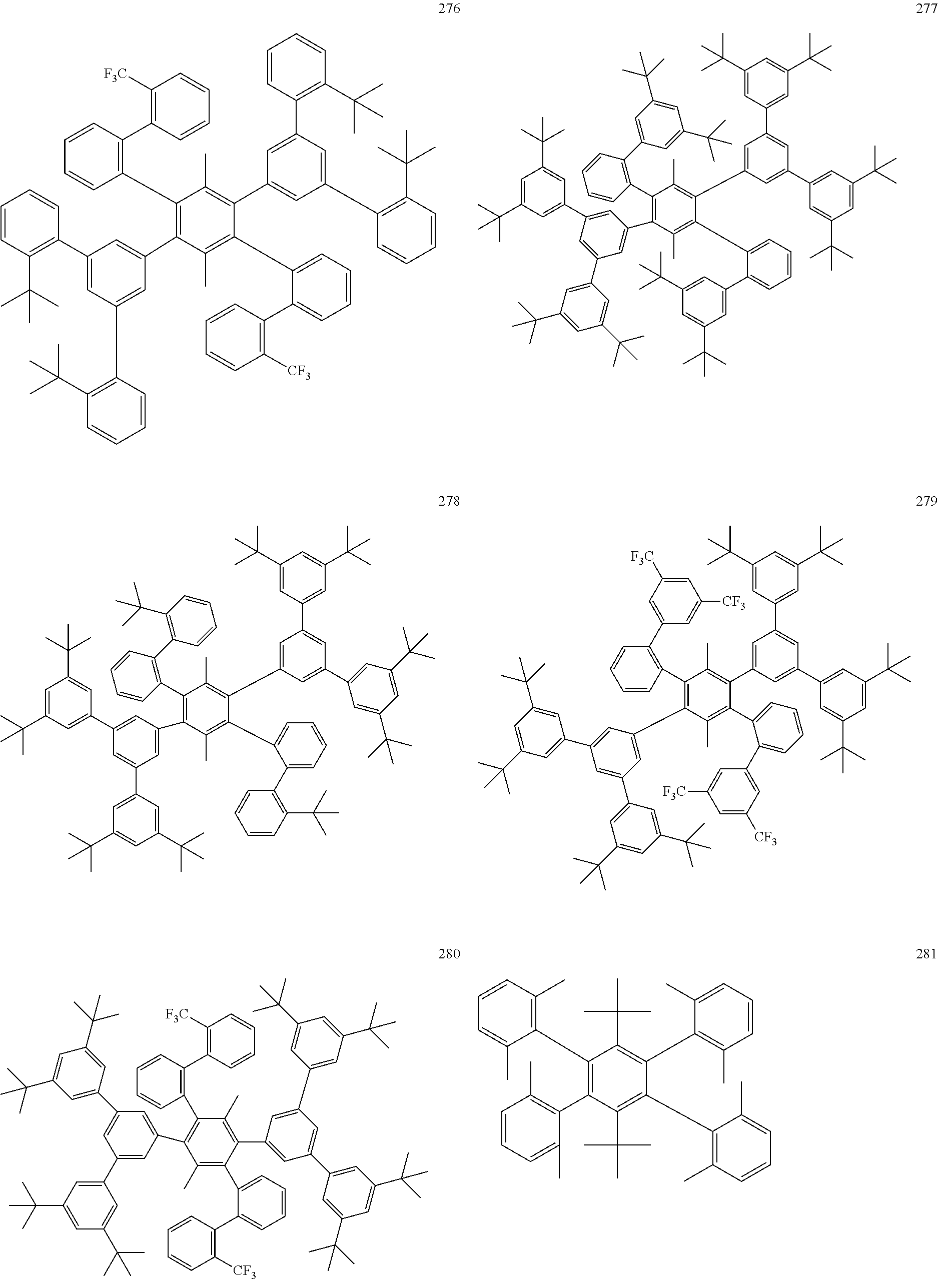

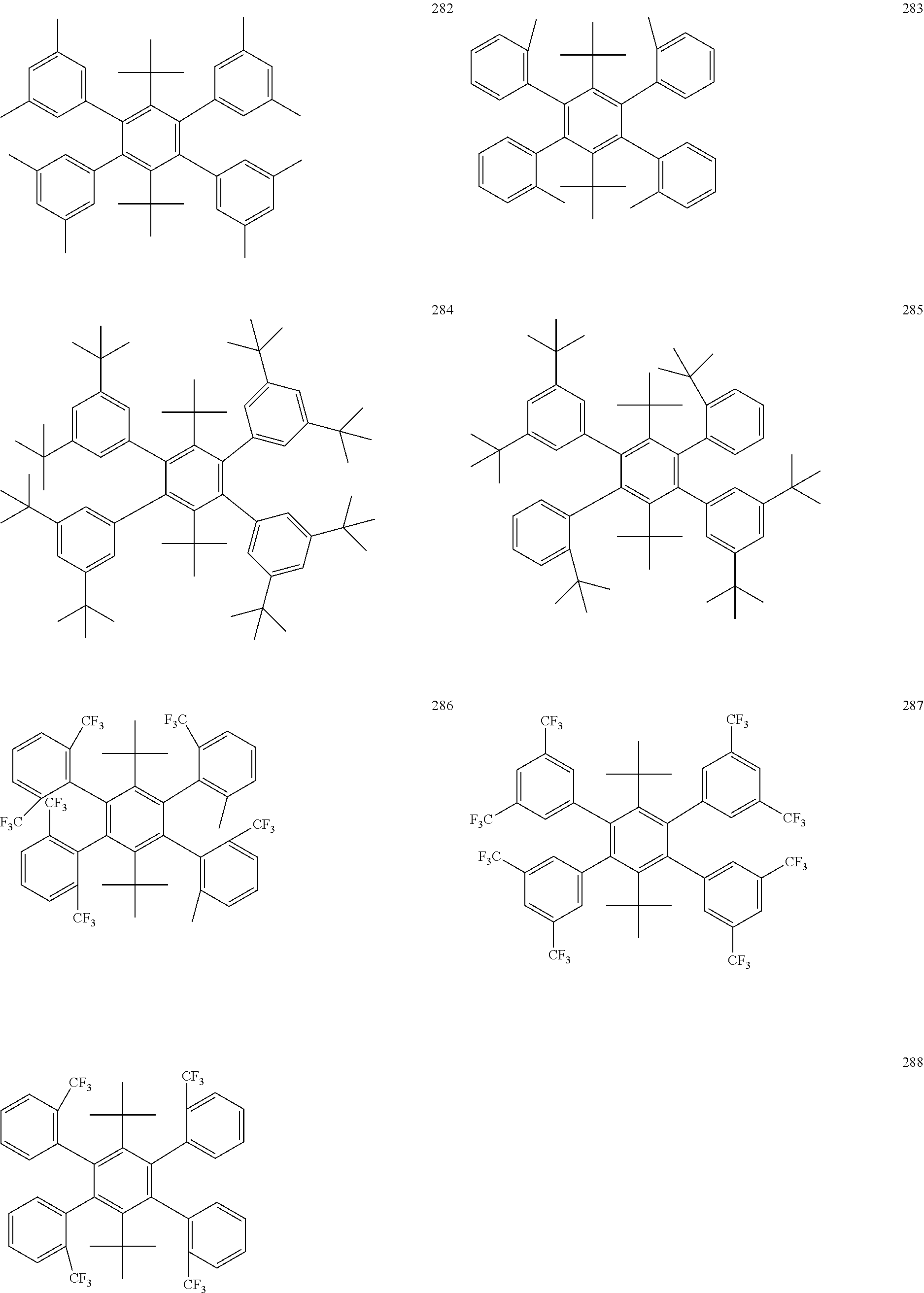

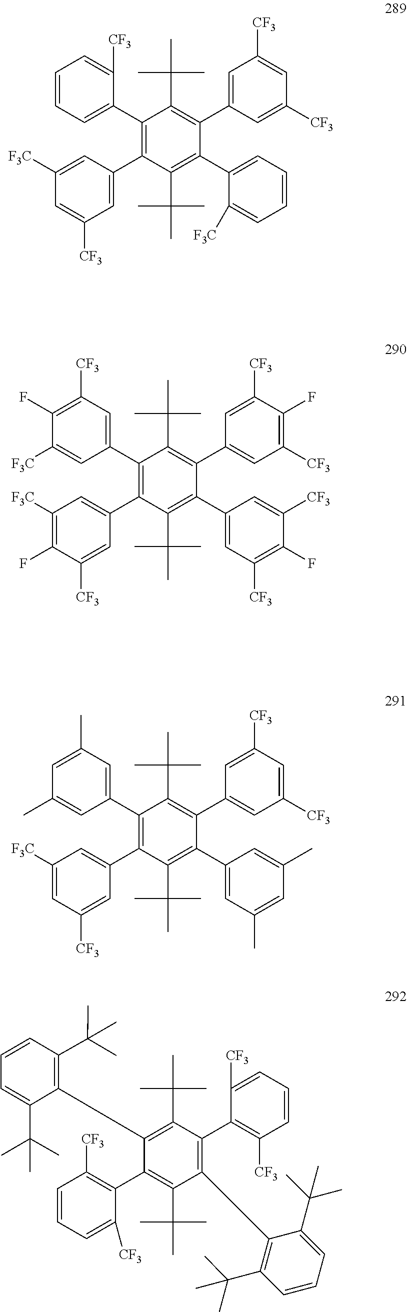

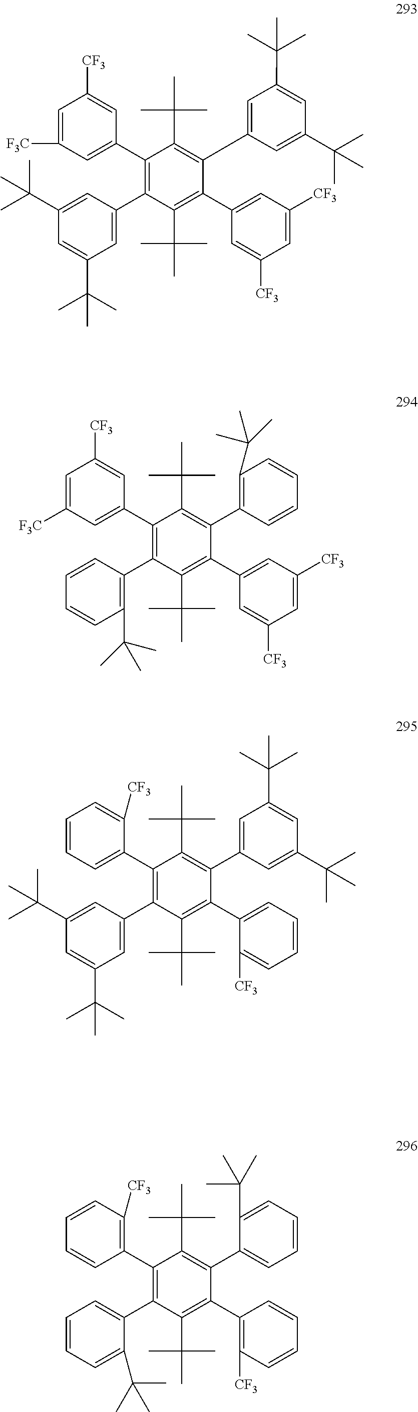

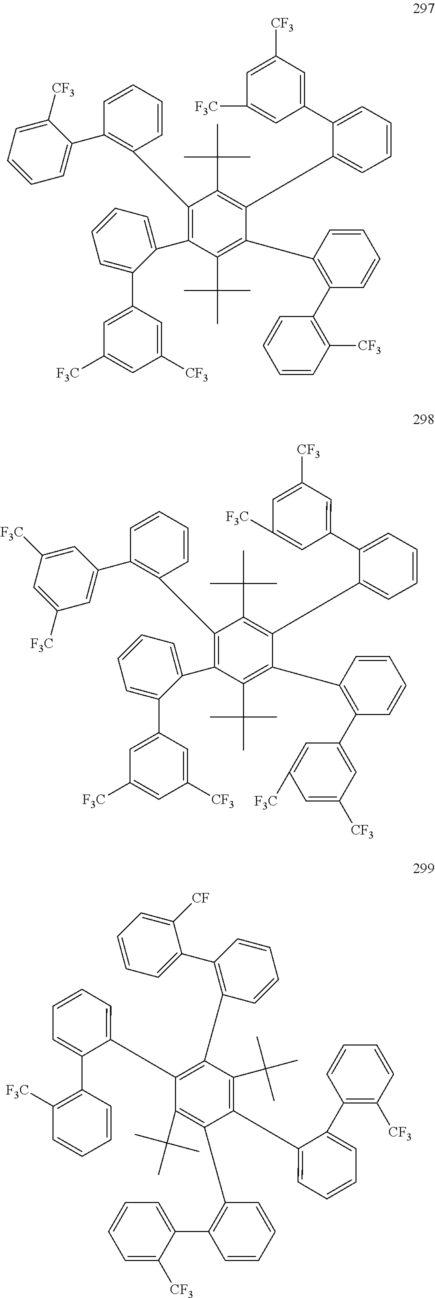

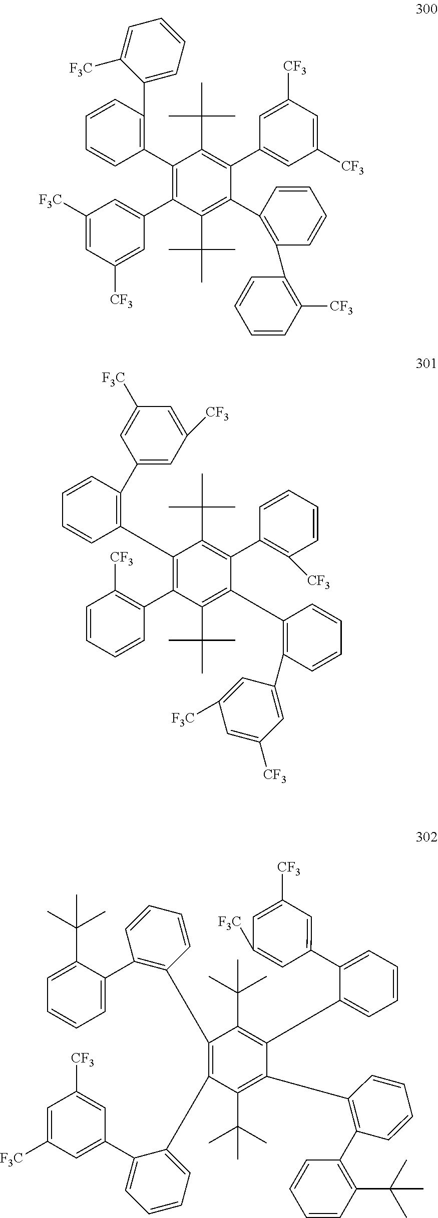 [SMILES: CC(C)(C)c1c(-c2cc(C(F)(F)F)cc(C(F)(F)F)c2)c(-c2ccccc2-c2ccccc2C(F)(F)F)c(C(C)(C)C)c(-c2cc(C(F)(F)F)cc(C(F)(F)F)c2)c1-c1ccccc1-c1ccccc1C(F)(F)F ; CC(C)(C)c1c(-c2ccccc2-c2cc(C(F)(F)F)cc(C(F)(F)F)c2)c(-c2ccccc2C(F)(F)F)c(C(C)(C)C)c(-c2ccccc2-c2cc(C(F)(F)F)cc(C(F)(F)F)c2)c1-c1ccccc1C(F)(F)F ; CC(C)(C)c1ccccc1-c1ccccc1-c1c(-c2ccccc2-c2cc(C(F)(F)F)cc(C(F)(F)F)c2)c(C(C)(C)C)c(-c2ccccc2-c2ccccc2C(C)(C)C)c(-c2ccccc2-c2cc(C(F)(F)F)cc(C(F)(F)F)c2)c1C(C)(C)C]

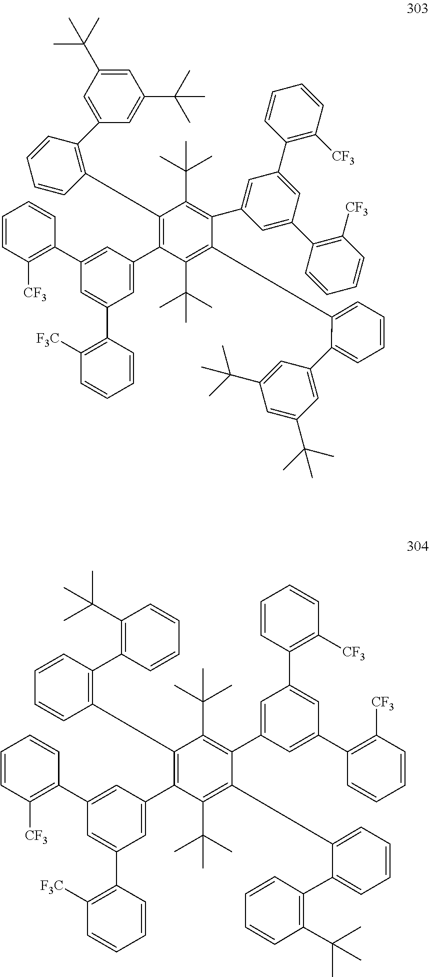 [SMILES: CC(C)(C)c1cc(-c2ccccc2-c2c(-c3cc(-c4ccccc4C(F)(F)F)cc(-c4ccccc4C(F)(F)F)c3)c(C(C)(C)C)c(-c3ccccc3-c3cc(C(C)(C)C)cc(C(C)(C)C)c3)c(-c3cc(-c4ccccc4C(F)(F)F)cc(-c4ccccc4C(F)(F)F)c3)c2C(C)(C)C)cc(C(C)(C)C)c1 ; CC(C)(C)c1ccccc1-c1ccccc1-c1c(-c2cc(-c3ccccc3C(F)(F)F)cc(-c3ccccc3C(F)(F)F)c2)c(C(C)(C)C)c(-c2ccccc2-c2ccccc2C(C)(C)C)c(-c2cc(-c3ccccc3C(F)(F)F)cc(-c3ccccc3C(F)(F)F)c2)c1C(C)(C)C]

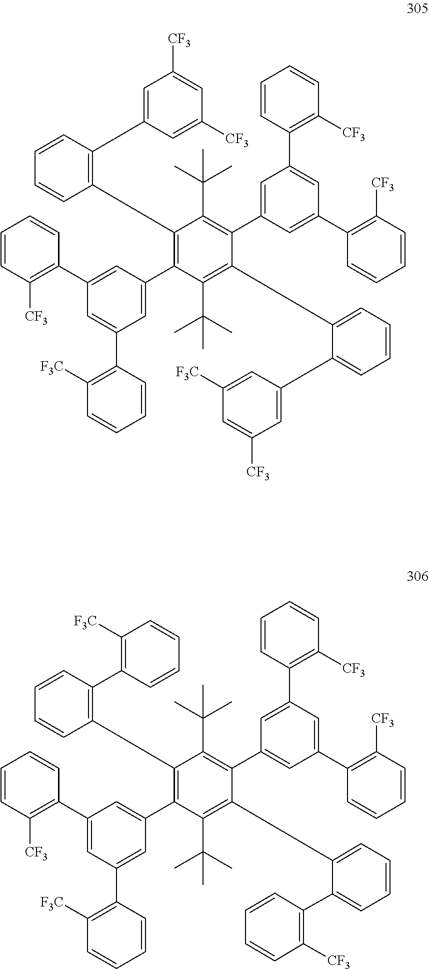 [SMILES: CC(C)(C)c1c(-c2cc(-c3ccccc3C(F)(F)F)cc(-c3ccccc3C(F)(F)F)c2)c(-c2ccccc2-c2cc(C(F)(F)F)cc(C(F)(F)F)c2)c(C(C)(C)C)c(-c2cc(-c3ccccc3C(F)(F)F)cc(-c3ccccc3C(F)(F)F)c2)c1-c1ccccc1-c1cc(C(F)(F)F)cc(C(F)(F)F)c1 ; CC(C)(C)c1c(-c2cc(-c3ccccc3C(F)(F)F)cc(-c3ccccc3C(F)(F)F)c2)c(-c2ccccc2-c2ccccc2C(F)(F)F)c(C(C)(C)C)c(-c2cc(-c3ccccc3C(F)(F)F)cc(-c3ccccc3C(F)(F)F)c2)c1-c1ccccc1-c1ccccc1C(F)(F)F]

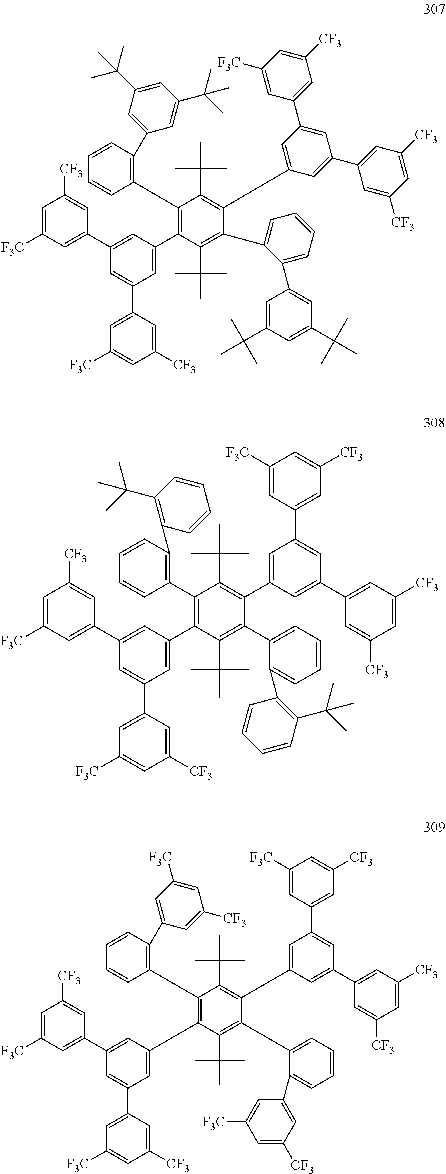

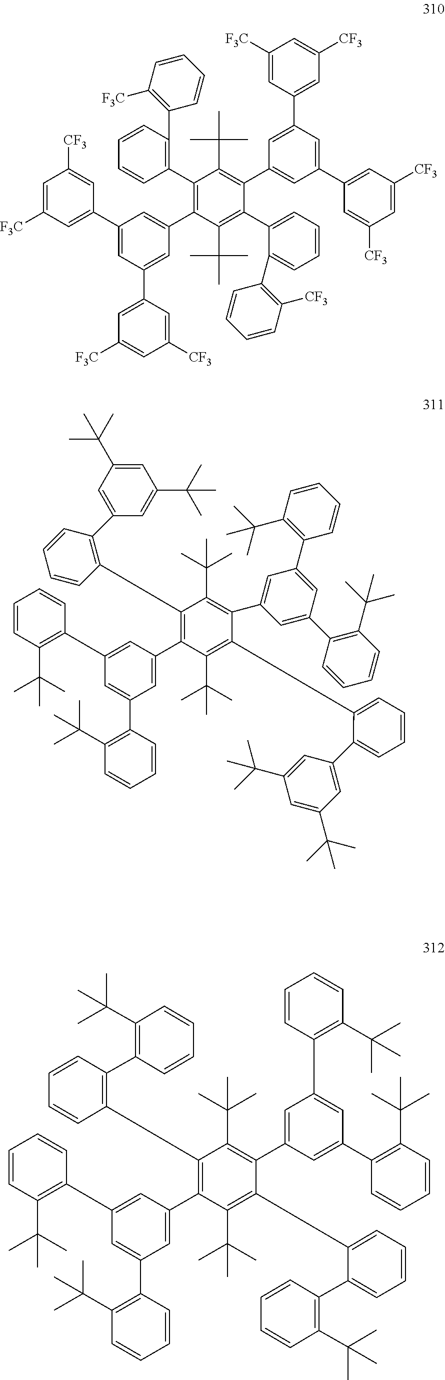

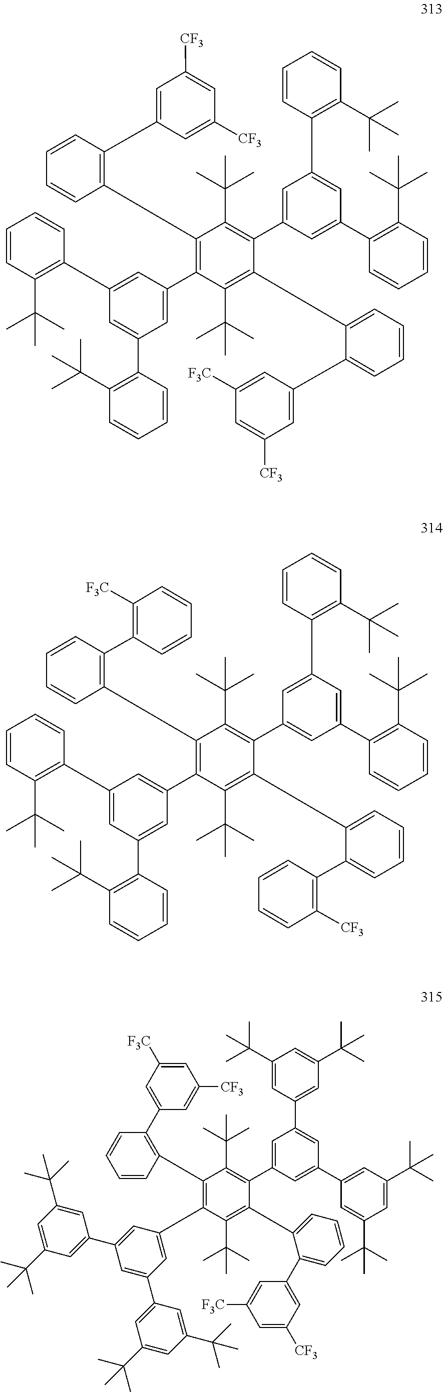

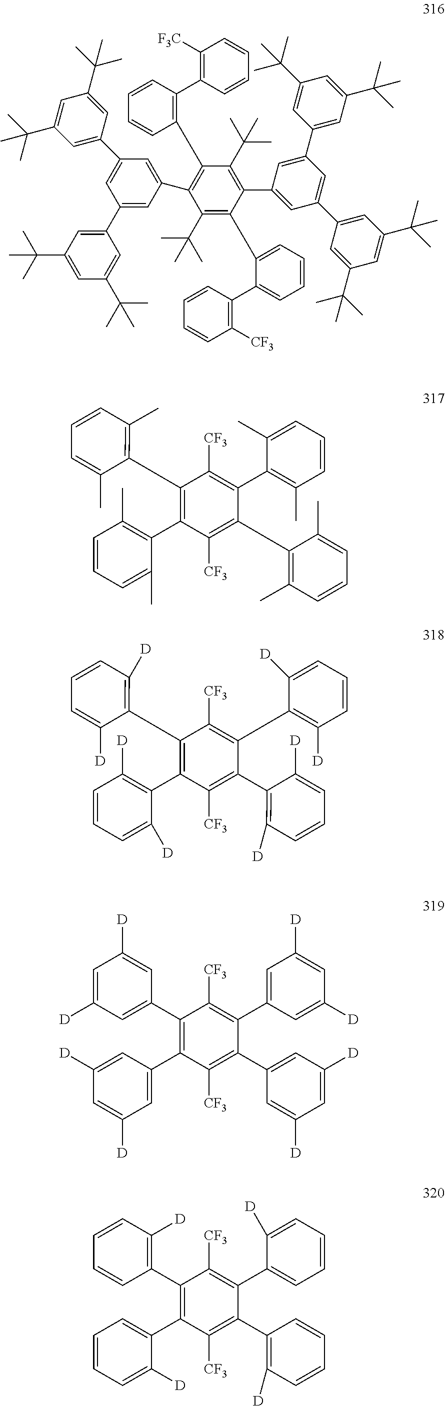

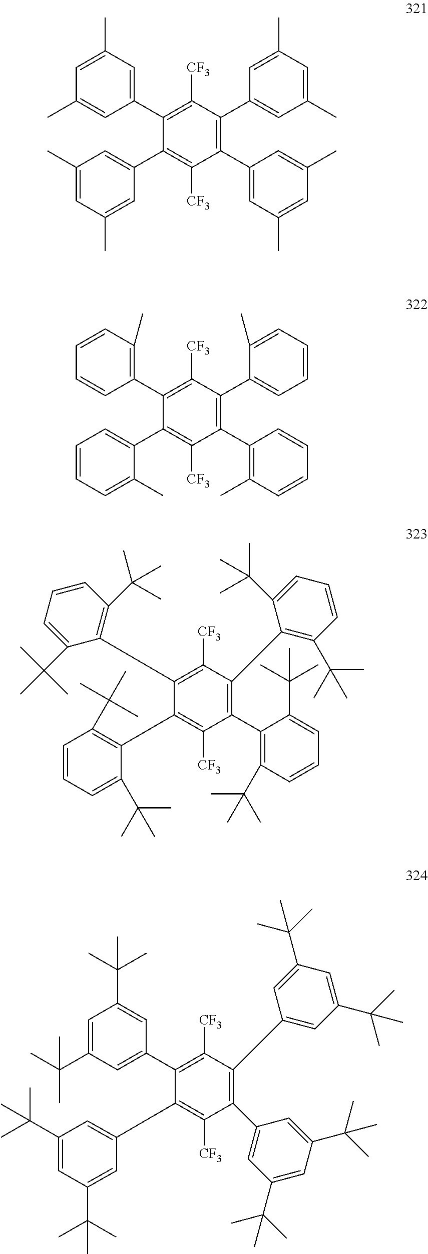

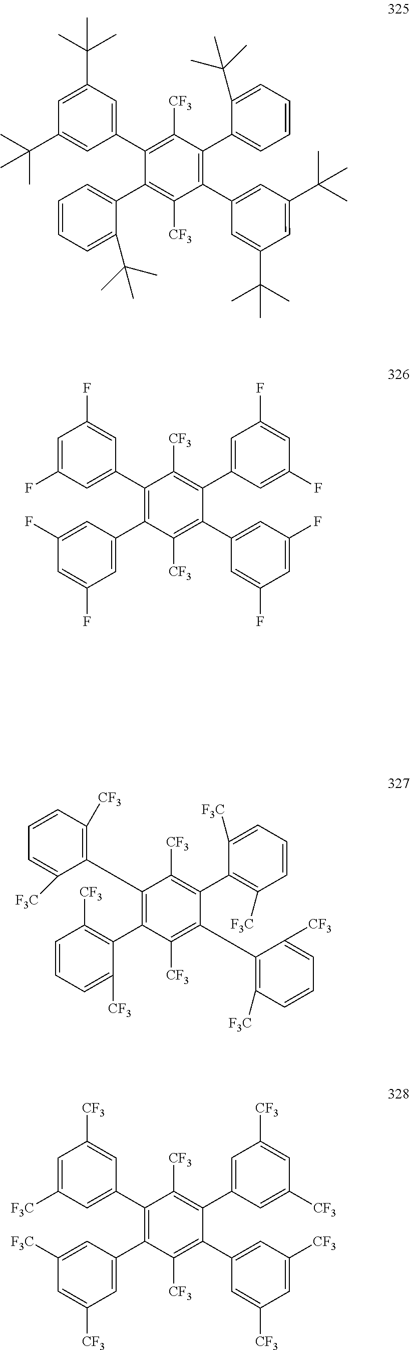

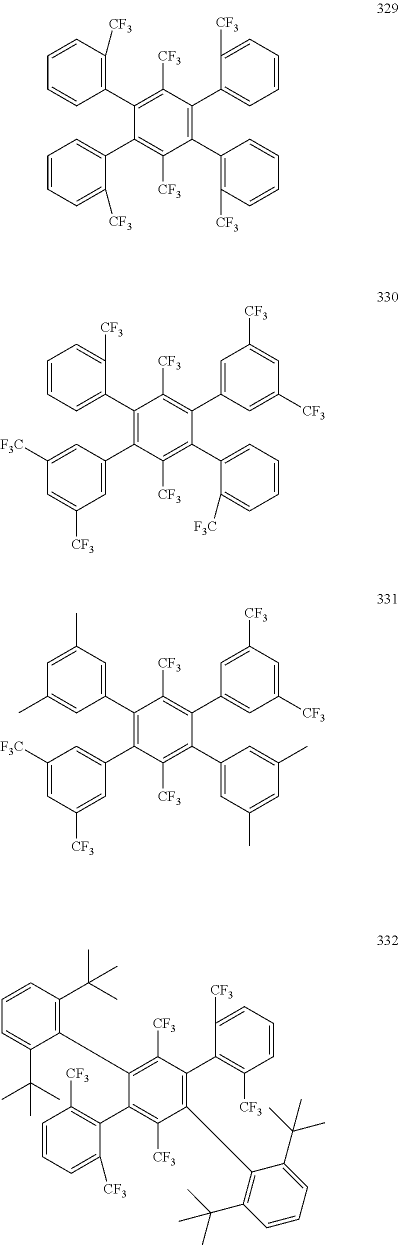 [SMILES: FC(F)(F)c1ccccc1-c1c(-c2ccccc2C(F)(F)F)c(C(F)(F)F)c(-c2ccccc2C(F)(F)F)c(-c2ccccc2C(F)(F)F)c1C(F)(F)F ; FC(F)(F)c1cc(-c2c(-c3ccccc3C(F)(F)F)c(C(F)(F)F)c(-c3cc(C(F)(F)F)cc(C(F)(F)F)c3)c(-c3ccccc3C(F)(F)F)c2C(F)(F)F)cc(C(F)(F)F)c1 ; Cc1cc(C)cc(-c2c(-c3cc(C(F)(F)F)cc(C(F)(F)F)c3)c(C(F)(F)F)c(-c3cc(C)cc(C)c3)c(-c3cc(C(F)(F)F)cc(C(F)(F)F)c3)c2C(F)(F)F)c1 ; CC(C)(C)c1cccc(C(C)(C)C)c1-c1c(-c2c(C(F)(F)F)cccc2C(F)(F)F)c(C(F)(F)F)c(-c2c(C(C)(C)C)cccc2C(C)(C)C)c(-c2c(C(F)(F)F)cccc2C(F)(F)F)c1C(F)(F)F]

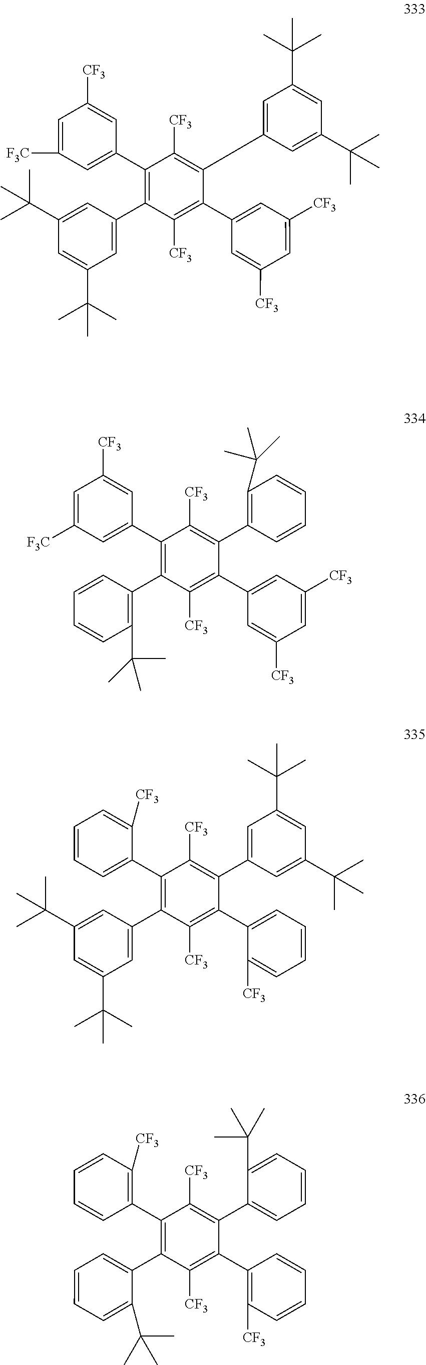

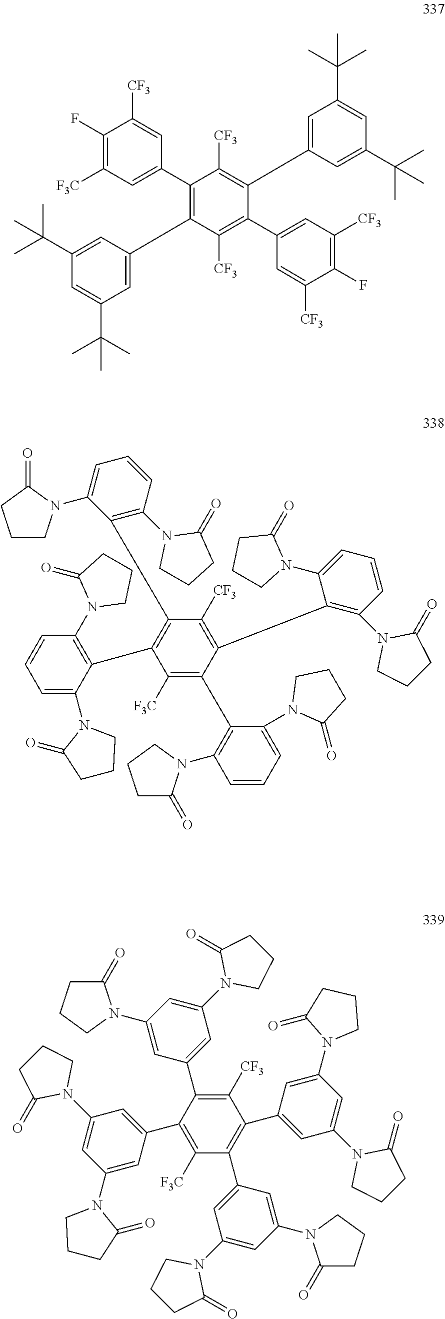 [SMILES: CC(C)(C)c1cc(-c2c(-c3cc(C(F)(F)F)c(F)c(C(F)(F)F)c3)c(C(F)(F)F)c(-c3cc(C(C)(C)C)cc(C(C)(C)C)c3)c(-c3cc(C(F)(F)F)c(F)c(C(F)(F)F)c3)c2C(F)(F)F)cc(C(C)(C)C)c1 ; O=C1CCCN1c1cccc(N2CCCC2=O)c1-c1c(-c2c(N3CCCC3=O)cccc2N2CCCC2=O)c(C(F)(F)F)c(-c2c(N3CCCC3=O)cccc2N2CCCC2=O)c(-c2c(N3CCCC3=O)cccc2N2CCCC2=O)c1C(F)(F)F ; O=C1CCCN1c1cc(-c2c(-c3cc(N4CCCC4=O)cc(N4CCCC4=O)c3)c(C(F)(F)F)c(-c3cc(N4CCCC4=O)cc(N4CCCC4=O)c3)c(-c3cc(N4CCCC4=O)cc(N4CCCC4=O)c3)c2C(F)(F)F)cc(N2CCCC2=O)c1]

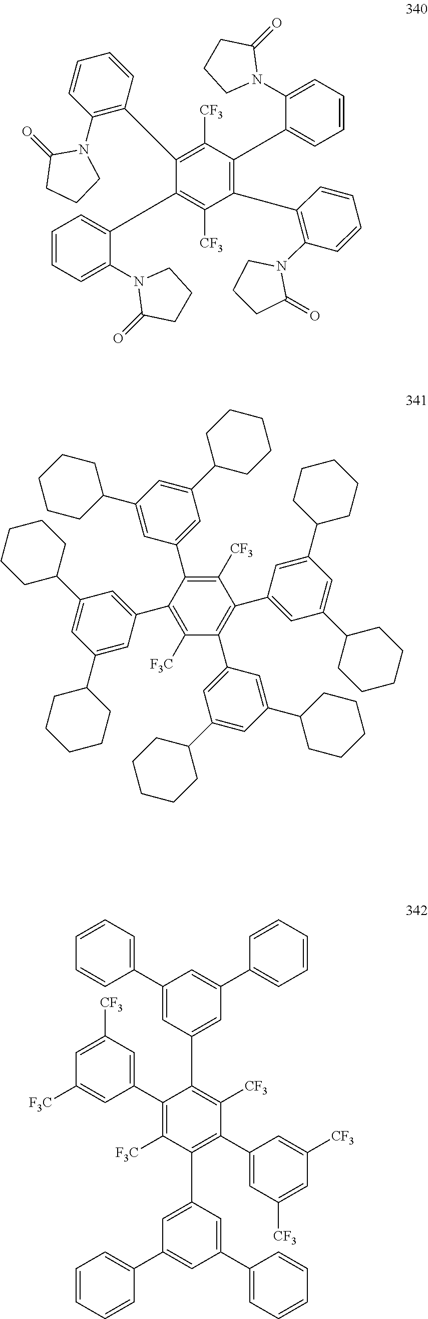 [SMILES: O=C1CCCN1c1ccccc1-c1c(-c2ccccc2N2CCCC2=O)c(C(F)(F)F)c(-c2ccccc2N2CCCC2=O)c(-c2ccccc2N2CCCC2=O)c1C(F)(F)F ; FC(F)(F)c1c(-c2cc(C3CCCCC3)cc(C3CCCCC3)c2)c(-c2cc(C3CCCCC3)cc(C3CCCCC3)c2)c(C(F)(F)F)c(-c2cc(C3CCCCC3)cc(C3CCCCC3)c2)c1-c1cc(C2CCCCC2)cc(C2CCCCC2)c1 ; FC(F)(F)c1cc(-c2c(-c3cc(-c4ccccc4)cc(-c4ccccc4)c3)c(C(F)(F)F)c(-c3cc(C(F)(F)F)cc(C(F)(F)F)c3)c(-c3cc(-c4ccccc4)cc(-c4ccccc4)c3)c2C(F)(F)F)cc(C(F)(F)F)c1]

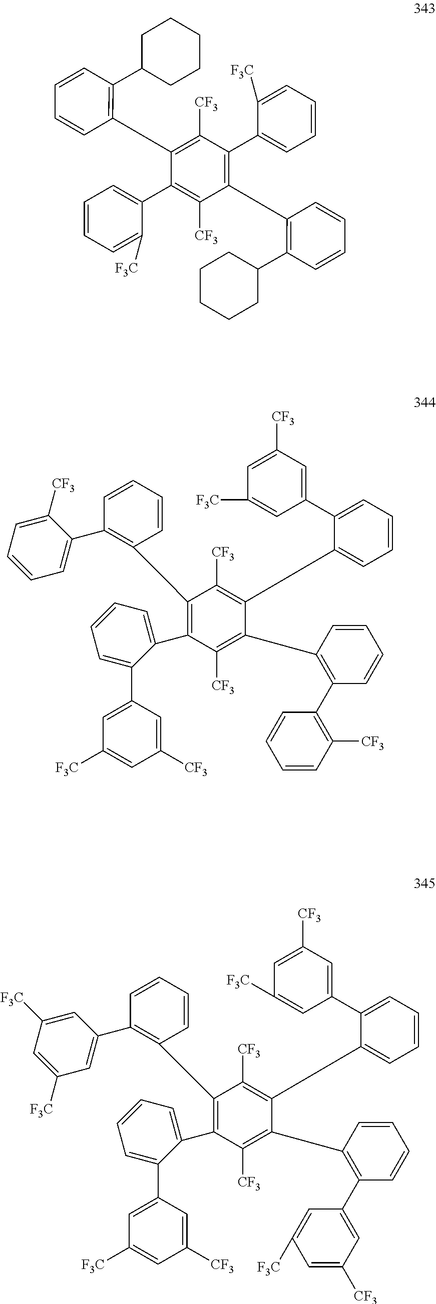 [SMILES: FC(F)(F)c1ccccc1-c1c(-c2ccccc2C2CCCCC2)c(C(F)(F)F)c(-c2ccccc2C(F)(F)F)c(-c2ccccc2C2CCCCC2)c1C(F)(F)F ; FC(F)(F)c1cc(-c2ccccc2-c2c(-c3ccccc3-c3ccccc3C(F)(F)F)c(C(F)(F)F)c(-c3ccccc3-c3cc(C(F)(F)F)cc(C(F)(F)F)c3)c(-c3ccccc3-c3ccccc3C(F)(F)F)c2C(F)(F)F)cc(C(F)(F)F)c1 ; FC(F)(F)c1cc(-c2ccccc2-c2c(-c3ccccc3-c3cc(C(F)(F)F)cc(C(F)(F)F)c3)c(C(F)(F)F)c(-c3ccccc3-c3cc(C(F)(F)F)cc(C(F)(F)F)c3)c(-c3ccccc3-c3cc(C(F)(F)F)cc(C(F)(F)F)c3)c2C(F)(F)F)cc(C(F)(F)F)c1]

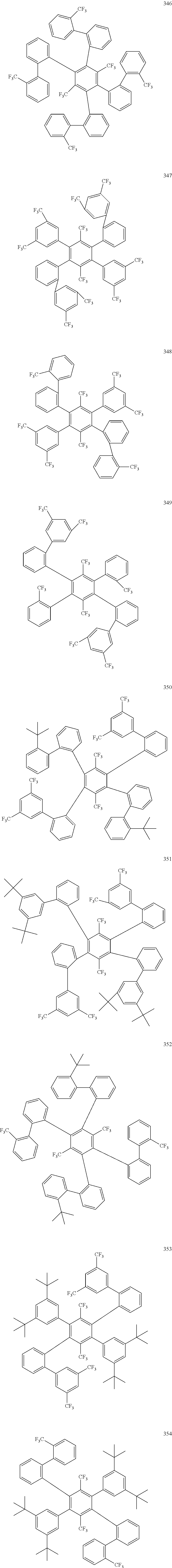

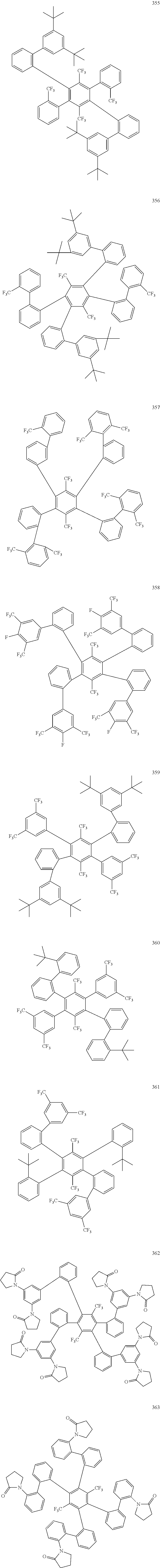

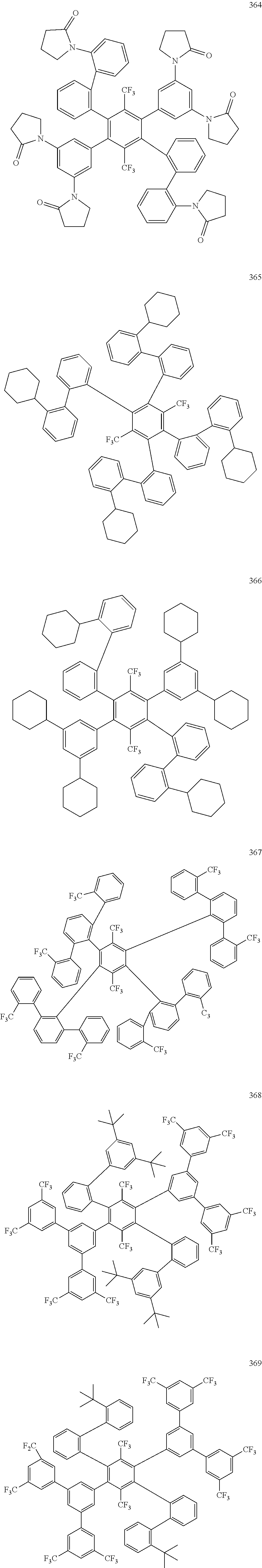

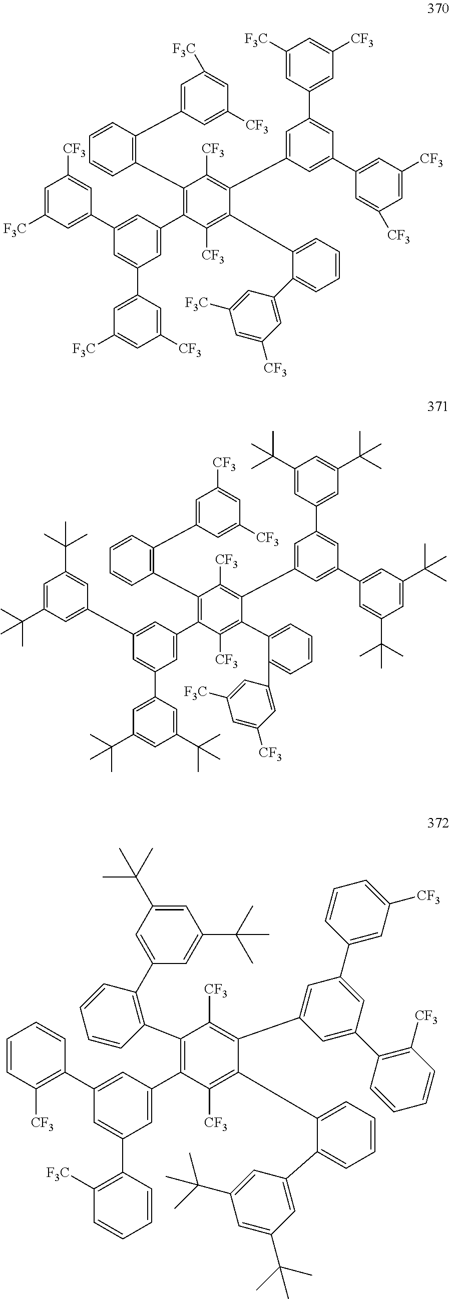

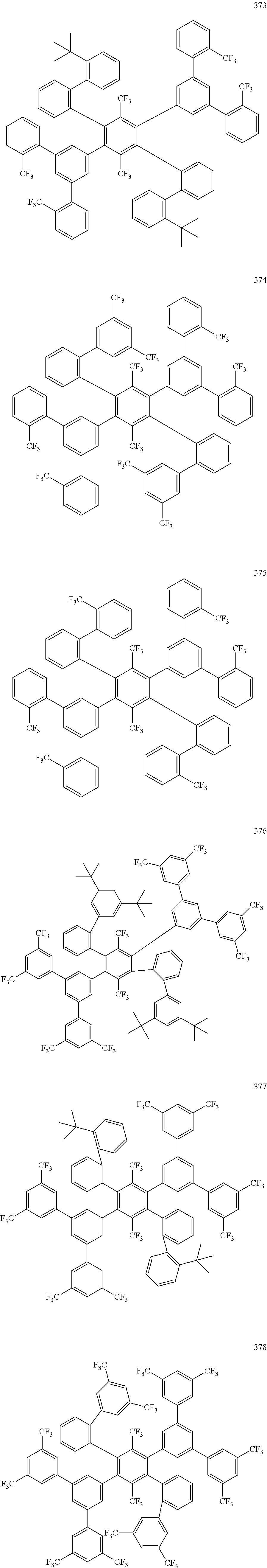

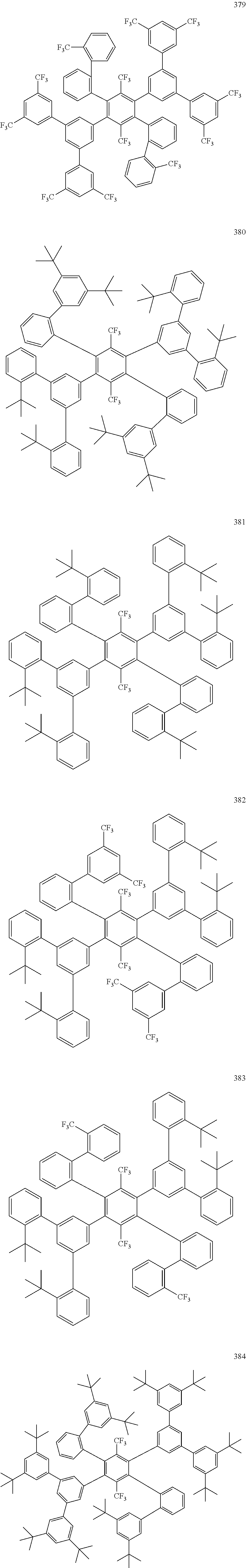

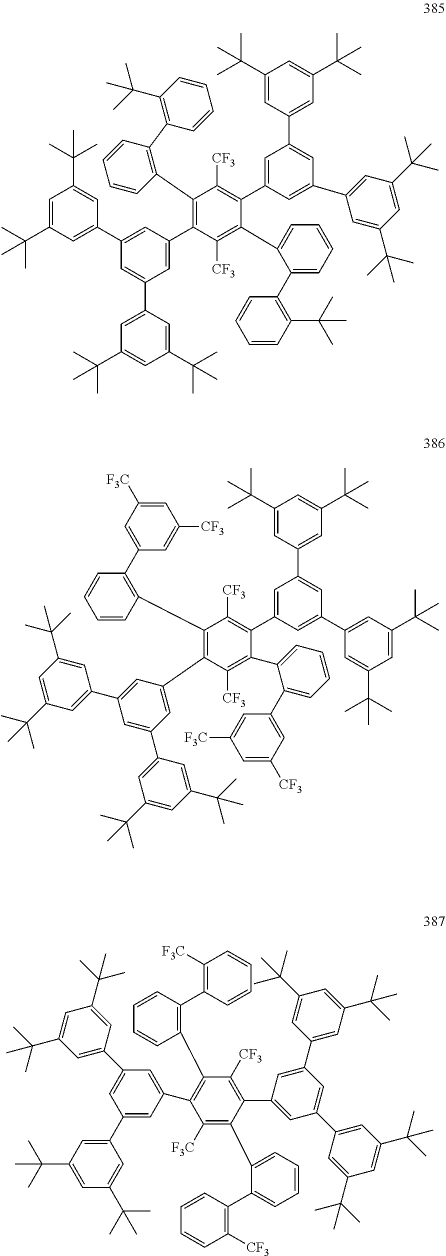

As such, the organic compound according to the present invention can synthesize organic compounds with various properties using moieties with unique properties. As a result, when the organic compound according to the present invention is applied to the light efficiency improving layer provided in the organic light-emitting device, it is possible to further improve the luminescent properties such as luminous efficiency, etc. of the device.

In addition, the compound of an embodiment of the present invention may be applied to a device according to a general method for manufacturing an organic light-emitting device.

An organic light-emitting device according to an embodiment of the present invention may include a first electrode, a second electrode, and an organic layer arranged therebetween. The organic light-emitting device may be manufactured using a general device manufacturing method and material, except that the organic compound of an embodiment of the present invention is used to form the organic layer of the device.

The organic layer of the organic light-emitting device according to an embodiment of the present invention may have a monolayer structure or a multilayer structure in which two or more organic layers are stacked. For example, the structure of the organic layers may include a hole injecting layer, a hole transport layer, a light emitting layer, an electron transport layer, an electron injecting layer, an electron blocking layer, a hole blocking layer, and a light efficiency improving layer (capping layer). The number of the organic layers is not limited and may be increased or decreased.

In addition, the organic light-emitting device may include a substrate, a first electrode (anode), an organic layer, a second electrode (cathode), and a light efficiency improving layer, and the light efficiency improving layer may be formed under the first electrode (bottom emission) or on the second electrode (top emission).

When the light efficiency improving layer is formed on the second electrode (top emission), light from the light emitting layer is emitted to the cathode and passes through the light efficiency improving layer (CPL) formed using the compound according to an embodiment of the present invention having a relatively high refractive index, so that the wavelength of the light is amplified, resulting in an increase in luminous efficiency.

Preferred structures of the organic layers of the organic light-emitting device according to an embodiment of the present invention will be explained in more detail in the examples to be described later.

In addition, the organic light-emitting device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light-emitting device may be fabricated by depositing a cathode material, organic layer materials, and an anode material in this order on a substrate. The organic layers may have a multilayer structure including a hole injecting layer, a hole transport layer, a light emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO: Al and SnO₂: Sb; and conductive polymers such as poly(3-methylthiophene), poly [3,4-(ethylene-1,2-dioxy)thiophene] (PEDT), polypyrrole, and polyaniline.

As the cathode material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport material is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light-emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light-emitting device.

### MODE FOR CARRYING OUT INVENTION

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 5

### (1) Preparation Example 1: Synthesis of Compound 5

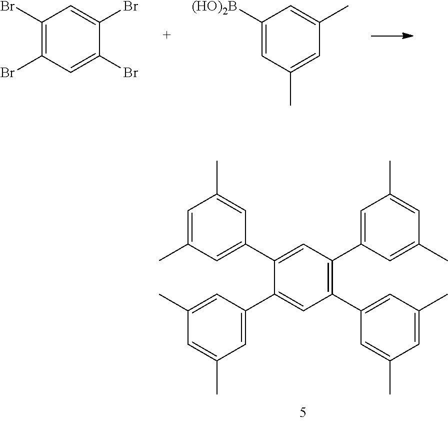

1,2,4,5-Tetrabromobenzene (10.0 g, 0.025 mol), 3,5-Dimethylphenyl boronic acid (18.3 g, 0.122 mol), K₂CO₃ (42.1 g, 0.305 mol), and Pd(PPh₃)₄ (2.3 g, 0.002 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 9.5 g of Compound 5 (yield 75.6%).

LC/MS: m/z=494 [(M)⁺]

### Synthesis Example 2: Synthesis of Compound 21

### (1) Preparation Example 1: Synthesis of Compound 21

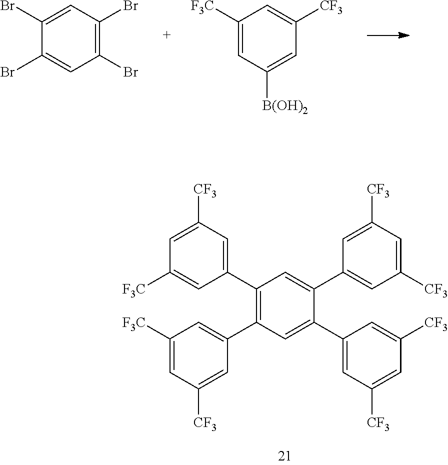 [SMILES: Brc1cc(Br)c(Br)cc1Br ; OB(O)c1cc(C(F)(F)F)cc(C(F)(F)F)c1 ; FC(F)(F)c1cc(-c2cc(-c3cc(C(F)(F)F)cc(C(F)(F)F)c3)c(-c3cc(C(F)(F)F)cc(C(F)(F)F)c3)cc2-c2cc(C(F)(F)F)cc(C(F)(F)F)c2)cc(C(F)(F)F)c1]

1,2,4,5-Tetrabromobenzene (10.0 g, 0.025 mol), 3,5-Bis(trifluoromethyl) phenylboronic acid (31.5 g, 0.122 mol), K₂CO₃ (42.1 g, 0.305 mol), and Pd(PPh₃)₄ (2.3 g, 0.002 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 16.9 g of Compound 21 (yield 71.8%).

LC/MS: m/z=926 [(M)⁺]

### Synthesis Example 3: Synthesis of Compound 27

### (1) Preparation Example 1: Synthesis of Intermediate 27-1

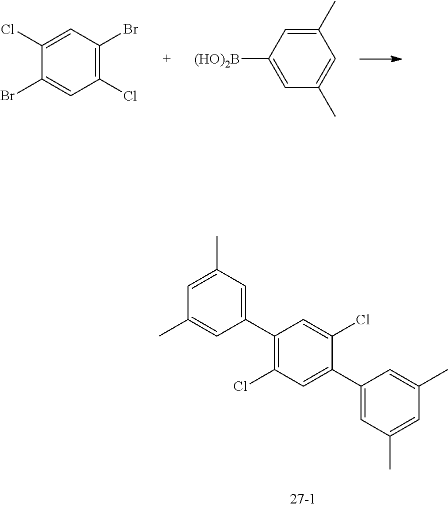

1,4-Dibromo-2,5-dichlorobenzene (10.0 g, 0.033 mol), 3,5-Dimethylphenyl boronic acid (11.8 g, 0.079 mol), K₂CO₃ (27.2 g, 0.197 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 7.4 g of Intermediate 27-1 (yield 63.5%).

### (2) Preparation Example 2: Synthesis of Compound 27

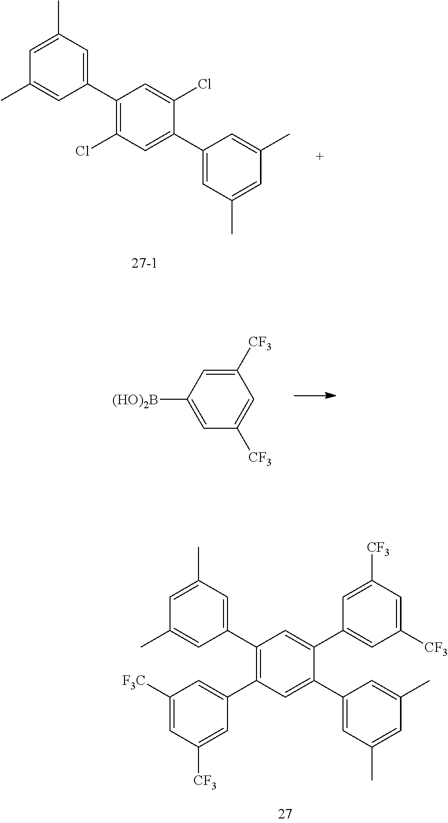

Intermediate 27-1 (10.0 g, 0.028 mol), 3,5-Bis(trifluoromethyl) phenylboronic acid (17.4 g, 0.068 mol), K₂CO₃ (19.5 g, 0.141 mol), Pd(OAc) 2 (3.3 g, 0.003 mol), X-Phos (2.7 g, 0.006 mol), THF 200 mL, and H₂O 50 mL were added and reacted while stirring at 70° C. for 6 hours. After completion of the reaction, the reaction mixture was extracted, concentrated, and then column and recrystallized to obtain 13.3 g of Compound 27 (yield 66.5%).

LC/MS: m/z=710 [(M)⁺]

### Synthesis Example 4: Synthesis of Compound 34

### (1) Preparation Example 1: Synthesis of Intermediate 34-1

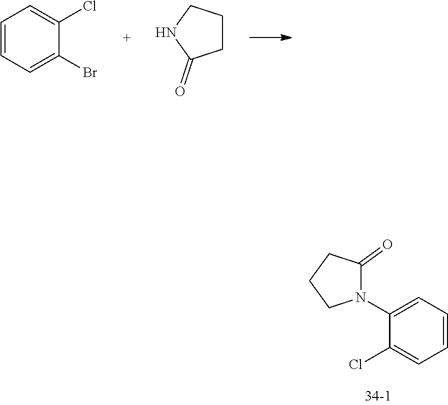 [SMILES: Clc1ccccc1Br ; O=C1CCCN1 ; O=C1CCCN1c1ccccc1Cl]

1-Bromo-2-chlorobenzene (10.0 g, 0.052 mol), 2-Pyrrolidinone (5.3 g, 0.063 mol), Cs₂CO₃ (23.8 g, 0.073 mol), Pd (dba) 2 (1.5 g, 0.003 mol), Xant-Phos (5.4 g, 0.009 mol), and dioxane were added and reacted while stirring under reflux for 16 hours. After completion of the reaction, the reaction mixture was extracted, concentrated, and then columned to obtain 8.9 g of Intermediate 34-1 (yield 87.1%).

### (2) Preparation Example 2: Synthesis of Intermediate 34-2

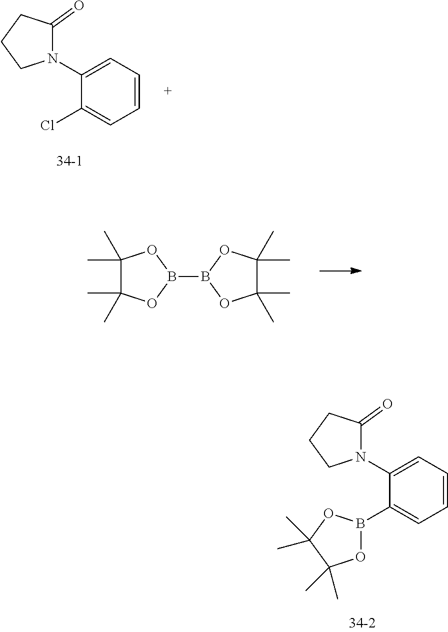

Intermediate 34-1 (10.0 g, 0.051 mol), Bis(pinacolato) diboron (15.6 g, 0.061 mol), CH₃COOK (15.1 g, 0.153 mol), Pd (dppf) Cl₂ (1.9 g, 0.003 mol), and XPhos (2.2 g, 0.005 mol) were added with dioxane and reacted while stirring at 100° C. for 12 hours. After completion of the reaction, the reaction mixture was extracted, concentrated, and then column and recrystallized to obtain 10.5 g of Intermediate 34-2 (yield 71.5%).

### (3) Preparation Example 3: Synthesis of Compound 34

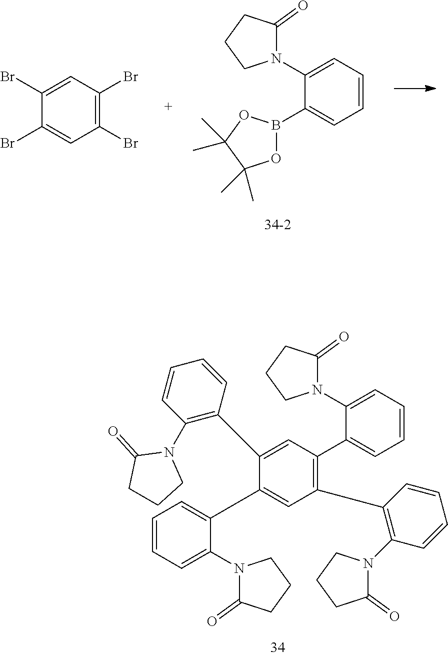 [SMILES: Brc1cc(Br)c(Br)cc1Br ; CC1(C)OB(c2ccccc2N2CCCC2=O)OC1(C)C ; O=C1CCCN1c1ccccc1-c1cc(-c2ccccc2N2CCCC2=O)c(-c2ccccc2N2CCCC2=O)cc1-c1ccccc1N1CCCC1=O]

1,2,4,5-Tetrabromobenzene (10.0 g, 0.025 mol), Intermediate 34-2 (35.0 g, 0.122 mol), K₂CO₃ (42.1 g, 0.305 mol), and Pd(PPh₃)₄ (2.4 g, 0.002 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 12.2 g of Compound 34 (yield 67.2%).

LC/MS: m/z=714 [(M)⁺]

### Synthesis Example 5: Synthesis of Compound 54

### (1) Preparation Example 1: Synthesis of Intermediate 54-1

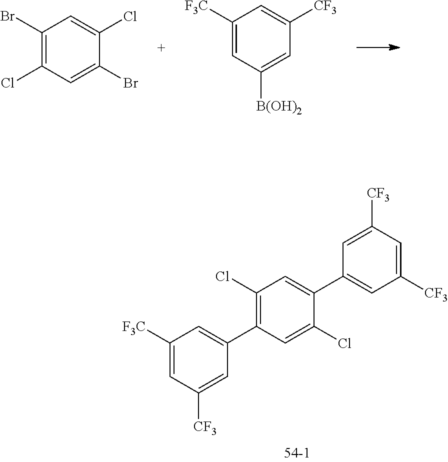 [SMILES: Clc1cc(Br)c(Cl)cc1Br ; OB(O)c1cc(C(F)(F)F)cc(C(F)(F)F)c1 ; FC(F)(F)c1cc(-c2cc(Cl)c(-c3cc(C(F)(F)F)cc(C(F)(F)F)c3)cc2Cl)cc(C(F)(F)F)c1]

1,4-Dibromo-2,5-dichlorobenzene (10.0 g, 0.033 mol), 3,5-Bis(trifluoromethyl) phenylboronic acid (20.3 g, 0.079 mol), K₂CO₃ (27.2 g, 0.197 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 11.5 g of Intermediate 54-1 (yield 61.4%).

### (2) Preparation Example 2: Synthesis of Intermediate 54-2

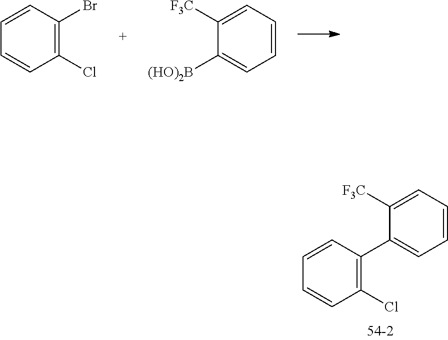 [SMILES: Clc1ccccc1Br ; OB(O)c1ccccc1C(F)(F)F ; FC(F)(F)c1ccccc1-c1ccccc1Cl]

1-Bromo-2-chlorobenzene (10.0 g, 0.052 mol), 2-Trifluoromethylphenyl boronic acid (11.9 g, 0.063 mol), K₂CO₃ (21.7 g, 0.157 mol), and Pd(PPh₃)₄ (1.2 g, 0.001 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 9.1 g of Intermediate 54-2 (yield 67.9%).

### (3) Preparation Example 3: Synthesis of Intermediate 54-3

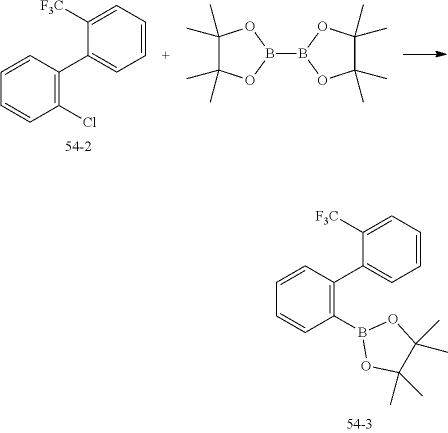

Intermediate 54-2 (10.0 g, 0.039 mol), Bis(pinacolato) diboron (11.9 g, 0.047 mol), CH₃COOK (11.5 g, 0.117 mol), Pd (dppf) Cl₂ (1.4 g, 0.002 mol), and XPhos (1.7 g, 0.004 mol) were added with dioxane and reacted while stirring at 100° C. for 12 hours. After completion of the reaction, the reaction mixture was extracted, concentrated, and then column and recrystallized to obtain 9.5 g of Intermediate 54-3 (yield 70.0%).

### (4) Preparation Example 4: Synthesis of Compound 54

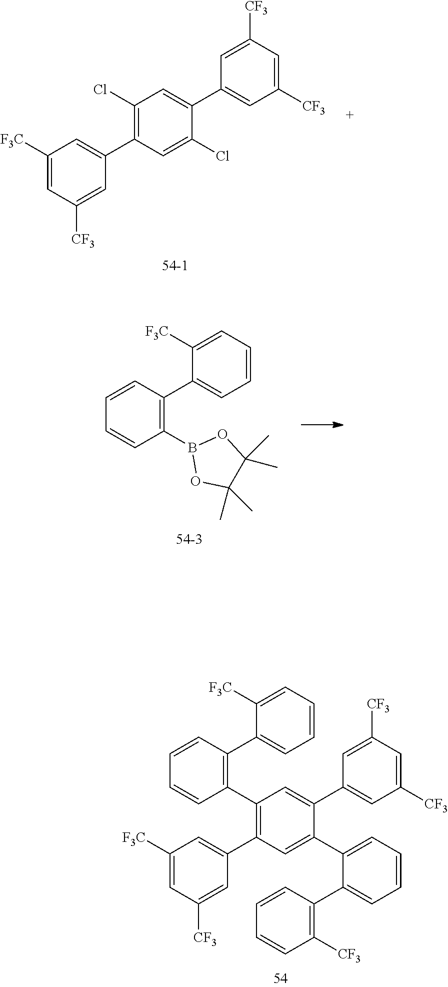 [SMILES: FC(F)(F)c1cc(-c2cc(Cl)c(-c3cc(C(F)(F)F)cc(C(F)(F)F)c3)cc2Cl)cc(C(F)(F)F)c1 ; CC1(C)OB(c2ccccc2-c2ccccc2C(F)(F)F)OC1(C)C ; FC(F)(F)c1cc(-c2cc(-c3ccccc3-c3ccccc3C(F)(F)F)c(-c3cc(C(F)(F)F)cc(C(F)(F)F)c3)cc2-c2ccccc2-c2ccccc2C(F)(F)F)cc(C(F)(F)F)c1]

Intermediate 54-1 (10.0 g, 0.018 mol), Intermediate 54-3 (14.6 g, 0.042 mol), K₂CO₃ (12.1 g, 0.088 mol), Pd(OAc) 2 (2.0 g, 0.002 mol), X-Phos (1.7 g, 0.004 mol), THF 200 mL, and H₂O 50 mL were added and reacted while stirring at 70° C. for 6 hours. After completion of the reaction, the reaction mixture was extracted, concentrated, and then column and recrystallized to obtain 10.2 g of Compound 54 (yield 61.8%). LC/MS: m/z=942 [(M)⁺]

### Synthesis Example 6: Synthesis of Compound 59

### (1) Preparation Example 1: Synthesis of Intermediate 59-1

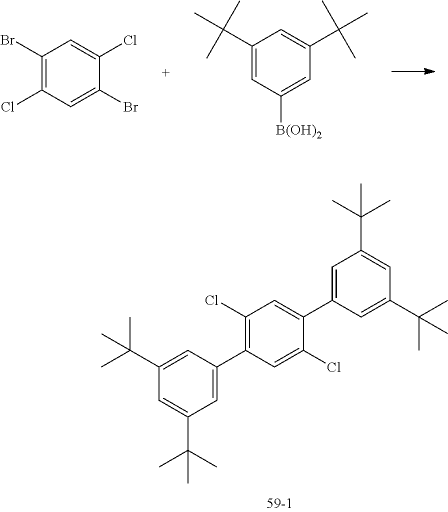 [SMILES: Clc1cc(Br)c(Cl)cc1Br ; CC(C)(C)c1cc(B(O)O)cc(C(C)(C)C)c1 ; CC(C)(C)c1cc(-c2cc(Cl)c(-c3cc(C(C)(C)C)cc(C(C)(C)C)c3)cc2Cl)cc(C(C)(C)C)c1]

1,4-Dibromo-2,5-dichlorobenzene (10.0 g, 0.033 mol), (3,5-Di-tert-butylphenyl) boronic acid (18.4 g, 0.079 mol), K₂CO₃ (27.2 g, 0.197 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 12.2 g of Intermediate 59-1 (yield 71.0%).

### (2) Preparation Example 2: Synthesis of Intermediate 59-2

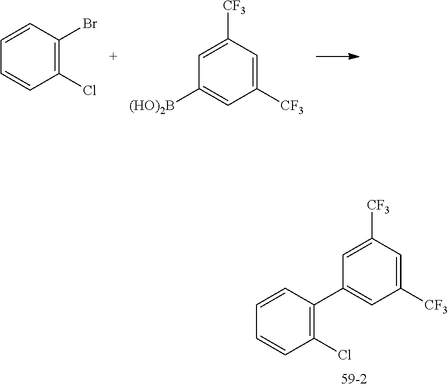 [SMILES: Clc1ccccc1Br ; OB(O)c1cc(C(F)(F)F)cc(C(F)(F)F)c1 ; FC(F)(F)c1cc(-c2ccccc2Cl)cc(C(F)(F)F)c1]

1-Bromo-2-chlorobenzene (10.0 g, 0.052 mol), 3,5-Bis(trifluoromethyl) phenylboronic acid (16.2 g, 0.063 mol), K₂CO₃ (21.7 g, 0.157 mol), and Pd(PPh₃)₄ (1.2 g, 0.001 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 10.8 g of Intermediate 59-2 (yield 63.7%).

### (3) Preparation Example 3: Synthesis of Intermediate 59-3

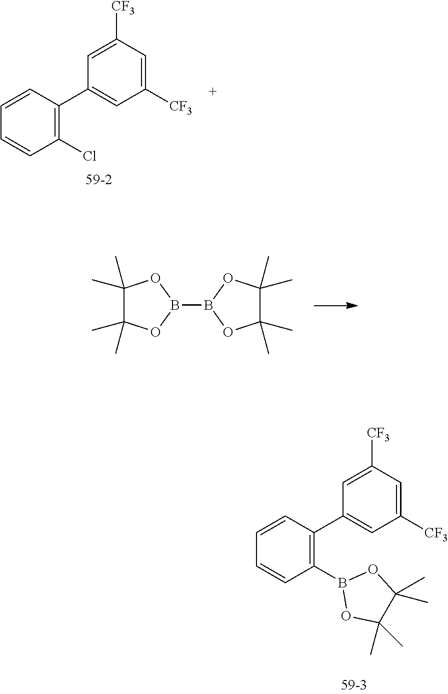

Intermediate 59-2 (10.0 g, 0.031 mol), Bis(pinacolato) diboron (9.4 g, 0.037 mol), CH₃COOK (9.1 g, 0.092 mol), Pd (dppf) Cl₂ (1.1 g, 0.002 mol), and XPhos (1.3 g, 0.003 mol) were added with dioxane and reacted while stirring at 100° C. for 12 hours. After completion of the reaction, the reaction mixture was extracted, concentrated, and then column and recrystallized to obtain 9.2 g of Intermediate 59-3 (yield 71.8%).

### (4) Preparation Example 4: Synthesis of Compound 59

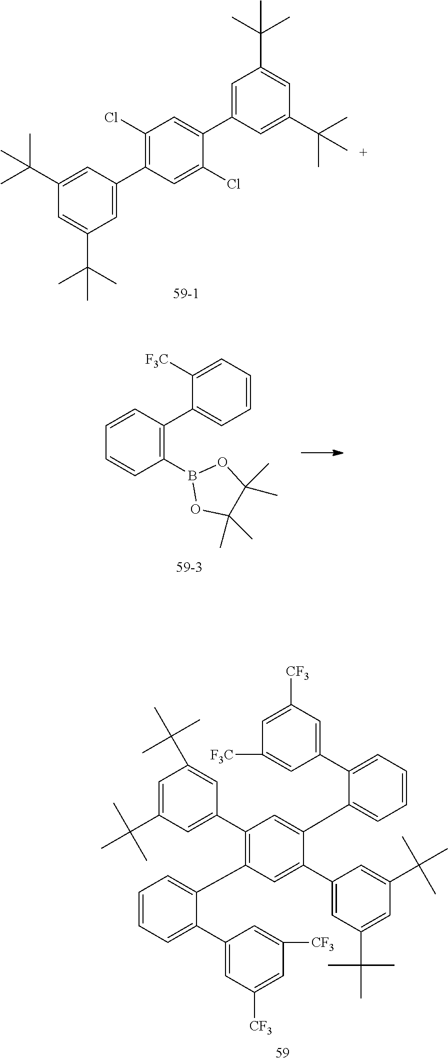

Intermediate 59-1 (10.0 g, 0.019 mol), Intermediate 59-3 (16.0 g, 0.046 mol), K₂CO₃ (13.2 g, 0.096 mol), Pd(OAc) 2 (2.2 g, 0.002 mol), X-Phos (1.8 g, 0.004 mol), THF 200 mL, and H₂O 50 mL were added and reacted while stirring at 70° C. for 6 hours. After completion of the reaction, the reaction mixture was extracted, concentrated, and then column and recrystallized to obtain 10.5 g of Compound 59 (yield 53.3%). LC/MS: m/z=1031 [(M)⁺]

### Synthesis Example 7: Synthesis of Compound 140

### (1) Preparation Example 1: Synthesis of Compound 140

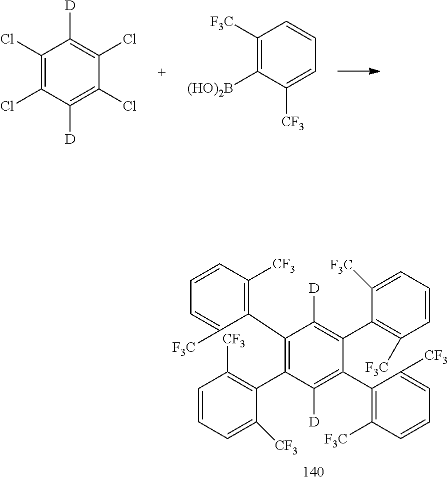 [SMILES: [2H]c1c(Cl)c(Cl)c([2H])c(Cl)c1Cl ; OB(O)c1c(C(F)(F)F)cccc1C(F)(F)F ; [2H]c1c(-c2c(C(F)(F)F)cccc2C(F)(F)F)c(-c2c(C(F)(F)F)cccc2C(F)(F)F)c([2H])c(-c2c(C(F)(F)F)cccc2C(F)(F)F)c1-c1c(C(F)(F)F)cccc1C(F)(F)F]

1,2,4,5-Tetrachlorobenzene-d2 (10.0 g, 0.046 mol), B-[2,6-Bis(trifluoromethyl) phenyl] boronic acid (56.8 g, 0.220 mol), K₂CO₃ (76.1 g, 0.551 mol), Pd(OAc) 2 (5.3 g, 0.005 mol), X-Phos (6.6 g, 0.014 mol), THF 200 mL and H₂O 50 mL were added and reacted while stirring at 70° C. for 6 hours. After completion of the reaction, the reaction mixture was extracted, concentrated, and then column and recrystallized to obtain 18.2 g of Compound 140 (yield 42.7%).

LC/MS: m/z=928 [(M)⁺]

### Synthesis Example 8: Synthesis of Compound 148

### (1) Preparation Example 1: Synthesis of Intermediate 148-1

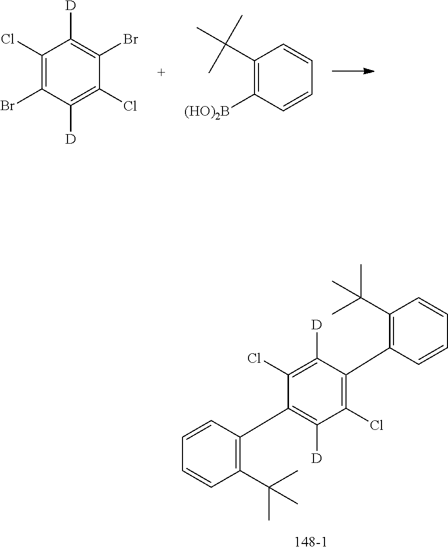 [SMILES: [2H]c1c(Cl)c(Br)c([2H])c(Cl)c1Br ; CC(C)(C)c1ccccc1B(O)O ; [2H]c1c(Cl)c(-c2ccccc2C(C)(C)C)c([2H])c(Cl)c1-c1ccccc1C(C)(C)C]

1,4-dibromo-2,5-dichloro-3,6-dideuteriobenzene (10.0 g, 0.033 mol), 4-tert-Butylphenylboronic acid (13.9 g, 0.078 mol), K₂CO₃ (27.0 g, 0.196 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 8.2 g of Intermediate 148-1 (yield 60.9%).

### (2) Preparation Example 2: Synthesis of Compound 148

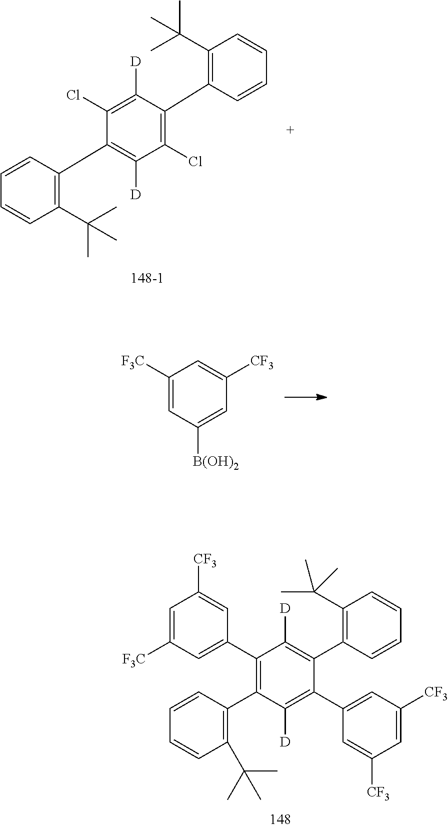 [SMILES: [2H]c1c(Cl)c(-c2ccccc2C(C)(C)C)c([2H])c(Cl)c1-c1ccccc1C(C)(C)C ; OB(O)c1cc(C(F)(F)F)cc(C(F)(F)F)c1 ; [2H]c1c(-c2cc(C(F)(F)F)cc(C(F)(F)F)c2)c(-c2ccccc2C(C)(C)C)c([2H])c(-c2cc(C(F)(F)F)cc(C(F)(F)F)c2)c1-c1ccccc1C(C)(C)C]

Intermediate 148-1 (10.0 g, 0.024 mol), 3,5-Bis(trifluoromethyl) phenylboronic acid (15.0 g, 0.058 mol), K₂CO₃ (16.7 g, 0.121 mol), Pd(OAc) 2 (2.8 g, 0.002 mol), X-Phos (2.3 g, 0.005 mol), THF 200 mL, and H₂O 50 mL were added and reacted while stirring at 70° C. for 6 hours. After completion of the reaction, the reaction mixture was extracted, concentrated, and then column and recrystallized to obtain 9.7 g of Compound 148 (yield 52.2%).

LC/MS: m/z=768 [(M)⁺]

### Synthesis Example 9: Synthesis of Compound 242

### (1) Preparation Example 1: Synthesis of Intermediate 242-1

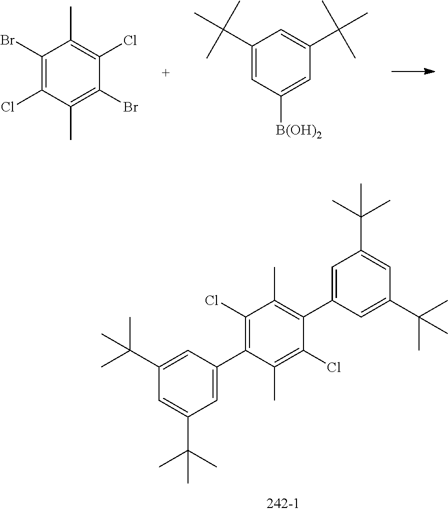 [SMILES: Cc1c(Cl)c(Br)c(C)c(Cl)c1Br ; CC(C)(C)c1cc(B(O)O)cc(C(C)(C)C)c1 ; Cc1c(Cl)c(-c2cc(C(C)(C)C)cc(C(C)(C)C)c2)c(C)c(Cl)c1-c1cc(C(C)(C)C)cc(C(C)(C)C)c1]

1,4-dibromo-2,5-dichloro-3,6-dimethylbenzene (10.0 g, 0.030 mol), (3,5-Di-tert-butylphenyl) boronic acid (16.9 g, 0.072 mol), K₂CO₃ (24.9 g, 0.180 mol), and Pd(PPh₃)₄ (0.7 g, 0.0006 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 7.9 g of Intermediate 242-1 (yield 47.7%).

### (2) Preparation Example 2: Synthesis of Compound 242

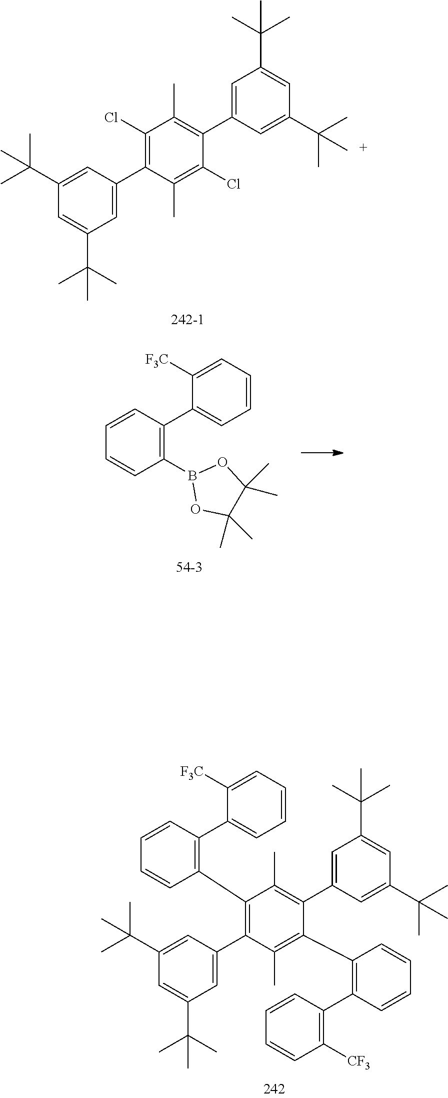

Intermediate 242-1 (10.0 g, 0.018 mol), Intermediate 54-3 (15.2 g, 0.044 mol), K₂CO₃ (12.5 g, 0.091 mol), Pd(OAc) 2 (2.1 g, 0.002 mol), X-Phos (1.7 g, 0.004 mol), THF 200 mL, and H₂O 50 mL were added and reacted while stirring at 70° C. for 6 hours. After completion of the reaction, the reaction mixture was extracted, concentrated, and then column and recrystallized to obtain 7.1 g of Compound 242 (yield 42.4%).

LC/MS: m/z=923 [(M)⁺]

### Synthesis Example 10: Synthesis of Compound 328

### (1) Preparation Example 1: Synthesis of Compound 328

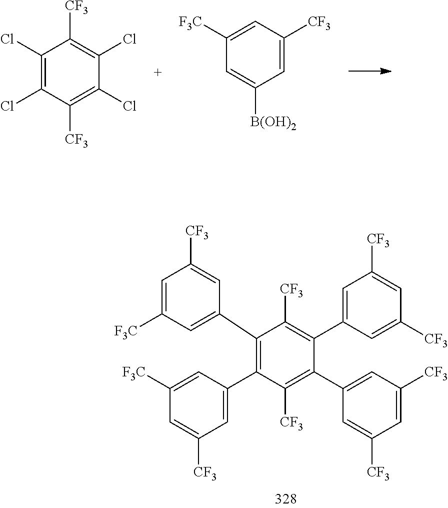 [SMILES: FC(F)(F)c1c(Cl)c(Cl)c(C(F)(F)F)c(Cl)c1Cl ; OB(O)c1cc(C(F)(F)F)cc(C(F)(F)F)c1 ; FC(F)(F)c1cc(-c2c(-c3cc(C(F)(F)F)cc(C(F)(F)F)c3)c(C(F)(F)F)c(-c3cc(C(F)(F)F)cc(C(F)(F)F)c3)c(-c3cc(C(F)(F)F)cc(C(F)(F)F)c3)c2C(F)(F)F)cc(C(F)(F)F)c1]

1,2,4,5-tetrachloro-3,6-bis-trifluoromethyl-benzene (10.0 g, 0.028 mol), 3,5-Bis(trifluoromethyl) phenylboronic acid (35.2 g, 0.136 mol), K₂CO₃ (47.1 g, 0.341 mol), Pd(OAc) 2 (3.3 g, 0.003 mol), X-Phos (4.1 g, 0.009 mol), THF 200 mL, and H₂O 50 mL were added and reacted while stirring at 70° C. for 6 hours. After completion of the reaction, the reaction mixture was extracted, concentrated, and then column and recrystallized to obtain 17.2 g of Compound 328 (yield 57.0%).

LC/MS: m/z=1062 [(M)⁺]

### Synthesis Example 11: Synthesis of Compound 345

### (1) Preparation Example 1: Synthesis of Compound 345

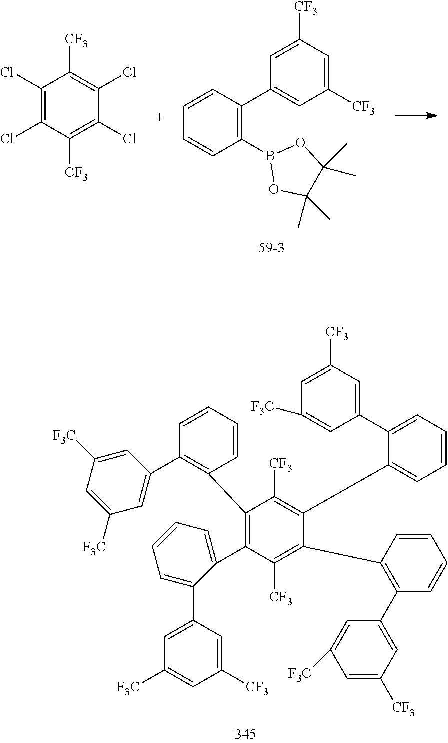 [SMILES: FC(F)(F)c1c(Cl)c(Cl)c(C(F)(F)F)c(Cl)c1Cl ; CC1(C)OB(c2ccccc2-c2cc(C(F)(F)F)cc(C(F)(F)F)c2)OC1(C)C ; FC(F)(F)c1cc(-c2ccccc2-c2c(-c3ccccc3-c3cc(C(F)(F)F)cc(C(F)(F)F)c3)c(C(F)(F)F)c(-c3ccccc3-c3cc(C(F)(F)F)cc(C(F)(F)F)c3)c(-c3ccccc3-c3cc(C(F)(F)F)cc(C(F)(F)F)c3)c2C(F)(F)F)cc(C(F)(F)F)c1]

1,2,4,5-tetrachloro-3,6-bis-trifluoromethyl-benzene (10.0 g, 0.028 mol), Intermediate 59-3 (56.8 g, 0.136 mol), K₂CO₃ (47.1 g, 0.341 mol), Pd(OAc) 2 (3.3 g, 0.003 mol), X-Phos (4.1 g, 0.009 mol), THF 200 mL, and H₂O 50 mL were added and reacted while stirring at 70° C. for 6 hours. After completion of the reaction, the reaction mixture was extracted, concentrated, and then column and recrystallized to obtain 12.5 g of Compound 345 (yield 67.3%).

LC/MS: m/z=1366 [(M)⁺]

### Device Example (CPL)

In Example according to the present invention, an Ag-containing ITO glass substrate having dimensions of 25 mm×25 mm×0.7 mm was used as an anode, patterned to have a light emitting area of 2 mm×2 mm, followed by cleaning. After the patterned ITO substrate was mounted in a vacuum chamber, organic materials and metals were deposited in the following structure on the substrate at a process pressure of 1×10⁻⁶ torr or more.

### Device Examples 1 to 91

The compound implemented according to the present invention was employed in a light efficiency improving layer to fabricate an organic light-emitting device having the following device structure, and then the luminescent and driving characteristics according to the compound implemented according to the present invention were measured.

Ag: ITO/hole injecting layer (HAT-CN, 5 nm)/hole transport layer (α-NPB, 100 nm)/electron blocking layer (TCTA, 10 nm)/light emitting layer (20 nm)/electron transport layer (201:Liq, 30 nm)/LiF (1 nm)/Mg:Ag (15 nm)/light efficiency improving layer (70 nm)

On a glass substrate, HAT-CN was formed into a 5 nm-thick film to form a hole injecting layer on an Ag-containing ITO transparent electrode. Thereafter, α-NPB was formed into a 100 nm-thick film to form a hole transport layer, and then TCTA was formed into a 10 nm-thick film to form an electron blocking layer. A 20 nm-thick light emitting layer was formed by co-depositing BH1 as a host compound and BD1 as a dopant compound, a 30 nm-thick electron transport layer (doped with Liq 50% of the following compound) was deposited, and then LiF was formed into a 1 nm-thick film to form an electron injecting layer, and Mg:Ag at a ratio of 1:9 was formed into a 15 nm-thick film to form a cathode. In addition, the light efficiency improving layer (capping layer) was formed to a 70 nm-thick film using the compounds according to the present invention shown in Table 1 below to fabricate an organic light-emitting device.

### Device Comparative Example 1

An organic light-emitting device for Device Comparative Example 1 was fabricated in the same manner, except that the light efficiency improving layer in the device structure of Example above was not provided.

### Device Comparative Example 2

An organic light-emitting device for Device Comparative Example 2 was fabricated in the same manner, except that Alq₃ was used instead of the compound according to the present invention as the light efficiency improving layer compound in the device structure of Example above.

### Device Comparative Example 3

An organic light-emitting device for Device Comparative Example 3 was fabricated in the same manner, except that CP 1 was used instead of the compound according to the present invention as the light efficiency improving layer compound in the device structure of Example above.

### Device Comparative Example 4

An organic light-emitting device for Device Comparative Example 4 was fabricated in the same manner, except that CP 2 was used instead of the compound according to the present invention as the light efficiency improving layer compound in the device structure of Example above.

### Device Comparative Example 5

An organic light-emitting device for Device Comparative Example 5 was fabricated in the same manner, except that CP 3 was used instead of the compound according to the present invention as the light efficiency improving layer compound in the device structure of Example above.

### Experimental Example 1: Luminescent Properties of Device Examples 1 to 91

The driving voltages, current efficiencies, and color coordinates of the organic light-emitting devices fabricated in Examples and Comparative Examples above were measured using a source meter (Model 237, Keithley) and a luminance meter (PR-650, Photo Research). The result values at 1,000 nits were shown in Table 1 below.

TABLE 1

| | Light | | | | |
|---|---|---|---|---|---|
| | efficiency | | | | |
| | improving | | | | |
| Example | layer | V | cd/A | CIEx | CIEy |
| 1 | Formula 2 | 3.43 | 9.25 | 0.1291 | 0.0579 |
| 2 | Formula 5 | 3.39 | 8.57 | 0.1338 | 0.0535 |
| 3 | Formula 8 | 3.41 | 8.67 | 0.1385 | 0.0493 |
| 4 | Formula 10 | 3.42 | 8.39 | 0.1381 | 0.0497 |
| 5 | Formula 11 | 3.43 | 8.29 | 0.1382 | 0.0473 |
| 6 | Formula 12 | 3.39 | 8.83 | 0.1374 | 0.0537 |
| 7 | Formula 13 | 3.42 | 8.53 | 0.1382 | 0.0522 |
| 8 | Formula 15 | 3.35 | 8.48 | 0.1409 | 0.0466 |
| 9 | Formula 18 | 3.46 | 9.15 | 0.1385 | 0.0492 |
| 10 | Formula 21 | 3.39 | 8.67 | 0.1404 | 0.0459 |
| 11 | Formula 22 | 3.40 | 8.69 | 0.1412 | 0.0460 |
| 12 | Formula 23 | 3.42 | 9.47 | 0.1321 | 0.0579 |
| 13 | Formula 25 | 3.48 | 8.51 | 0.1351 | 0.0501 |
| 14 | Formula 26 | 3.28 | 8.90 | 0.1372 | 0.0553 |
| 15 | Formula 27 | 3.29 | 8.67 | 0.1363 | 0.0568 |
| 16 | Formula 28 | 3.30 | 8.69 | 0.1379 | 0.0491 |
| 17 | Formula 29 | 3.41 | 8.46 | 0.1345 | 0.0507 |
| 18 | Formula 32 | 3.37 | 8.77 | 0.1373 | 0.0568 |
| 19 | Formula 34 | 3.38 | 8.68 | 0.1386 | 0.0487 |
| 20 | Formula 36 | 3.53 | 8.95 | 0.1331 | 0.0569 |
| 21 | Formula 40 | 3.30 | 8.90 | 0.1374 | 0.0520 |
| 22 | Formula 45 | 3.31 | 8.76 | 0.1389 | 0.0469 |
| 23 | Formula 51 | 3.32 | 8.95 | 0.1392 | 0.0480 |
| 24 | Formula 54 | 3.35 | 8.76 | 0.1389 | 0.0490 |
| 25 | Formula 55 | 3.43 | 9.03 | 0.1321 | 0.0569 |
| 26 | Formula 56 | 3.41 | 8.73 | 0.1410 | 0.0460 |
| 27 | Formula 57 | 3.35 | 8.86 | 0.1340 | 0.0583 |
| 28 | Formula 59 | 3.36 | 8.81 | 0.1405 | 0.0479 |
| 29 | Formula 60 | 3.37 | 8.90 | 0.1369 | 0.0561 |
| 30 | Formula 62 | 3.41 | 8.81 | 0.1375 | 0.0509 |
| 31 | Formula 65 | 3.43 | 8.63 | 0.1372 | 0.0513 |
| 32 | Formula 66 | 3.31 | 8.84 | 0.1369 | 0.0568 |
| 33 | Formula 67 | 3.32 | 8.93 | 0.1373 | 0.0538 |
| 34 | Formula 86 | 3.55 | 8.41 | 0.1369 | 0.0516 |
| 35 | Formula 100 | 3.42 | 8.71 | 0.1380 | 0.0500 |
| 36 | Formula 102 | 3.23 | 8.60 | 0.1360 | 0.0541 |
| 37 | Formula 108 | 3.54 | 9.22 | 0.1310 | 0.0581 |
| 38 | Formula 119 | 3.25 | 8.65 | 0.1353 | 0.0573 |
| 39 | Formula 123 | 3.50 | 8.97 | 0.1331 | 0.0609 |
| 40 | Formula 140 | 3.51 | 8.67 | 0.1372 | 0.0495 |
| 41 | Formula 141 | 3.53 | 9.05 | 0.1331 | 0.0589 |
| 42 | Formula 147 | 3.54 | 8.25 | 0.1370 | 0.0473 |
| 43 | Formula 148 | 3.53 | 8.93 | 0.1331 | 0.0579 |
| 44 | Formula 159 | 3.41 | 8.49 | 0.1353 | 0.0498 |
| 45 | Formula 171 | 3.51 | 8.85 | 0.1331 | 0.0589 |
| 46 | Formula 186 | 3.55 | 8.74 | 0.1382 | 0.0510 |
| 47 | Formula 191 | 3.41 | 9.02 | 0.1311 | 0.0579 |
| 48 | Formula 196 | 3.63 | 8.75 | 0.1351 | 0.0539 |
| 49 | Formula 201 | 3.52 | 8.53 | 0.1389 | 0.0480 |
| 50 | Formula 202 | 3.23 | 8.61 | 0.1373 | 0.0569 |
| 51 | Formula 205 | 3.53 | 8.91 | 0.1331 | 0.0589 |
| 52 | Formula 217 | 3.63 | 8.75 | 0.1351 | 0.0529 |
| 53 | Formula 221 | 3.55 | 8.78 | 0.1381 | 0.0496 |
| 54 | Formula 223 | 3.56 | 8.93 | 0.1365 | 0.0567 |
| 55 | Formula 234 | 3.67 | 8.21 | 0.1392 | 0.0436 |
| 56 | Formula 240 | 3.67 | 8.64 | 0.1381 | 0.0488 |
| 57 | Formula 242 | 3.69 | 8.75 | 0.1351 | 0.0549 |
| 58 | Formula 246 | 3.52 | 8.57 | 0.1348 | 0.0478 |
| 59 | Formula 252 | 3.43 | 9.35 | 0.1311 | 0.0549 |
| 60 | Formula 268 | 3.44 | 8.34 | 0.1385 | 0.0454 |
| 61 | Formula 272 | 3.45 | 8.58 | 0.1397 | 0.0472 |
| 62 | Formula 285 | 3.62 | 8.98 | 0.1331 | 0.0589 |
| 63 | Formula 287 | 3.63 | 8.33 | 0.1382 | 0.0486 |
| 64 | Formula 300 | 3.53 | 8.83 | 0.1331 | 0.0589 |
| 65 | Formula 306 | 3.62 | 8.83 | 0.1355 | 0.0579 |
| 66 | Formula 308 | 3.56 | 8.83 | 0.1376 | 0.0516 |
| 67 | Formula 310 | 3.56 | 8.91 | 0.1376 | 0.0506 |
| 68 | Formula 312 | 3.63 | 8.34 | 0.1384 | 0.0460 |
| 69 | Formula 313 | 3.53 | 8.85 | 0.1321 | 0.0589 |
| 70 | Formula 328 | 3.67 | 8.33 | 0.1370 | 0.0445 |
| 71 | Formula 333 | 3.63 | 8.75 | 0.1351 | 0.0517 |
| 72 | Formula 335 | 3.64 | 8.74 | 0.1364 | 0.0523 |
| 73 | Formula 337 | 3.55 | 9.04 | 0.1363 | 0.0575 |
| 74 | Formula 338 | 3.56 | 8.27 | 0.1359 | 0.0515 |
| 75 | Formula 345 | 3.64 | 9.02 | 0.1340 | 0.0561 |
| 76 | Formula 349 | 3.53 | 8.85 | 0.1331 | 0.0589 |
| 77 | Formula 353 | 3.63 | 8.65 | 0.1351 | 0.0519 |
| 78 | Formula 354 | 3.52 | 8.88 | 0.1331 | 0.0589 |
| 79 | Formula 359 | 3.64 | 8.92 | 0.1350 | 0.0581 |
| 80 | Formula 360 | 3.52 | 8.95 | 0.1321 | 0.0569 |
| 81 | Formula 365 | 3.53 | 8.94 | 0.1353 | 0.0561 |
| 82 | Formula 367 | 3.53 | 8.75 | 0.1341 | 0.0509 |
| 83 | Formula 369 | 3.63 | 8.65 | 0.1361 | 0.0529 |
| 84 | Formula 371 | 3.58 | 8.64 | 0.1395 | 0.0496 |
| 85 | Formula 372 | 3.59 | 8.41 | 0.1374 | 0.0440 |
| 86 | Formula 375 | 3.66 | 8.23 | 0.1378 | 0.0470 |
| 87 | Formula 377 | 3.61 | 8.11 | 0.1371 | 0.0518 |
| 88 | Formula 378 | 3.60 | 8.78 | 0.1358 | 0.0573 |
| 89 | Formula 381 | 3.63 | 8.65 | 0.1351 | 0.0529 |
| 90 | Formula 385 | 3.61 | 8.15 | 0.1366 | 0.0523 |
| 91 | Formula 386 | 3.61 | 8.75 | 0.1353 | 0.0519 |
| Comparative | Not used | 4.68 | 7.03 | 0.1502 | 0.1412 |
| Exmaple 1 | | | | | |
| Comparative | Alq₃ | 4.33 | 7.84 | 0.1471 | 0.0583 |
| Exmaple 2 | | | | | |
| Comparative | CP 1 | 4.26 | 8.06 | 0.1420 | 0.0635 |
| Exmaple 3 | | | | | |
| Comparative | CP 2 | 4.05 | 7.62 | 0.1391 | 0.0642 |
| Exmaple 4 | | | | | |
| Comparative | CP 3 | 4.19 | 7.78 | 0.1401 | 0.0613 |
| Exmaple 5 | | | | | |

When describing the results shown in Table 1, it can be seen that in the case of the organic light-emitting device in which the compound according to the present invention is employed in the light efficiency improving layer provided in the organic light-emitting device, there are low driving voltage and improved current efficiency, compared to a conventional device without a light efficiency improving layer (Comparative Example 1), a conventional device employed with a compound used as a light efficiency improving layer (Comparative Example 2), and conventional devices employed with a compound that contrasts with the characteristic structure of the compound according to the present invention (Comparative Examples 3 to 5).

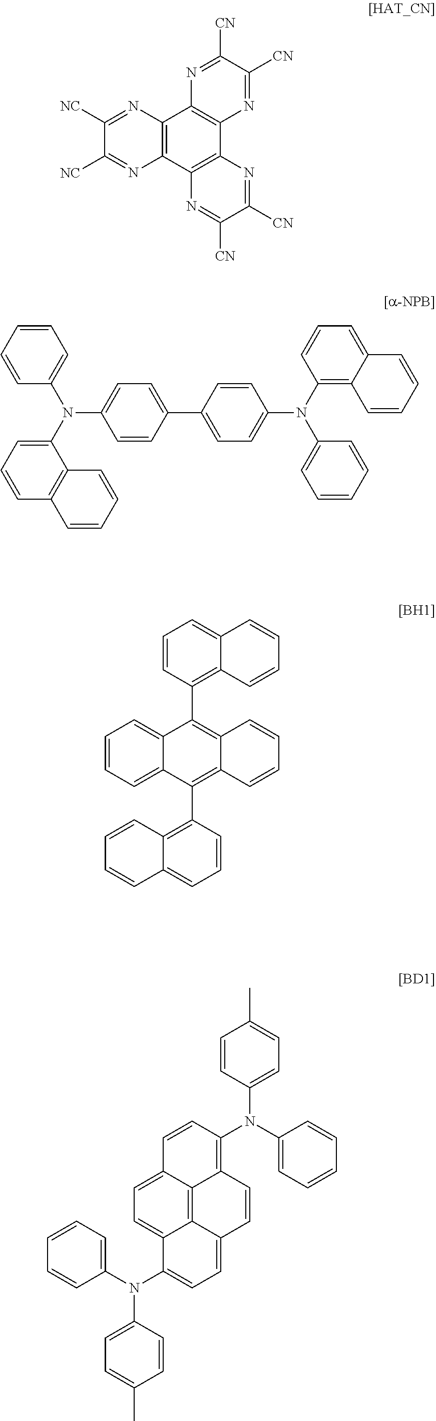

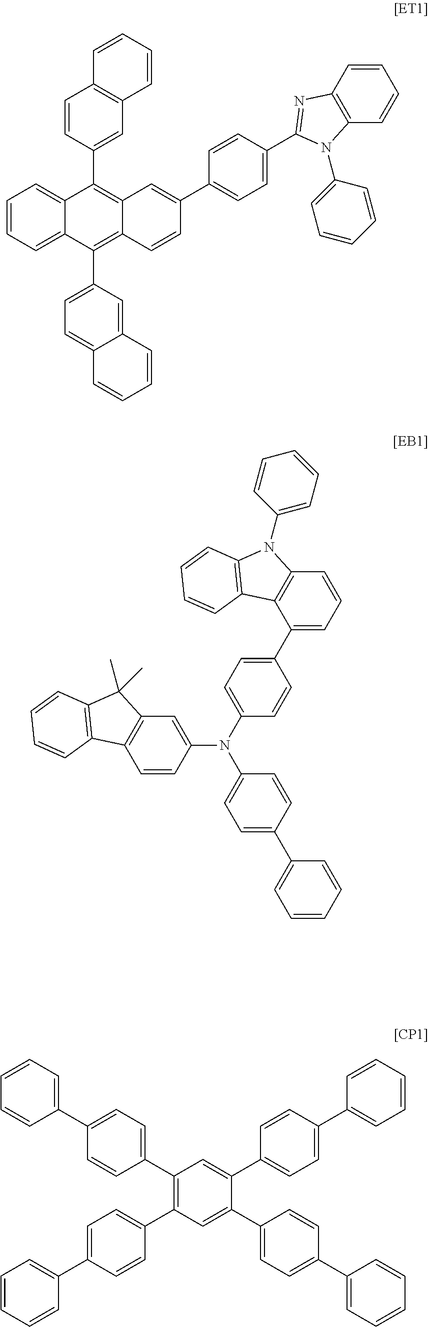

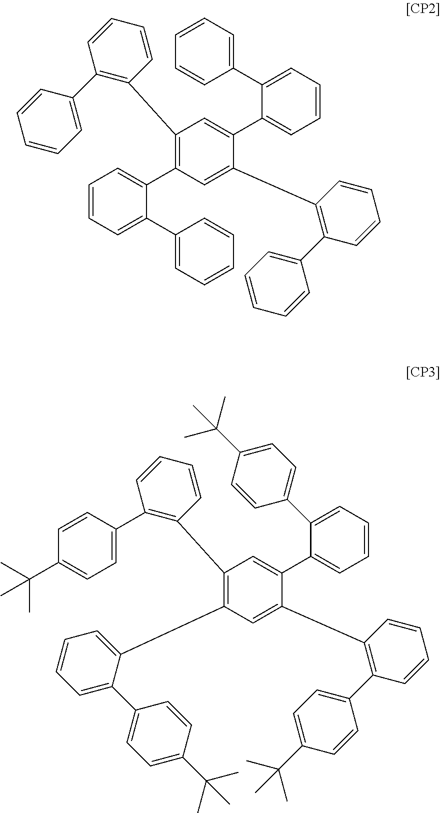

### INDUSTRIAL APPLICABILITY

According to the present invention, an organic compound is employed as a material for a light efficiency improving layer provided in an organic light-emitting device to achieve low-voltage driving and improved luminescent properties such as excellent luminous efficiency, color purity, etc. by increasing the light efficiency of the organic light-emitting device, and thus the present invention can be effectively used industrially in various lighting and display devices.

1. A compound represented by Formula I below: 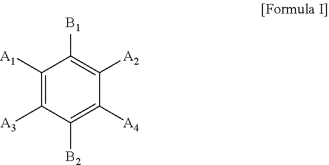
In Formula I above,
(i) A₁ and A₂ are the same or different from each other, and each independently selected from Structural Formula 1 below, 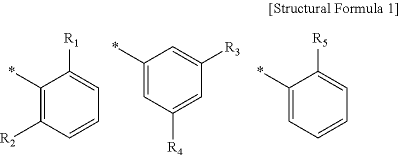
in Structural Formula 1 above,
R₁ to R₅ are the same as or different from each other, and each independently any one selected from deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted heterocycloalkyl group having 2 to 20 carbon atoms, a substituted or unsubstituted aryl group having 6 to 30 carbon atoms, a substituted or unsubstituted heteroaryl group having 2 to 30 carbon atoms, and Structural Formula 2 below, 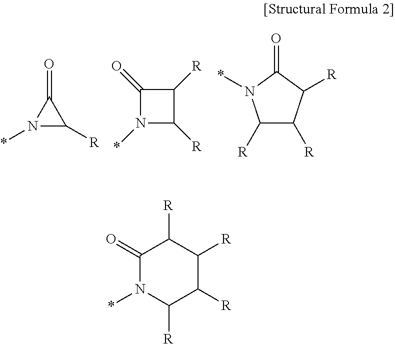
in Structural Formula 2 above,
in each structure, R is each independently any one selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms, and
in each structure of Structural Formula 2, a plurality of R are the same as or different from each other,
(ii) B₁ and B₂ are the same as or different from each other, and each independently any one selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or an unsubstituted halogenated alkoxy group having 1 to 20 carbon atoms and a substituted or unsubstituted cycloalkyl group having 3 to 20 carbon atoms.

2. The compound of claim 1, wherein in the definition of R, R₁ to R₅, B₁ and B₂, the ‘substituted or unsubstituted’ means substitution of R, R₁ to R₅, B₁ and B₂ above with one or at least two substituents selected from the group consisting of deuterium, a halogen group, a cyano group, an alkyl group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylsilyl group and an arylsilyl group, substitution with a substituent to which two or more of the substituents are linked, or having no substituent.

3. The compound of claim 1, wherein Formula 1 above is selected from Compounds 1 to 387 below:

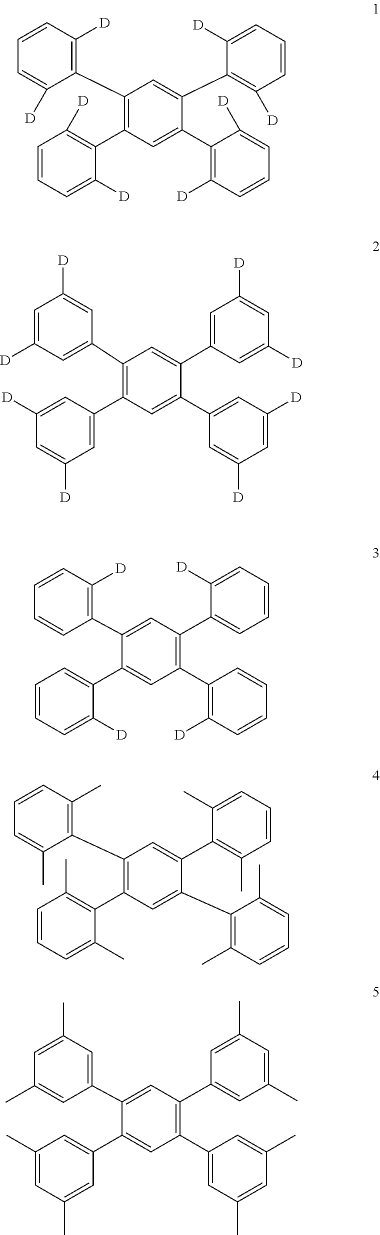

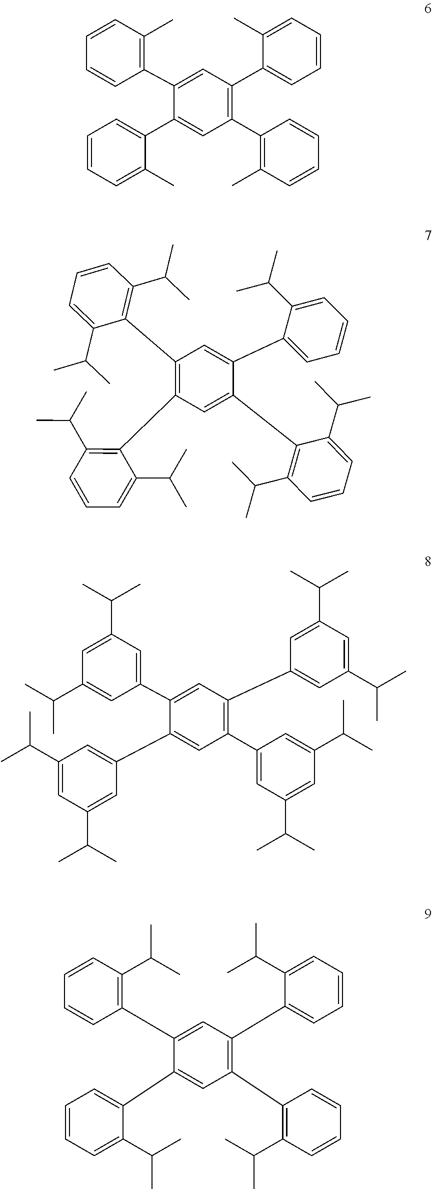

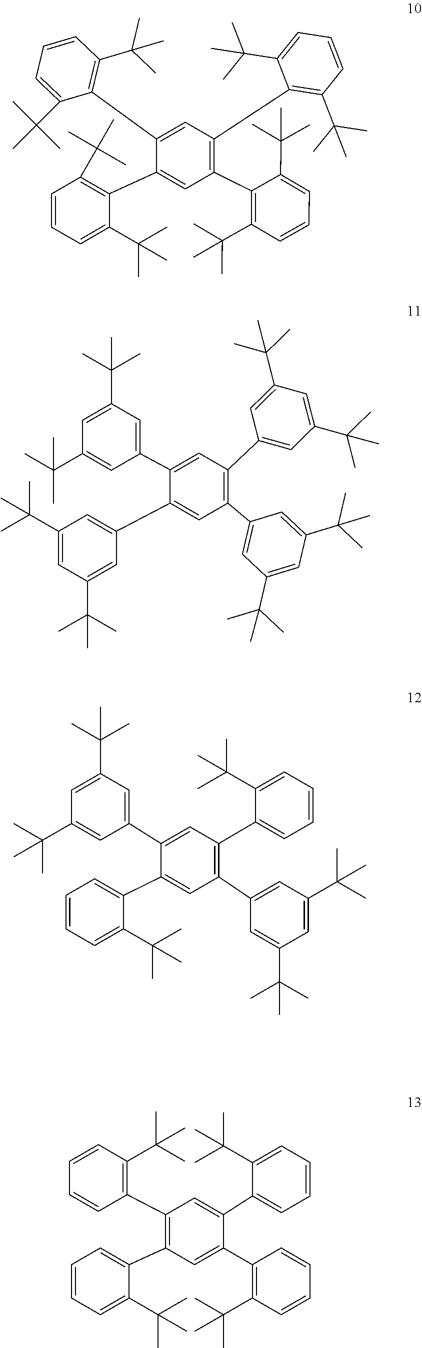

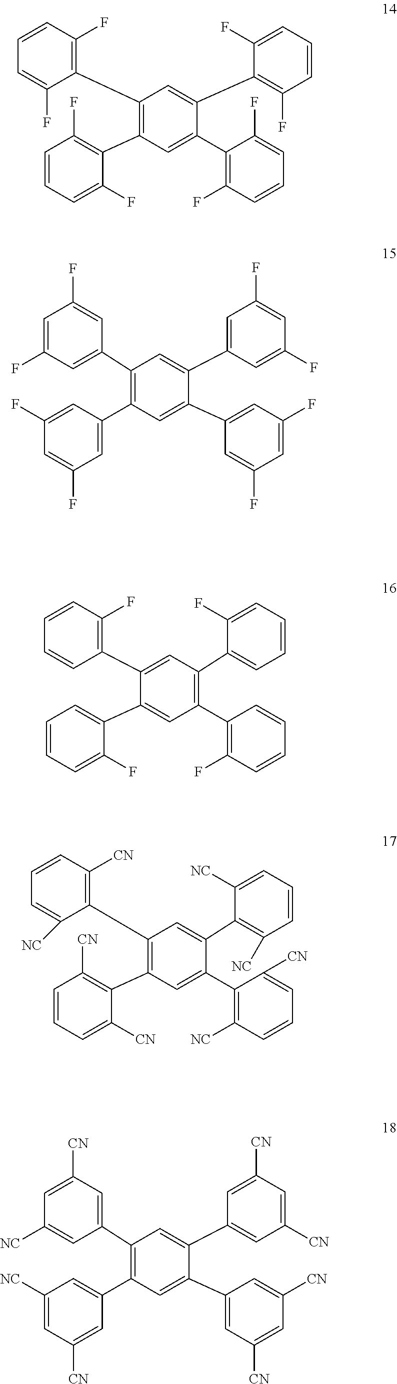 [SMILES: Fc1cccc(F)c1-c1cc(-c2c(F)cccc2F)c(-c2c(F)cccc2F)cc1-c1c(F)cccc1F ; Fc1cc(F)cc(-c2cc(-c3cc(F)cc(F)c3)c(-c3cc(F)cc(F)c3)cc2-c2cc(F)cc(F)c2)c1 ; Fc1ccccc1-c1cc(-c2ccccc2F)c(-c2ccccc2F)cc1-c1ccccc1F ; N#Cc1cccc(C#N)c1-c1cc(-c2c(C#N)cccc2C#N)c(-c2c(C#N)cccc2C#N)cc1-c1c(C#N)cccc1C#N ; N#Cc1cc(C#N)cc(-c2cc(-c3cc(C#N)cc(C#N)c3)c(-c3cc(C#N)cc(C#N)c3)cc2-c2cc(C#N)cc(C#N)c2)c1]

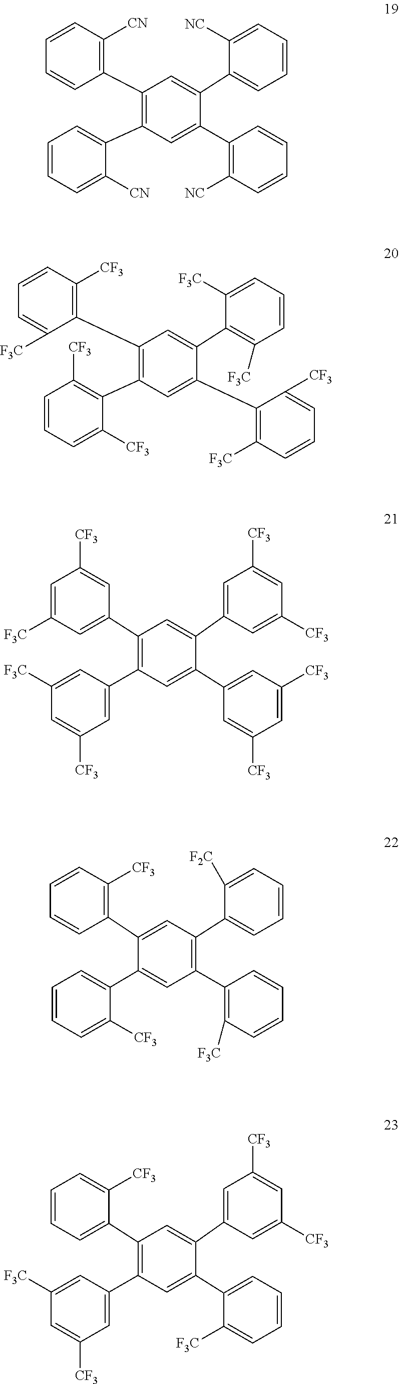

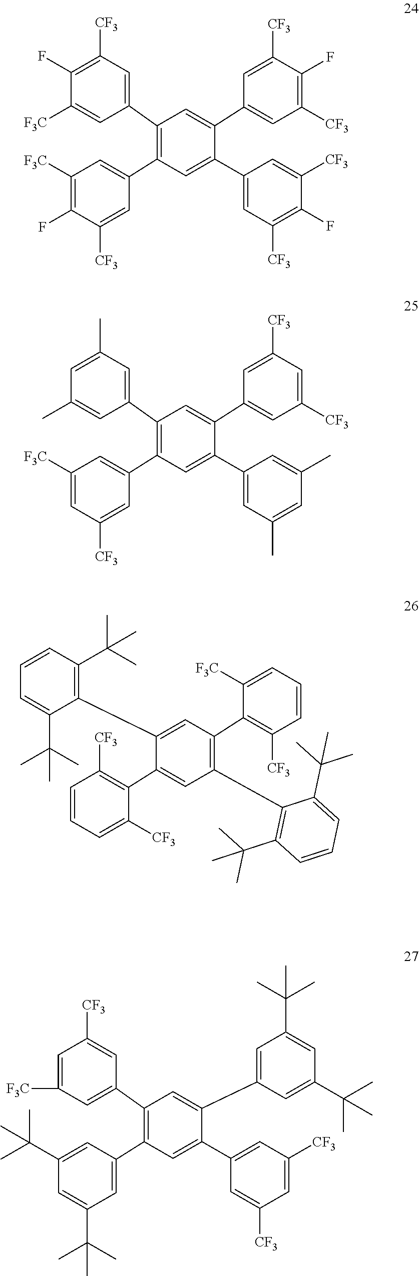

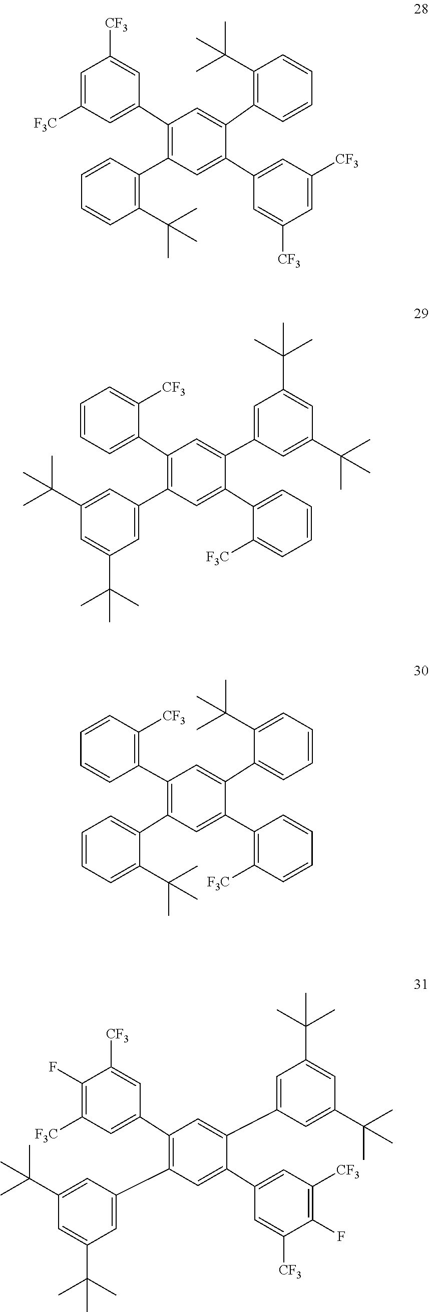

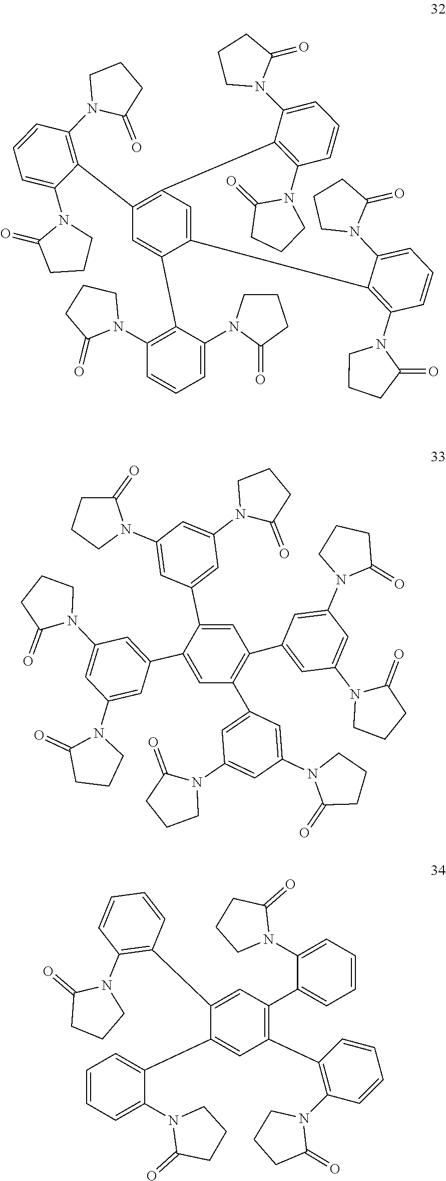 [SMILES: O=C1CCCN1c1cccc(N2CCCC2=O)c1-c1cc(-c2c(N3CCCC3=O)cccc2N2CCCC2=O)c(-c2c(N3CCCC3=O)cccc2N2CCCC2=O)cc1-c1c(N2CCCC2=O)cccc1N1CCCC1=O ; O=C1CCCN1c1cc(-c2cc(-c3cc(N4CCCC4=O)cc(N4CCCC4=O)c3)c(-c3cc(N4CCCC4=O)cc(N4CCCC4=O)c3)cc2-c2cc(N3CCCC3=O)cc(N3CCCC3=O)c2)cc(N2CCCC2=O)c1 ; O=C1CCCN1c1ccccc1-c1cc(-c2ccccc2N2CCCC2=O)c(-c2ccccc2N2CCCC2=O)cc1-c1ccccc1N1CCCC1=O]

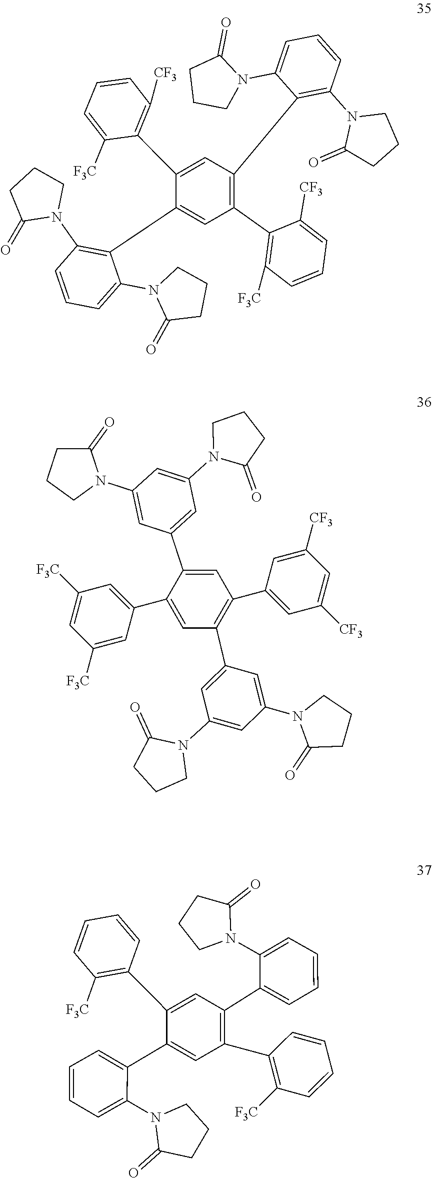 [SMILES: O=C1CCCN1c1cccc(N2CCCC2=O)c1-c1cc(-c2c(C(F)(F)F)cccc2C(F)(F)F)c(-c2c(N3CCCC3=O)cccc2N2CCCC2=O)cc1-c1c(C(F)(F)F)cccc1C(F)(F)F ; O=C1CCCN1c1cc(-c2cc(-c3cc(C(F)(F)F)cc(C(F)(F)F)c3)c(-c3cc(N4CCCC4=O)cc(N4CCCC4=O)c3)cc2-c2cc(C(F)(F)F)cc(C(F)(F)F)c2)cc(N2CCCC2=O)c1 ; O=C1CCCN1c1ccccc1-c1cc(-c2ccccc2C(F)(F)F)c(-c2ccccc2N2CCCC2=O)cc1-c1ccccc1C(F)(F)F]

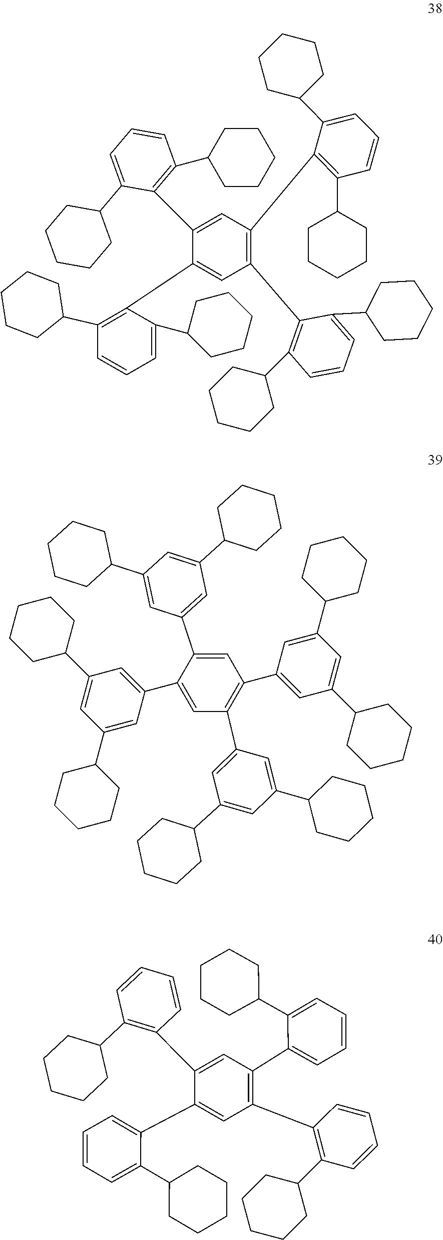

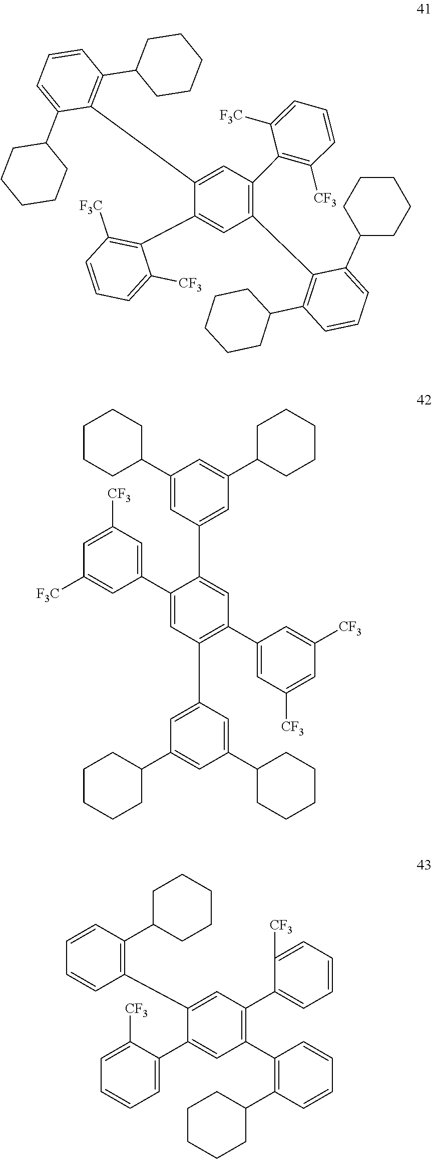 [SMILES: FC(F)(F)c1cccc(C(F)(F)F)c1-c1cc(-c2c(C3CCCCC3)cccc2C2CCCCC2)c(-c2c(C(F)(F)F)cccc2C(F)(F)F)cc1-c1c(C2CCCCC2)cccc1C1CCCCC1 ; FC(F)(F)c1cc(-c2cc(-c3cc(C4CCCCC4)cc(C4CCCCC4)c3)c(-c3cc(C(F)(F)F)cc(C(F)(F)F)c3)cc2-c2cc(C3CCCCC3)cc(C3CCCCC3)c2)cc(C(F)(F)F)c1 ; FC(F)(F)c1ccccc1-c1cc(-c2ccccc2C2CCCCC2)c(-c2ccccc2C(F)(F)F)cc1-c1ccccc1C1CCCCC1]

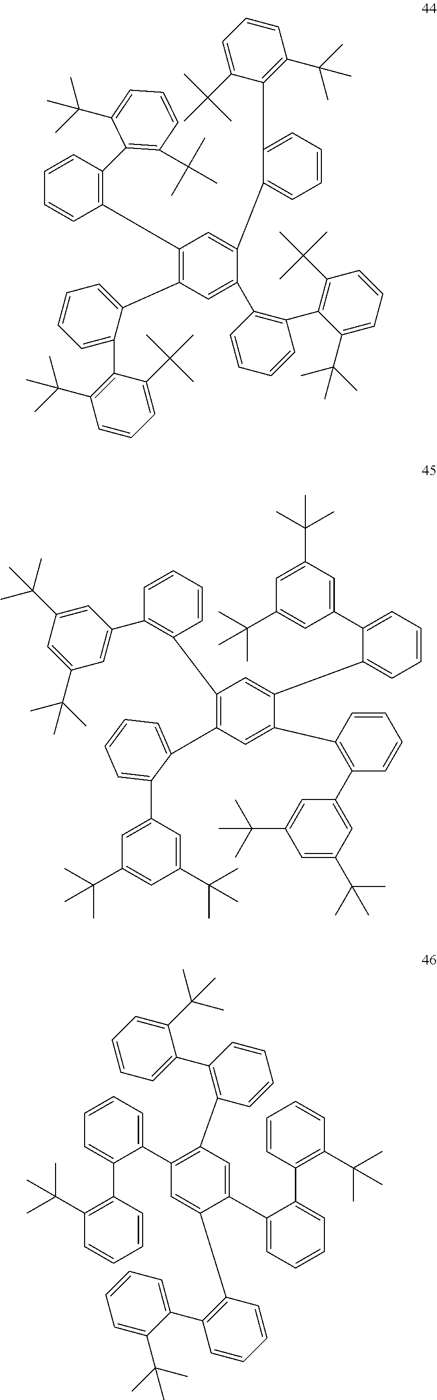

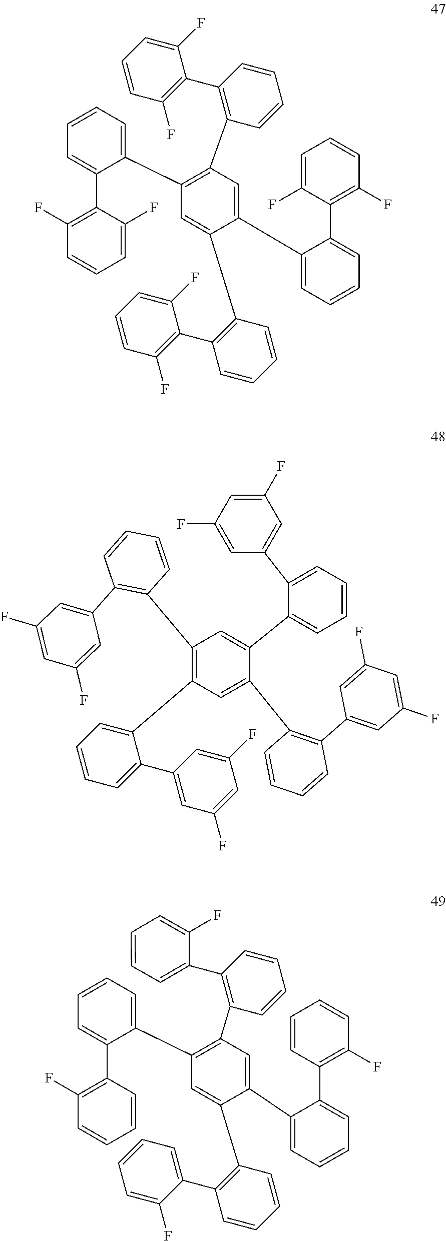 [SMILES: Fc1cccc(F)c1-c1ccccc1-c1cc(-c2ccccc2-c2c(F)cccc2F)c(-c2ccccc2-c2c(F)cccc2F)cc1-c1ccccc1-c1c(F)cccc1F ; Fc1cc(F)cc(-c2ccccc2-c2cc(-c3ccccc3-c3cc(F)cc(F)c3)c(-c3ccccc3-c3cc(F)cc(F)c3)cc2-c2ccccc2-c2cc(F)cc(F)c2)c1 ; Fc1ccccc1-c1ccccc1-c1cc(-c2ccccc2-c2ccccc2F)c(-c2ccccc2-c2ccccc2F)cc1-c1ccccc1-c1ccccc1F]

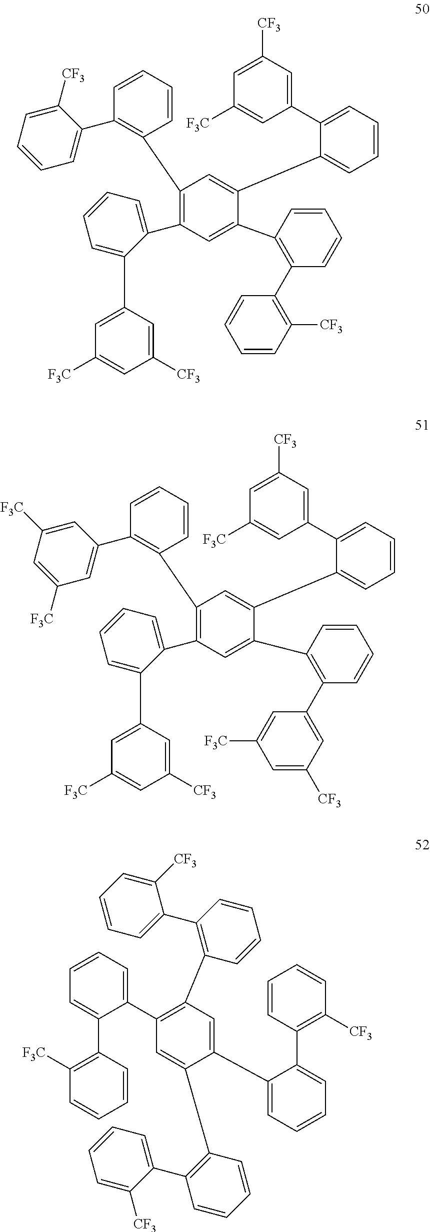 [SMILES: FC(F)(F)c1cc(-c2ccccc2-c2cc(-c3ccccc3-c3ccccc3C(F)(F)F)c(-c3ccccc3-c3cc(C(F)(F)F)cc(C(F)(F)F)c3)cc2-c2ccccc2-c2ccccc2C(F)(F)F)cc(C(F)(F)F)c1 ; FC(F)(F)c1cc(-c2ccccc2-c2cc(-c3ccccc3-c3cc(C(F)(F)F)cc(C(F)(F)F)c3)c(-c3ccccc3-c3cc(C(F)(F)F)cc(C(F)(F)F)c3)cc2-c2ccccc2-c2cc(C(F)(F)F)cc(C(F)(F)F)c2)cc(C(F)(F)F)c1 ; FC(F)(F)c1ccccc1-c1ccccc1-c1cc(-c2ccccc2-c2ccccc2C(F)(F)F)c(-c2ccccc2-c2ccccc2C(F)(F)F)cc1-c1ccccc1-c1ccccc1C(F)(F)F]

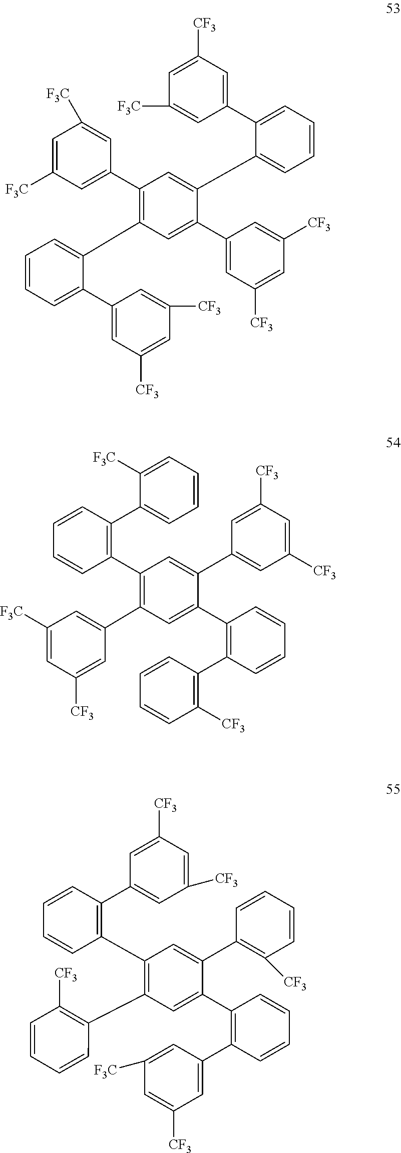 [SMILES: FC(F)(F)c1cc(-c2ccccc2-c2cc(-c3cc(C(F)(F)F)cc(C(F)(F)F)c3)c(-c3ccccc3-c3cc(C(F)(F)F)cc(C(F)(F)F)c3)cc2-c2cc(C(F)(F)F)cc(C(F)(F)F)c2)cc(C(F)(F)F)c1 ; FC(F)(F)c1cc(-c2cc(-c3ccccc3-c3ccccc3C(F)(F)F)c(-c3cc(C(F)(F)F)cc(C(F)(F)F)c3)cc2-c2ccccc2-c2ccccc2C(F)(F)F)cc(C(F)(F)F)c1 ; FC(F)(F)c1cc(-c2ccccc2-c2cc(-c3ccccc3C(F)(F)F)c(-c3ccccc3-c3cc(C(F)(F)F)cc(C(F)(F)F)c3)cc2-c2ccccc2C(F)(F)F)cc(C(F)(F)F)c1]

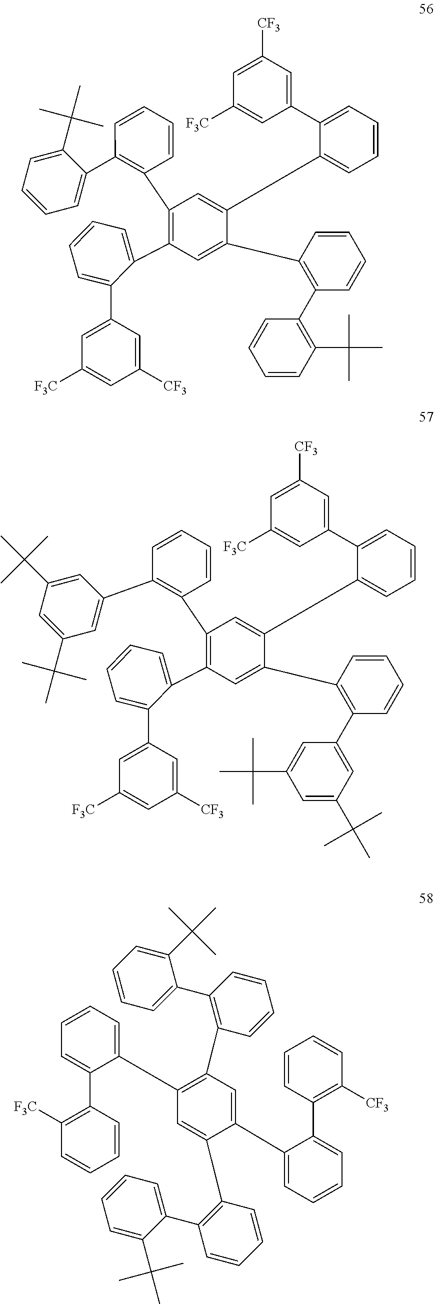

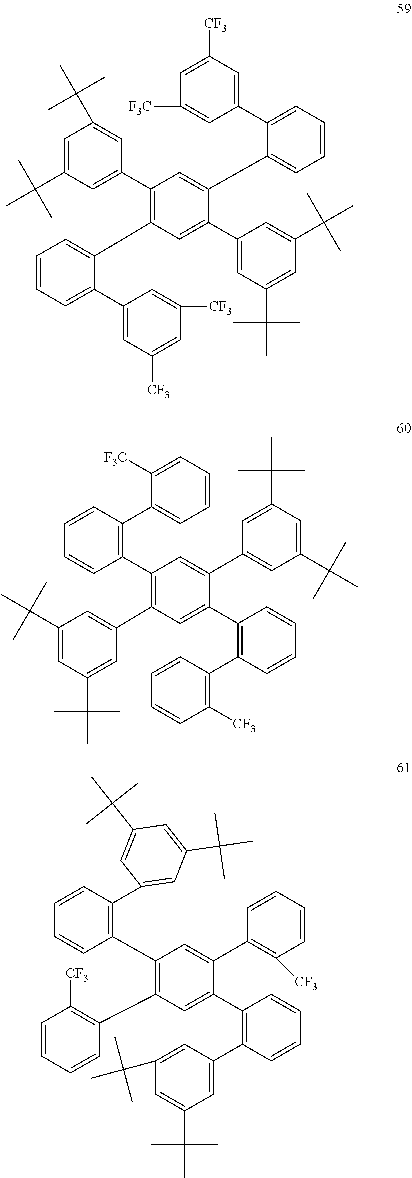

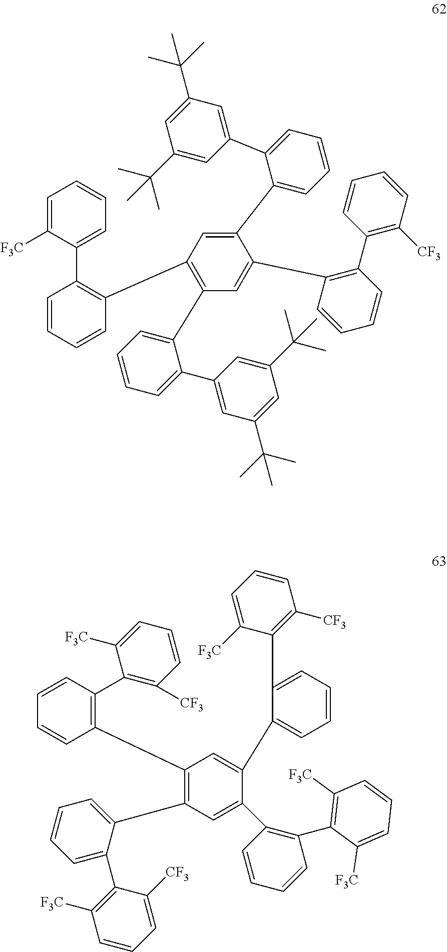 [SMILES: CC(C)(C)c1cc(-c2ccccc2-c2cc(-c3ccccc3-c3ccccc3C(F)(F)F)c(-c3ccccc3-c3cc(C(C)(C)C)cc(C(C)(C)C)c3)cc2-c2ccccc2-c2ccccc2C(F)(F)F)cc(C(C)(C)C)c1 ; FC(F)(F)c1cccc(C(F)(F)F)c1-c1ccccc1-c1cc(-c2ccccc2-c2c(C(F)(F)F)cccc2C(F)(F)F)c(-c2ccccc2-c2c(C(F)(F)F)cccc2C(F)(F)F)cc1-c1ccccc1-c1c(C(F)(F)F)cccc1C(F)(F)F]

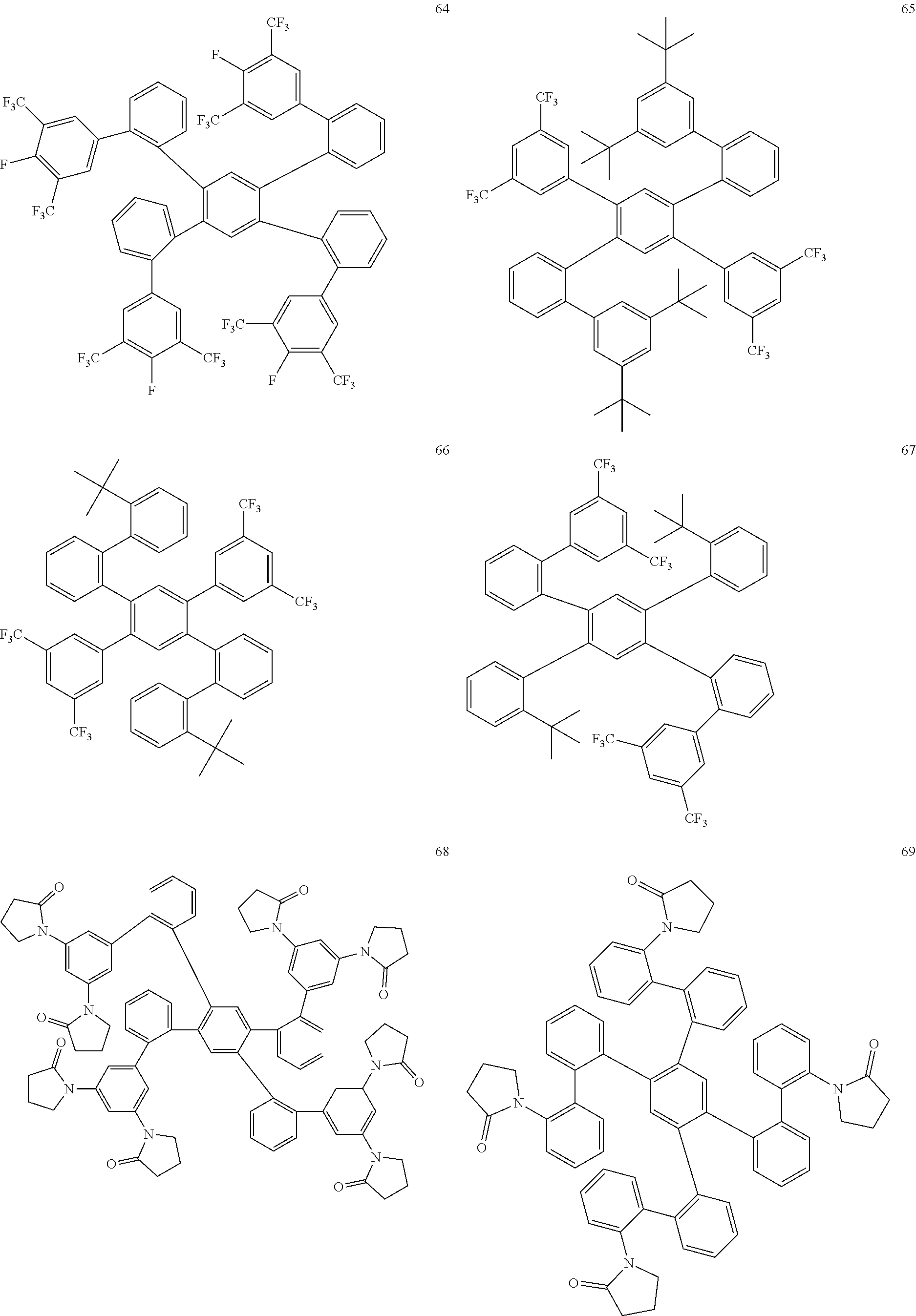

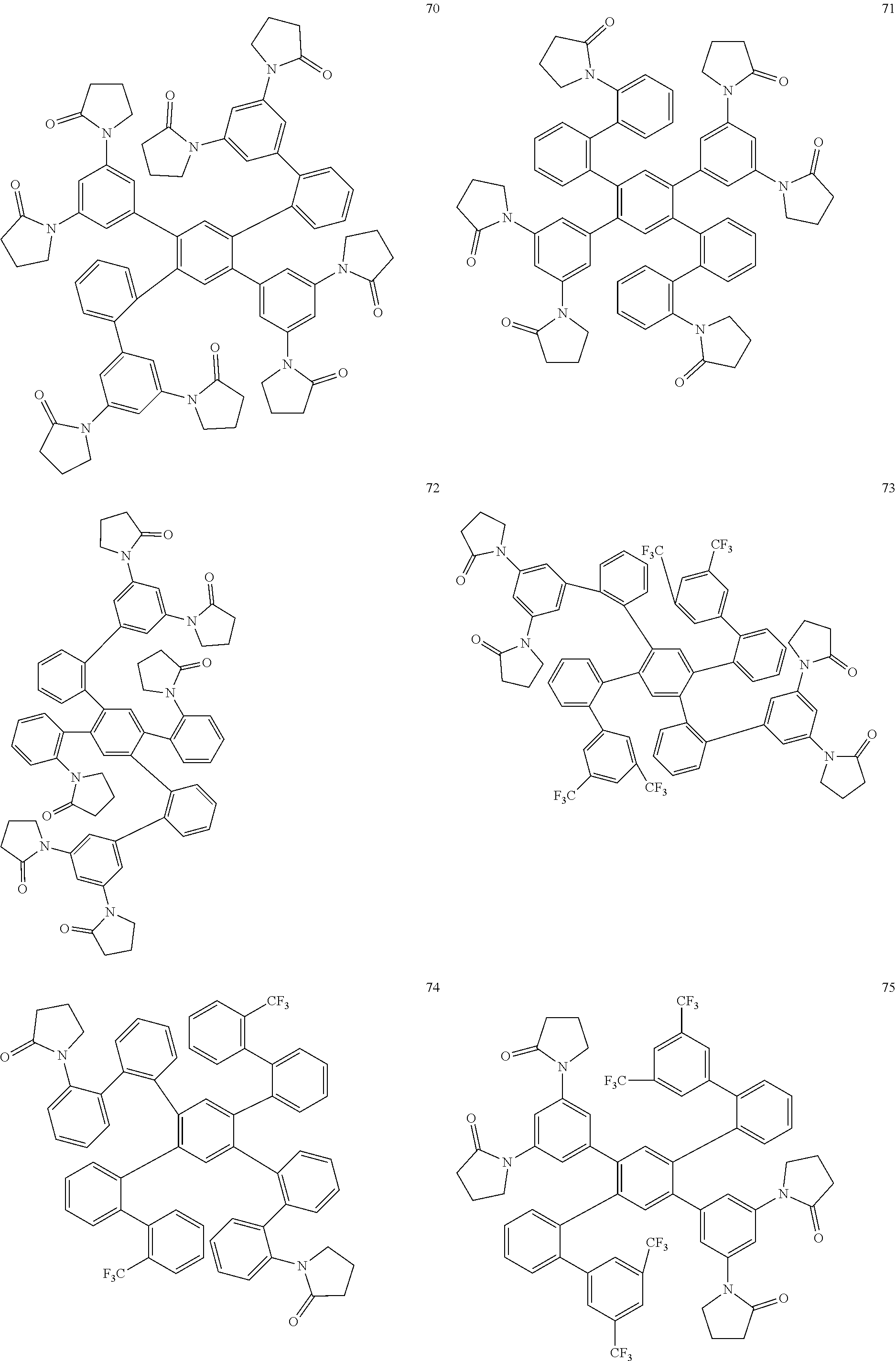

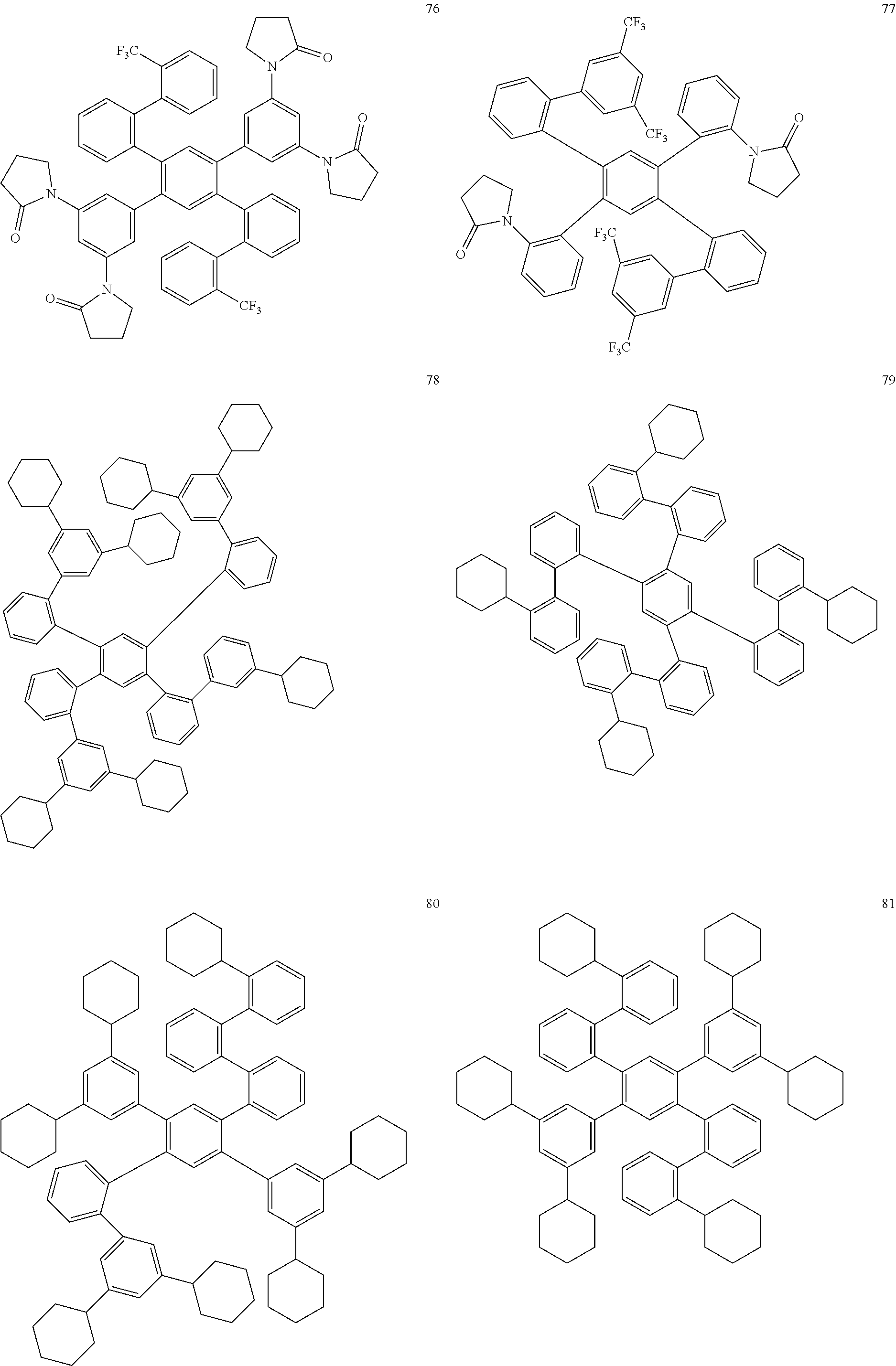

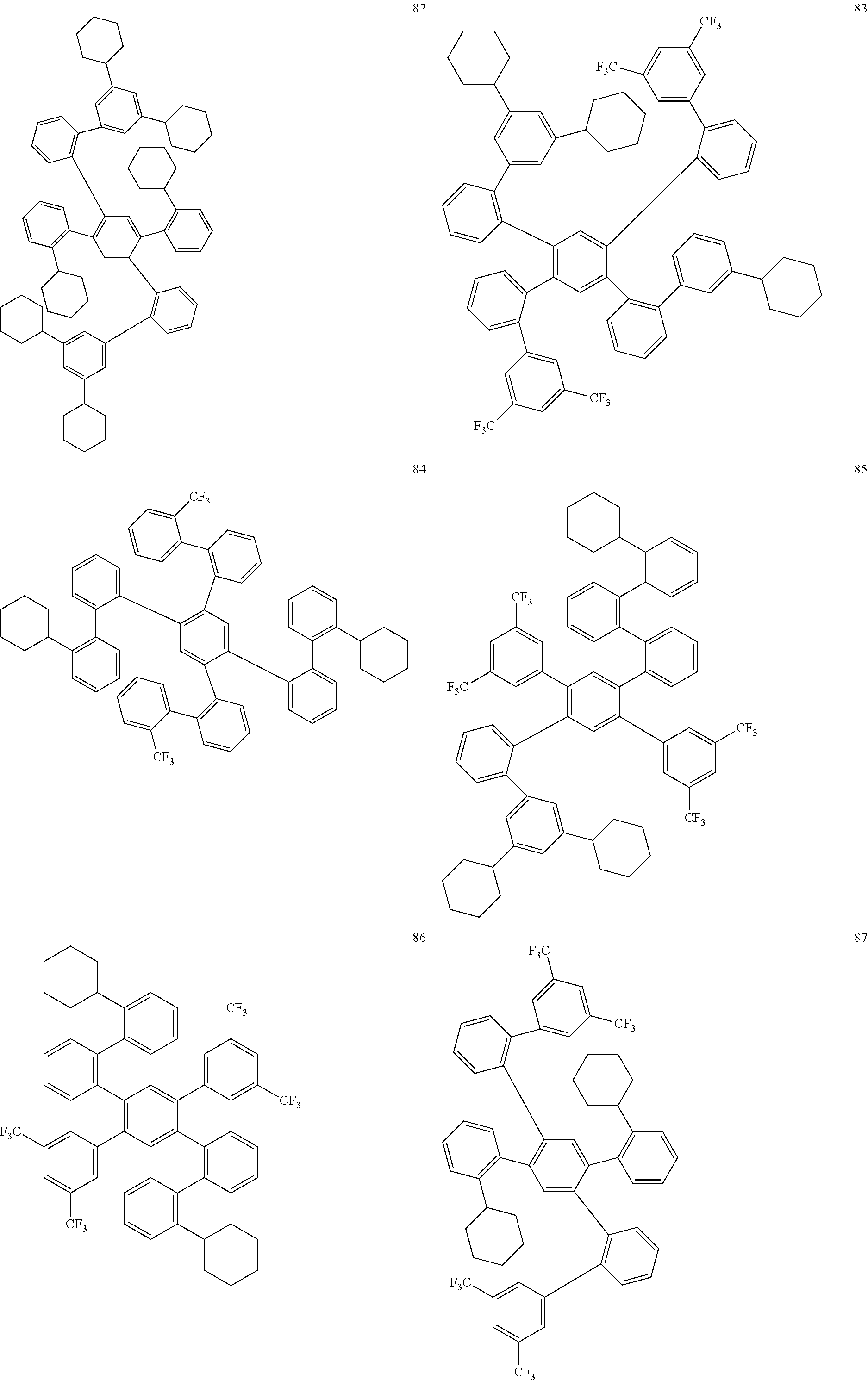

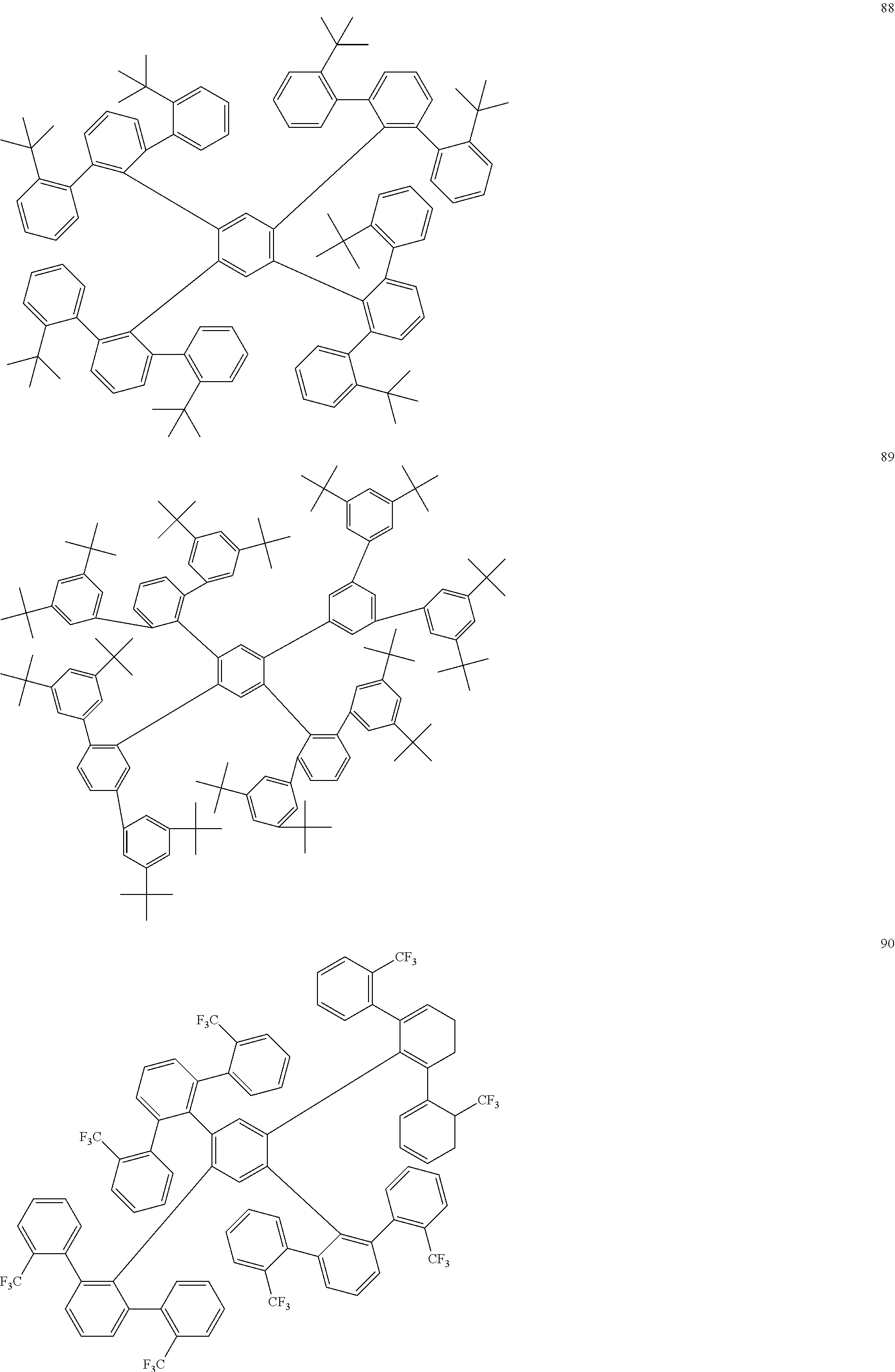

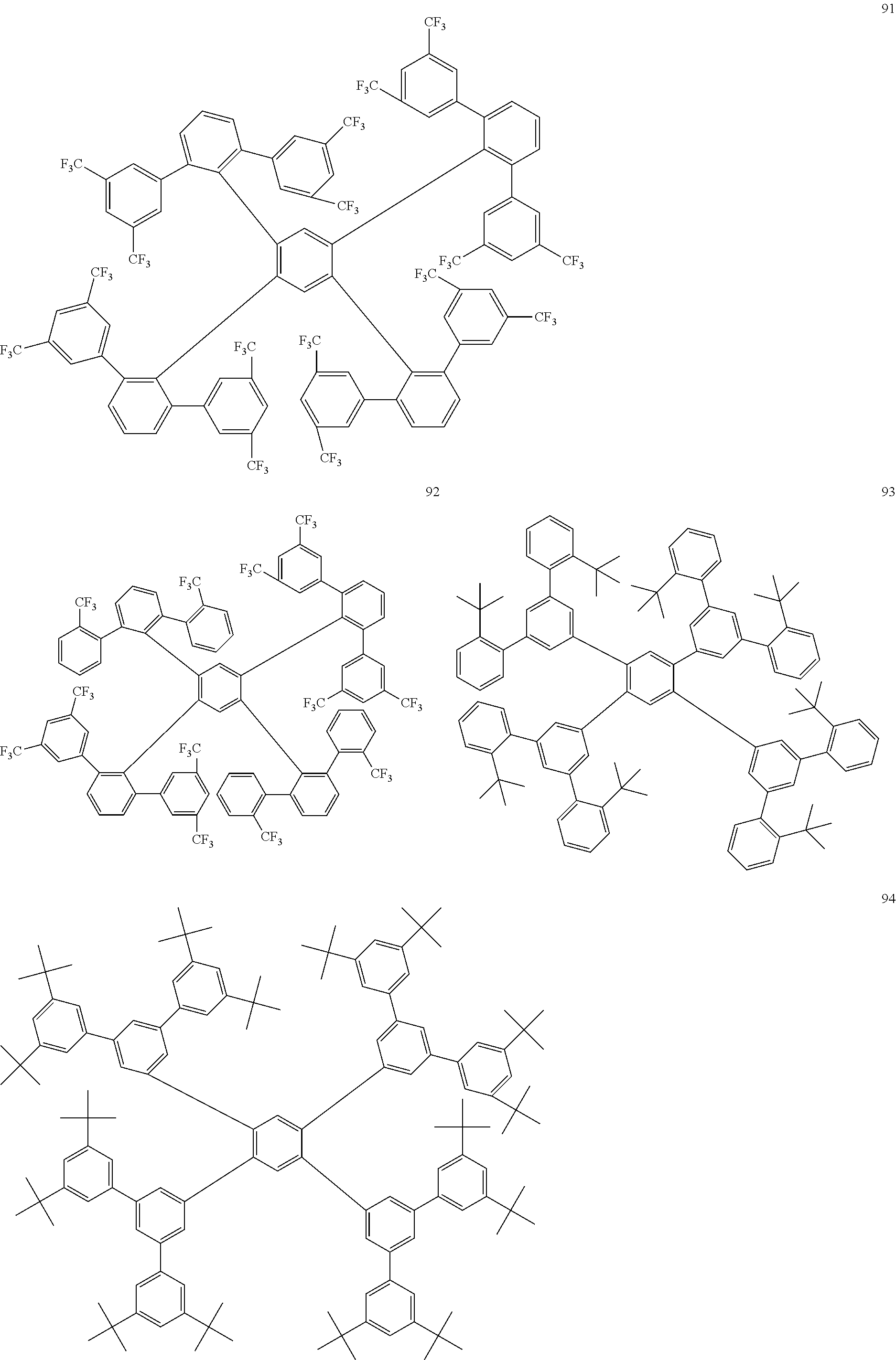

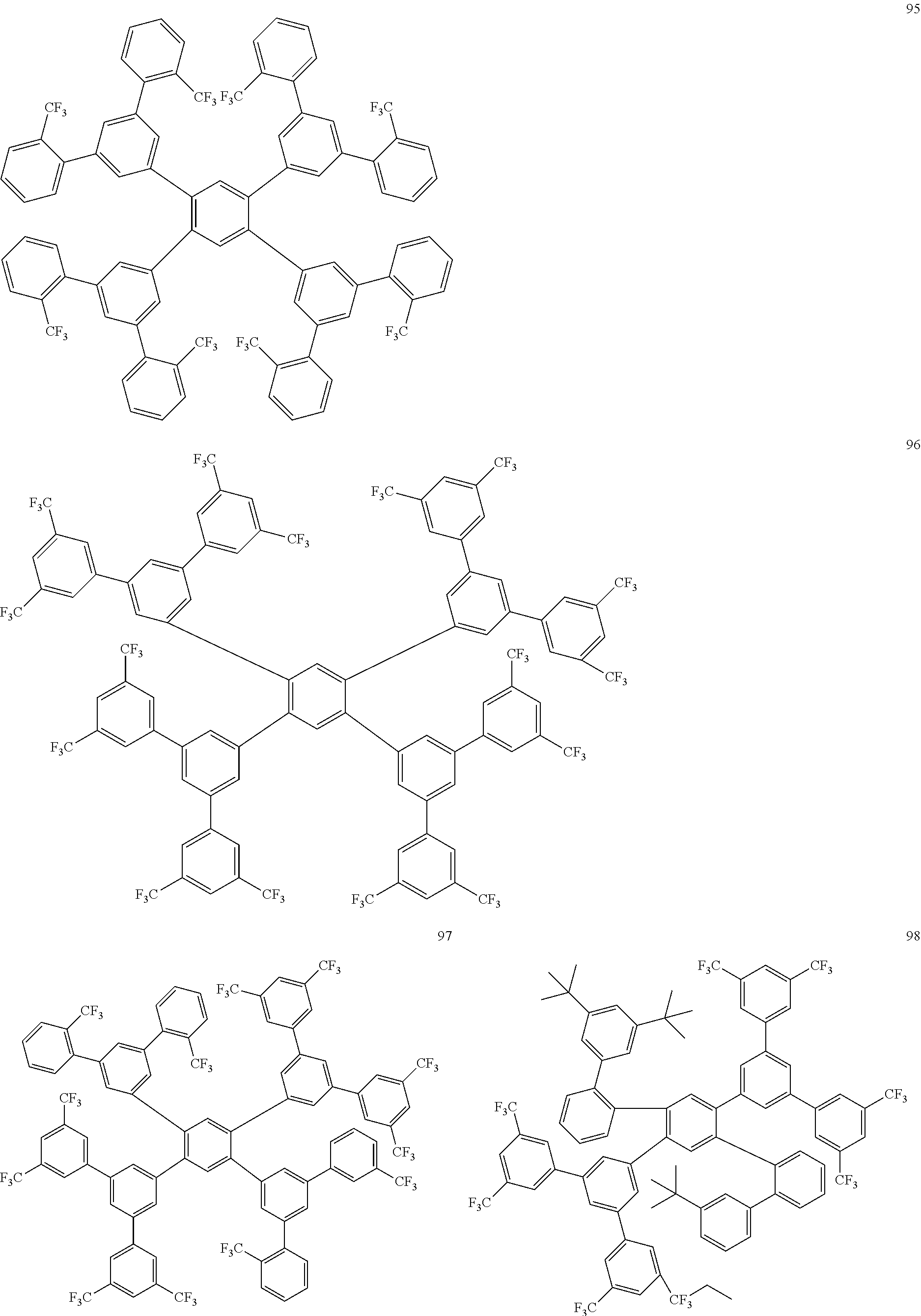

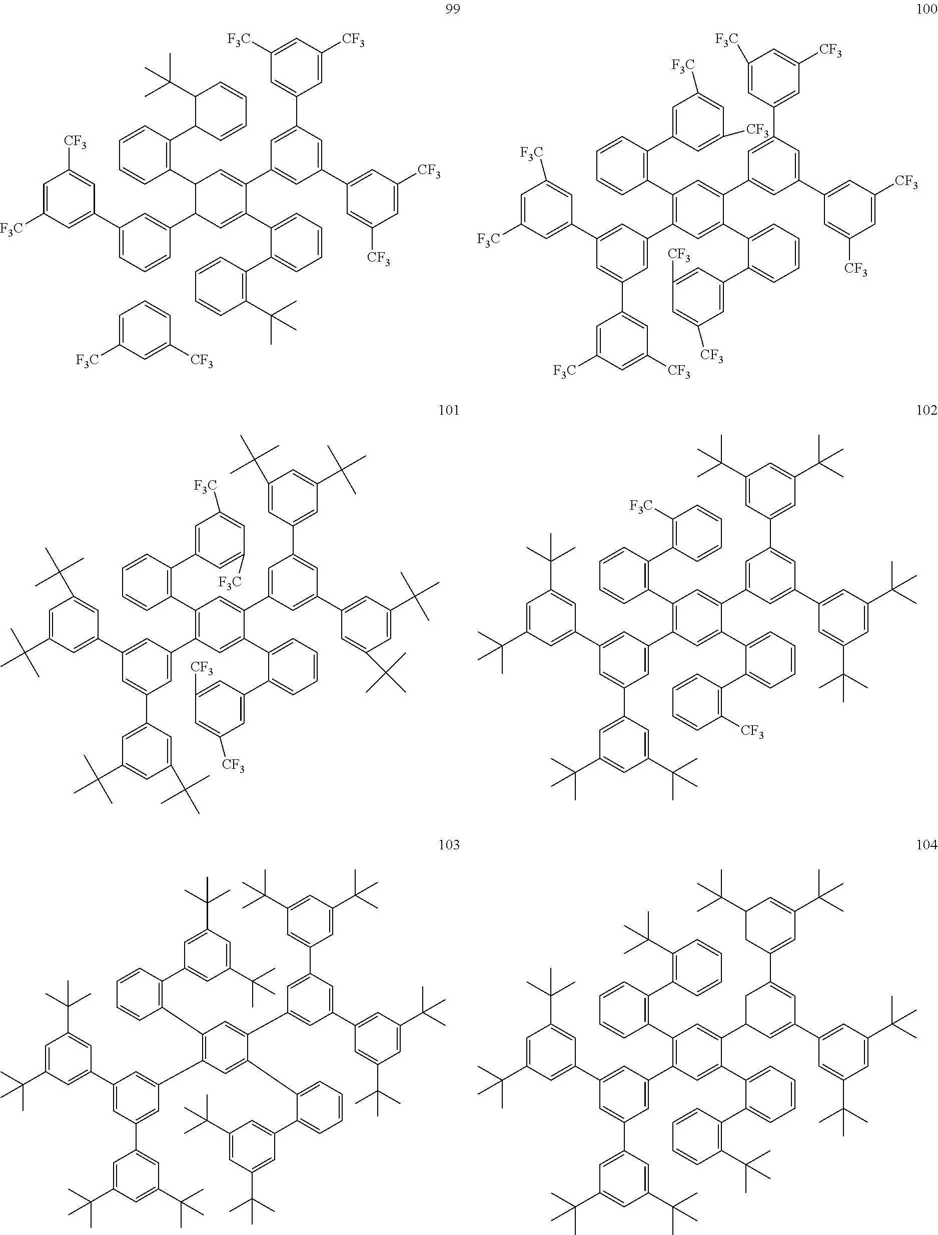

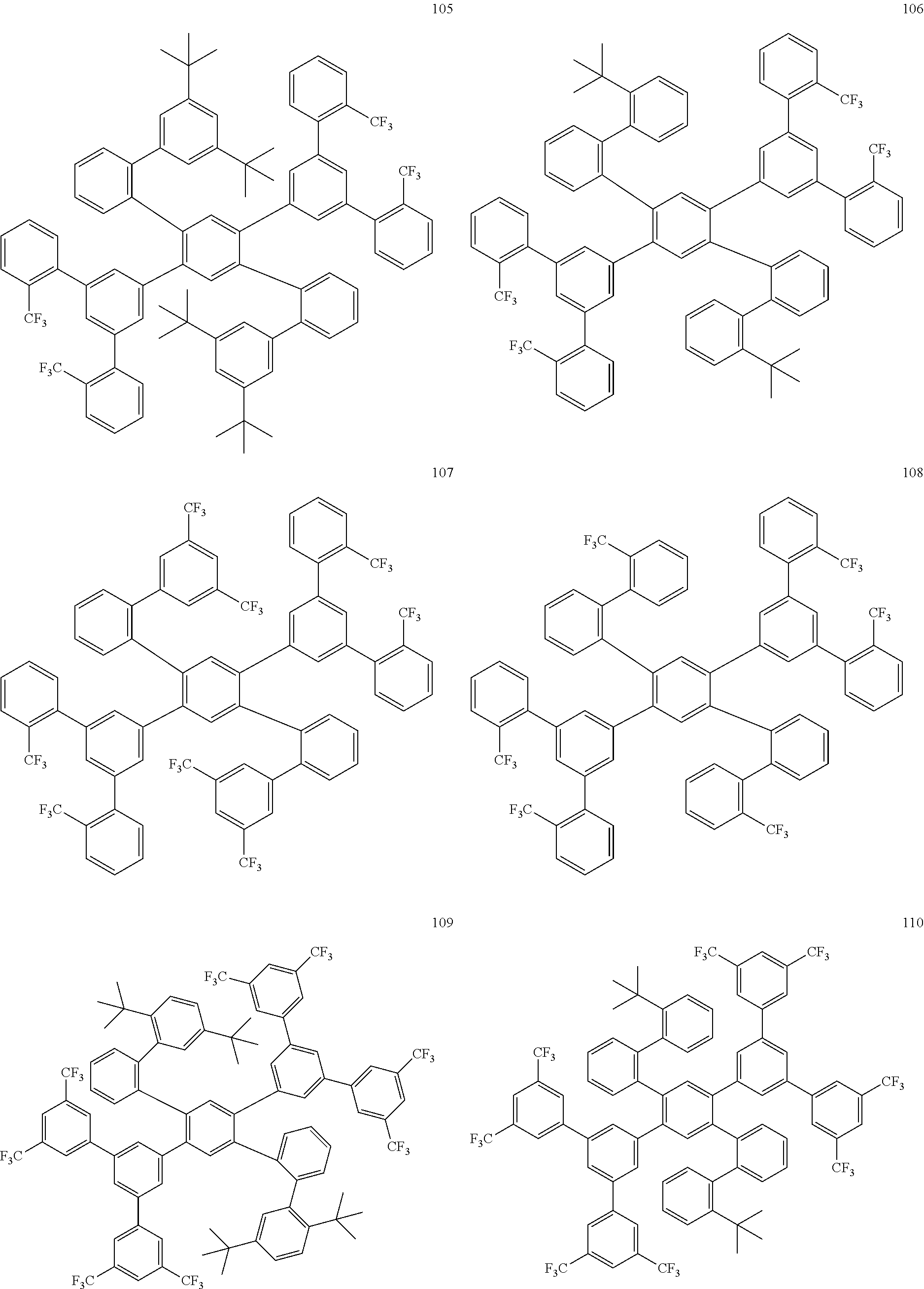

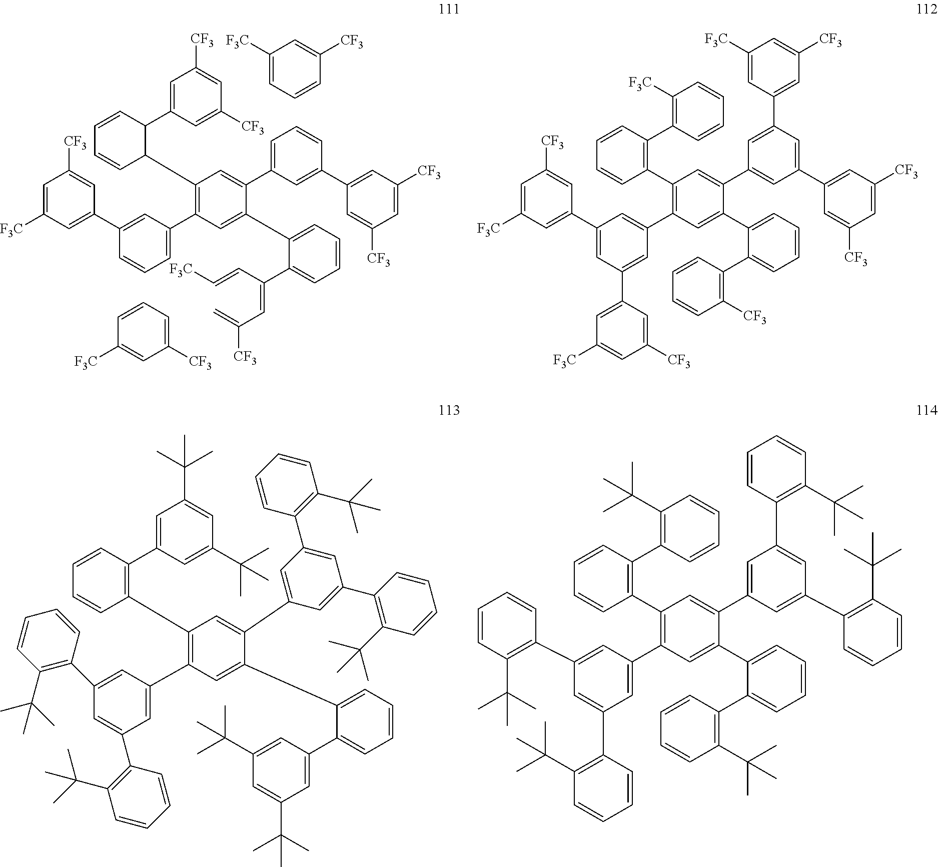

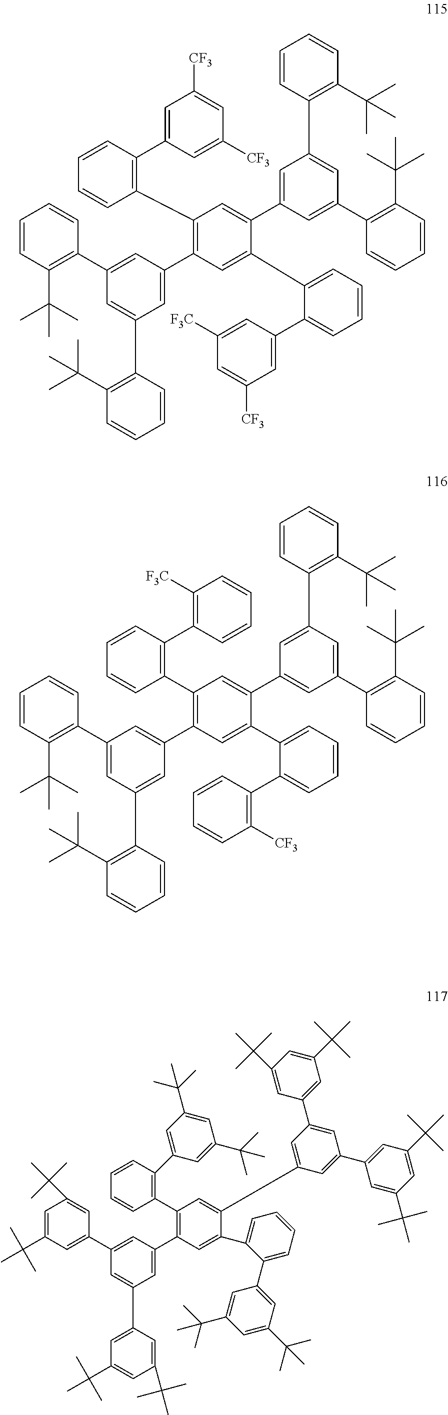

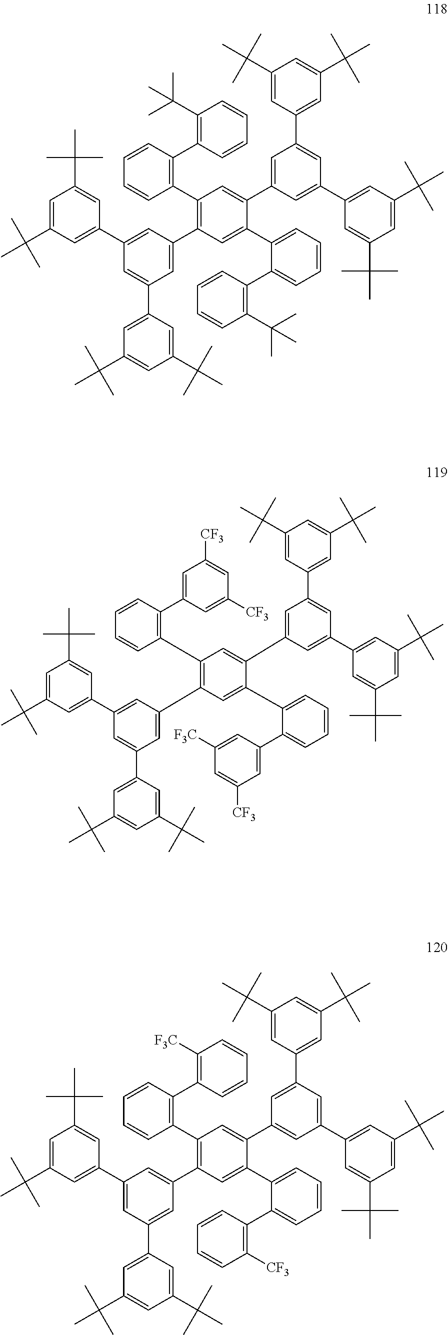

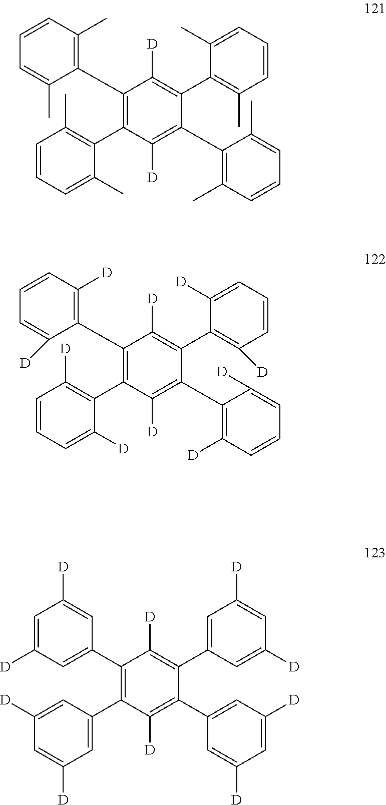 [SMILES: [2H]c1c(-c2c(C)cccc2C)c(-c2c(C)cccc2C)c([2H])c(-c2c(C)cccc2C)c1-c1c(C)cccc1C ; [2H]c1cccc([2H])c1-c1c([2H])c(-c2c([2H])cccc2[2H])c(-c2c([2H])cccc2[2H])c([2H])c1-c1c([2H])cccc1[2H] ; [2H]c1cc([2H])cc(-c2c([2H])c(-c3cc([2H])cc([2H])c3)c(-c3cc([2H])cc([2H])c3)c([2H])c2-c2cc([2H])cc([2H])c2)c1]

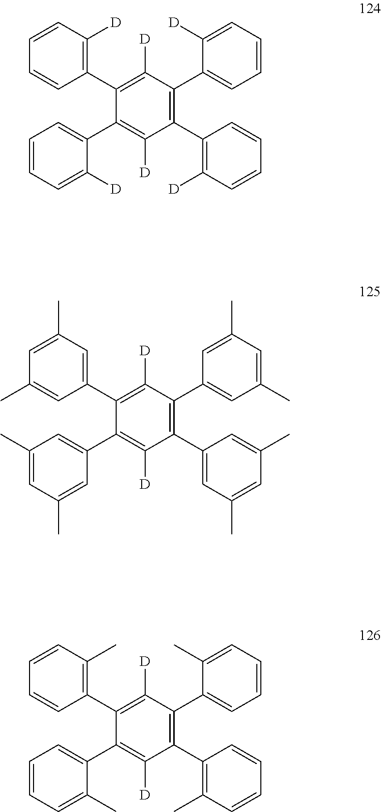

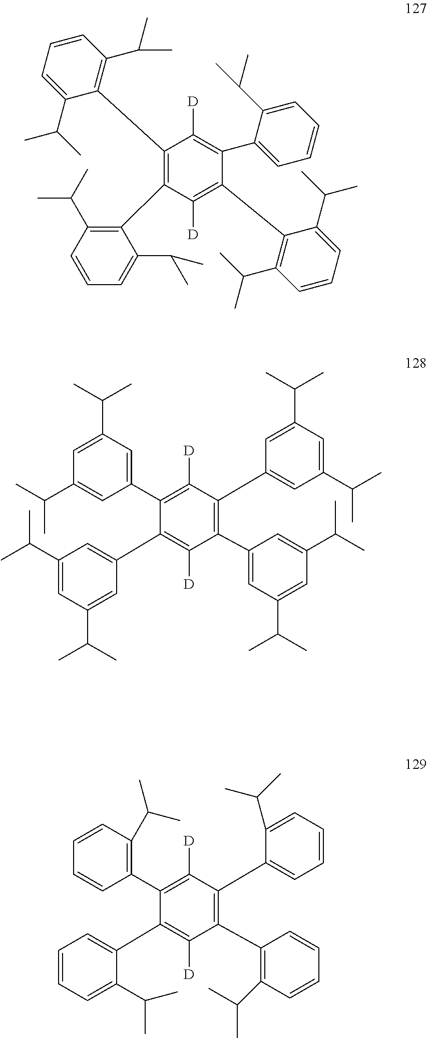

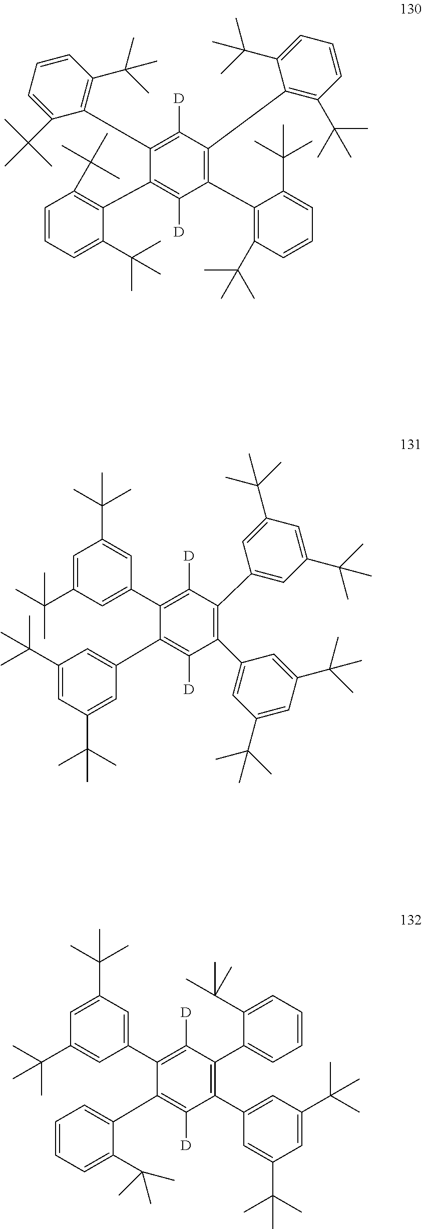

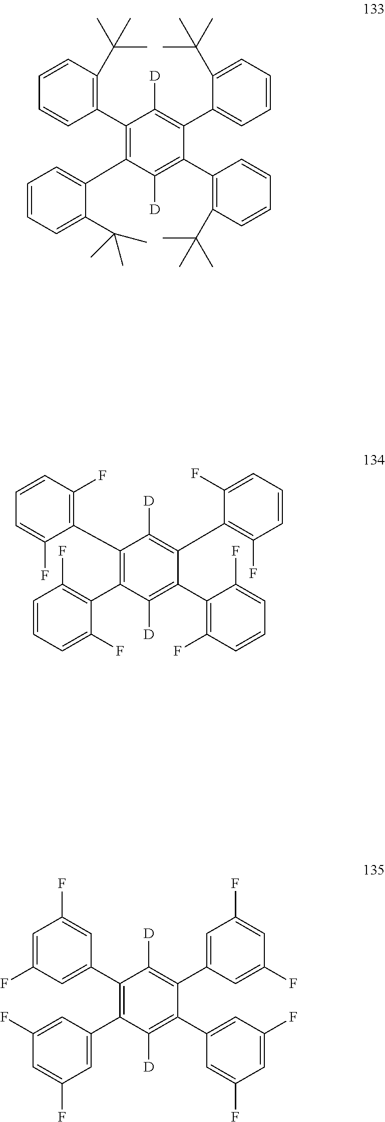 [SMILES: [2H]c1c(-c2ccccc2C(C)(C)C)c(-c2ccccc2C(C)(C)C)c([2H])c(-c2ccccc2C(C)(C)C)c1-c1ccccc1C(C)(C)C ; [2H]c1c(-c2c(F)cccc2F)c(-c2c(F)cccc2F)c([2H])c(-c2c(F)cccc2F)c1-c1c(F)cccc1F ; [2H]c1c(-c2cc(F)cc(F)c2)c(-c2cc(F)cc(F)c2)c([2H])c(-c2cc(F)cc(F)c2)c1-c1cc(F)cc(F)c1]

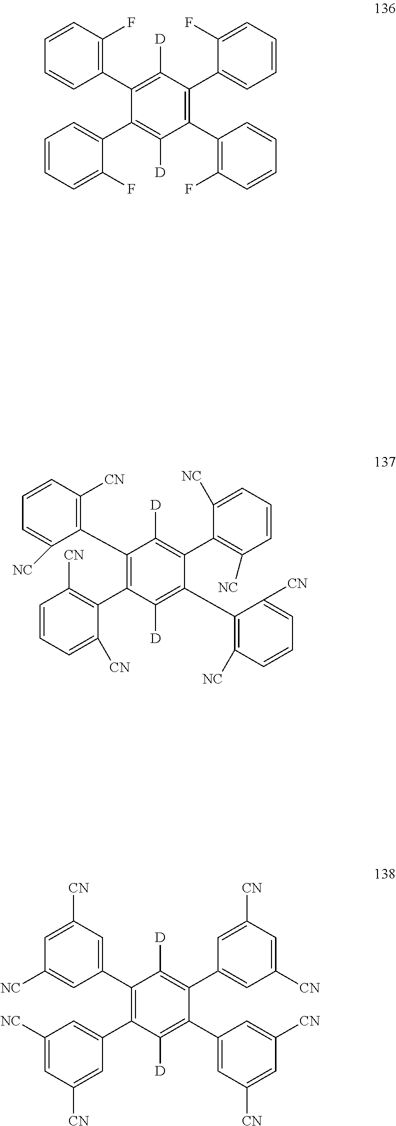 [SMILES: [2H]c1c(-c2ccccc2F)c(-c2ccccc2F)c([2H])c(-c2ccccc2F)c1-c1ccccc1F ; [2H]c1c(-c2c(C#N)cccc2C#N)c(-c2c(C#N)cccc2C#N)c([2H])c(-c2c(C#N)cccc2C#N)c1-c1c(C#N)cccc1C#N ; [2H]c1c(-c2cc(C#N)cc(C#N)c2)c(-c2cc(C#N)cc(C#N)c2)c([2H])c(-c2cc(C#N)cc(C#N)c2)c1-c1cc(C#N)cc(C#N)c1]

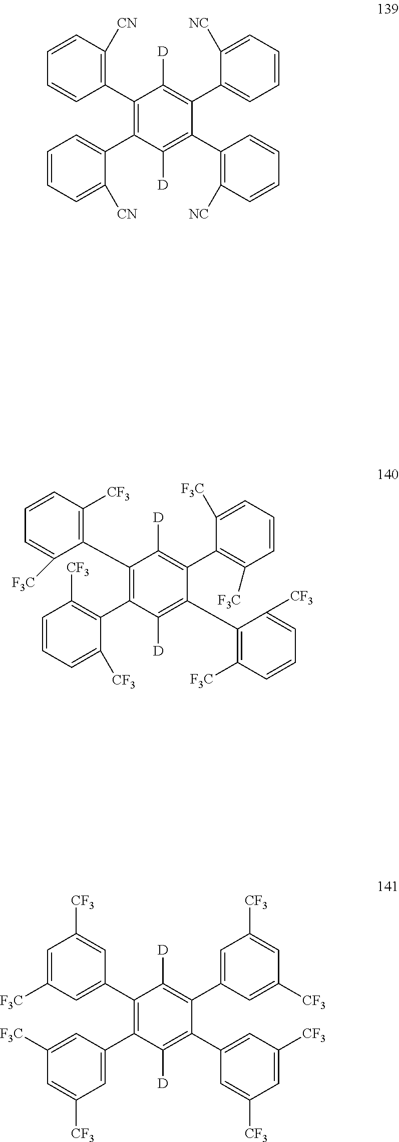 [SMILES: [2H]c1c(-c2ccccc2C#N)c(-c2ccccc2C#N)c([2H])c(-c2ccccc2C#N)c1-c1ccccc1C#N ; [2H]c1c(-c2c(C(F)(F)F)cccc2C(F)(F)F)c(-c2c(C(F)(F)F)cccc2C(F)(F)F)c([2H])c(-c2c(C(F)(F)F)cccc2C(F)(F)F)c1-c1c(C(F)(F)F)cccc1C(F)(F)F ; [2H]c1c(-c2cc(C(F)(F)F)cc(C(F)(F)F)c2)c(-c2cc(C(F)(F)F)cc(C(F)(F)F)c2)c([2H])c(-c2cc(C(F)(F)F)cc(C(F)(F)F)c2)c1-c1cc(C(F)(F)F)cc(C(F)(F)F)c1]

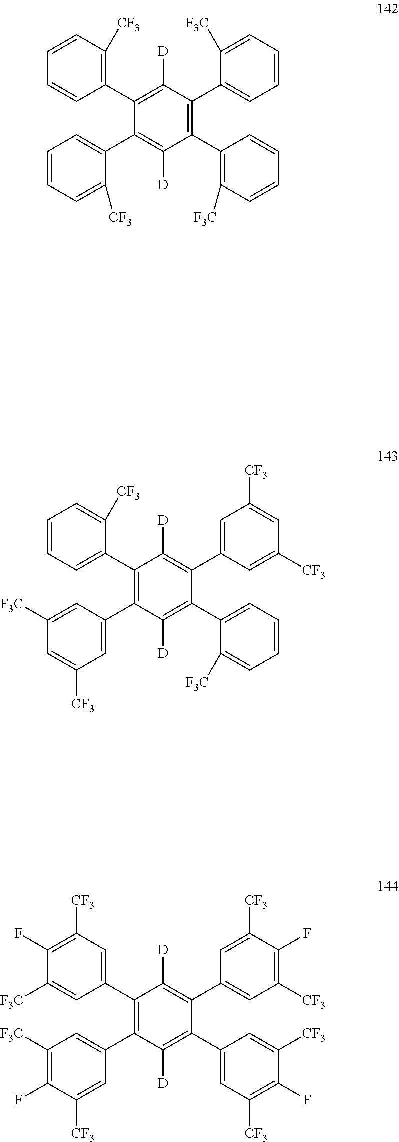 [SMILES: [2H]c1c(-c2ccccc2C(F)(F)F)c(-c2ccccc2C(F)(F)F)c([2H])c(-c2ccccc2C(F)(F)F)c1-c1ccccc1C(F)(F)F ; [2H]c1c(-c2cc(C(F)(F)F)cc(C(F)(F)F)c2)c(-c2ccccc2C(F)(F)F)c([2H])c(-c2cc(C(F)(F)F)cc(C(F)(F)F)c2)c1-c1ccccc1C(F)(F)F ; [2H]c1c(-c2cc(C(F)(F)F)c(F)c(C(F)(F)F)c2)c(-c2cc(C(F)(F)F)c(F)c(C(F)(F)F)c2)c([2H])c(-c2cc(C(F)(F)F)c(F)c(C(F)(F)F)c2)c1-c1cc(C(F)(F)F)c(F)c(C(F)(F)F)c1]

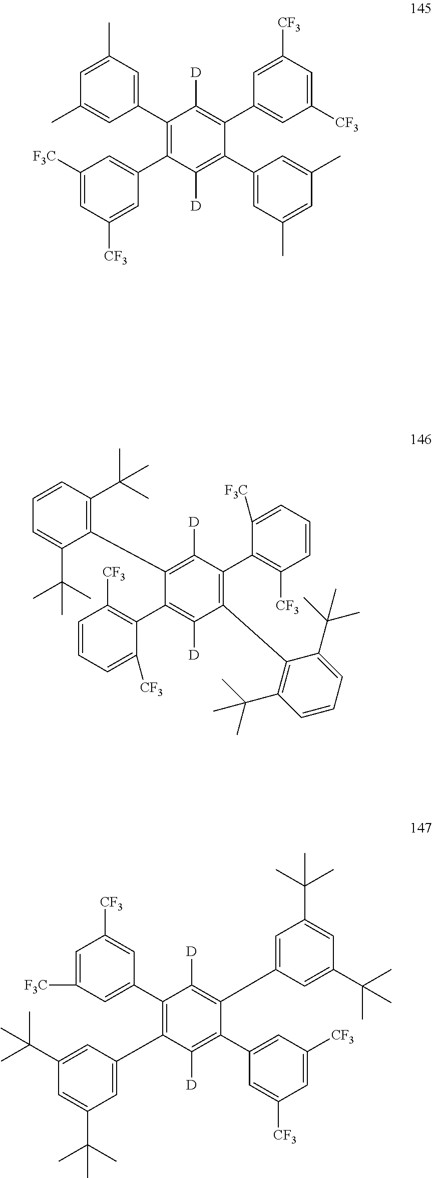

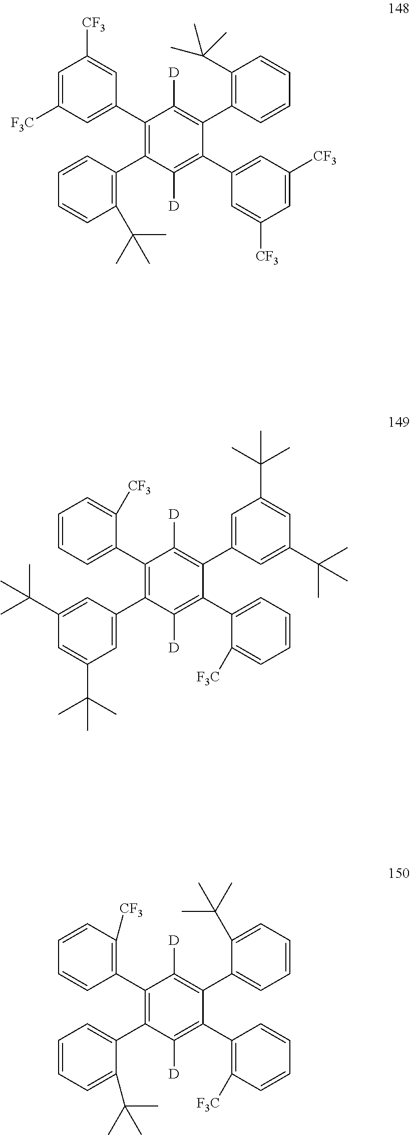 [SMILES: [2H]c1c(-c2cc(C(F)(F)F)cc(C(F)(F)F)c2)c(-c2ccccc2C(C)(C)C)c([2H])c(-c2cc(C(F)(F)F)cc(C(F)(F)F)c2)c1-c1ccccc1C(C)(C)C ; [2H]c1c(-c2cc(C(C)(C)C)cc(C(C)(C)C)c2)c(-c2ccccc2C(F)(F)F)c([2H])c(-c2cc(C(C)(C)C)cc(C(C)(C)C)c2)c1-c1ccccc1C(F)(F)F ; [2H]c1c(-c2ccccc2C(C)(C)C)c(-c2ccccc2C(F)(F)F)c([2H])c(-c2ccccc2C(C)(C)C)c1-c1ccccc1C(F)(F)F]

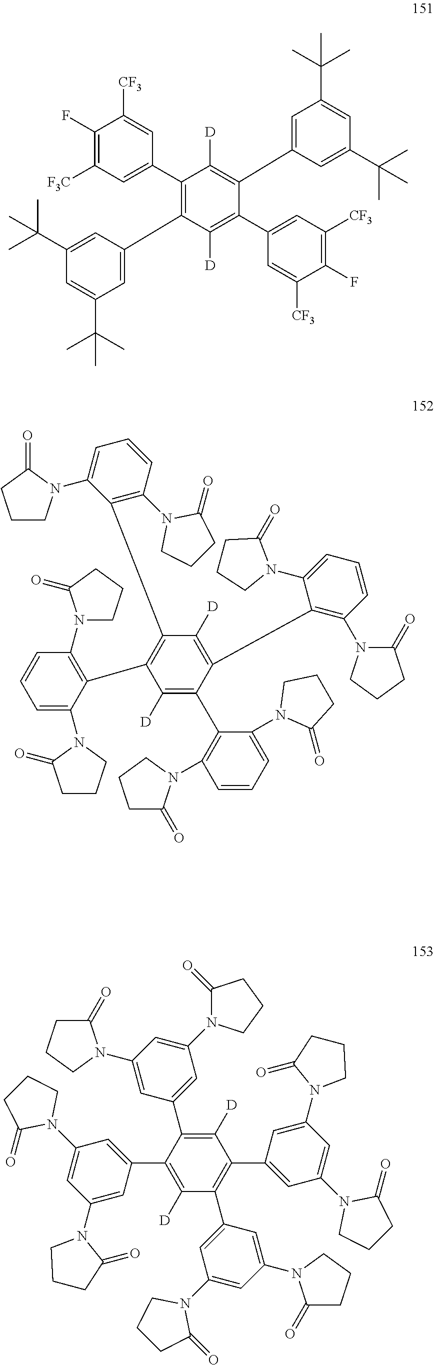 [SMILES: [2H]c1c(-c2cc(C(C)(C)C)cc(C(C)(C)C)c2)c(-c2cc(C(F)(F)F)c(F)c(C(F)(F)F)c2)c([2H])c(-c2cc(C(C)(C)C)cc(C(C)(C)C)c2)c1-c1cc(C(F)(F)F)c(F)c(C(F)(F)F)c1 ; [2H]c1c(-c2c(N3CCCC3=O)cccc2N2CCCC2=O)c(-c2c(N3CCCC3=O)cccc2N2CCCC2=O)c([2H])c(-c2c(N3CCCC3=O)cccc2N2CCCC2=O)c1-c1c(N2CCCC2=O)cccc1N1CCCC1=O ; [2H]c1c(-c2cc(N3CCCC3=O)cc(N3CCCC3=O)c2)c(-c2cc(N3CCCC3=O)cc(N3CCCC3=O)c2)c([2H])c(-c2cc(N3CCCC3=O)cc(N3CCCC3=O)c2)c1-c1cc(N2CCCC2=O)cc(N2CCCC2=O)c1]

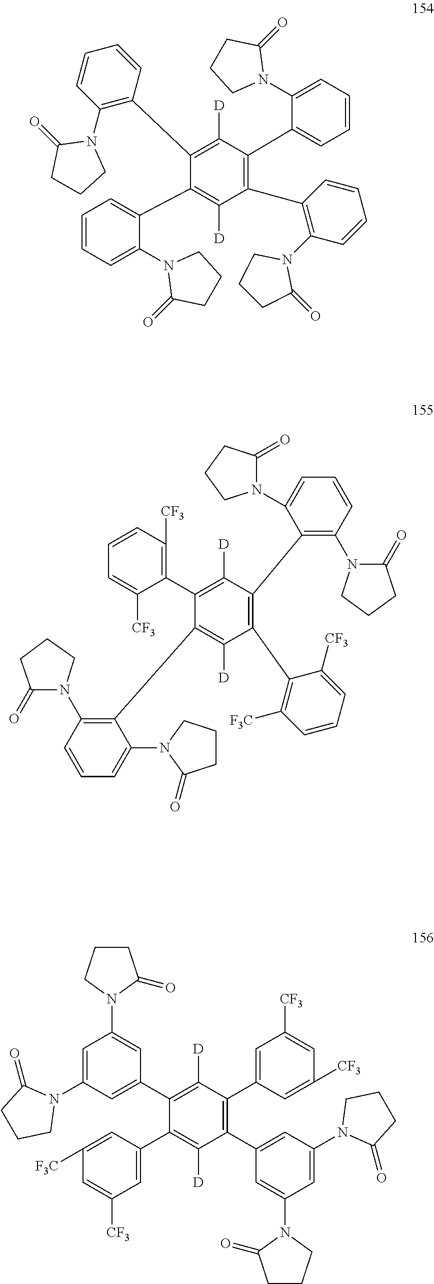 [SMILES: [2H]c1c(-c2ccccc2N2CCCC2=O)c(-c2ccccc2N2CCCC2=O)c([2H])c(-c2ccccc2N2CCCC2=O)c1-c1ccccc1N1CCCC1=O ; [2H]c1c(-c2c(N3CCCC3=O)cccc2N2CCCC2=O)c(-c2c(C(F)(F)F)cccc2C(F)(F)F)c([2H])c(-c2c(N3CCCC3=O)cccc2N2CCCC2=O)c1-c1c(C(F)(F)F)cccc1C(F)(F)F ; [2H]c1c(-c2cc(N3CCCC3=O)cc(N3CCCC3=O)c2)c(-c2cc(C(F)(F)F)cc(C(F)(F)F)c2)c([2H])c(-c2cc(N3CCCC3=O)cc(N3CCCC3=O)c2)c1-c1cc(C(F)(F)F)cc(C(F)(F)F)c1]

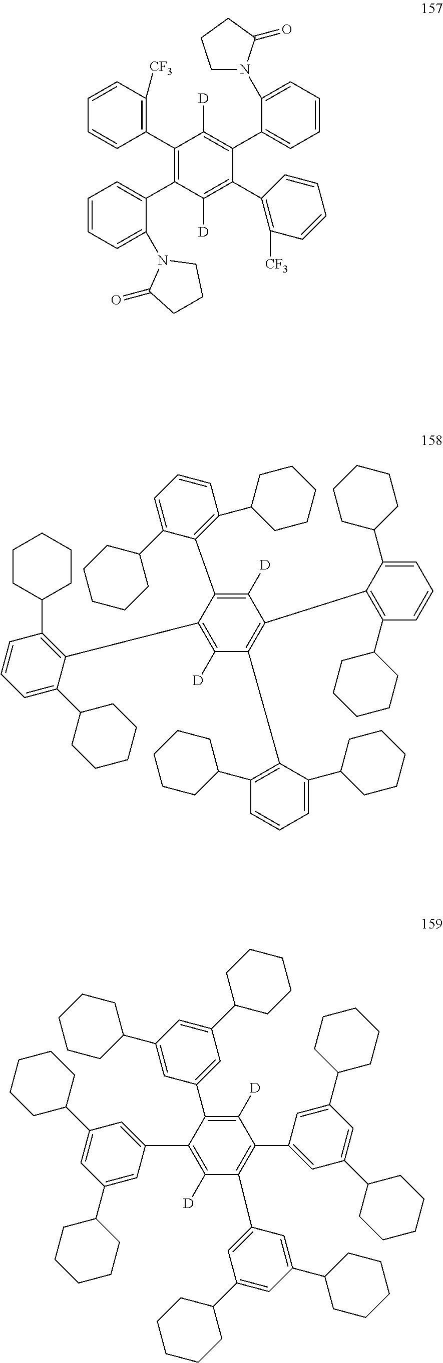 [SMILES: [2H]c1c(-c2ccccc2N2CCCC2=O)c(-c2ccccc2C(F)(F)F)c([2H])c(-c2ccccc2N2CCCC2=O)c1-c1ccccc1C(F)(F)F ; [2H]c1c(-c2c(C3CCCCC3)cccc2C2CCCCC2)c(-c2c(C3CCCCC3)cccc2C2CCCCC2)c([2H])c(-c2c(C3CCCCC3)cccc2C2CCCCC2)c1-c1c(C2CCCCC2)cccc1C1CCCCC1 ; [2H]c1c(-c2cc(C3CCCCC3)cc(C3CCCCC3)c2)c(-c2cc(C3CCCCC3)cc(C3CCCCC3)c2)c([2H])c(-c2cc(C3CCCCC3)cc(C3CCCCC3)c2)c1-c1cc(C2CCCCC2)cc(C2CCCCC2)c1]

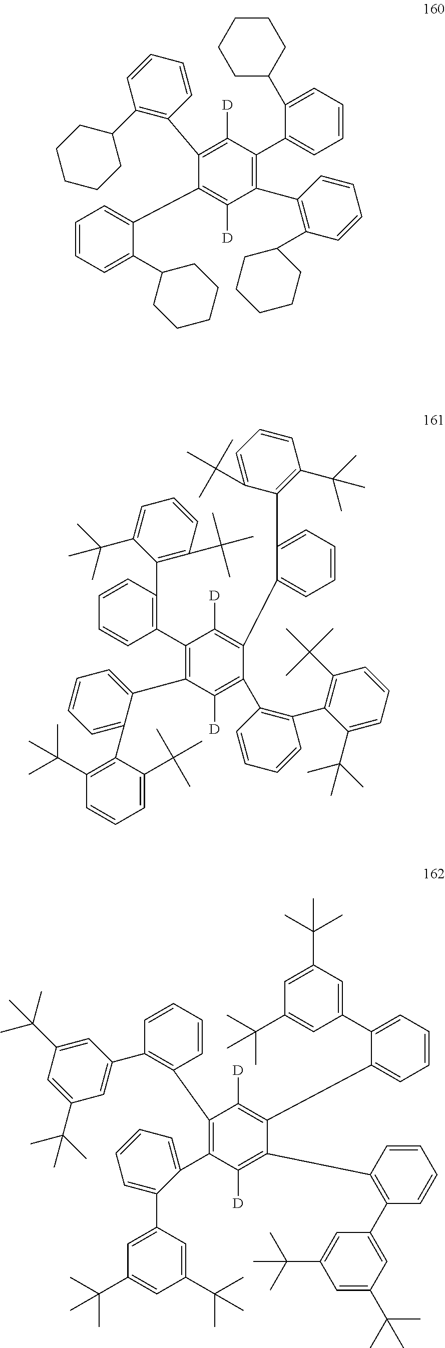 [SMILES: [2H]c1c(-c2ccccc2C2CCCCC2)c(-c2ccccc2C2CCCCC2)c([2H])c(-c2ccccc2C2CCCCC2)c1-c1ccccc1C1CCCCC1 ; [2H]c1c(-c2ccccc2-c2c(C(C)(C)C)cccc2C(C)(C)C)c(-c2ccccc2-c2c(C(C)(C)C)cccc2C(C)(C)C)c([2H])c(-c2ccccc2-c2c(C(C)(C)C)cccc2C(C)(C)C)c1-c1ccccc1-c1c(C(C)(C)C)cccc1C(C)(C)C ; [2H]c1c(-c2ccccc2-c2cc(C(C)(C)C)cc(C(C)(C)C)c2)c(-c2ccccc2-c2cc(C(C)(C)C)cc(C(C)(C)C)c2)c([2H])c(-c2ccccc2-c2cc(C(C)(C)C)cc(C(C)(C)C)c2)c1-c1ccccc1-c1cc(C(C)(C)C)cc(C(C)(C)C)c1]

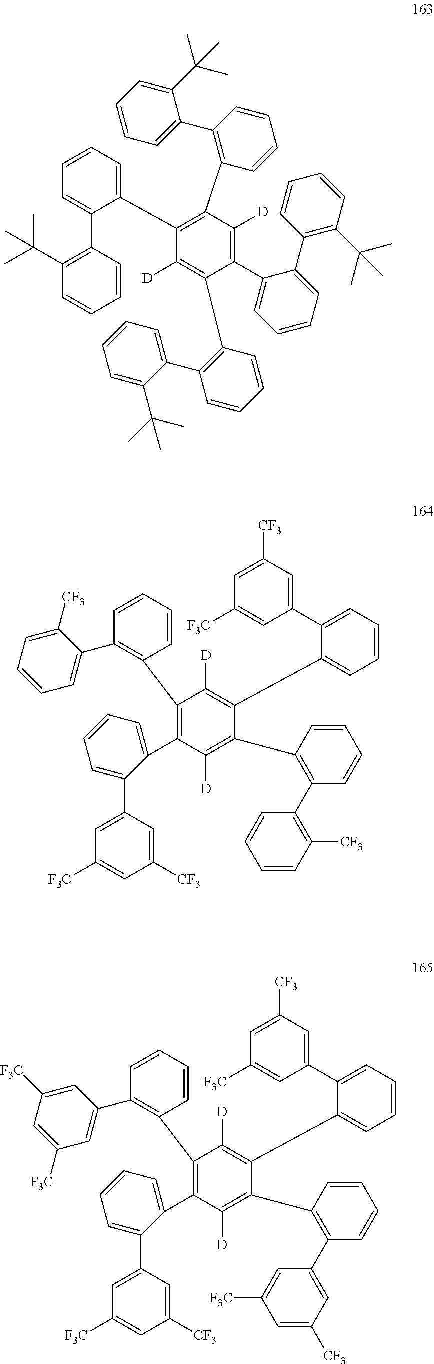 [SMILES: [2H]c1c(-c2ccccc2-c2ccccc2C(C)(C)C)c(-c2ccccc2-c2ccccc2C(C)(C)C)c([2H])c(-c2ccccc2-c2ccccc2C(C)(C)C)c1-c1ccccc1-c1ccccc1C(C)(C)C ; [2H]c1c(-c2ccccc2-c2cc(C(F)(F)F)cc(C(F)(F)F)c2)c(-c2ccccc2-c2ccccc2C(F)(F)F)c([2H])c(-c2ccccc2-c2cc(C(F)(F)F)cc(C(F)(F)F)c2)c1-c1ccccc1-c1ccccc1C(F)(F)F ; [2H]c1c(-c2ccccc2-c2cc(C(F)(F)F)cc(C(F)(F)F)c2)c(-c2ccccc2-c2cc(C(F)(F)F)cc(C(F)(F)F)c2)c([2H])c(-c2ccccc2-c2cc(C(F)(F)F)cc(C(F)(F)F)c2)c1-c1ccccc1-c1cc(C(F)(F)F)cc(C(F)(F)F)c1]

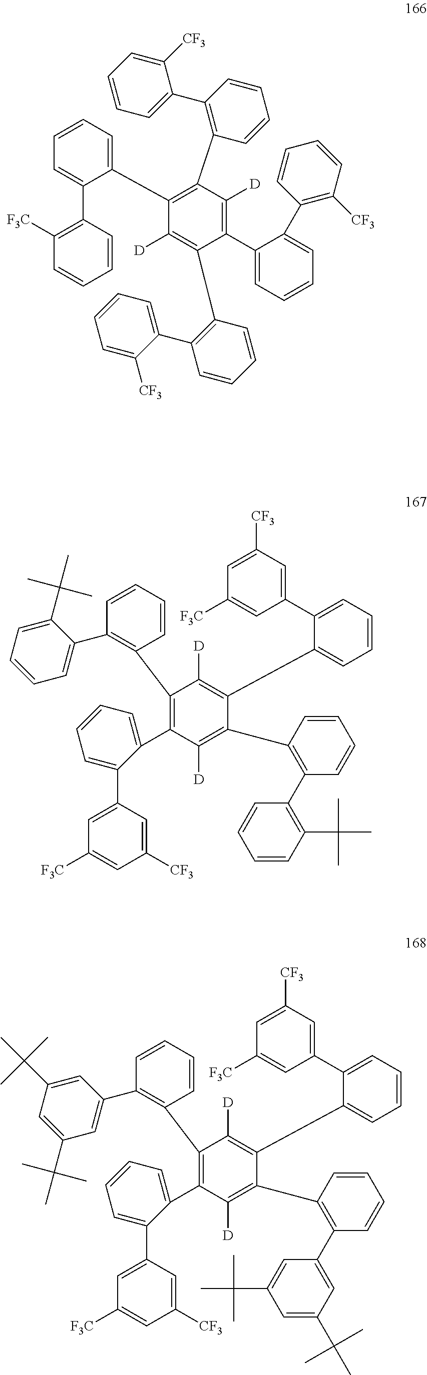 [SMILES: [2H]c1c(-c2ccccc2-c2ccccc2C(F)(F)F)c(-c2ccccc2-c2ccccc2C(F)(F)F)c([2H])c(-c2ccccc2-c2ccccc2C(F)(F)F)c1-c1ccccc1-c1ccccc1C(F)(F)F ; [2H]c1c(-c2ccccc2-c2cc(C(F)(F)F)cc(C(F)(F)F)c2)c(-c2ccccc2-c2ccccc2C(C)(C)C)c([2H])c(-c2ccccc2-c2cc(C(F)(F)F)cc(C(F)(F)F)c2)c1-c1ccccc1-c1ccccc1C(C)(C)C ; [2H]c1c(-c2ccccc2-c2cc(C(C)(C)C)cc(C(C)(C)C)c2)c(-c2ccccc2-c2cc(C(F)(F)F)cc(C(F)(F)F)c2)c([2H])c(-c2ccccc2-c2cc(C(C)(C)C)cc(C(C)(C)C)c2)c1-c1ccccc1-c1cc(C(F)(F)F)cc(C(F)(F)F)c1]

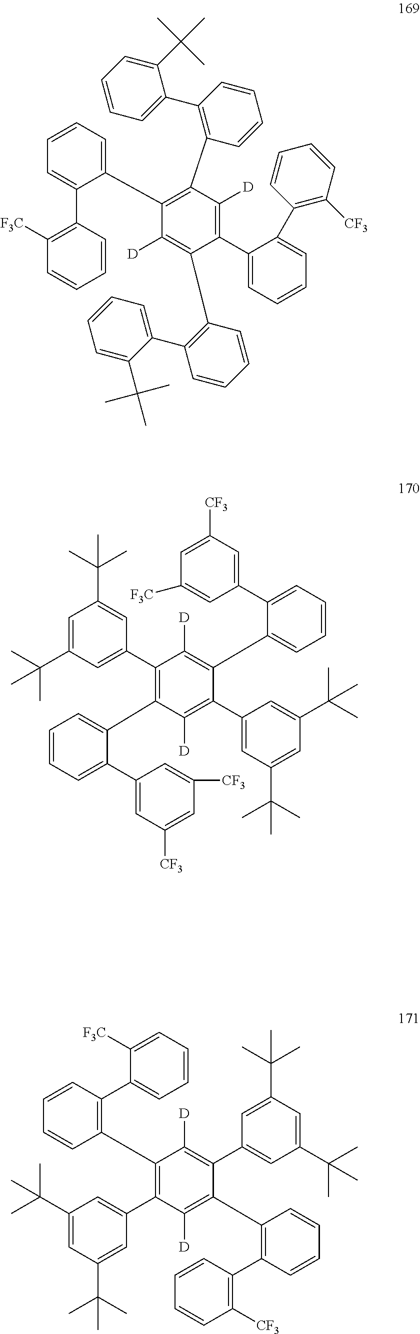

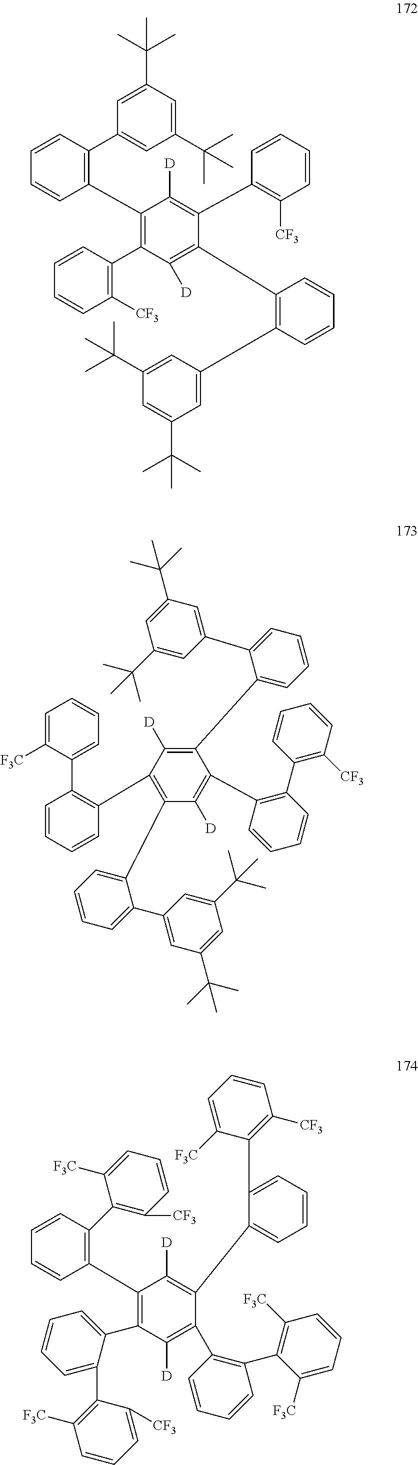 [SMILES: [2H]c1c(-c2ccccc2-c2cc(C(C)(C)C)cc(C(C)(C)C)c2)c(-c2ccccc2C(F)(F)F)c([2H])c(-c2ccccc2-c2cc(C(C)(C)C)cc(C(C)(C)C)c2)c1-c1ccccc1C(F)(F)F ; [2H]c1c(-c2ccccc2-c2cc(C(C)(C)C)cc(C(C)(C)C)c2)c(-c2ccccc2-c2ccccc2C(F)(F)F)c([2H])c(-c2ccccc2-c2cc(C(C)(C)C)cc(C(C)(C)C)c2)c1-c1ccccc1-c1ccccc1C(F)(F)F ; [2H]c1c(-c2ccccc2-c2c(C(F)(F)F)cccc2C(F)(F)F)c(-c2ccccc2-c2c(C(F)(F)F)cccc2C(F)(F)F)c([2H])c(-c2ccccc2-c2c(C(F)(F)F)cccc2C(F)(F)F)c1-c1ccccc1-c1c(C(F)(F)F)cccc1C(F)(F)F]

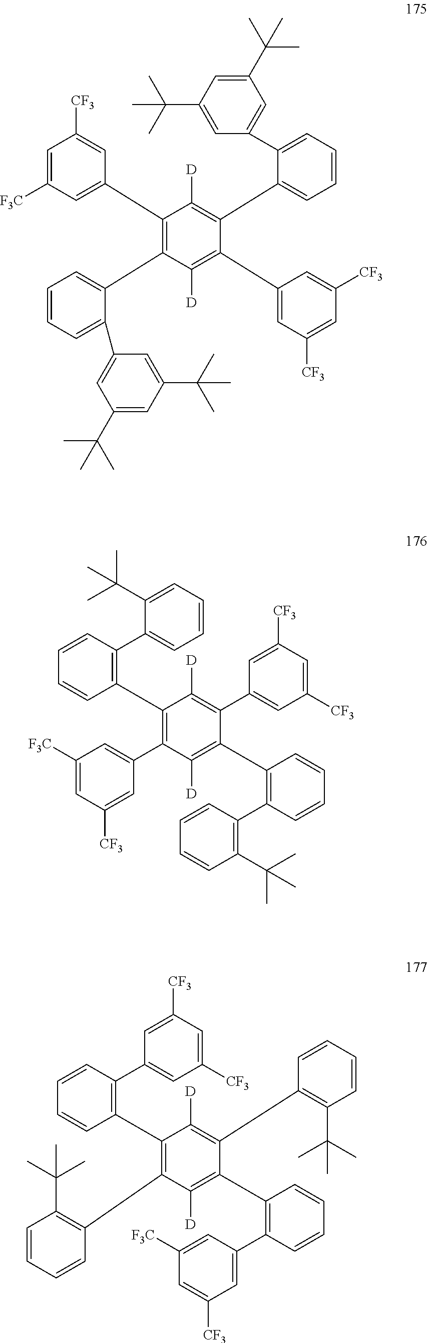 [SMILES: [2H]c1c(-c2cc(C(F)(F)F)cc(C(F)(F)F)c2)c(-c2ccccc2-c2cc(C(C)(C)C)cc(C(C)(C)C)c2)c([2H])c(-c2cc(C(F)(F)F)cc(C(F)(F)F)c2)c1-c1ccccc1-c1cc(C(C)(C)C)cc(C(C)(C)C)c1 ; [2H]c1c(-c2cc(C(F)(F)F)cc(C(F)(F)F)c2)c(-c2ccccc2-c2ccccc2C(C)(C)C)c([2H])c(-c2cc(C(F)(F)F)cc(C(F)(F)F)c2)c1-c1ccccc1-c1ccccc1C(C)(C)C ; [2H]c1c(-c2ccccc2-c2cc(C(F)(F)F)cc(C(F)(F)F)c2)c(-c2ccccc2C(C)(C)C)c([2H])c(-c2ccccc2-c2cc(C(F)(F)F)cc(C(F)(F)F)c2)c1-c1ccccc1C(C)(C)C]

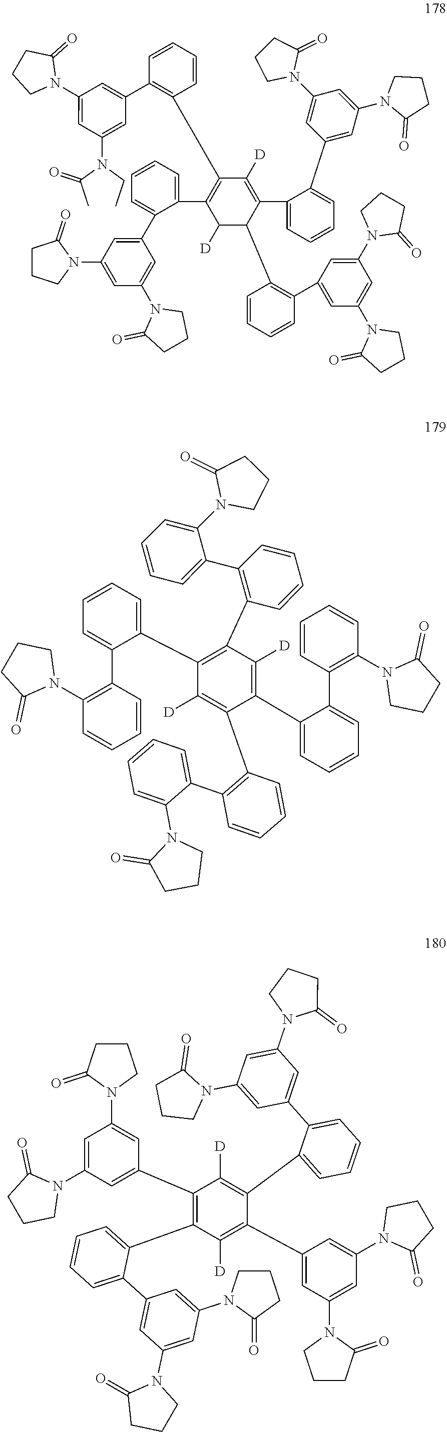 [SMILES: [2H]C1=C(c2ccccc2-c2cc(N3CCCC3=O)cc(N3CCCC3=O)c2)C(c2ccccc2-c2cc(N3CCCC3=O)cc(N3CCCC3=O)c2)C([2H])C(c2ccccc2-c2cc(N3CCCC3=O)cc(N3CCCC3=O)c2)=C1c1ccccc1-c1cc(N(CC)C(C)=O)cc(N2CCCC2=O)c1 ; [2H]c1c(-c2ccccc2-c2ccccc2N2CCCC2=O)c(-c2ccccc2-c2ccccc2N2CCCC2=O)c([2H])c(-c2ccccc2-c2ccccc2N2CCCC2=O)c1-c1ccccc1-c1ccccc1N1CCCC1=O ; [2H]c1c(-c2cc(N3CCCC3=O)cc(N3CCCC3=O)c2)c(-c2ccccc2-c2cc(N3CCCC3=O)cc(N3CCCC3=O)c2)c([2H])c(-c2cc(N3CCCC3=O)cc(N3CCCC3=O)c2)c1-c1ccccc1-c1cc(N2CCCC2=O)cc(N2CCCC2=O)c1]

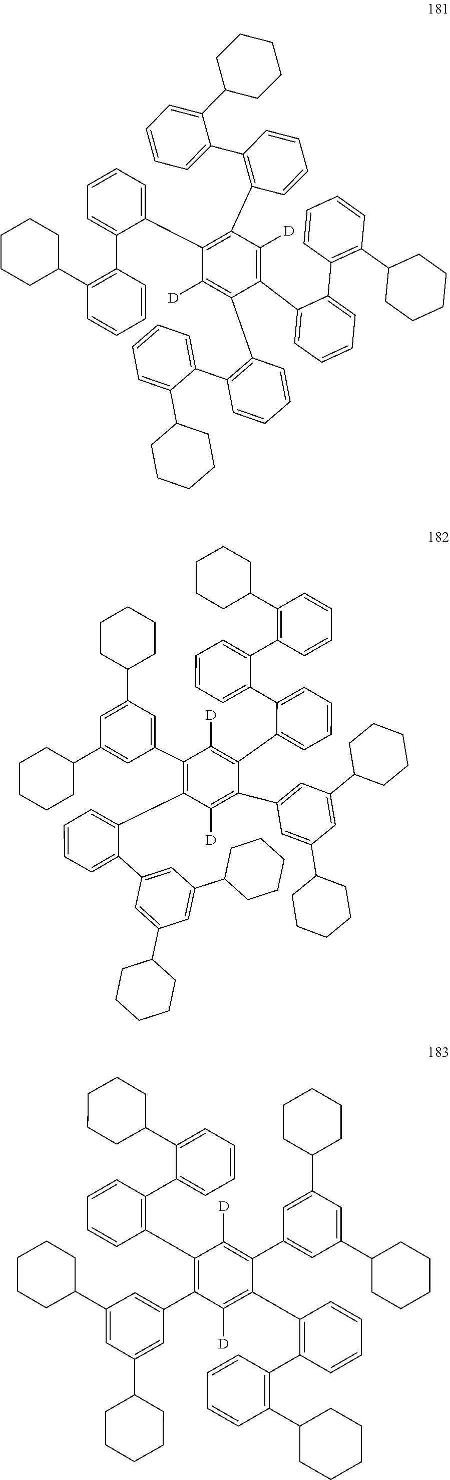 [SMILES: [2H]c1c(-c2ccccc2-c2ccccc2C2CCCCC2)c(-c2ccccc2-c2ccccc2C2CCCCC2)c([2H])c(-c2ccccc2-c2ccccc2C2CCCCC2)c1-c1ccccc1-c1ccccc1C1CCCCC1 ; [2H]c1c(-c2cc(C3CCCCC3)cc(C3CCCCC3)c2)c(-c2ccccc2-c2ccccc2-c2ccccc2C2CCCCC2)c([2H])c(-c2cc(C3CCCCC3)cc(C3CCCCC3)c2)c1-c1ccccc1-c1cc(C2CCCCC2)cc(C2CCCCC2)c1 ; [2H]c1c(-c2cc(C3CCCCC3)cc(C3CCCCC3)c2)c(-c2ccccc2-c2ccccc2C2CCCCC2)c([2H])c(-c2cc(C3CCCCC3)cc(C3CCCCC3)c2)c1-c1ccccc1-c1ccccc1C1CCCCC1]

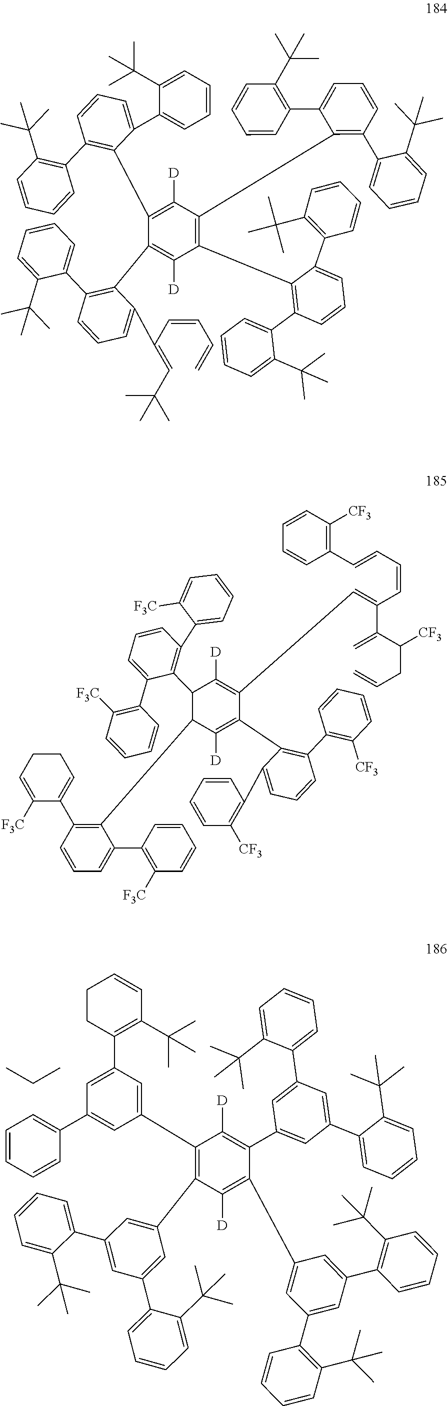

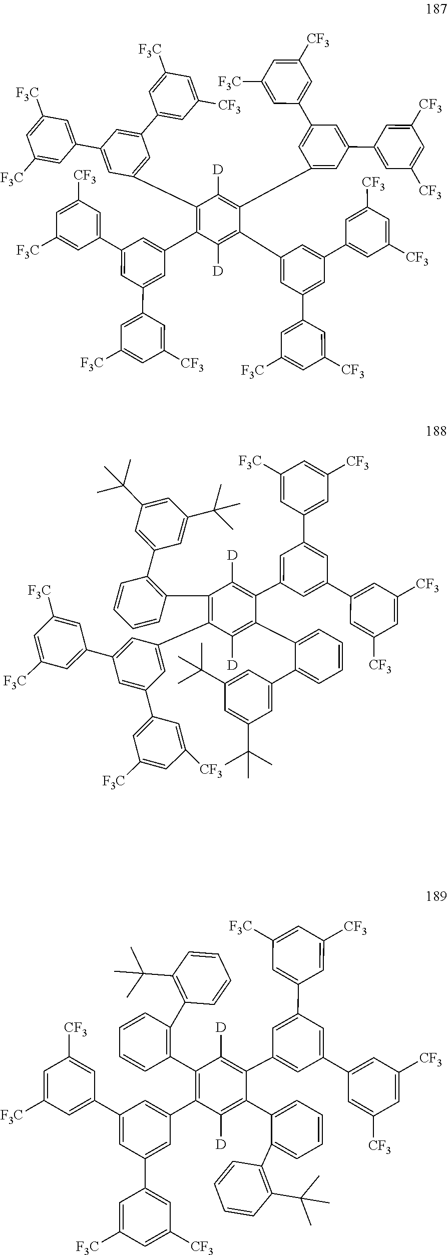

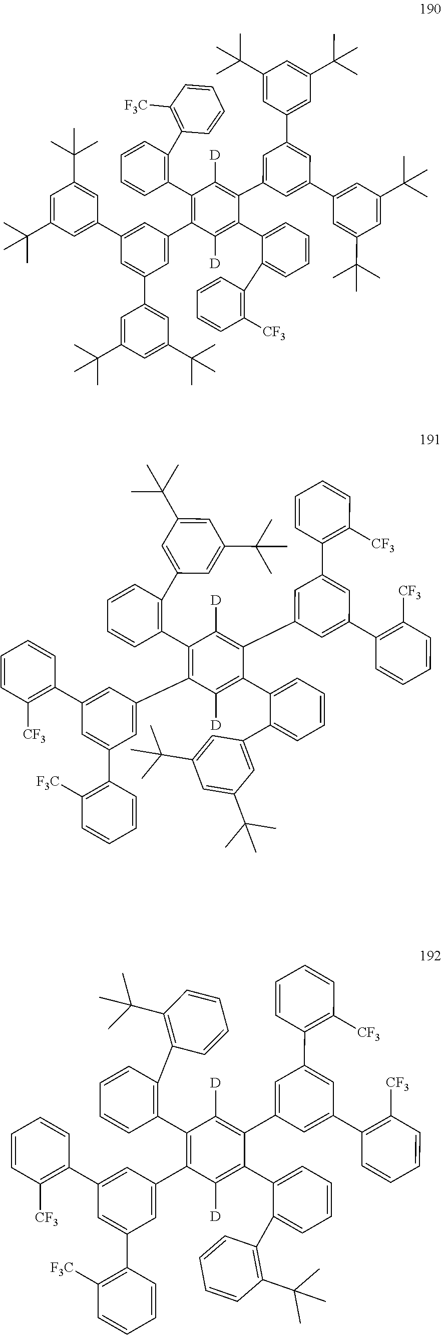

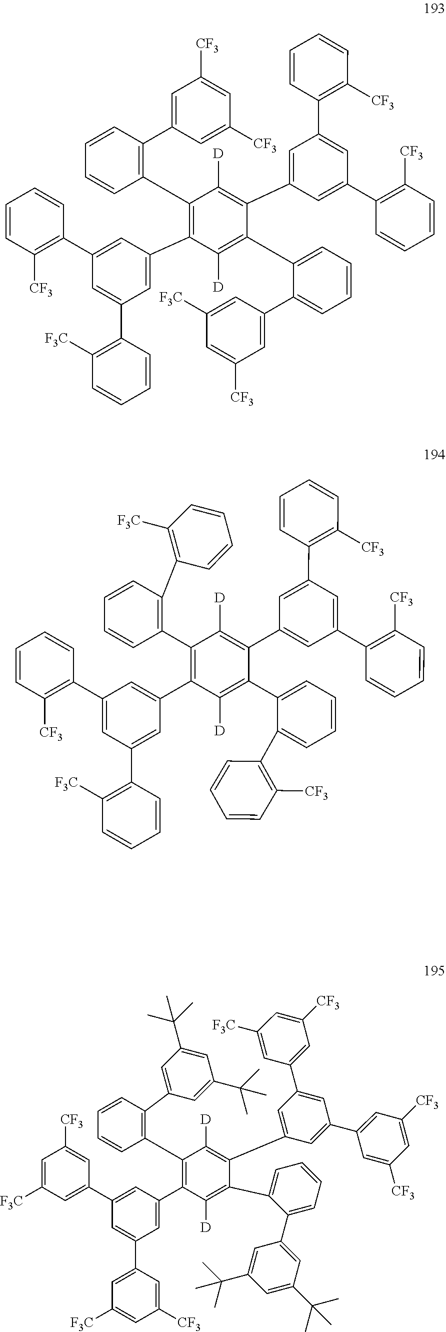

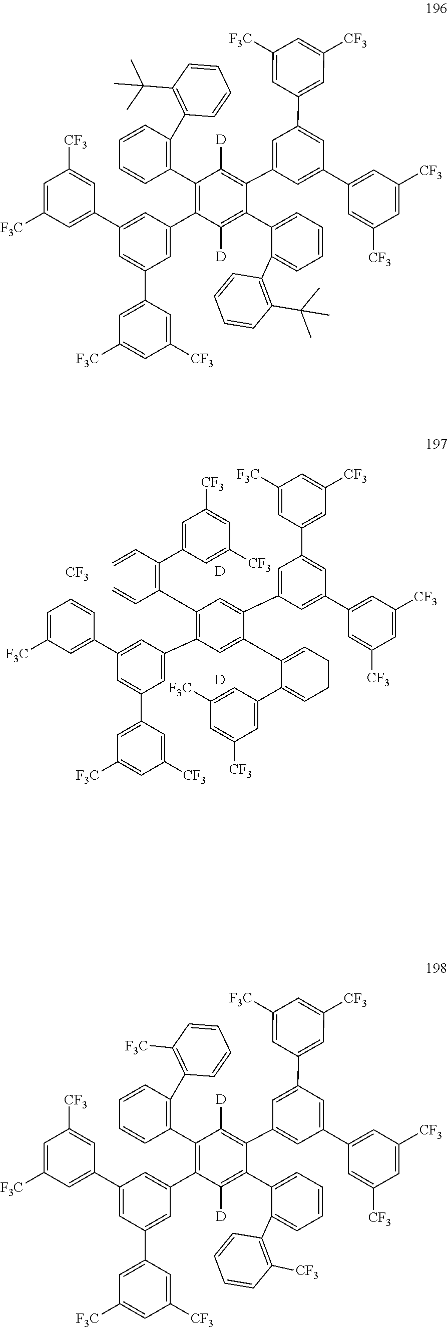

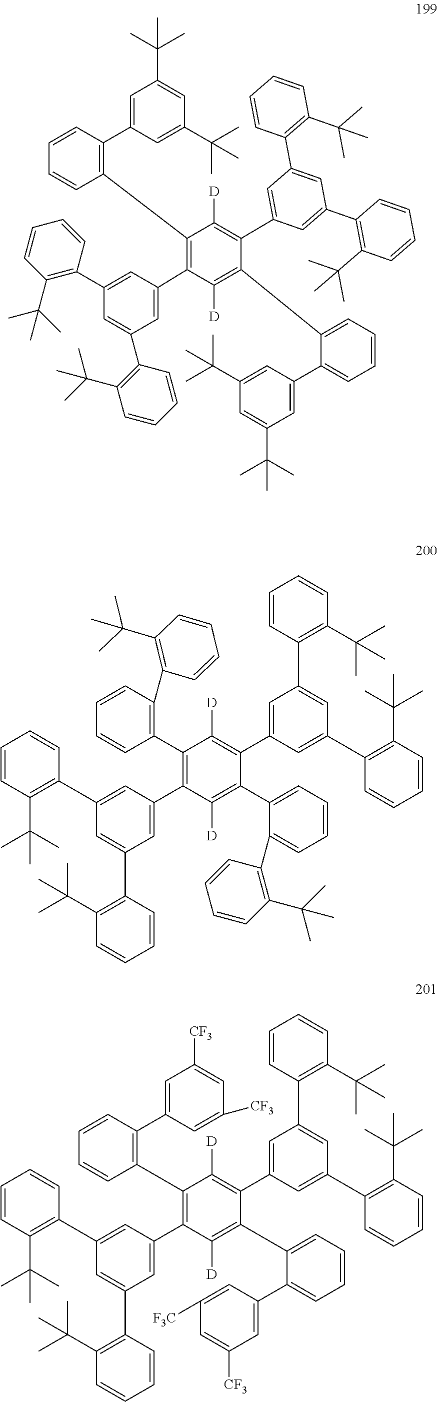

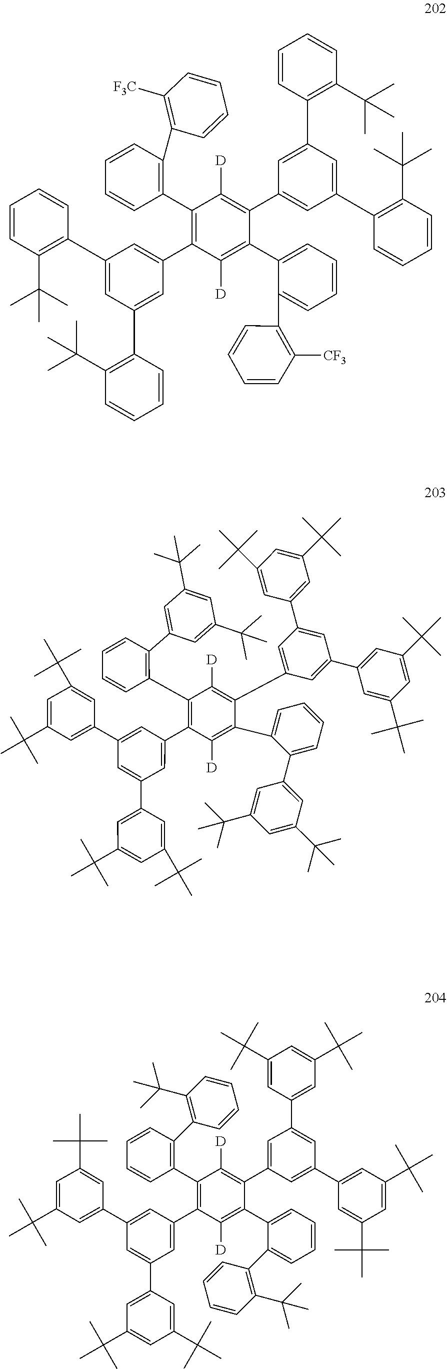

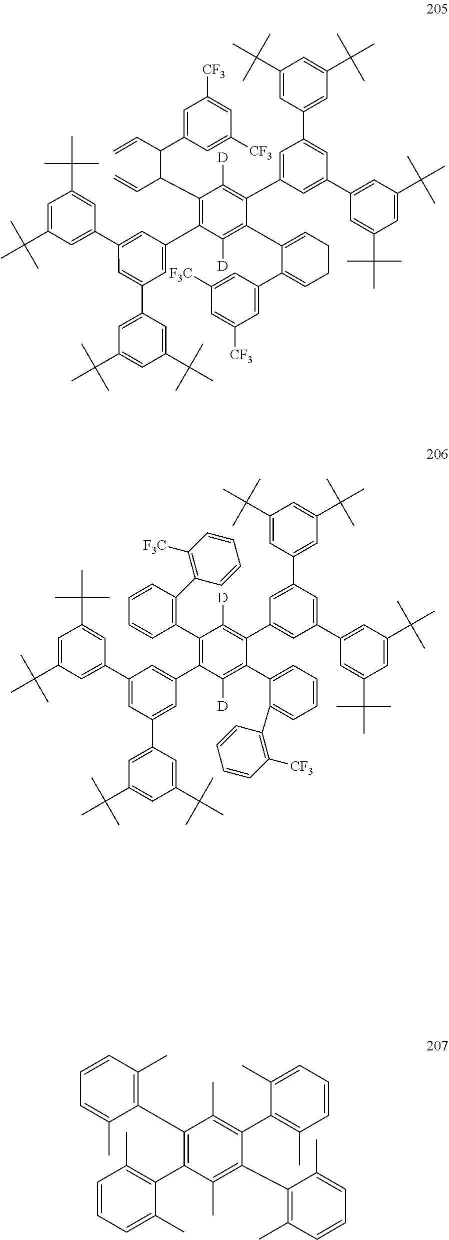

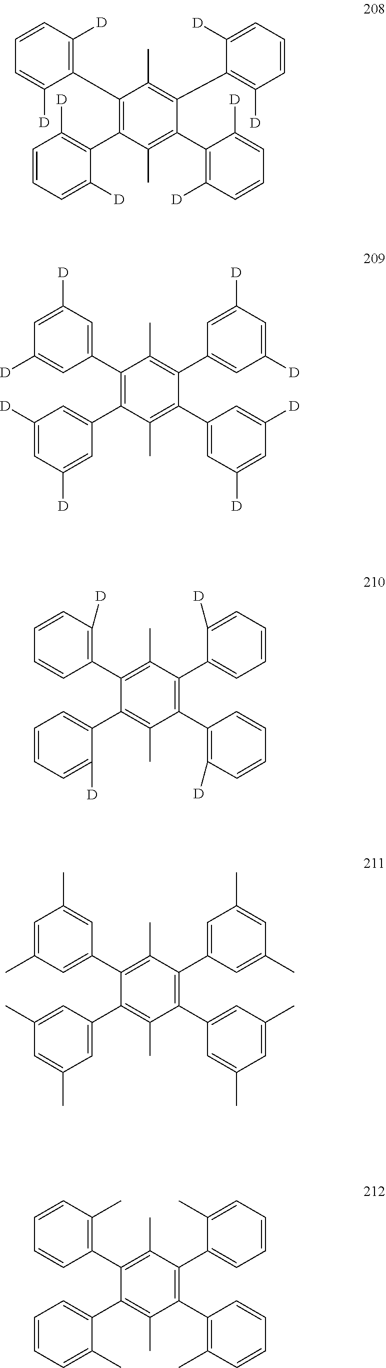

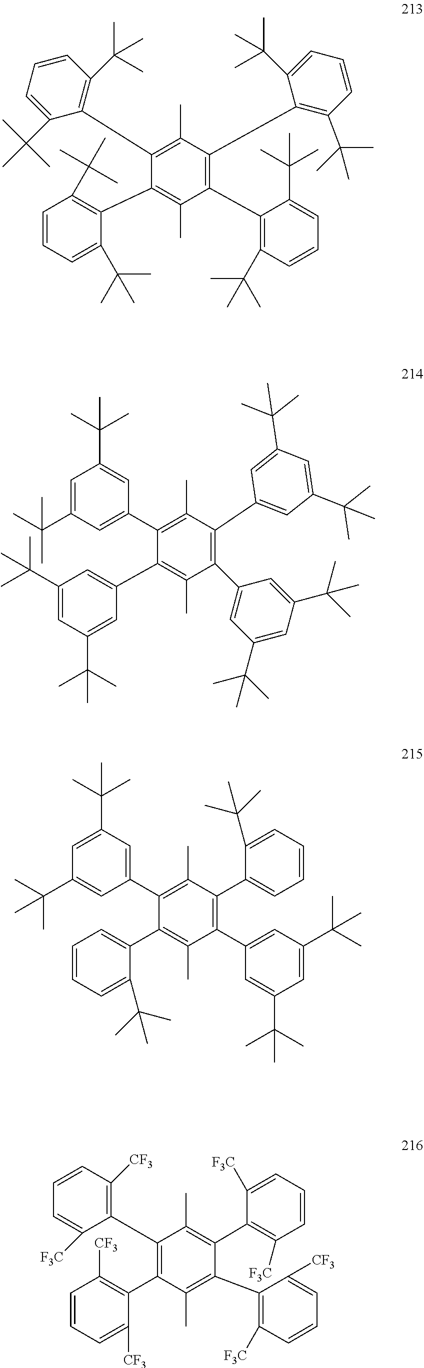

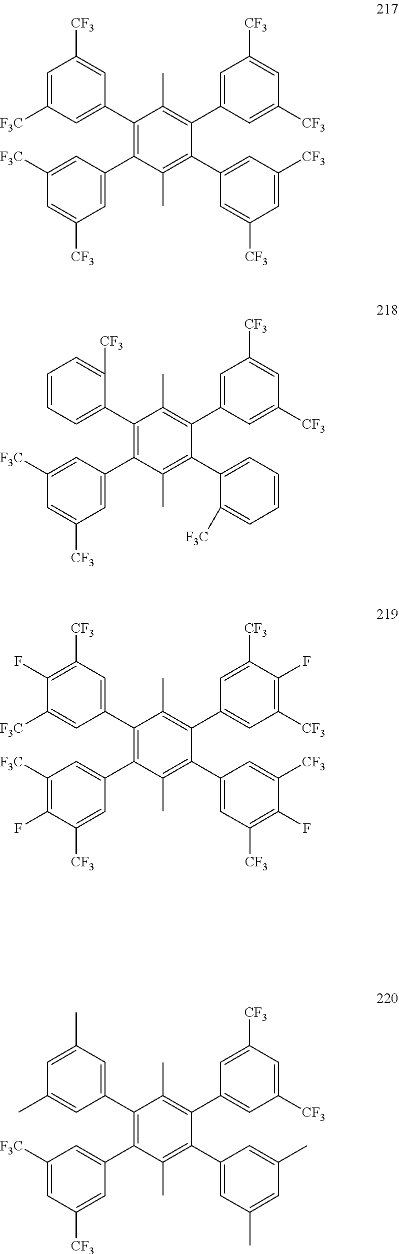

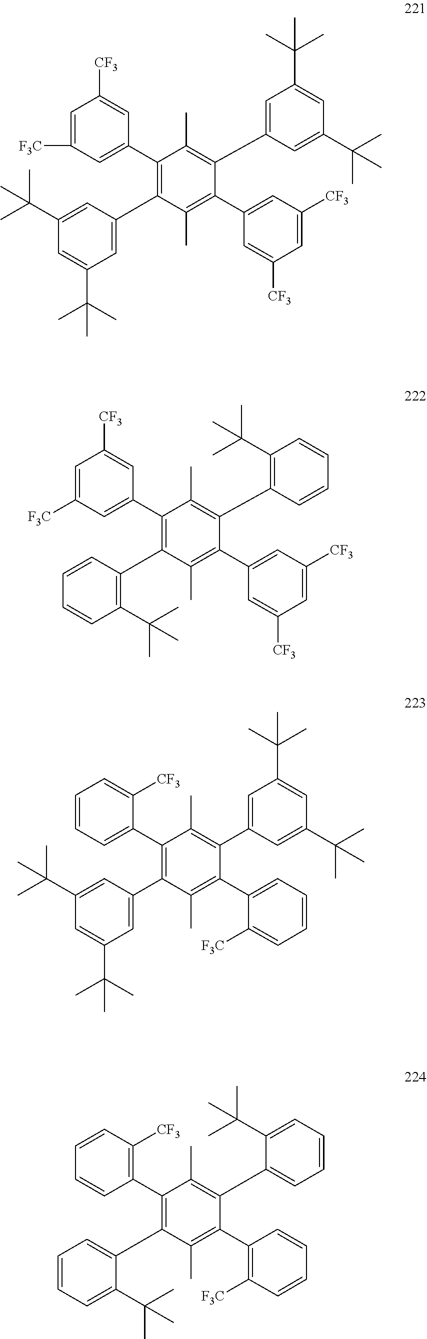

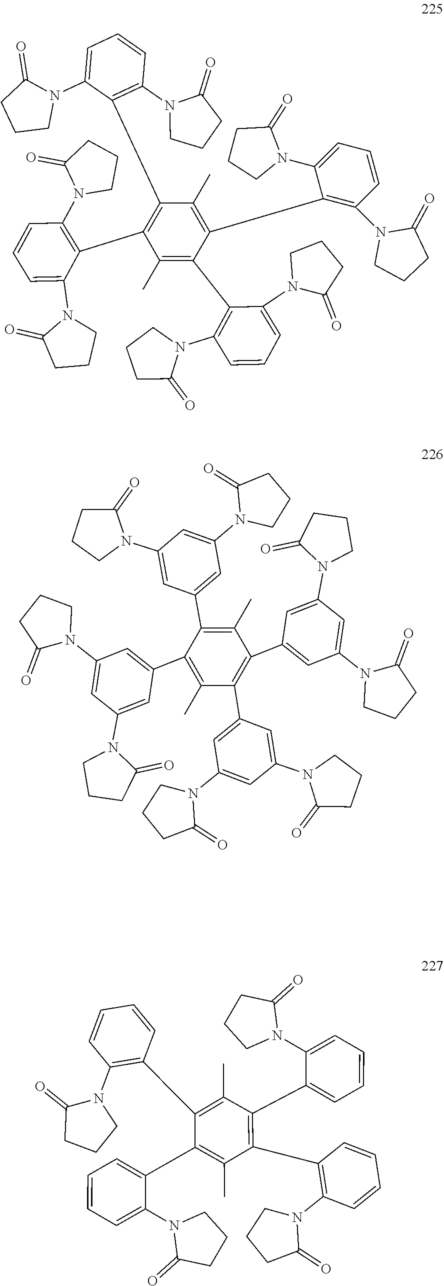 [SMILES: Cc1c(-c2c(N3CCCC3=O)cccc2N2CCCC2=O)c(-c2c(N3CCCC3=O)cccc2N2CCCC2=O)c(C)c(-c2c(N3CCCC3=O)cccc2N2CCCC2=O)c1-c1c(N2CCCC2=O)cccc1N1CCCC1=O ; Cc1c(-c2cc(N3CCCC3=O)cc(N3CCCC3=O)c2)c(-c2cc(N3CCCC3=O)cc(N3CCCC3=O)c2)c(C)c(-c2cc(N3CCCC3=O)cc(N3CCCC3=O)c2)c1-c1cc(N2CCCC2=O)cc(N2CCCC2=O)c1 ; Cc1c(-c2ccccc2N2CCCC2=O)c(-c2ccccc2N2CCCC2=O)c(C)c(-c2ccccc2N2CCCC2=O)c1-c1ccccc1N1CCCC1=O]

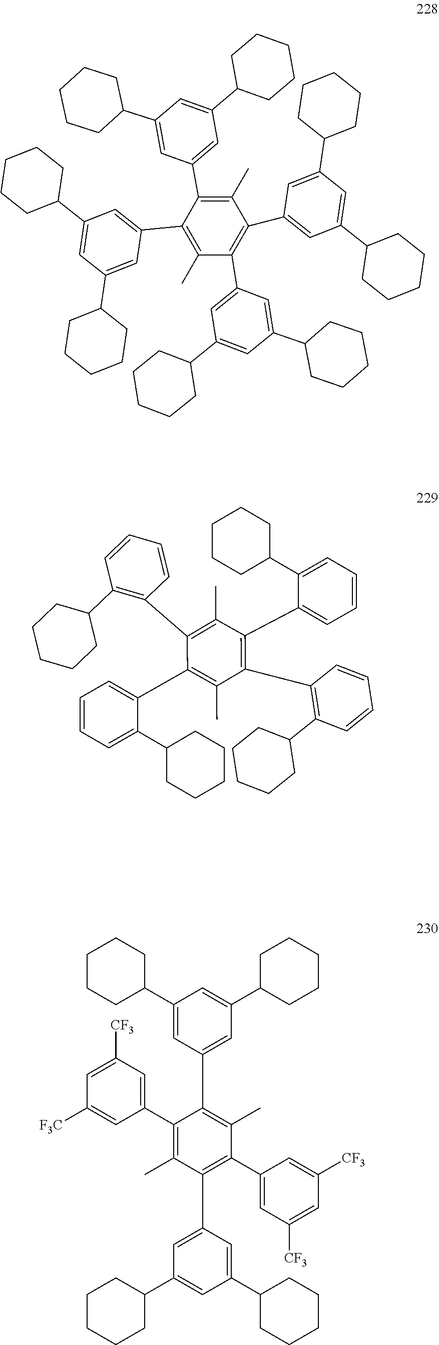

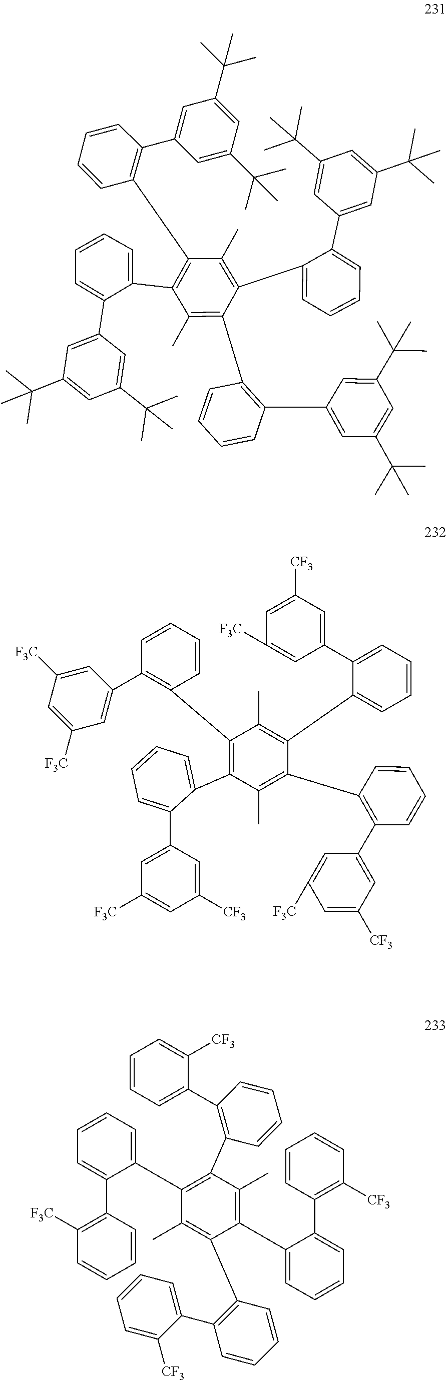

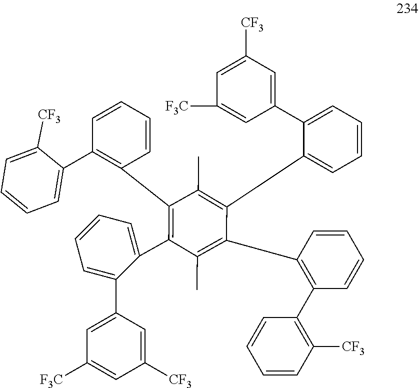 [SMILES: Cc1c(-c2ccccc2-c2cc(C(F)(F)F)cc(C(F)(F)F)c2)c(-c2ccccc2-c2ccccc2C(F)(F)F)c(C)c(-c2ccccc2-c2cc(C(F)(F)F)cc(C(F)(F)F)c2)c1-c1ccccc1-c1ccccc1C(F)(F)F]

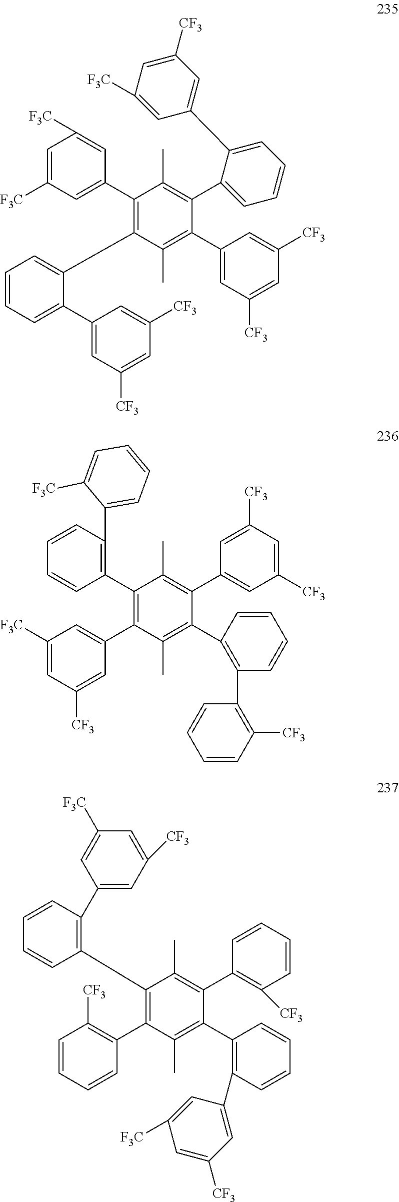

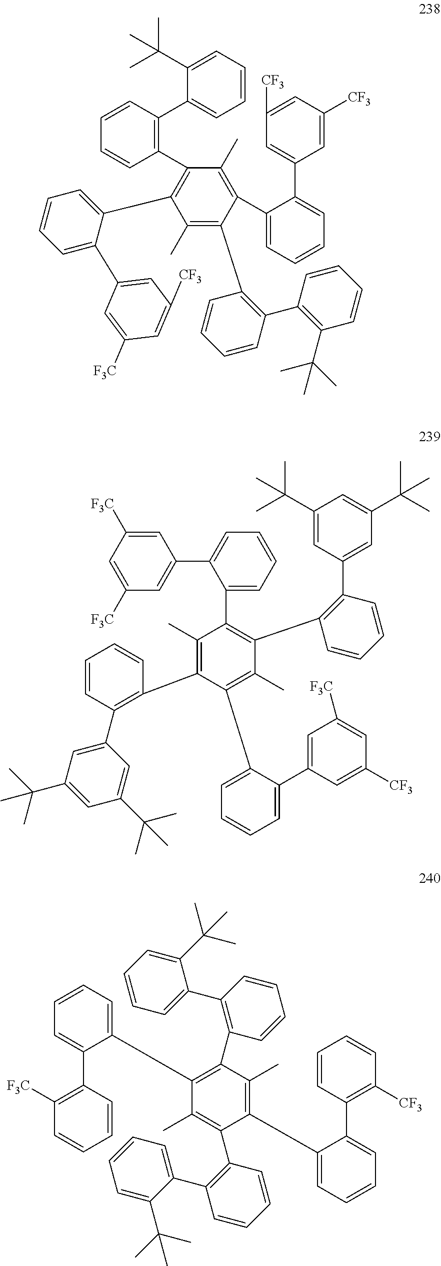 [SMILES: Cc1c(-c2ccccc2-c2cc(C(F)(F)F)cc(C(F)(F)F)c2)c(-c2ccccc2-c2ccccc2C(C)(C)C)c(C)c(-c2ccccc2-c2cc(C(F)(F)F)cc(C(F)(F)F)c2)c1-c1ccccc1-c1ccccc1C(C)(C)C ; Cc1c(-c2ccccc2-c2cc(C(C)(C)C)cc(C(C)(C)C)c2)c(-c2ccccc2-c2cc(C(F)(F)F)cc(C(F)(F)F)c2)c(C)c(-c2ccccc2-c2cc(C(C)(C)C)cc(C(C)(C)C)c2)c1-c1ccccc1-c1cc(C(F)(F)F)cc(C(F)(F)F)c1 ; Cc1c(-c2ccccc2-c2ccccc2C(C)(C)C)c(-c2ccccc2-c2ccccc2C(F)(F)F)c(C)c(-c2ccccc2-c2ccccc2C(C)(C)C)c1-c1ccccc1-c1ccccc1C(F)(F)F]

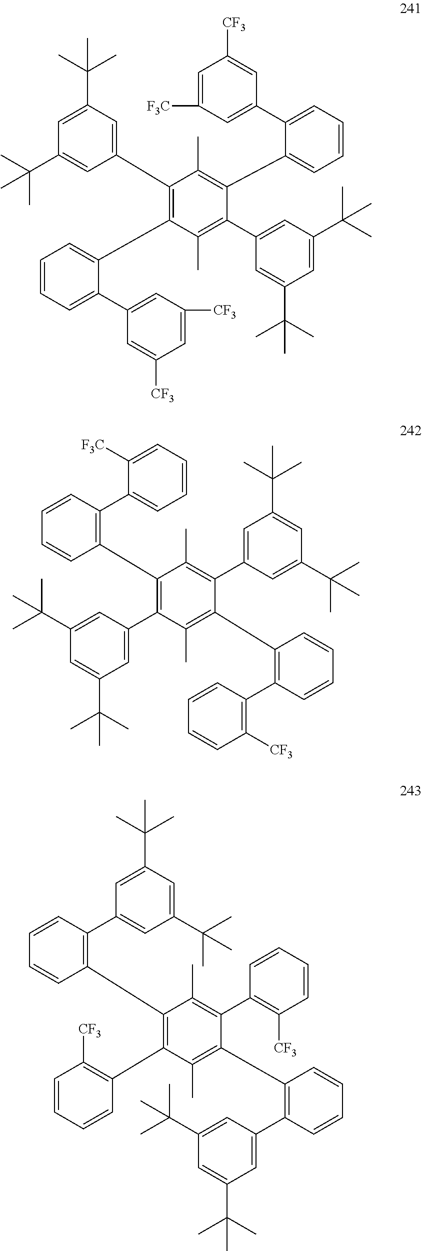

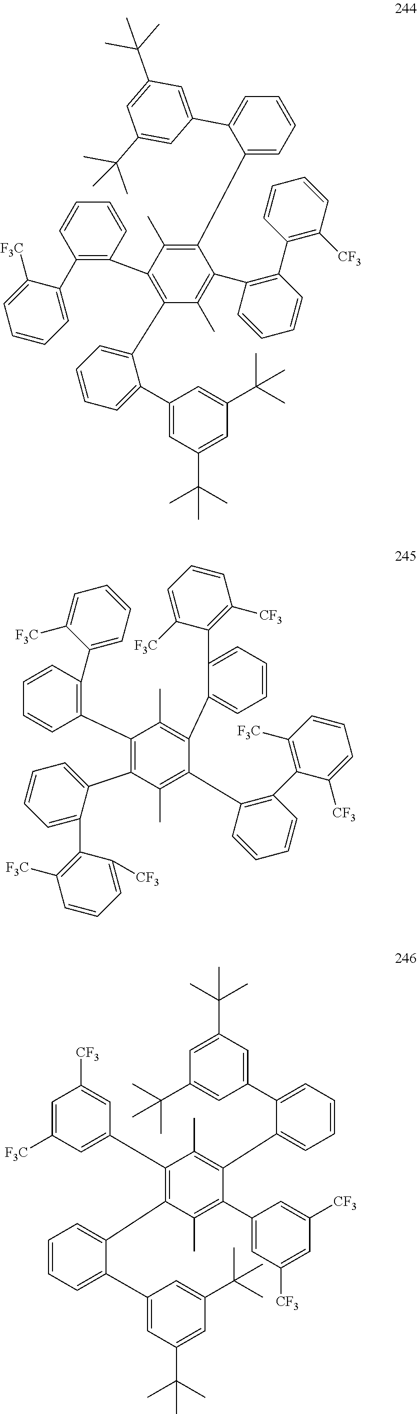

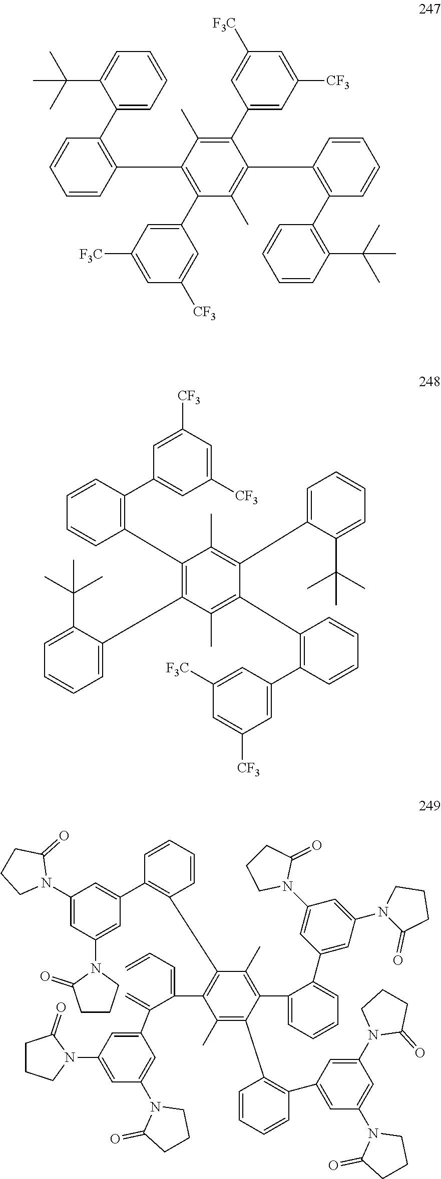 [SMILES: Cc1c(-c2cc(C(F)(F)F)cc(C(F)(F)F)c2)c(-c2ccccc2-c2ccccc2C(C)(C)C)c(C)c(-c2cc(C(F)(F)F)cc(C(F)(F)F)c2)c1-c1ccccc1-c1ccccc1C(C)(C)C ; Cc1c(-c2ccccc2-c2cc(C(F)(F)F)cc(C(F)(F)F)c2)c(-c2ccccc2C(C)(C)C)c(C)c(-c2ccccc2-c2cc(C(F)(F)F)cc(C(F)(F)F)c2)c1-c1ccccc1C(C)(C)C ; C=C/C=C(\C(=C)c1cc(N2CCCC2=O)cc(N2CCCC2=O)c1)c1c(C)c(-c2ccccc2-c2cc(N3CCCC3=O)cc(N3CCCC3=O)c2)c(-c2ccccc2-c2cc(N3CCCC3=O)cc(N3CCCC3=O)c2)c(C)c1-c1ccccc1-c1cc(N2CCCC2=O)cc(N2CCCC2=O)c1]

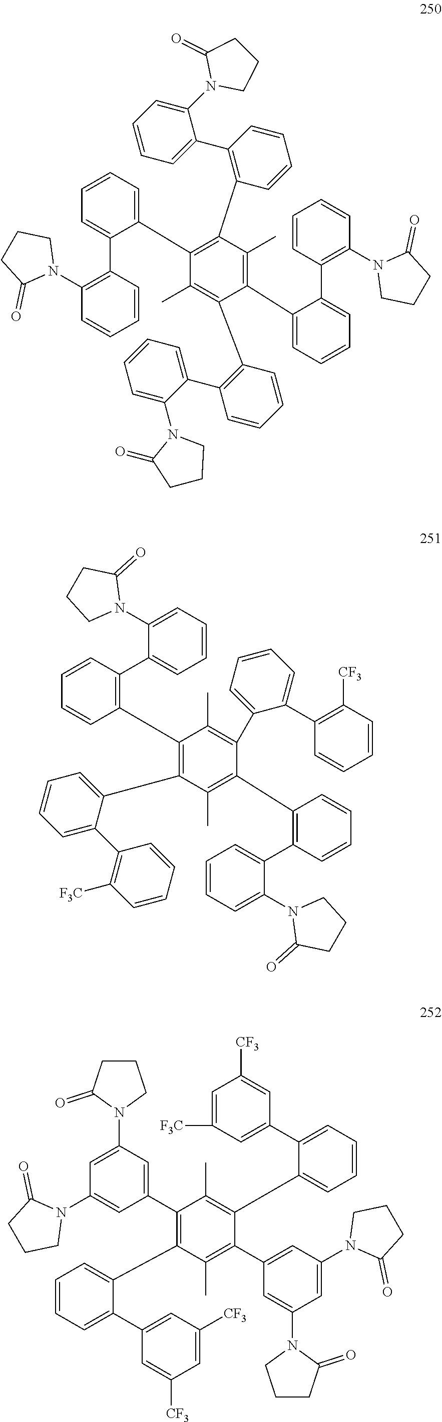 [SMILES: Cc1c(-c2ccccc2-c2ccccc2N2CCCC2=O)c(-c2ccccc2-c2ccccc2N2CCCC2=O)c(C)c(-c2ccccc2-c2ccccc2N2CCCC2=O)c1-c1ccccc1-c1ccccc1N1CCCC1=O ; Cc1c(-c2ccccc2-c2ccccc2N2CCCC2=O)c(-c2ccccc2-c2ccccc2C(F)(F)F)c(C)c(-c2ccccc2-c2ccccc2N2CCCC2=O)c1-c1ccccc1-c1ccccc1C(F)(F)F ; Cc1c(-c2cc(N3CCCC3=O)cc(N3CCCC3=O)c2)c(-c2ccccc2-c2cc(C(F)(F)F)cc(C(F)(F)F)c2)c(C)c(-c2cc(N3CCCC3=O)cc(N3CCCC3=O)c2)c1-c1ccccc1-c1cc(C(F)(F)F)cc(C(F)(F)F)c1]

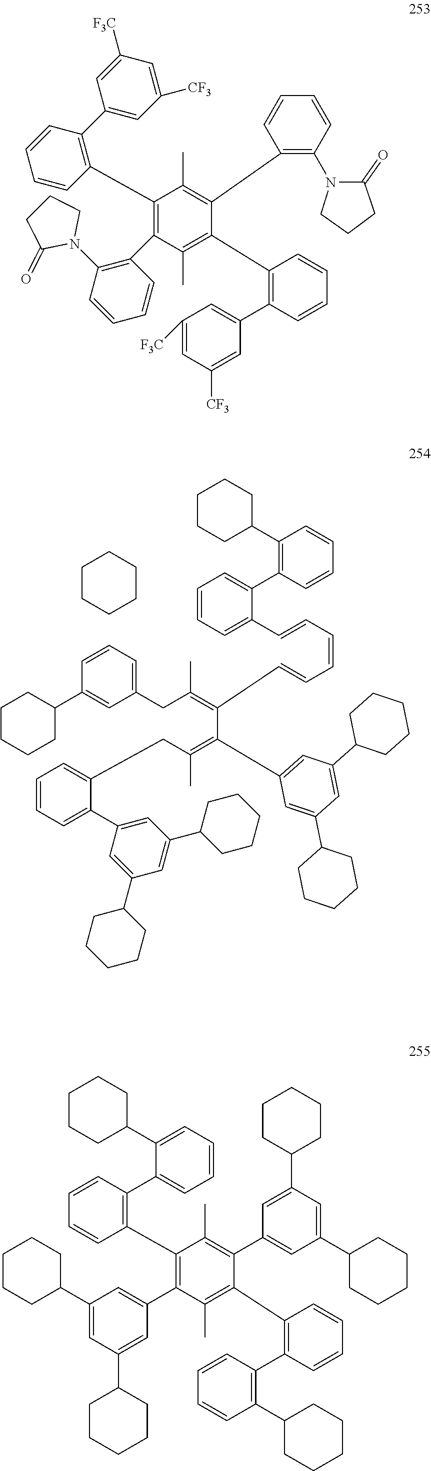

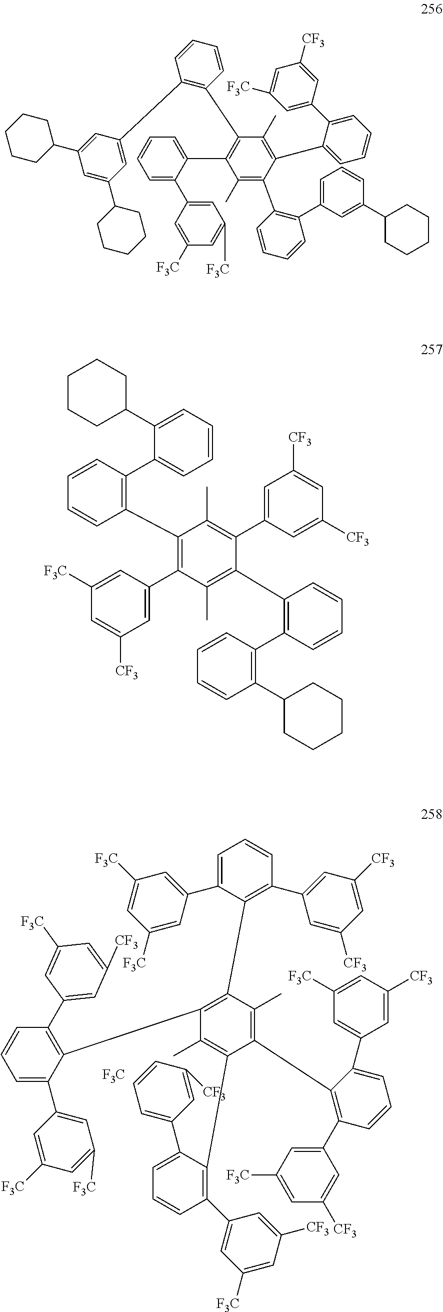

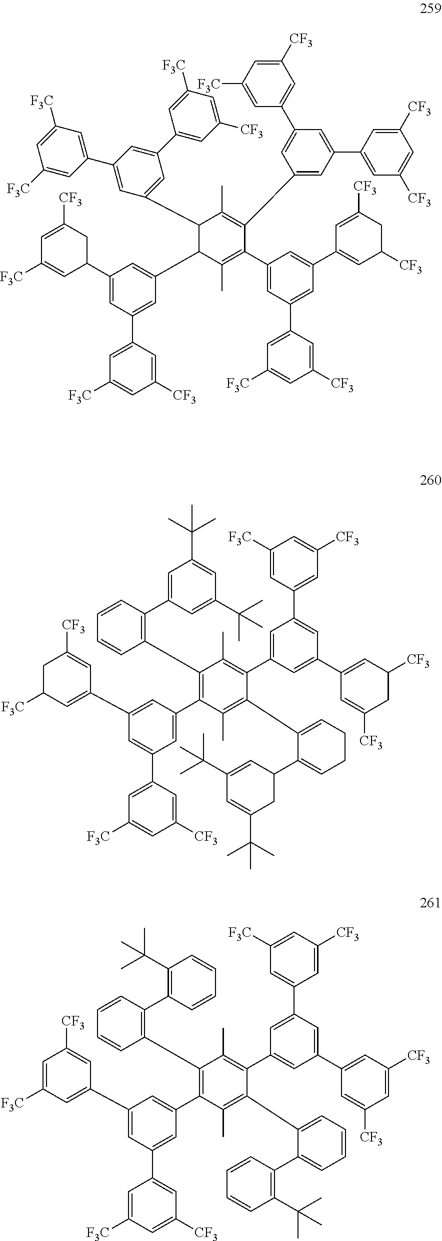

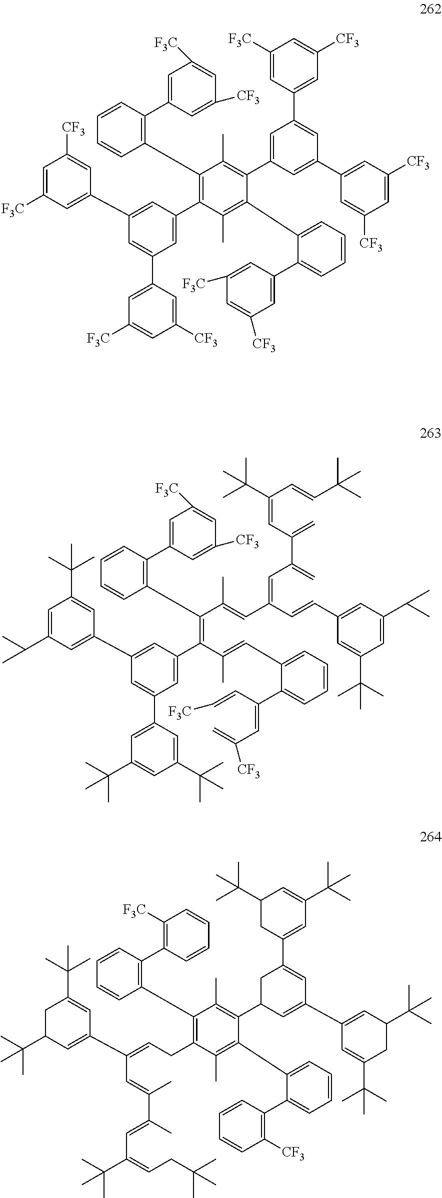

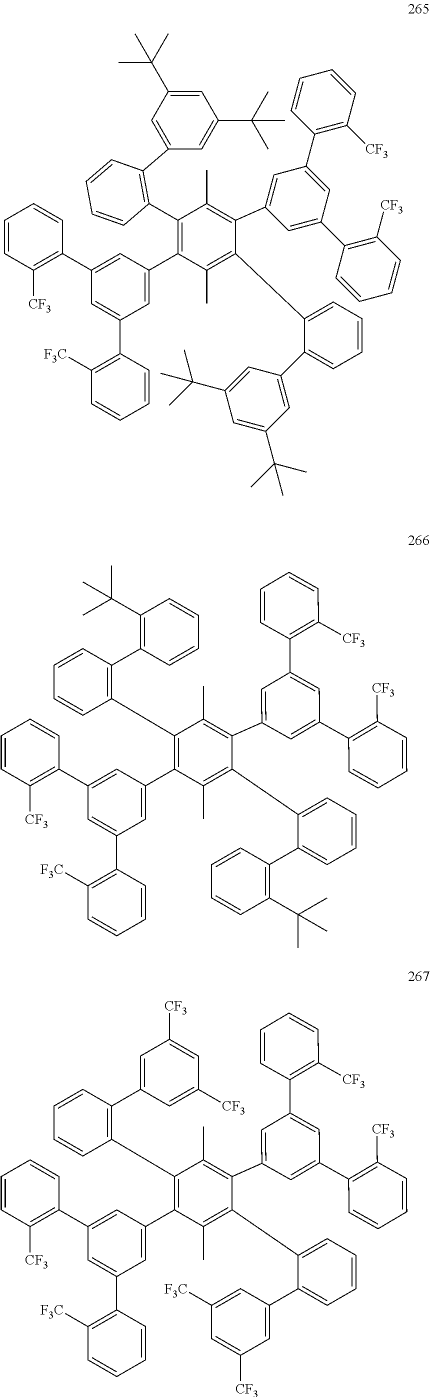

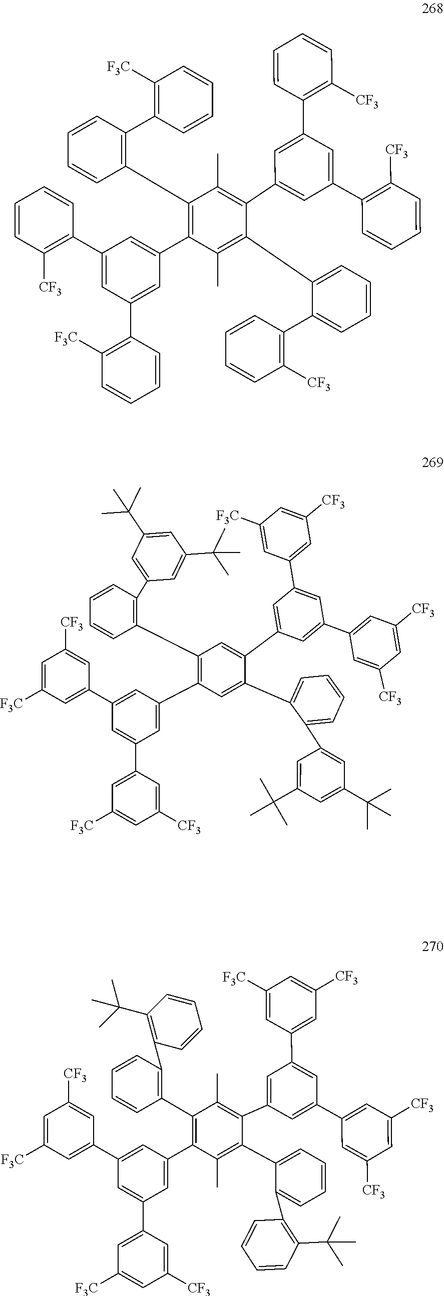

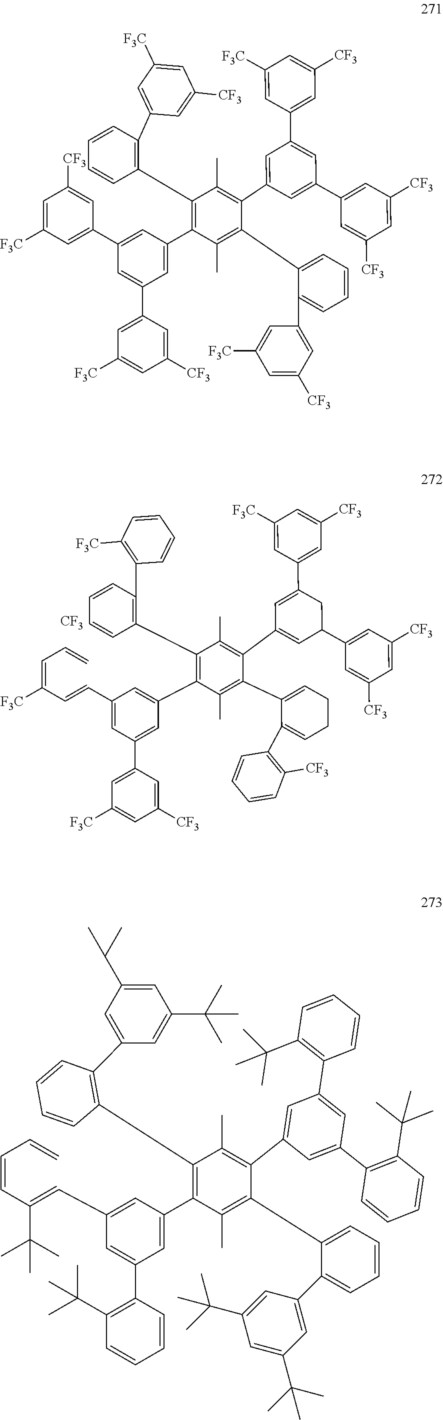

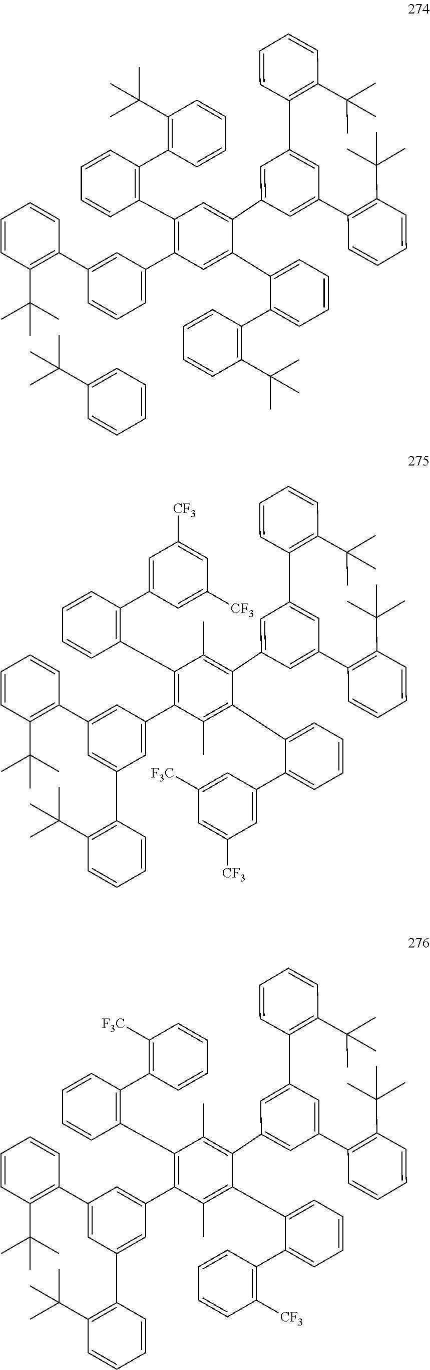

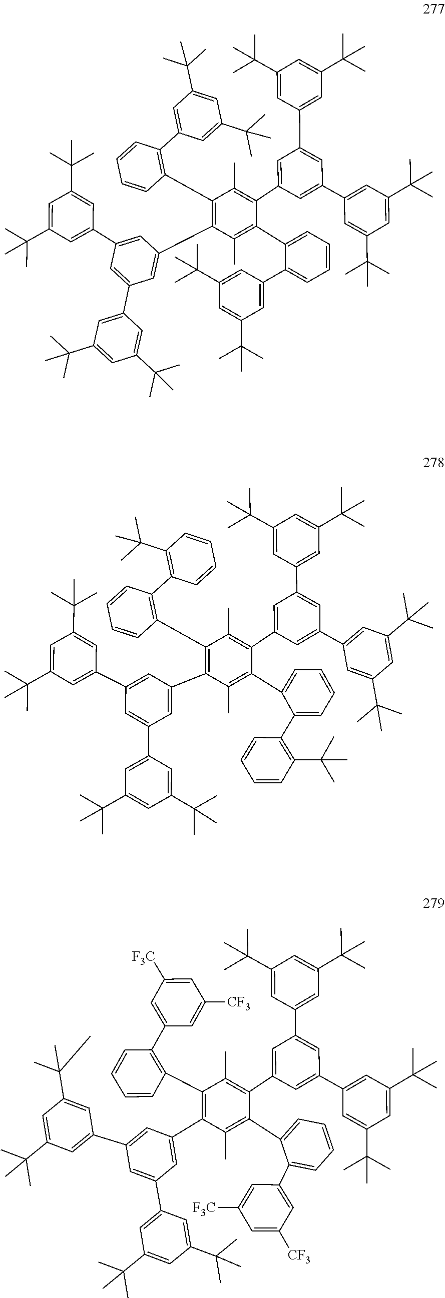

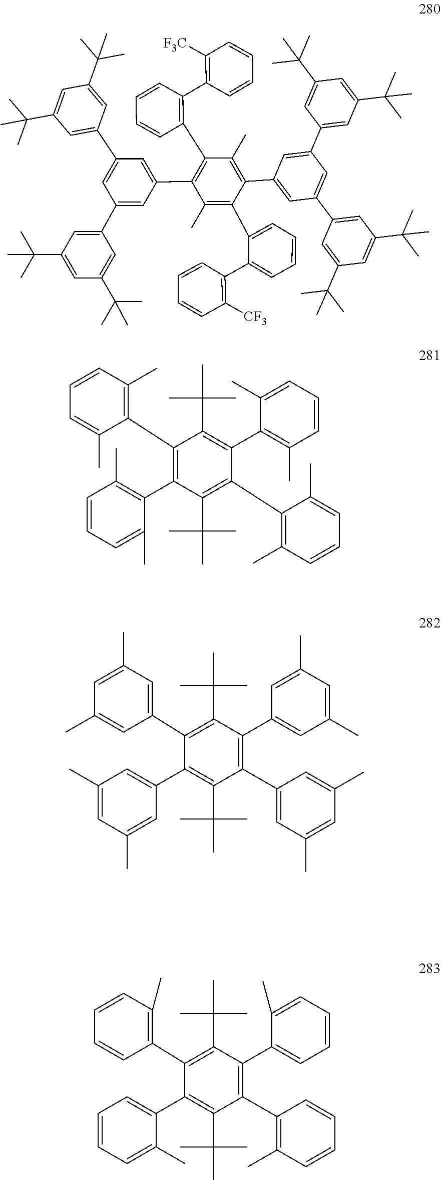

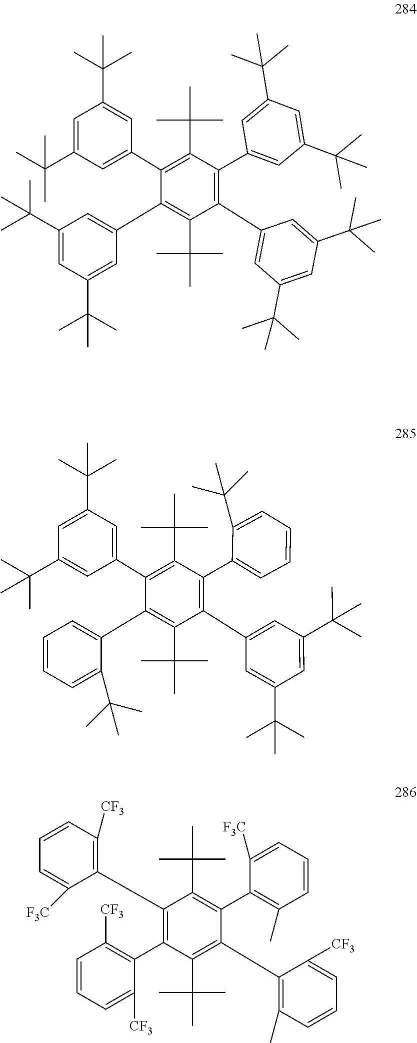

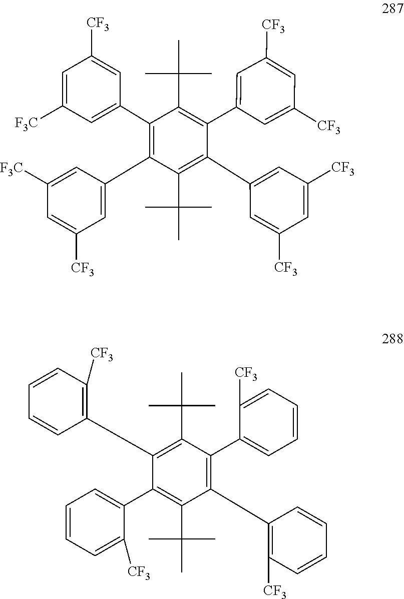 [SMILES: CC(C)(C)c1c(-c2cc(C(F)(F)F)cc(C(F)(F)F)c2)c(-c2cc(C(F)(F)F)cc(C(F)(F)F)c2)c(C(C)(C)C)c(-c2cc(C(F)(F)F)cc(C(F)(F)F)c2)c1-c1cc(C(F)(F)F)cc(C(F)(F)F)c1 ; CC(C)(C)c1c(-c2ccccc2C(F)(F)F)c(-c2ccccc2C(F)(F)F)c(C(C)(C)C)c(-c2ccccc2C(F)(F)F)c1-c1ccccc1C(F)(F)F]

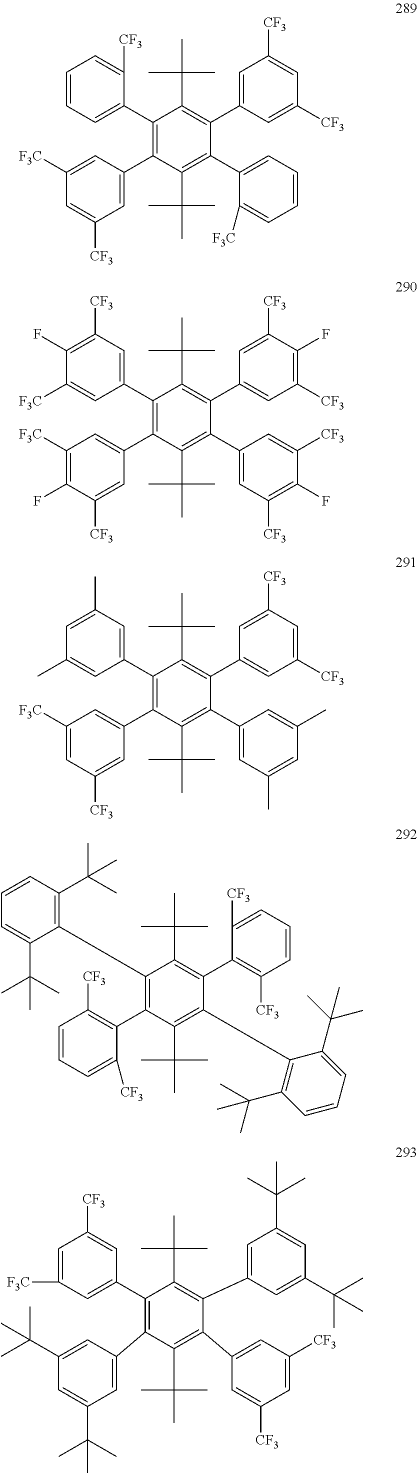

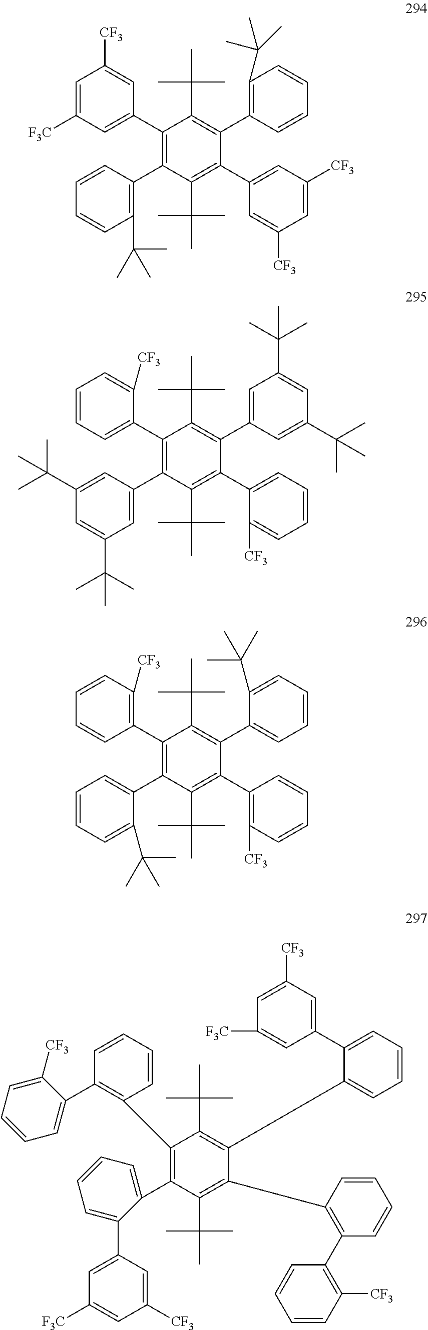

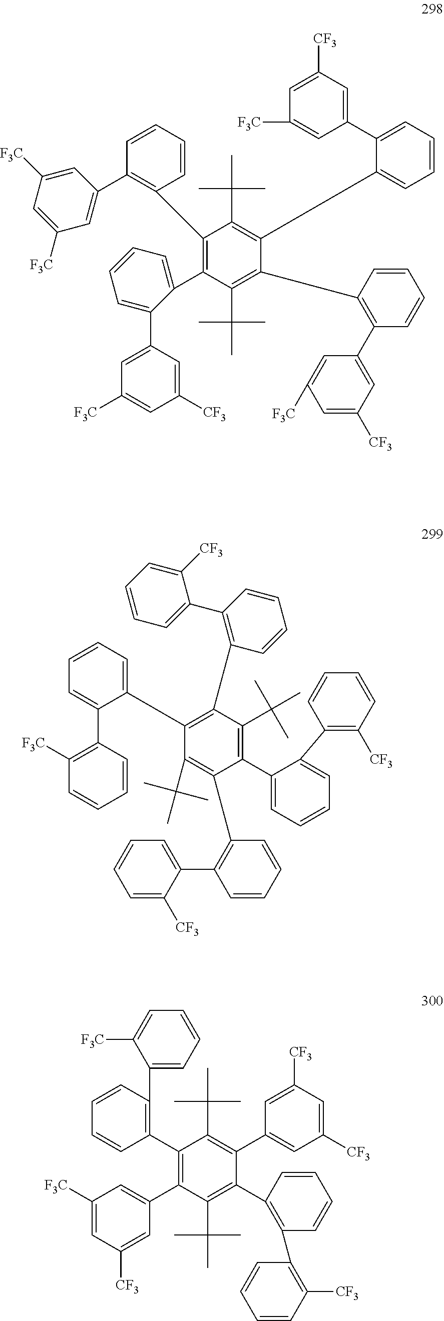 [SMILES: CC(C)(C)c1c(-c2ccccc2-c2cc(C(F)(F)F)cc(C(F)(F)F)c2)c(-c2ccccc2-c2cc(C(F)(F)F)cc(C(F)(F)F)c2)c(C(C)(C)C)c(-c2ccccc2-c2cc(C(F)(F)F)cc(C(F)(F)F)c2)c1-c1ccccc1-c1cc(C(F)(F)F)cc(C(F)(F)F)c1 ; CC(C)(C)c1c(-c2ccccc2-c2ccccc2C(F)(F)F)c(-c2ccccc2-c2ccccc2C(F)(F)F)c(C(C)(C)C)c(-c2ccccc2-c2ccccc2C(F)(F)F)c1-c1ccccc1-c1ccccc1C(F)(F)F ; CC(C)(C)c1c(-c2cc(C(F)(F)F)cc(C(F)(F)F)c2)c(-c2ccccc2-c2ccccc2C(F)(F)F)c(C(C)(C)C)c(-c2cc(C(F)(F)F)cc(C(F)(F)F)c2)c1-c1ccccc1-c1ccccc1C(F)(F)F]

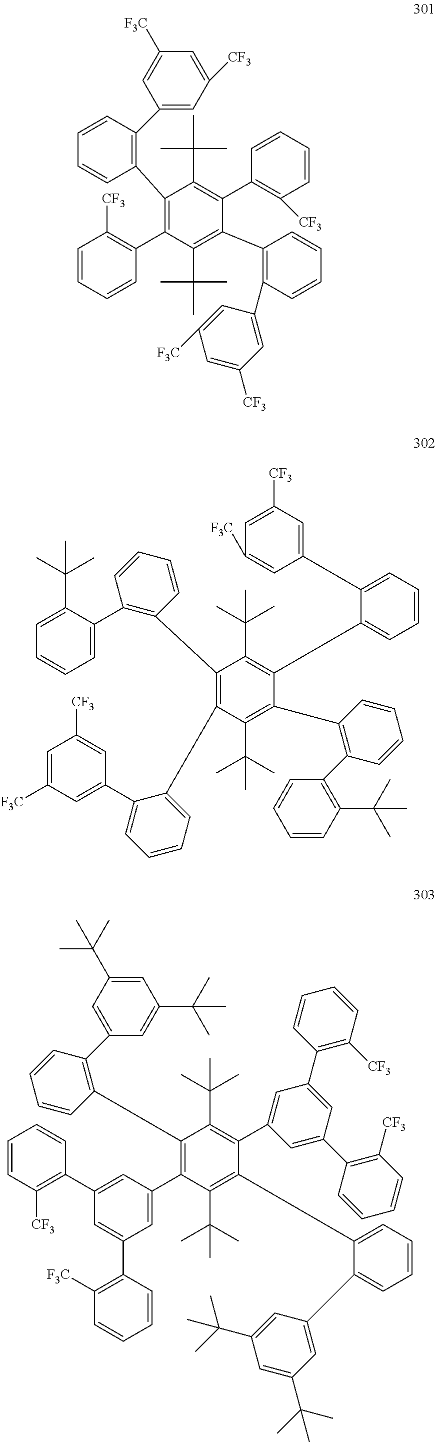

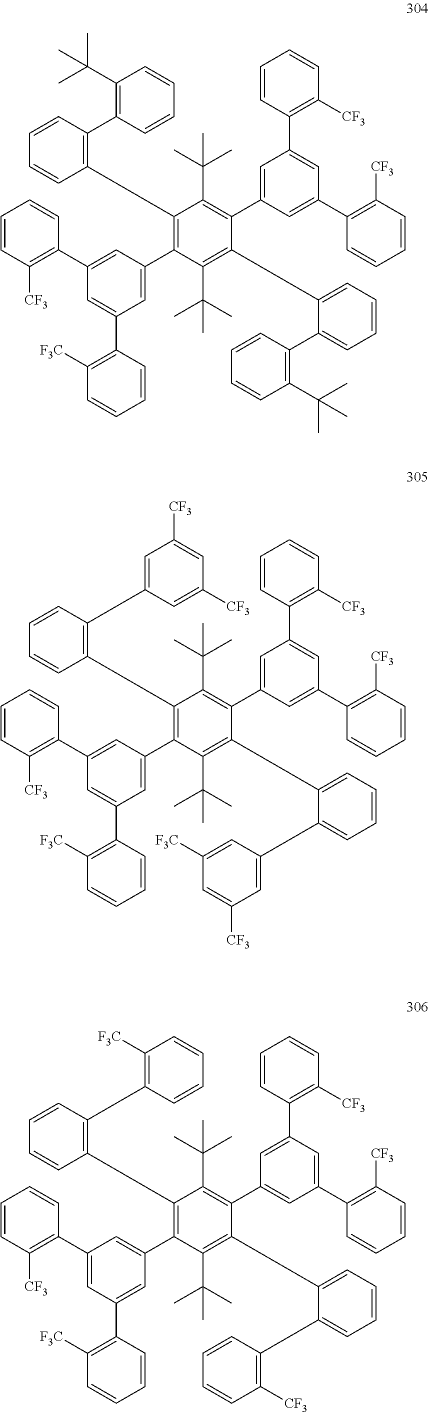

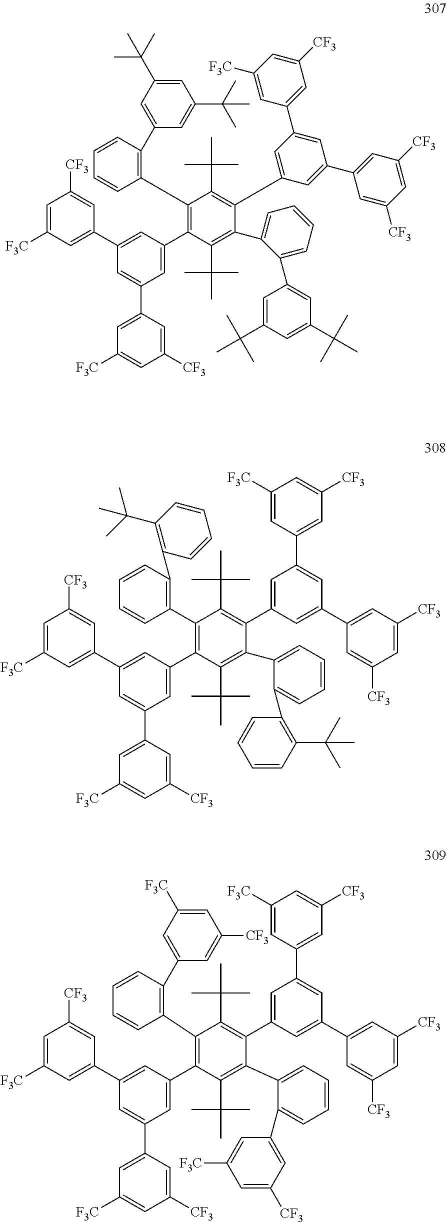

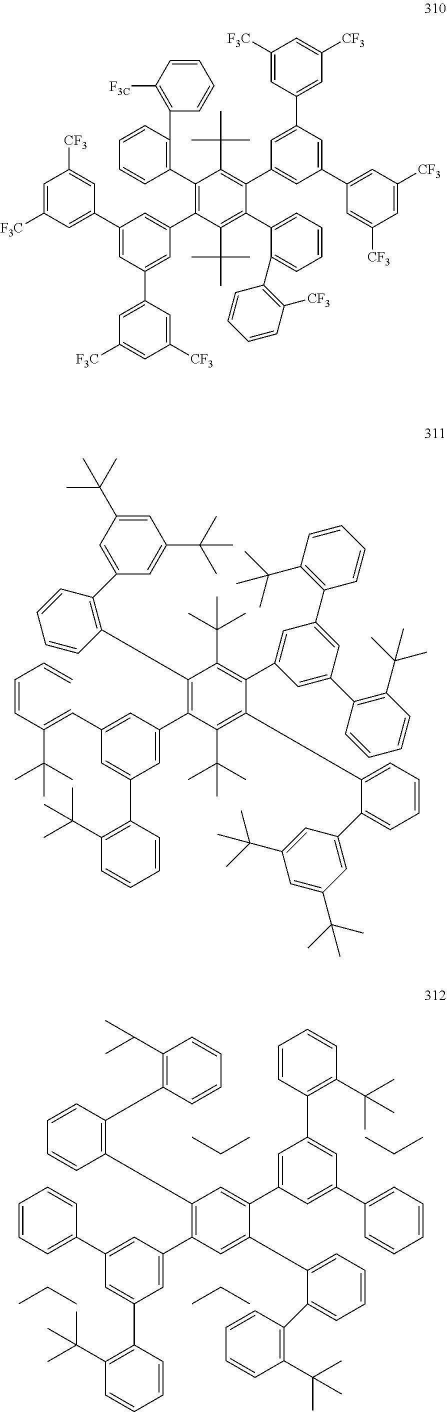

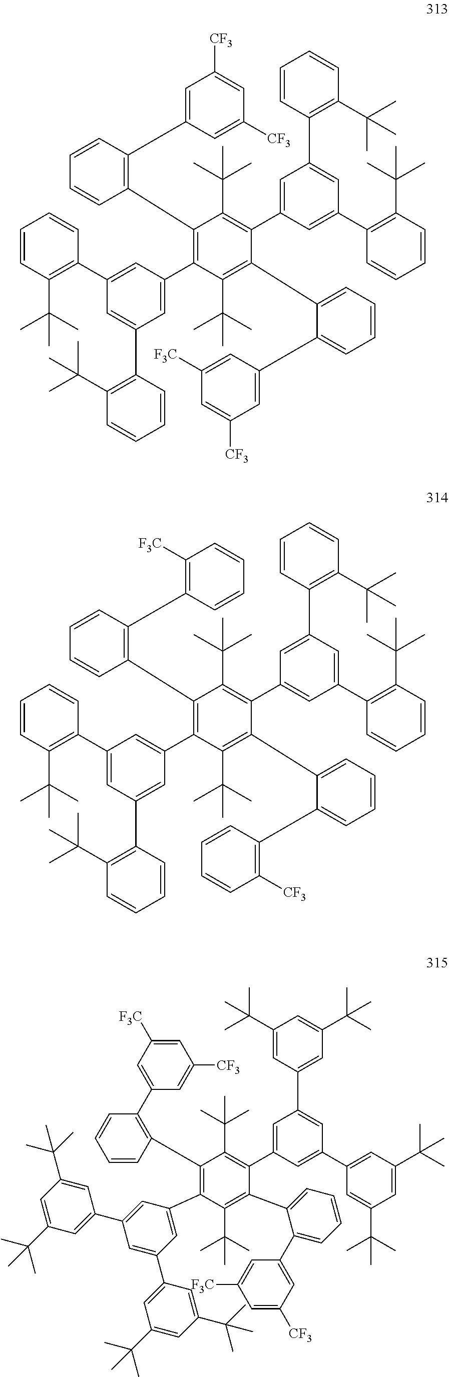

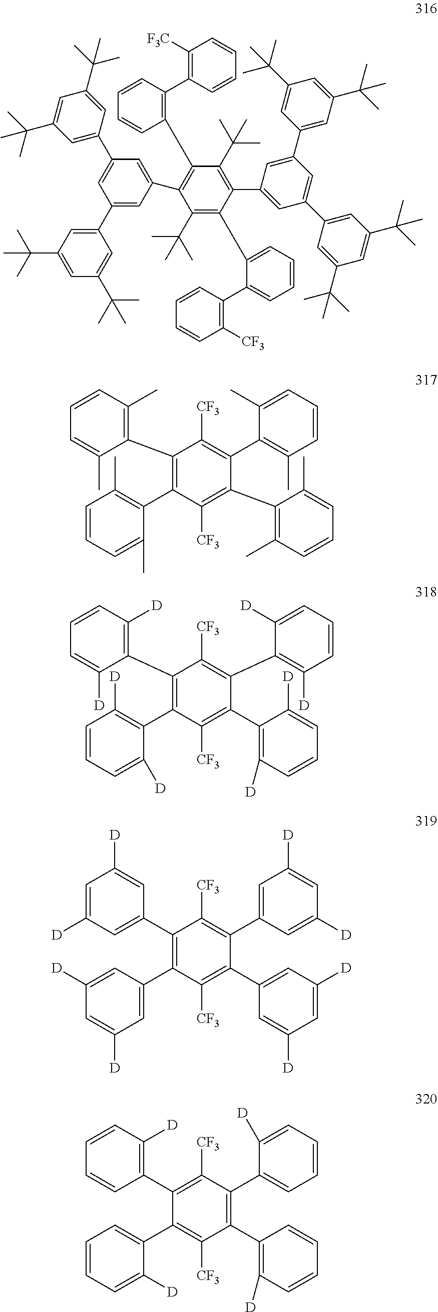

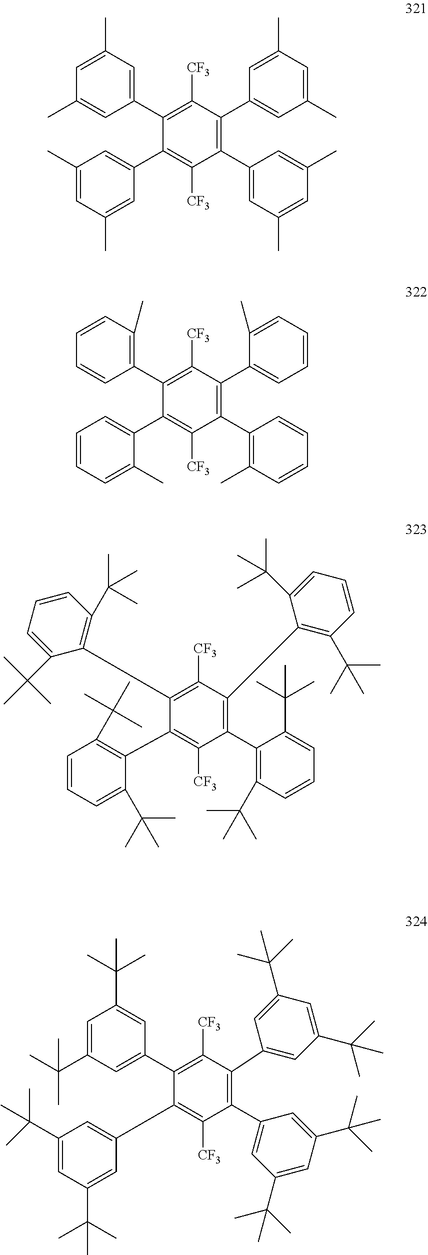

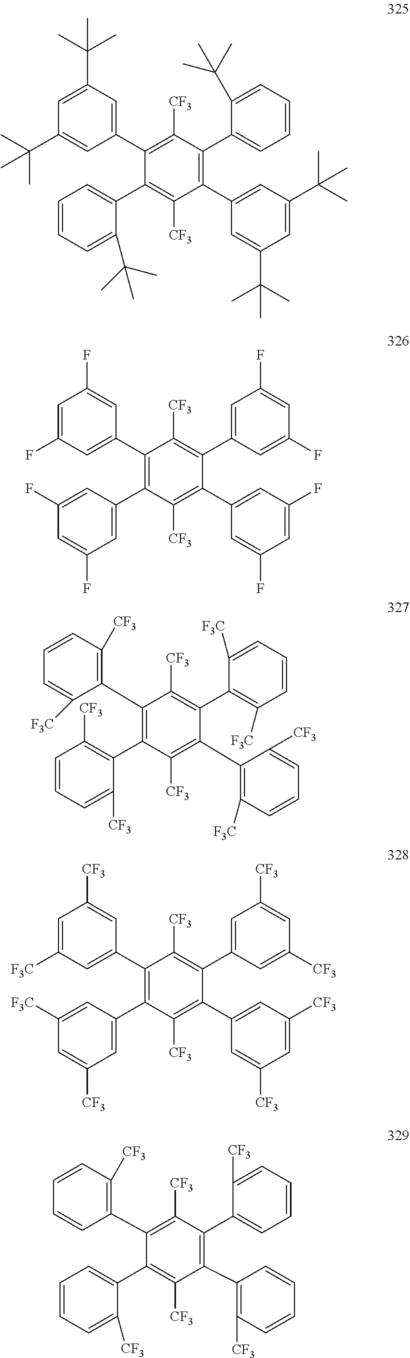

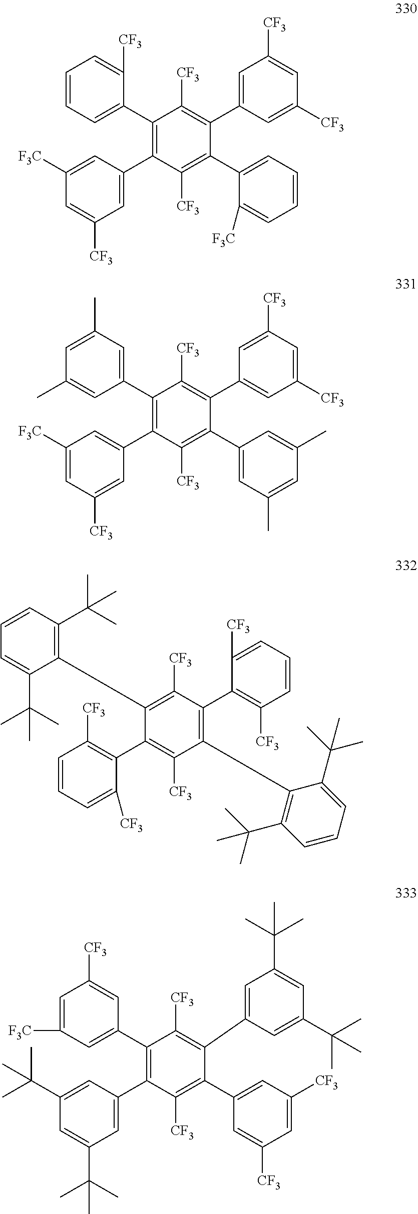

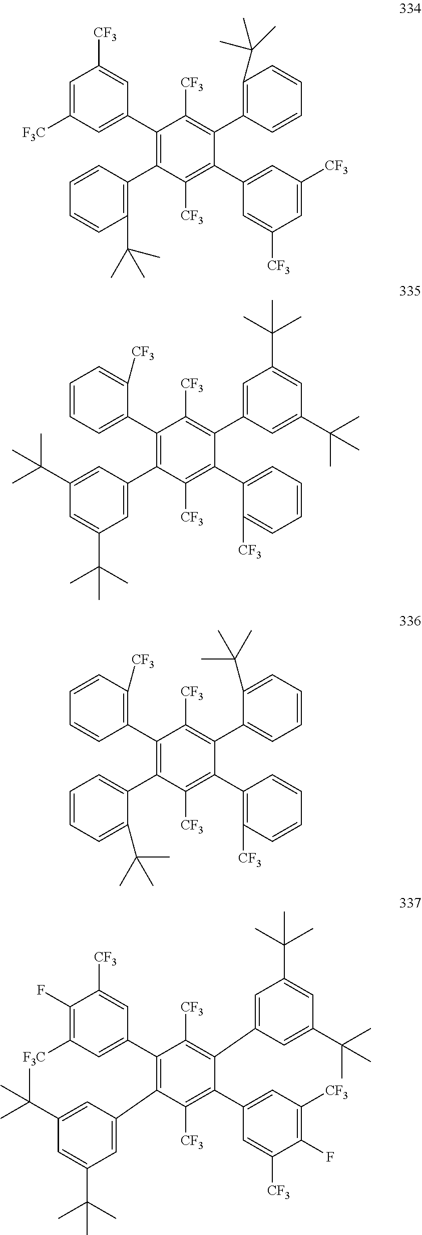

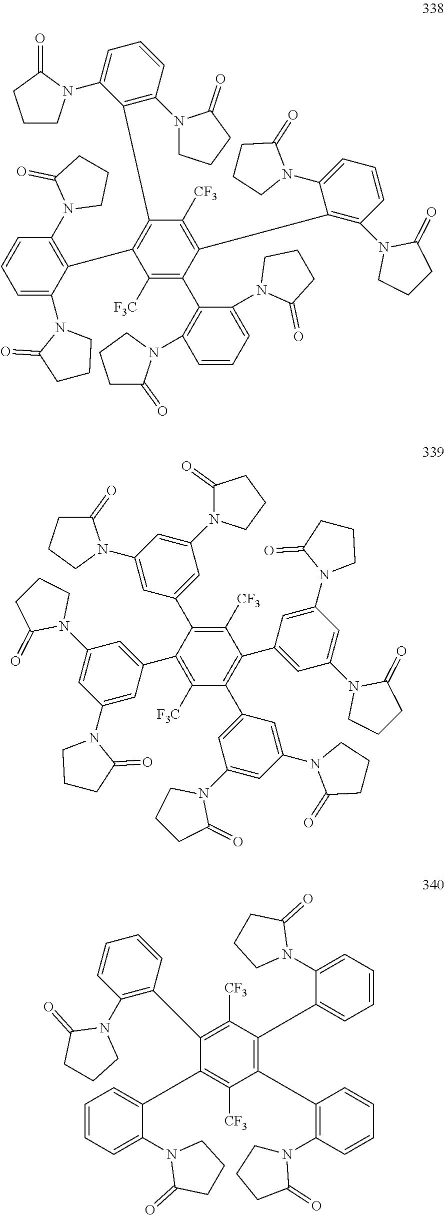 [SMILES: O=C1CCCN1c1cccc(N2CCCC2=O)c1-c1c(-c2c(N3CCCC3=O)cccc2N2CCCC2=O)c(C(F)(F)F)c(-c2c(N3CCCC3=O)cccc2N2CCCC2=O)c(-c2c(N3CCCC3=O)cccc2N2CCCC2=O)c1C(F)(F)F ; O=C1CCCN1c1cc(-c2c(-c3cc(N4CCCC4=O)cc(N4CCCC4=O)c3)c(C(F)(F)F)c(-c3cc(N4CCCC4=O)cc(N4CCCC4=O)c3)c(-c3cc(N4CCCC4=O)cc(N4CCCC4=O)c3)c2C(F)(F)F)cc(N2CCCC2=O)c1 ; O=C1CCCN1c1ccccc1-c1c(-c2ccccc2N2CCCC2=O)c(C(F)(F)F)c(-c2ccccc2N2CCCC2=O)c(-c2ccccc2N2CCCC2=O)c1C(F)(F)F]

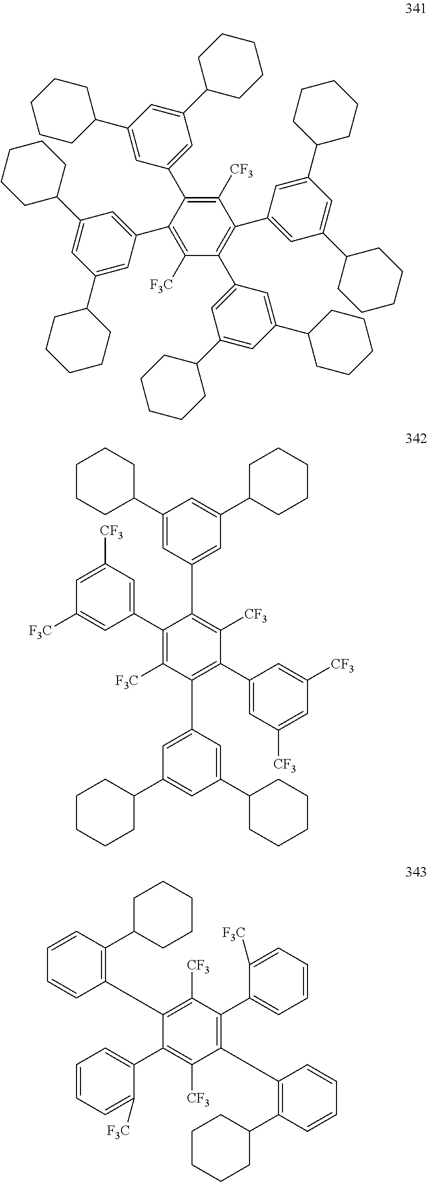 [SMILES: FC(F)(F)c1c(-c2cc(C3CCCCC3)cc(C3CCCCC3)c2)c(-c2cc(C3CCCCC3)cc(C3CCCCC3)c2)c(C(F)(F)F)c(-c2cc(C3CCCCC3)cc(C3CCCCC3)c2)c1-c1cc(C2CCCCC2)cc(C2CCCCC2)c1 ; FC(F)(F)c1cc(-c2c(-c3cc(C4CCCCC4)cc(C4CCCCC4)c3)c(C(F)(F)F)c(-c3cc(C(F)(F)F)cc(C(F)(F)F)c3)c(-c3cc(C4CCCCC4)cc(C4CCCCC4)c3)c2C(F)(F)F)cc(C(F)(F)F)c1 ; FC(F)(F)c1ccccc1-c1c(-c2ccccc2C2CCCCC2)c(C(F)(F)F)c(-c2ccccc2C(F)(F)F)c(-c2ccccc2C2CCCCC2)c1C(F)(F)F]

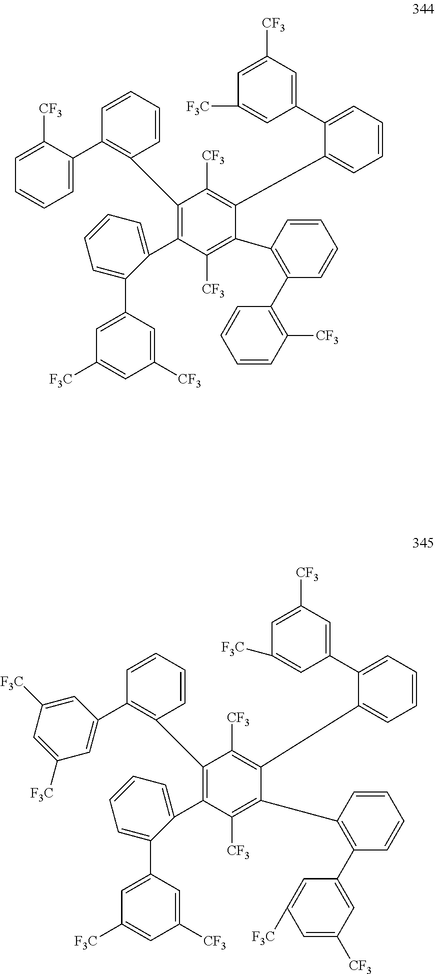 [SMILES: FC(F)(F)c1cc(-c2ccccc2-c2c(-c3ccccc3-c3ccccc3C(F)(F)F)c(C(F)(F)F)c(-c3ccccc3-c3cc(C(F)(F)F)cc(C(F)(F)F)c3)c(-c3ccccc3-c3ccccc3C(F)(F)F)c2C(F)(F)F)cc(C(F)(F)F)c1 ; FC(F)(F)c1cc(-c2ccccc2-c2c(-c3ccccc3-c3cc(C(F)(F)F)cc(C(F)(F)F)c3)c(C(F)(F)F)c(-c3ccccc3-c3cc(C(F)(F)F)cc(C(F)(F)F)c3)c(-c3ccccc3-c3cc(C(F)(F)F)cc(C(F)(F)F)c3)c2C(F)(F)F)cc(C(F)(F)F)c1]

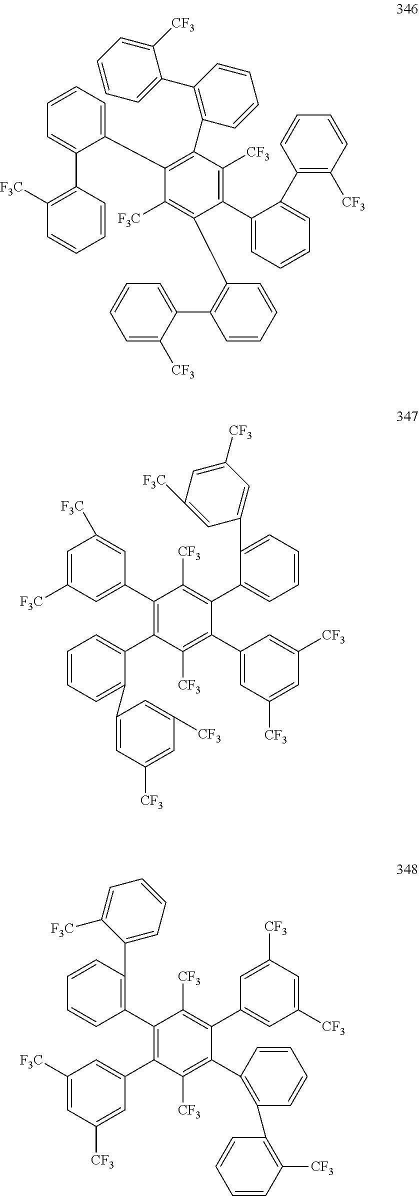 [SMILES: FC(F)(F)c1ccccc1-c1ccccc1-c1c(-c2ccccc2-c2ccccc2C(F)(F)F)c(C(F)(F)F)c(-c2ccccc2-c2ccccc2C(F)(F)F)c(-c2ccccc2-c2ccccc2C(F)(F)F)c1C(F)(F)F ; FC(F)(F)c1cc(-c2ccccc2-c2c(-c3cc(C(F)(F)F)cc(C(F)(F)F)c3)c(C(F)(F)F)c(-c3ccccc3-c3cc(C(F)(F)F)cc(C(F)(F)F)c3)c(-c3cc(C(F)(F)F)cc(C(F)(F)F)c3)c2C(F)(F)F)cc(C(F)(F)F)c1 ; FC(F)(F)c1cc(-c2c(-c3ccccc3-c3ccccc3C(F)(F)F)c(C(F)(F)F)c(-c3cc(C(F)(F)F)cc(C(F)(F)F)c3)c(-c3ccccc3-c3ccccc3C(F)(F)F)c2C(F)(F)F)cc(C(F)(F)F)c1]

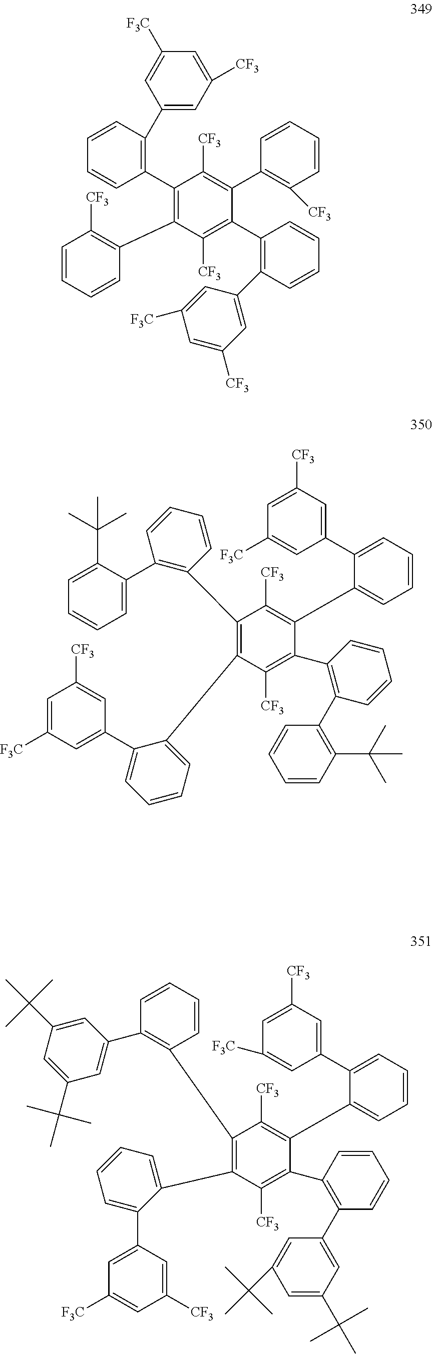 [SMILES: FC(F)(F)c1cc(-c2ccccc2-c2c(-c3ccccc3C(F)(F)F)c(C(F)(F)F)c(-c3ccccc3-c3cc(C(F)(F)F)cc(C(F)(F)F)c3)c(-c3ccccc3C(F)(F)F)c2C(F)(F)F)cc(C(F)(F)F)c1 ; CC(C)(C)c1ccccc1-c1ccccc1-c1c(-c2ccccc2-c2cc(C(F)(F)F)cc(C(F)(F)F)c2)c(C(F)(F)F)c(-c2ccccc2-c2ccccc2C(C)(C)C)c(-c2ccccc2-c2cc(C(F)(F)F)cc(C(F)(F)F)c2)c1C(F)(F)F ; CC(C)(C)c1cc(-c2ccccc2-c2c(-c3ccccc3-c3cc(C(F)(F)F)cc(C(F)(F)F)c3)c(C(F)(F)F)c(-c3ccccc3-c3cc(C(C)(C)C)cc(C(C)(C)C)c3)c(-c3ccccc3-c3cc(C(F)(F)F)cc(C(F)(F)F)c3)c2C(F)(F)F)cc(C(C)(C)C)c1]

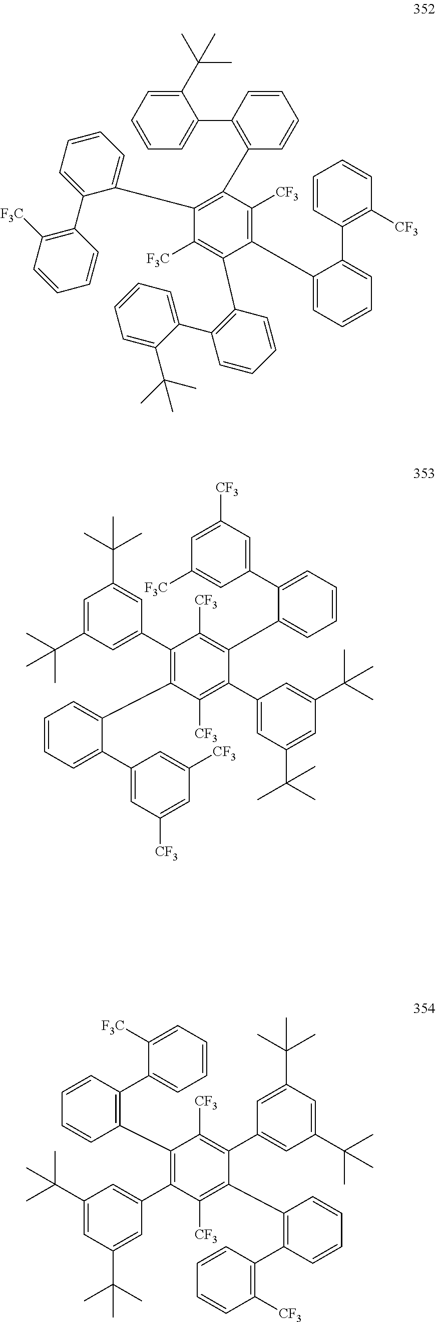

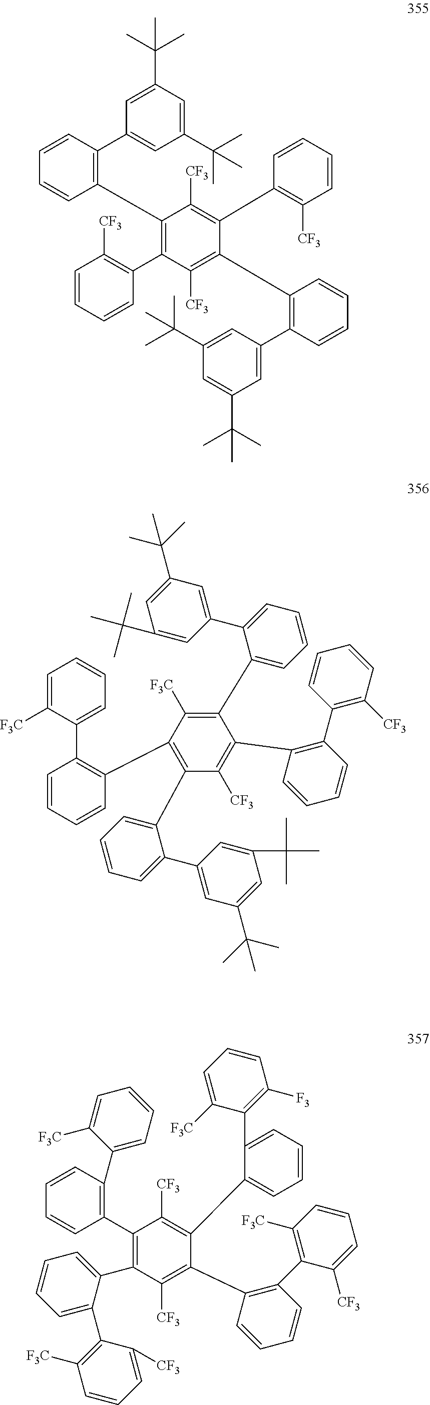

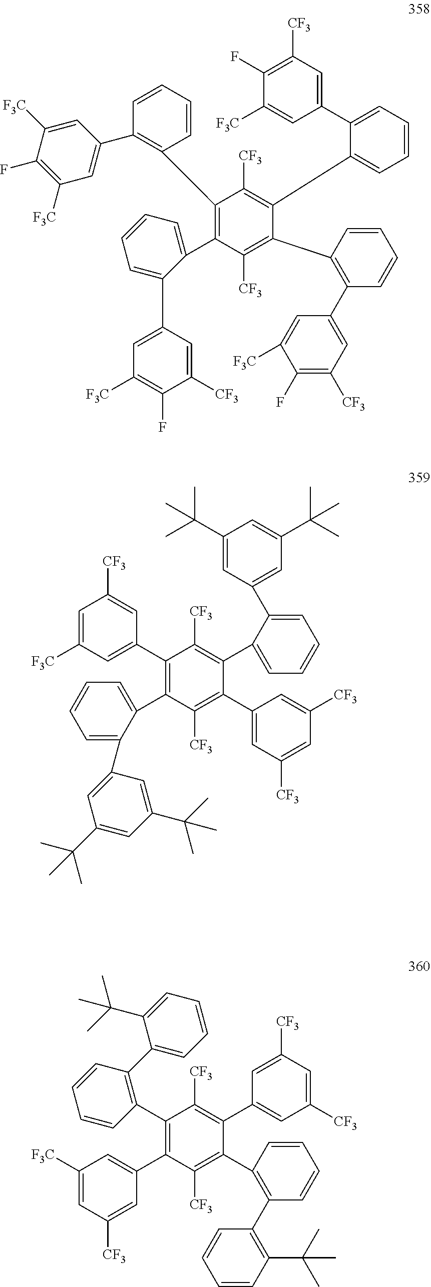

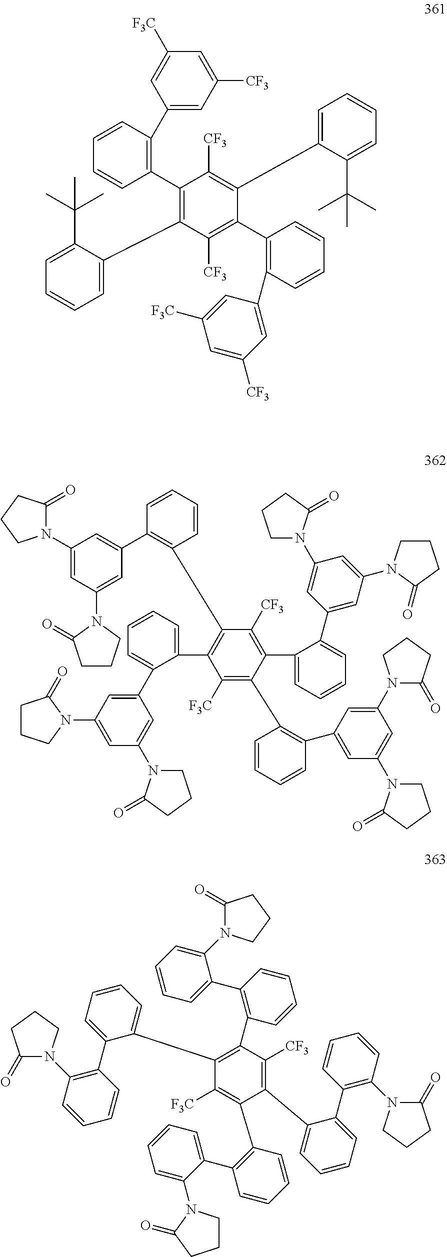 [SMILES: CC(C)(C)c1ccccc1-c1c(-c2ccccc2-c2cc(C(F)(F)F)cc(C(F)(F)F)c2)c(C(F)(F)F)c(-c2ccccc2C(C)(C)C)c(-c2ccccc2-c2cc(C(F)(F)F)cc(C(F)(F)F)c2)c1C(F)(F)F ; O=C1CCCN1c1cc(-c2ccccc2-c2c(-c3ccccc3-c3cc(N4CCCC4=O)cc(N4CCCC4=O)c3)c(C(F)(F)F)c(-c3ccccc3-c3cc(N4CCCC4=O)cc(N4CCCC4=O)c3)c(-c3ccccc3-c3cc(N4CCCC4=O)cc(N4CCCC4=O)c3)c2C(F)(F)F)cc(N2CCCC2=O)c1 ; O=C1CCCN1c1ccccc1-c1ccccc1-c1c(-c2ccccc2-c2ccccc2N2CCCC2=O)c(C(F)(F)F)c(-c2ccccc2-c2ccccc2N2CCCC2=O)c(-c2ccccc2-c2ccccc2N2CCCC2=O)c1C(F)(F)F]

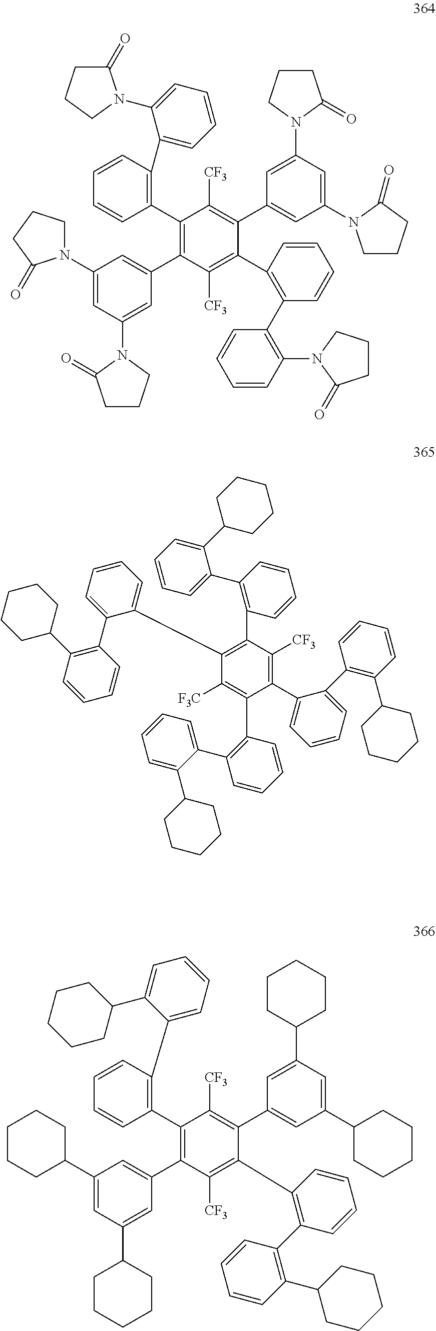 [SMILES: O=C1CCCN1c1cc(-c2c(-c3ccccc3-c3ccccc3N3CCCC3=O)c(C(F)(F)F)c(-c3cc(N4CCCC4=O)cc(N4CCCC4=O)c3)c(-c3ccccc3-c3ccccc3N3CCCC3=O)c2C(F)(F)F)cc(N2CCCC2=O)c1 ; FC(F)(F)c1c(-c2ccccc2-c2ccccc2C2CCCCC2)c(-c2ccccc2-c2ccccc2C2CCCCC2)c(C(F)(F)F)c(-c2ccccc2-c2ccccc2C2CCCCC2)c1-c1ccccc1-c1ccccc1C1CCCCC1 ; FC(F)(F)c1c(-c2cc(C3CCCCC3)cc(C3CCCCC3)c2)c(-c2ccccc2-c2ccccc2C2CCCCC2)c(C(F)(F)F)c(-c2cc(C3CCCCC3)cc(C3CCCCC3)c2)c1-c1ccccc1-c1ccccc1C1CCCCC1]

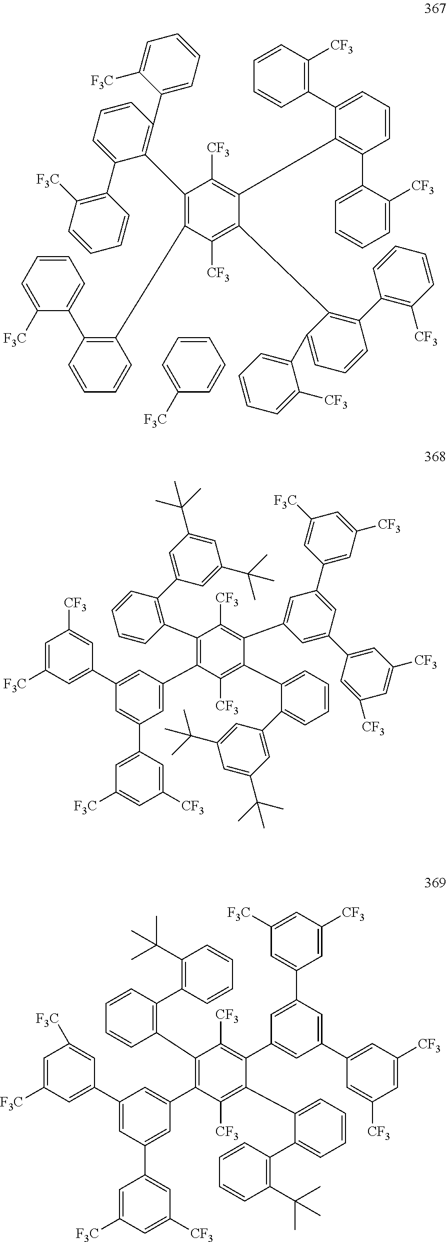

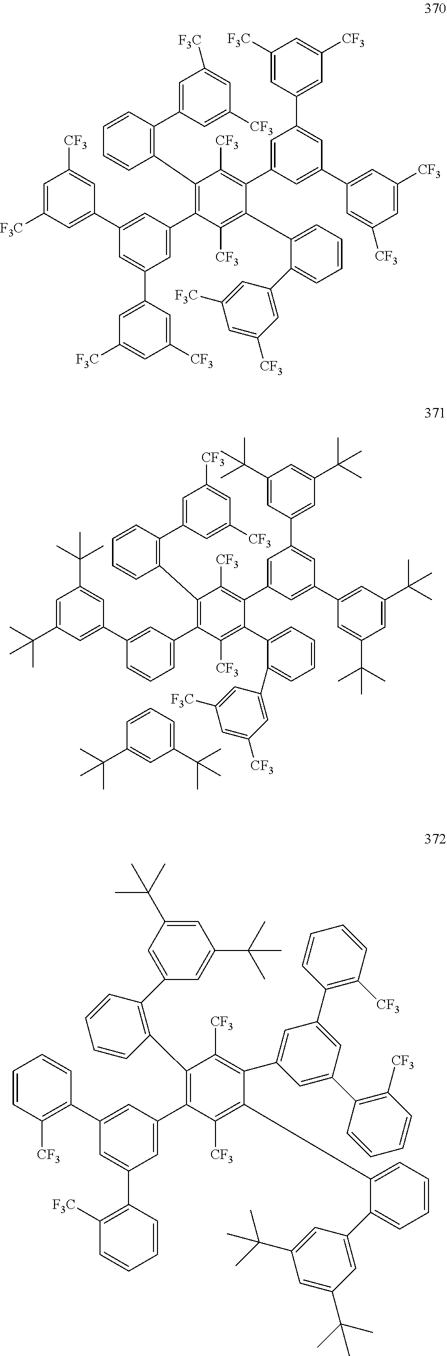

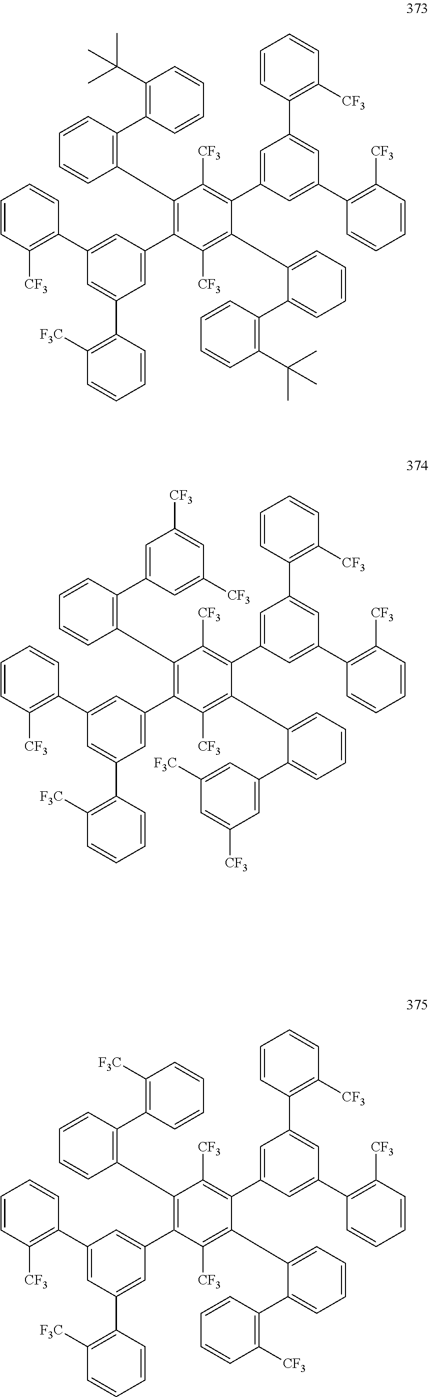

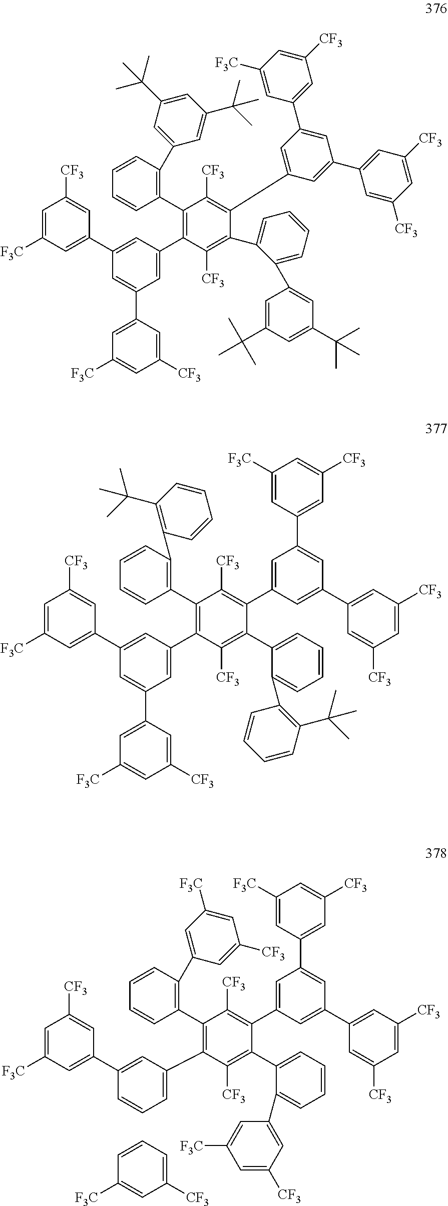

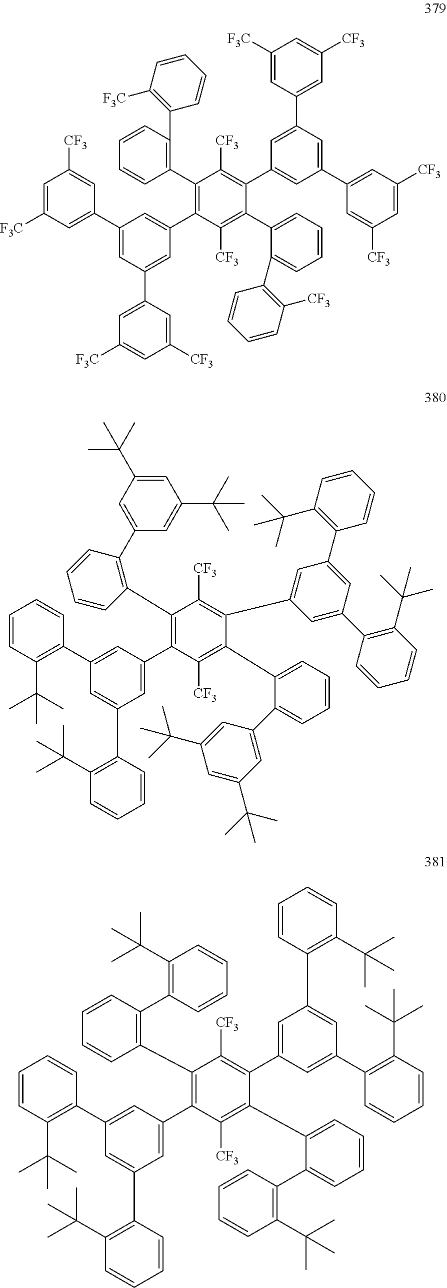

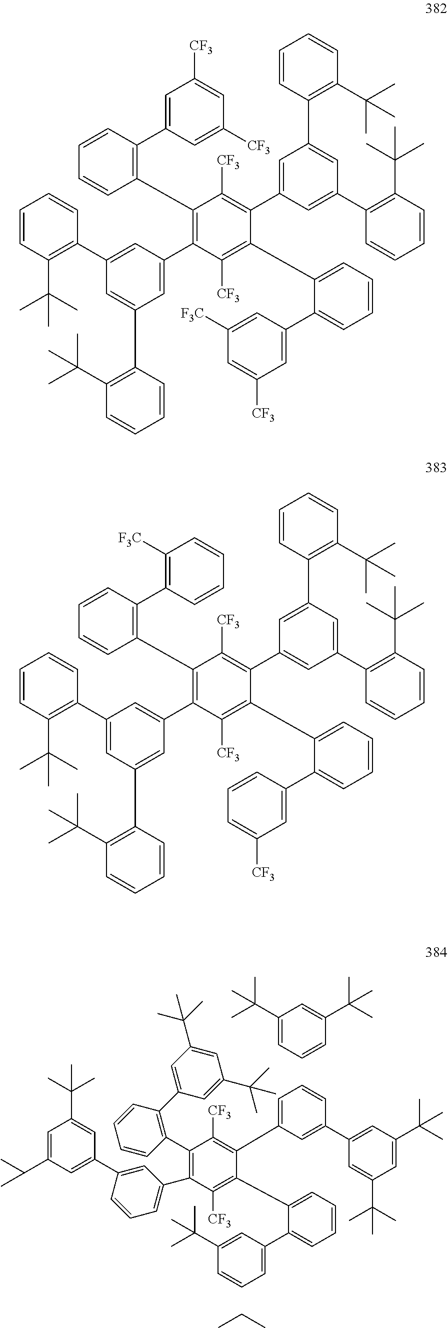

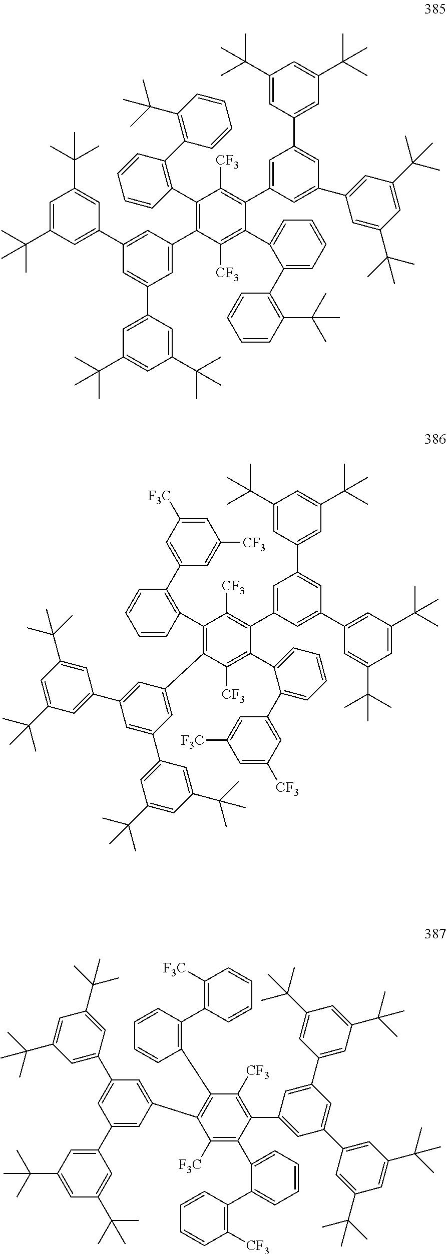

4. An organic light-emitting device comprising a first electrode, a second electrode, and one or more organic layers disposed between the first and second electrodes,
further comprising a light efficiency improving layer (capping layer) formed on at least one side opposite to the organic layer among upper or lower portions of the first electrode and the second electrode,
wherein the light efficiency improving layer includes a compound represented by Formula I above.

5. The organic light-emitting device of claim 4, wherein the light efficiency improving layer is formed on at least one of a lower portion of the first electrode or an upper portion of the second electrode.
